(19) United States
(12) Patent Application Publication
(10) Pub. No.: US 2026/0234123 A1
(43) Pub. Date: Aug. 13, 2026

(54) Inhibitors of NLRP3

(71) Applicant: Mwyngil Therapeutics, Inc., Dover, DE (US)

(72) Inventors: Boris Rogovoy, Needham, MA (US); Volodymyr Kysil, San Diego, CA (US); Nikolay Savchuk, San Diego, CA (US)

(21) Appl. No.: 19/151,317

(22) PCT Filed: Jan. 24, 2024
(86) PCT No.: PCT/US2024/012821

§ 371 Date: Jul. 26, 2025

Related U.S. Application Data
(60) Provisional application No. 63/441,687, filed on Jan. 27, 2023.

(51) Int. Cl. C07D 401/12; A61K 31/501; A61K 31/502; A61P 29/00; C07D 401/14; C07D 405/14; C07D 417/14; C07D 453/02; C07D 455/02

(52) U.S. Cl. CPC C07D 401/12; A61K 31/501; A61K 31/502; A61P 29/00; C07D 401/14; C07D 405/14; C07D 417/14; C07D 453/02; C07D 455/02

(57) ABSTRACT

The present invention is directed to the compounds of Formula (A)-inhibitors of NLRP3. The inhibitors described herein can be useful in the treatment of diseases or disorders associated with NLRP3, such as Autoimmune Disease, Inflammation Disease. In particular, the invention is concerned with compounds and pharmaceutical compositions inhibiting NLRP3, methods of treating diseases or disorders associated with NLRP3, and methods of synthesizing these compounds.

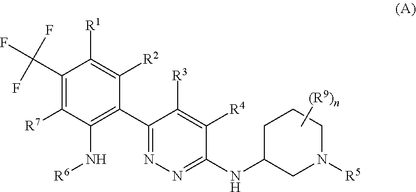

### CROSS-REFERENCE TO RELATED APPLICATIONS

This application claims priority to and benefit of U.S. Provisional Patent Application Ser. No. 63/441,687 filed Jan. 27, 2023, entitled “Inhibitors of NLRP3” the disclosure of which are incorporated by reference in its entirety for all purposes.

### FIELD OF INVENTION

The present invention is directed to inhibitors of NLRP3. The inhibitors described herein can be useful in the treatment of diseases or disorders associated with NLRP3, such as autoimmune disease, Alzheimer disease, and coronavirus infection by inducing durable responses. In particular, the invention is concerned with compounds and pharmaceutical compositions inhibiting NLRP3, methods of treating diseases or disorders associated with NLRP3, and methods of synthesizing these compounds.

### BACKGROUND

NLRP3 (NOD-, LRR- and pyrin domain-containing protein 3) is an intracellular sensor that detects a broad range of microbial motifs, endogenous danger signals and environmental irritants, resulting in the formation and activation of the NLRP3 inflammasome. Assembly of the NLRP3 inflammasome leads to caspase 1-dependent release of the pro-inflammatory cytokines IL-1β and IL-18, as well as to gasdermin D-mediated pyroptotic cell death.

Inflammasomes play a crucial role in innate immunity by serving as signaling platforms which deal with a plethora of pathogenic products and cellular products associated with stress and damage. By far, the best studied and most characterized inflammasome is NLRP3 inflammasome, which consists of NLRP3 (nucleotide-binding domain leucine-rich repeat (NLR) and pyrin domain containing receptor 3), ASC (apoptosis-associated speck-like protein containing a caspase recruitment domain), and procaspase-1. Activation of NLRP3 inflammasome is mediated by highly diverse stimuli. Upon activation, NLRP3 protein recruits the adapter ASC protein, which recruits the procaspase-1 resulting in its cleavage and activation, inducing the maturation, and secretion of inflammatory cytokines and pyroptosis. However, aberrant activation of the NLRP3 inflammasome is implicated in various diseases including diabetes, atherosclerosis, metabolic syndrome, cardiovascular, and neurodegenerative diseases; raising a tremendous clinical interest in exploring the potential inhibitors of NLRP3 inflammasome. Recent investigations have disclosed various inhibitors of the NLRP3 inflammasome pathway which were validated through in vitro studies and in vivo experiments in animal models of NLRP3-associated disorders. Some of these inhibitors directly target the NLRP3 protein whereas some are aimed at other components and products of the inflammasome. Direct targeting of NLRP3 protein can be a better choice because it can prevent off target immunosuppressive effects, thus restrain tissue destruction.

Although many studies have been done on NLRP3 inhibitors up to date, work is still underway to overcome NLRP3 inhibitor resistance, inhibitor specificity that is significant problems in patients' treatment.

### SUMMARY

A first aspect of the invention relates to compounds of Formula (A):

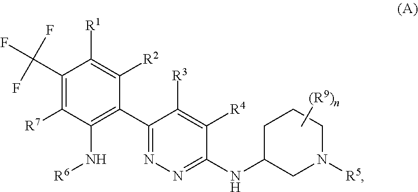

or a pharmaceutically acceptable salt, prodrug, stereoisomer, solvate, or tautomer thereof, wherein:
R¹ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R² is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R¹ and R² together with the atoms to which they are attached and any intervening atoms, form a 5-8 membered cycloalkane, a 5-14 membered heterocycle, arene, or heteroarene wherein the cycloalkane, heterocycle, arene, or heteroarene is optionally substituted with one or more substituents independently selected from halogen, OH, CN, C₁-C₆ alkyl, C₁-C₆ alkoxy;
R³ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R⁴ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R³ and R⁴ together with the atoms to which they are attached and any intervening atoms, form a 5-8 membered cycloalkane, a 5-14 membered heterocycle, wherein the cycloalkane, heterocycle, is optionally substituted with one or more substituents independently selected from halogen, OH, CN, C₁-C₆ alkyl, C₁-C₆ alkoxy;
R⁵ is selected from H, C₁-C₆ alkyl, C₃-C₁₀ cycloalkyl, C₂-C₆ alkenyl, C₂-C₆ alkynyl, C₁-C₆ acyl, SO₂—C₁-C₆ alkyl, 4-10 membered heterocyclyl, wherein the alkyl, cycloalkyl, alkenyl, alkynyl, acyl, or heterocyclyl is optionally substituted with one or more substituents independently selected from halogen, oxo, OH, CN, C₁-C₆ alkoxy, C₃-C₁₀ cycloalkyl, aryl;
R⁶ is selected from H, SO₂R⁸, NH₂, C₁-C₆ alkyl, C₁-C₆ alkyl-C(O)—, aryl, wherein alkyl or aryl is optionally substituted with one or more substituents independently selected from halogen, CN, OH;
R⁷ is H, C₁-C₆ alkyl, or NH₂; or
R⁶ and R⁷ together with the atoms to which they are attached and any intervening atoms, form a 5-6 membered heterocycle or heteroarene;
R⁸ is selected from C₁-C₆ alkyl, C₃-C₈ cycloalkyl, 5-6 membered heteroaryl, wherein alkyl, cycloalkyl or heteroaryl is optionally substituted with one or more substituents independently selected from halogen, CN, OH;
each R⁹ is independently selected from halogen, OH, C₁-C₆ alkyl, C₁-C₆ alkoxy, wherein alkyl or alkoxy optionally substituted with one or more halogen, OH; or two R⁹ together with the atoms to which they are attached and any intervening atoms, form a 5-6 membered heterocycle; or
R⁵ and R⁹ together with the atoms to which they are attached and any intervening atoms, form a 5-6 membered heterocycle;
n is an integer selected from 0, 1, and 2;
provided that when R⁵ is methyl then R⁶ is not H or CH₃CO—.

Another aspect of the invention relates to compounds of Formula (I):

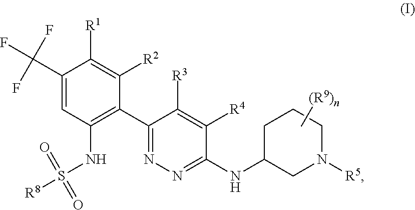

or a pharmaceutically acceptable salt, prodrug, stereoisomer, solvate, or tautomer thereof, wherein:
R¹ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R² is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R¹ and R² together with the atoms to which they are attached and any intervening atoms, form a 5-8 membered cycloalkane, a 5-14 membered heterocycle, arene, or heteroarene wherein the cycloalkane, heterocycle, arene, or heteroarene is optionally substituted with one or more substituents independently selected from halogen, OH, CN, C₁-C₆ alkyl, C₁-C₆ alkoxy;
R³ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R⁴ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R³ and R⁴ together with the atoms to which they are attached and any intervening atoms, form a 5-8 membered cycloalkane, a 5-14 membered heterocycle, wherein the cycloalkane, heterocycle, is optionally substituted with one or more substituents independently selected from halogen, OH, CN, C₁-C₆ alkyl, C₁-C₆ alkoxy;
R⁵ is selected from H, C₁-C₆ alkyl, C₃-C₁₀ cycloalkyl, C₂-C₆ alkenyl, C₂-C₆ alkynyl, C₁-C₆ acyl, SO₂—C₁-C₆ alkyl, 4-10 membered heterocyclyl, wherein the alkyl, cycloalkyl, alkenyl, alkynyl, acyl, or heterocyclyl is optionally substituted with one or more substituents independently selected from halogen, oxo, OH, CN, C₁-C₆ alkoxy, C₃-C₁₀ cycloalkyl, aryl;
R⁸ is selected from C₁-C₆ alkyl, C₃-C₈ cycloalkyl, 5-6 membered heteroaryl, wherein alkyl, cycloalkyl or heteroaryl is optionally substituted with one or more substituents independently selected from halogen, CN, OH;
each R⁹ is independently selected from halogen, OH, C₁-C₆ alkyl, C₁-C₆ alkoxy, wherein alkyl or alkoxy optionally substituted with one or more halogen, OH; or two R⁹ together with the atoms to which they are attached and any intervening atoms, form a 5-6 membered heterocycle; or
R⁵ and R⁹ together with the atoms to which they are attached and any intervening atoms, form a 5-6 membered heterocycle;
n is an integer selected from 0, 1, and 2.

Another aspect of the invention relates to compounds of Formula (II):

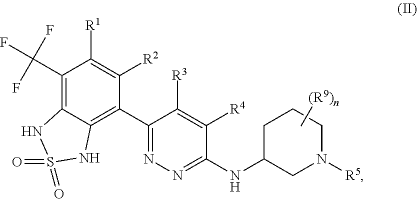

or a pharmaceutically acceptable salt, prodrug, stereoisomer, solvate, or tautomer thereof, wherein:
R¹ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R² is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R¹ and R² together with the atoms to which they are attached and any intervening atoms, form a 5-8 membered cycloalkane, a 5-14 membered heterocycle, arene, or heteroarene wherein the cycloalkane, heterocycle, arene, or heteroarene is optionally substituted with one or more substituents independently selected from halogen, OH, CN, C₁-C₆ alkyl, C₁-C₆ alkoxy;
R³ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R⁴ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R³ and R⁴ together with the atoms to which they are attached and any intervening atoms, form a 5-8 membered cycloalkane, a 5-14 membered heterocycle, wherein the cycloalkane, heterocycle, is optionally substituted with one or more substituents independently selected from halogen, OH, CN, C₁-C₆ alkyl, C₁-C₆ alkoxy;
R⁵ is selected from H, C₁-C₆ alkyl, C₃-C₁₀ cycloalkyl, C₂-C₆ alkenyl, C₂-C₆ alkynyl, C₁-C₆ acyl, SO₂—C₁-C₆ alkyl, 4-10 membered heterocyclyl, wherein the alkyl, cycloalkyl, alkenyl, alkynyl, acyl, or heterocyclyl is optionally substituted with one or more substituents independently selected from halogen, oxo, OH, CN, C₁-C₆ alkoxy, C₃-C₁₀ cycloalkyl, aryl;
each R⁹ is independently selected from halogen, OH, C₁-C₆ alkyl, C₁-C₆ alkoxy, wherein alkyl or alkoxy optionally substituted with one or more halogen, OH; or two R⁹ together with the atoms to which they are attached and any intervening atoms, form a 5-6 membered heterocycle; or
R⁵ and R⁹ together with the atoms to which they are attached and any intervening atoms, form a 5-6 membered heterocycle;
n is an integer selected from 0, 1, and 2.

Another aspect of the invention relates to compounds of Formula (III):

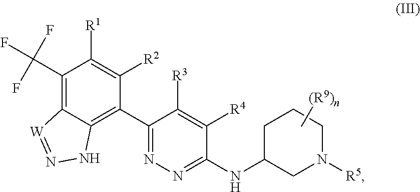

or a pharmaceutically acceptable salt, prodrug, stereoisomer, solvate, or tautomer thereof, wherein:
W is selected from CH, N;
R¹ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R² is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R¹ and R² together with the atoms to which they are attached and any intervening atoms, form a 5-8 membered cycloalkane, a 5-14 membered heterocycle, arene, or heteroarene wherein the cycloalkane, heterocycle, arene, or heteroarene is optionally substituted with one or more substituents independently selected from halogen, OH, CN, C₁-C₆ alkyl, C₁-C₆ alkoxy;
R³ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R⁴ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R³ and R⁴ together with the atoms to which they are attached and any intervening atoms, form a 5-8 membered cycloalkane, a 5-14 membered heterocycle, wherein the cycloalkane, heterocycle, is optionally substituted with one or more substituents independently selected from halogen, OH, CN, C₁-C₆ alkyl, C₁-C₆ alkoxy;
R⁵ is selected from H, C₁-C₆ alkyl, C₃-C₁₀ cycloalkyl, C₂-C₆ alkenyl, C₂-C₆ alkynyl, C₁-C₆ acyl, SO₂—C₁-C₆ alkyl, 4-10 membered heterocyclyl, wherein the alkyl, cycloalkyl, alkenyl, alkynyl, acyl, or heterocyclyl is optionally substituted with one or more substituents independently selected from halogen, oxo, OH, CN, C₁-C₆ alkoxy, C₃-C₁₀ cycloalkyl, aryl;
each R⁹ is independently selected from halogen, OH, C₁-C₆ alkyl, C₁-C₆ alkoxy, wherein alkyl or alkoxy optionally substituted with one or more halogen, OH; or two R⁹ together with the atoms to which they are attached and any intervening atoms, form a 5-6 membered heterocycle; or
R⁵ and R⁹ together with the atoms to which they are attached and any intervening atoms, form a 5-6 membered heterocycle;
n is an integer selected from 0, 1, and 2.

Another aspect of the invention relates to compounds of Formula (IV):

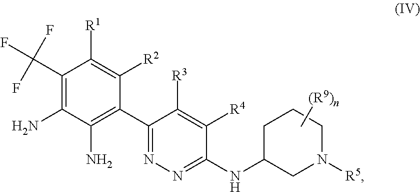

or a pharmaceutically acceptable salt, prodrug, stereoisomer, solvate, or tautomer thereof, wherein:
R¹ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R² is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R¹ and R² together with the atoms to which they are attached and any intervening atoms, form a 5-8 membered cycloalkane, a 5-14 membered heterocycle, arene, or heteroarene wherein the cycloalkane, heterocycle, arene, or heteroarene is optionally substituted with one or more substituents independently selected from halogen, OH, CN, C₁-C₆ alkyl, C₁-C₆ alkoxy;
R³ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R⁴ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R³ and R⁴ together with the atoms to which they are attached and any intervening atoms, form a 5-8 membered cycloalkane, a 5-14 membered heterocycle, wherein the cycloalkane, heterocycle, is optionally substituted with one or more substituents independently selected from halogen, OH, CN, C₁-C₆ alkyl, C₁-C₆ alkoxy;
R⁵ is selected from H, C₁-C₆ alkyl, C₃-C₁₀ cycloalkyl, C₂-C₆ alkenyl, C₂-C₆ alkynyl, C₁-C₆ acyl, SO₂—C₁-C₆ alkyl, 4-10 membered heterocyclyl, wherein the alkyl, cycloalkyl, alkenyl, alkynyl, acyl, or heterocyclyl is optionally substituted with one or more substituents independently selected from halogen, oxo, OH, CN, C₁-C₆ alkoxy, C₃-C₁₀ cycloalkyl, aryl;
each R⁹ is independently selected from halogen, OH, C₁-C₆ alkyl, C₁-C₆ alkoxy, wherein alkyl or alkoxy optionally substituted with one or more halogen, OH; or two R⁹ together with the atoms to which they are attached and any intervening atoms, form a 5-6 membered heterocycle; or
R⁵ and R⁹ together with the atoms to which they are attached and any intervening atoms, form a 5-6 membered heterocycle;
n is an integer selected from 0, 1, and 2.

Another aspect of the invention relates to compound of Formula (V):

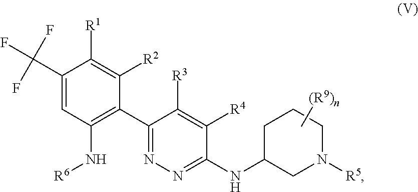

or a pharmaceutically acceptable salt, prodrug, stereoisomer, solvate, or tautomer thereof, wherein:
R¹ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R² is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R¹ and R² together with the atoms to which they are attached and any intervening atoms, form a 5-8 membered cycloalkane, a 5-14 membered heterocycle, arene, or heteroarene wherein the cycloalkane, heterocycle, arene, or heteroarene is optionally substituted with one or more substituents independently selected from halogen, OH, CN, C₁-C₆ alkyl, C₁-C₆ alkoxy;
R³ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R⁴ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R³ and R⁴ together with the atoms to which they are attached and any intervening atoms, form a 5-8 membered cycloalkane, a 5-14 membered heterocycle, wherein the cycloalkane, heterocycle, is optionally substituted with one or more substituents independently selected from halogen, OH, CN, C₁-C₆ alkyl, C₁-C₆ alkoxy;
R⁵ is selected from H, C₁-C₆ alkyl, C₃-C₁₀ cycloalkyl, C₂-C₆ alkenyl, C₂-C₆ alkynyl, C₁-C₆ acyl, SO₂—C₁-C₆ alkyl, 4-10 membered heterocyclyl, wherein the alkyl, cycloalkyl, alkenyl, alkynyl, acyl, or heterocyclyl is optionally substituted with one or more substituents independently selected from halogen, oxo, OH, CN, C₁-C₆ alkoxy, C₃-C₁₀ cycloalkyl, aryl;
R⁶ is selected from H, C₁-C₆ alkyl, C₁-C₆ alkyl-C(O)—, aryl, wherein alkyl or aryl is optionally substituted with one or more substituents independently selected from halogen, CN, OH;
each R⁹ is independently selected from halogen, OH, C₁-C₆ alkyl, C₁-C₆ alkoxy, wherein alkyl or alkoxy optionally substituted with one or more halogen, OH; or two R⁹ together with the atoms to which they are attached and any intervening atoms, form a 5-6 membered heterocycle; or
R⁵ and R⁹ together with the atoms to which they are attached and any intervening atoms, form a 5-6 membered heterocycle;
n is an integer selected from 0, 1, and 2;
provided that when R⁵ is methyl then R⁶ is not H or CH₃CO—.

Another aspect of the invention is directed to pharmaceutical compositions comprising a compound of Formula (A), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention is directed to pharmaceutical compositions comprising a compound of Formula (I), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention is directed to pharmaceutical compositions comprising a compound of Formula (II), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention is directed to pharmaceutical compositions comprising a compound of Formula (III), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention is directed to pharmaceutical compositions comprising a compound of Formula (IV), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention is directed to pharmaceutical compositions comprising a compound of Formula (V), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention relates to a method of treating an inflammatory disease or disorder. The method comprises administering to a patient in need of a treatment for inflammatory diseases or disorders an effective amount of a compound of Formula (A), or a pharmaceutically acceptable salt, hydrate, solvate, prodrug, stereoisomer, tautomer, or pharmaceutical composition thereof.

Another aspect of the invention relates to a method of treating an inflammatory disease or disorder. The method comprises administering to a patient in need of a treatment for inflammatory diseases or disorders an effective amount of a compound of Formula (I), or a pharmaceutically acceptable salt, hydrate, solvate, prodrug, stereoisomer, tautomer, or pharmaceutical composition thereof.

Another aspect of the invention relates to a method of treating an inflammatory disease or disorder. The method comprises administering to a patient in need of a treatment for inflammatory diseases or disorders an effective amount of a compound of Formula (II), or a pharmaceutically acceptable salt, hydrate, solvate, prodrug, stereoisomer, tautomer, or pharmaceutical composition thereof.

Another aspect of the invention relates to a method of treating an inflammatory disease or disorder. The method comprises administering to a patient in need of a treatment for inflammatory diseases or disorders an effective amount of a compound of Formula (III), or a pharmaceutically acceptable salt, hydrate, solvate, prodrug, stereoisomer, tautomer, or pharmaceutical composition thereof.

Another aspect of the invention relates to a method of treating an inflammatory disease or disorder. The method comprises administering to a patient in need of a treatment for inflammatory diseases or disorders an effective amount of a compound of Formula (IV), or a pharmaceutically acceptable salt, hydrate, solvate, prodrug, stereoisomer, tautomer, or pharmaceutical composition thereof.

Another aspect of the invention relates to a method of treating an inflammatory disease or disorder. The method comprises administering to a patient in need of a treatment for inflammatory diseases or disorders an effective amount of a compound of Formula (V), or a pharmaceutically acceptable salt, hydrate, solvate, prodrug, stereoisomer, tautomer, or pharmaceutical composition thereof.

Another aspect of the invention is directed to a method of inhibiting of NLRP3. The method involves administering to a patient in need thereof an effective amount of a compound of Formula (A), or a pharmaceutically acceptable salt, hydrate, solvate, prodrug, stereoisomer, tautomer, or pharmaceutical composition thereof.

Another aspect of the invention is directed to a method of inhibiting of NLRP3. The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the invention is directed to a method of inhibiting of NLRP3. The method involves administering to a patient in need thereof an effective amount of a compound of Formula (II), or a pharmaceutically acceptable salt, hydrate, solvate, prodrug, stereoisomer, tautomer, or pharmaceutical composition thereof.

Another aspect of the invention is directed to a method of inhibiting of NLRP3. The method involves administering to a patient in need thereof an effective amount of a compound of Formula (III), or a pharmaceutically acceptable salt, hydrate, solvate, prodrug, stereoisomer, tautomer, or pharmaceutical composition thereof.

Another aspect of the invention is directed to a method of inhibiting of NLRP3. The method involves administering to a patient in need thereof an effective amount of a compound of Formula (IV), or a pharmaceutically acceptable salt, hydrate, solvate, prodrug, stereoisomer, tautomer, or pharmaceutical composition thereof.

Another aspect of the invention is directed to a method of inhibiting of NLRP3. The method involves administering to a patient in need thereof an effective amount of a compound of Formula (V), or a pharmaceutically acceptable salt, hydrate, solvate, prodrug, stereoisomer, tautomer, or pharmaceutical composition thereof.

Another aspect of the present invention relates to compounds of Formula (A), or pharmaceutically acceptable salts, hydrates, solvates, prodrugs, stereoisomers, tautomers, or pharmaceutical compositions thereof, for use in the manufacture of a medicament for inhibiting NLRP3.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for inhibiting NLRP3.

Another aspect of the present invention relates to compounds of Formula (II), or pharmaceutically acceptable salts, hydrates, solvates, prodrugs, stereoisomers, tautomers, or pharmaceutical compositions thereof, for use in the manufacture of a medicament for inhibiting NLRP3.

Another aspect of the present invention relates to compounds of Formula (III), or pharmaceutically acceptable salts, hydrates, solvates, prodrugs, stereoisomers, tautomers, or pharmaceutical compositions thereof, for use in the manufacture of a medicament for inhibiting NLRP3.

Another aspect of the present invention relates to compounds of Formula (IV), or pharmaceutically acceptable salts, hydrates, solvates, prodrugs, stereoisomers, tautomers, or pharmaceutical compositions thereof, for use in the manufacture of a medicament for inhibiting NLRP3.

Another aspect of the present invention relates to compounds of Formula (V), or pharmaceutically acceptable salts, hydrates, solvates, prodrugs, stereoisomers, tautomers, or pharmaceutical compositions thereof, for use in the manufacture of a medicament for inhibiting NLRP3.

Another aspect of the present invention relates to the use of compounds of Formula (A), or pharmaceutically acceptable salts, hydrates, solvates, prodrugs, stereoisomers, tautomers, or pharmaceutical compositions thereof, in the treatment of diseases and disorders associated with NLRP3.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of diseases and disorders associated with NLRP3.

Another aspect of the present invention relates to the use of compounds of Formula (II), or pharmaceutically acceptable salts, hydrates, solvates, prodrugs, stereoisomers, tautomers, or pharmaceutical compositions thereof, in the treatment of diseases and disorders associated with NLRP3.

Another aspect of the present invention relates to the use of compounds of Formula (III), or pharmaceutically acceptable salts, hydrates, solvates, prodrugs, stereoisomers, tautomers, or pharmaceutical compositions thereof, in the treatment of diseases and disorders associated with NLRP3.

Another aspect of the present invention relates to the use of compounds of Formula (IV), or pharmaceutically acceptable salts, hydrates, solvates, prodrugs, stereoisomers, tautomers, or pharmaceutical compositions thereof, in the treatment of diseases and disorders associated with NLRP3.

Another aspect of the present invention relates to the use of compounds of Formula (V), or pharmaceutically acceptable salts, hydrates, solvates, prodrugs, stereoisomers, tautomers, or pharmaceutical compositions thereof, in the treatment of diseases and disorders associated with NLRP3.

Another aspect of the present invention relates to compounds of Formula (A), or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the present invention relates to compounds of Formula (II), or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the present invention relates to compounds of Formula (III), or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the present invention relates to compounds of Formula (IV), or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the present invention relates to compounds of Formula (V), or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the invention is directed to a method of treating or preventing a disease or disorder disclosed herein in a subject in need thereof. The method involves administering to a patient in need of the treatment an effective amount of a compound of Formula (A), or a pharmaceutically acceptable salt, hydrate, solvate, prodrug, stereoisomer, tautomer, or pharmaceutical composition thereof.

Another aspect of the invention is directed to a method of treating or preventing a disease or disorder disclosed herein in a subject in need thereof. The method involves administering to a patient in need of the treatment an effective amount of a compound of Formula (I), or a pharmaceutically acceptable salt, hydrate, solvate, prodrug, stereoisomer, tautomer, or pharmaceutical composition thereof.

Another aspect of the invention is directed to a method of treating or preventing a disease or disorder disclosed herein in a subject in need thereof. The method involves administering to a patient in need of the treatment an effective amount of a compound of Formula (II), or a pharmaceutically acceptable salt, hydrate, solvate, prodrug, stereoisomer, tautomer, or pharmaceutical composition thereof.

Another aspect of the invention is directed to a method of treating or preventing a disease or disorder disclosed herein in a subject in need thereof. The method involves administering to a patient in need of the treatment an effective amount of a compound of Formula (III), or a pharmaceutically acceptable salt, hydrate, solvate, prodrug, stereoisomer, tautomer, or pharmaceutical composition thereof.

Another aspect of the invention is directed to a method of treating or preventing a disease or disorder disclosed herein in a subject in need thereof. The method involves administering to a patient in need of the treatment an effective amount of a compound of Formula (IV), or a pharmaceutically acceptable salt, hydrate, solvate, prodrug, stereoisomer, tautomer, or pharmaceutical composition thereof.

Another aspect of the invention is directed to a method of treating or preventing a disease or disorder disclosed herein in a subject in need thereof. The method involves administering to a patient in need of the treatment an effective amount of a compound of Formula (V), or a pharmaceutically acceptable salt, hydrate, solvate, prodrug, stereoisomer, tautomer, or pharmaceutical composition thereof.

Another aspect of the present invention relates to the use of compounds of Formula (A), or pharmaceutically acceptable salts, hydrates, solvates, prodrugs, stereoisomers, tautomers, or pharmaceutical compositions thereof, in the treatment of a disease or disorder disclosed herein.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease or disorder disclosed herein.

Another aspect of the present invention relates to the use of compounds of Formula (II), or pharmaceutically acceptable salts, hydrates, solvates, prodrugs, stereoisomers, tautomers, or pharmaceutical compositions thereof, in the treatment of a disease or disorder disclosed herein.

Another aspect of the present invention relates to the use of compounds of Formula (III), or pharmaceutically acceptable salts, hydrates, solvates, prodrugs, stereoisomers, tautomers, or pharmaceutical compositions thereof, in the treatment of a disease or disorder disclosed herein.

Another aspect of the present invention relates to the use of compounds of Formula (IV), or pharmaceutically acceptable salts, hydrates, solvates, prodrugs, stereoisomers, tautomers, or pharmaceutical compositions thereof, in the treatment of a disease or disorder disclosed herein.

Another aspect of the present invention relates to the use of compounds of Formula (V), or pharmaceutically acceptable salts, hydrates, solvates, prodrugs, stereoisomers, tautomers, or pharmaceutical compositions thereof, in the treatment of a disease or disorder disclosed herein.

The present invention further provides methods of treating a disease or disorder associated with NLRP3, comprising administering to a patient suffering from at least one of said diseases or disorders a compound of Formula (A), or a pharmaceutically acceptable salt, hydrate, solvate, prodrug, stereoisomer, tautomer, or pharmaceutical composition thereof.

The present invention further provides methods of treating a disease or disorder associated with NLRP3, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention further provides methods of treating a disease or disorder associated with NLRP3, comprising administering to a patient suffering from at least one of said diseases or disorders a compound of Formula (II), or a pharmaceutically acceptable salt, hydrate, solvate, prodrug, stereoisomer, tautomer, or pharmaceutical composition thereof.

The present invention further provides methods of treating a disease or disorder associated with NLRP3, comprising administering to a patient suffering from at least one of said diseases or disorders a compound of Formula (III), or a pharmaceutically acceptable salt, hydrate, solvate, prodrug, stereoisomer, tautomer, or pharmaceutical composition thereof.

The present invention further provides methods of treating a disease or disorder associated with NLRP3, comprising administering to a patient suffering from at least one of said diseases or disorders a compound of Formula (IV), or a pharmaceutically acceptable salt, hydrate, solvate, prodrug, stereoisomer, tautomer, or pharmaceutical composition thereof.

The present invention further provides methods of treating a disease or disorder associated with NLRP3, comprising administering to a patient suffering from at least one of said diseases or disorders a compound of Formula (V), or a pharmaceutically acceptable salt, hydrate, solvate, prodrug, stereoisomer, tautomer, or pharmaceutical composition thereof.

The present invention provides inhibitors of NLRP3 that are therapeutic agents in the treatment of diseases and disorders.

The present invention further provides compounds and compositions with an improved efficacy and safety profile relative to known inhibitors of NLRP3. The present disclosure also provides agents with novel mechanisms of action toward NLRP3 in the treatment of various types of diseases.

The present invention further provides methods of treating a disease or disorder associated with NLRP3, comprising administering to a patient suffering from at least one of said diseases or disorders a compound of Formula (A), or a pharmaceutically acceptable salt, hydrate, solvate, prodrug, stereoisomer, tautomer, or pharmaceutical composition thereof.

The present invention further provides methods of treating a disease or disorder associated with NLRP3, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention further provides methods of treating a disease or disorder associated with NLRP3, comprising administering to a patient suffering from at least one of said diseases or disorders a compound of Formula (II), or a pharmaceutically acceptable salt, hydrate, solvate, prodrug, stereoisomer, tautomer, or pharmaceutical composition thereof.

The present invention further provides methods of treating a disease or disorder associated with NLRP3, comprising administering to a patient suffering from at least one of said diseases or disorders a compound of Formula (III), or a pharmaceutically acceptable salt, hydrate, solvate, prodrug, stereoisomer, tautomer, or pharmaceutical composition thereof.

The present invention further provides methods of treating a disease or disorder associated with NLRP3, comprising administering to a patient suffering from at least one of said diseases or disorders a compound of Formula (IV), or a pharmaceutically acceptable salt, hydrate, solvate, prodrug, stereoisomer, tautomer, or pharmaceutical composition thereof.

The present invention further provides methods of treating a disease or disorder associated with NLRP3, comprising administering to a patient suffering from at least one of said diseases or disorders a compound of Formula (V), or a pharmaceutically acceptable salt, hydrate, solvate, prodrug, stereoisomer, tautomer, or pharmaceutical composition thereof.

The present invention further provides methods of treating blood diseases, bone diseases, cancer diseases, cardiovascular diseases, ear diseases, endocrine diseases, eye diseases, fetal diseases, gastrointestinal diseases; genetic diseases, immune diseases, infectious diseases, liver diseases, mental diseases, metabolic diseases, muscle diseases, nephrological diseases, neuronal diseases, oral diseases, rare diseases, reproductive diseases, respiratory diseases, skin diseases, smell/taste diseases; comprising administering to a patient suffering from at least one of said diseases or disorders a compound of Formula (A), or a pharmaceutically acceptable salt, hydrate, solvate, prodrug, stereoisomer, tautomer, or pharmaceutical composition thereof.

The present invention further provides methods of treating blood diseases, bone diseases, cancer diseases, cardiovascular diseases, ear diseases, endocrine diseases, eye diseases, fetal diseases, gastrointestinal diseases; genetic diseases, immune diseases, infectious diseases, liver diseases, mental diseases, metabolic diseases, muscle diseases, nephrological diseases, neuronal diseases, oral diseases, rare diseases, reproductive diseases, respiratory diseases, skin diseases, smell/taste diseases; comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention further provides methods of treating blood diseases, bone diseases, cancer diseases, cardiovascular diseases, ear diseases, endocrine diseases, eye diseases, fetal diseases, gastrointestinal diseases; genetic diseases, immune diseases, infectious diseases, liver diseases, mental diseases, metabolic diseases, muscle diseases, nephrological diseases, neuronal diseases, oral diseases, rare diseases, reproductive diseases, respiratory diseases, skin diseases, smell/taste diseases; comprising administering to a patient suffering from at least one of said diseases or disorders a compound of Formula (II), or a pharmaceutically acceptable salt, hydrate, solvate, prodrug, stereoisomer, tautomer, or pharmaceutical composition thereof.

The present invention further provides methods of treating blood diseases, bone diseases, cancer diseases, cardiovascular diseases, ear diseases, endocrine diseases, eye diseases, fetal diseases, gastrointestinal diseases; genetic diseases, immune diseases, infectious diseases, liver diseases, mental diseases, metabolic diseases, muscle diseases, nephrological diseases, neuronal diseases, oral diseases, rare diseases, reproductive diseases, respiratory diseases, skin diseases, smell/taste diseases; comprising administering to a patient suffering from at least one of said diseases or disorders a compound of Formula (III), or a pharmaceutically acceptable salt, hydrate, solvate, prodrug, stereoisomer, tautomer, or pharmaceutical composition thereof.

The present invention further provides methods of treating blood diseases, bone diseases, cancer diseases, cardiovascular diseases, ear diseases, endocrine diseases, eye diseases, fetal diseases, gastrointestinal diseases; genetic diseases, immune diseases, infectious diseases, liver diseases, mental diseases, metabolic diseases, muscle diseases, nephrological diseases, neuronal diseases, oral diseases, rare diseases, reproductive diseases, respiratory diseases, skin diseases, smell/taste diseases; comprising administering to a patient suffering from at least one of said diseases or disorders a compound of Formula (IV), or a pharmaceutically acceptable salt, hydrate, solvate, prodrug, stereoisomer, tautomer, or pharmaceutical composition thereof.

The present invention further provides methods of treating blood diseases, bone diseases, cancer diseases, cardiovascular diseases, ear diseases, endocrine diseases, eye diseases, fetal diseases, gastrointestinal diseases; genetic diseases, immune diseases, infectious diseases, liver diseases, mental diseases, metabolic diseases, muscle diseases, nephrological diseases, neuronal diseases, oral diseases, rare diseases, reproductive diseases, respiratory diseases, skin diseases, smell/taste diseases; comprising administering to a patient suffering from at least one of said diseases or disorders a compound of Formula (V), or a pharmaceutically acceptable salt, hydrate, solvate, prodrug, stereoisomer, tautomer, or pharmaceutical composition thereof.

The present invention further provides methods of treating a disease, disorder, or condition selected from: Acne (ACNE); Acrokeratoderma, Hereditary Papulotranslucent; Adult-Onset Still's Disease (AOSD); Aicardi-Goutieres Syndrome (AGS); Alzheimer Disease, Familial, 1 (AD1); Amyloidosis; Amyotrophic Lateral Sclerosis 1 (ALS1); Anthracosis; Aphthous Stomatitis; Arthritis; Arthropathy; Aseptic Meningitis; Asthma (ASTHMA); Autoimmune Disease of Cardiovascular System; Autoimmune Disease of Gastrointestinal Tract; Autoinflammatory Syndrome; Behcet Syndrome (BD); Blau Syndrome (BLAUS); Body Mass Index Quantitative Trait Locus 11 (BMIQ11); Bone Inflammation Disease; Brain Edema; Brucellosis; Candidiasis; Cervical Adenitis; Chondrocalcinosis; Chronic Meningitis; Chronic Recurrent Multifocal Osteomyelitis (CRMO); Chronic Urticaria; Cinca Syndrome (CINCA); Conjunctival Disease; Conjunctivitis; Connective Tissue Disease; Corneal Disease; Coronavirus Infectious Disease; Covid-19; Crohn's Disease; Cryopyrin-Associated Periodic Syndrome (CAPS); Cystic Fibrosis (CF); Deafness, Autosomal Dominant 34, with or Without Inflammation (DFNA34); Dermatitis; Diabetes Mellitus; Diabetic Encephalopathy; Exanthem (RASH); Eye Disease; Familial Cold Autoinflammatory Syndrome (FCU); Familial Cold Autoinflammatory Syndrome 1 (FCAS1); Familial Cold Autoinflammatory Syndrome 2 (FCAS2); Familial Mediterranean Fever (FMF); Gout (GOUT); Hereditary Periodic Fever Syndrome; Hidradenitis; Hidradenitis Suppurativa; Hypertension, Essential (EHT); Hyperuricemia; Intellectual Developmental Disorder, Autosomal Dominant 30, with Speech Delay and Behavioral Abnormalities (MRD30); Interstitial Lung Disease 2 (ILD2); Keratitis, Hereditary (KERH); Keratoendotheliitis Fugax Hereditaria (KEFH); Lipoprotein Quantitative Trait Locus (LPAQTL); Lymph Node Disease; Macular Degeneration, Age-Related, 1 (ARMD1); Malaria (CM); Meningitis; Mevalonic Aciduria (MEVA); Muckle-Wells Syndrome (MWS); Mycobacterium Kansasii; Myocardial Infarction (MCI1); Nephrolithiasis; Nervous System Disease; Non-Alcoholic Steatohepatitis (NASH); Optic Papillitis; Osteomyelitis; Osteoporosis (OSTEOP); Otitis Media (OMS); Papilledema; Pericardial Effusion; Pericardium Disease; Pharyngitis; Polyradiculoneuropathy; Primary Bacterial Infectious Disease; Proteasome-Associated Autoinflammatory Syndrome 1 (PRAAS1); Pyoderma; Pyoderma Gangrenosum; Pyogenic Sterile Arthritis, Pyoderma Gangrenosum, and Acne (PAPAS); Respiratory Failure; Salmonellosis; Schnitzler Syndrome; Sebaceous Gland Disease; Sensorineural Hearing Loss; Serum Amyloid a Amyloidosis; Silicosis; Skin Disease; Stomatitis; Type 2 Diabetes Mellitus (T2D); Ureteral Disease; Urticaria (HIVES); Vulvovaginal Candidiasis; Wells Syndrome comprising administering to a patient suffering from at least one of said diseases or disorders a compound of Formula (A), or a pharmaceutically acceptable salt, hydrate, solvate, prodrug, stereoisomer, tautomer, or pharmaceutical composition thereof.

The present invention further provides methods of treating a disease, disorder, or condition selected from: Acne (ACNE); Acrokeratoderma, Hereditary Papulotranslucent; Adult-Onset Still's Disease (AOSD); Aicardi-Goutieres Syndrome (AGS); Alzheimer Disease, Familial, 1 (AD1); Amyloidosis; Amyotrophic Lateral Sclerosis 1 (ALS1); Anthracosis; Aphthous Stomatitis; Arthritis; Arthropathy; Aseptic Meningitis; Asthma (ASTHMA); Autoimmune Disease of Cardiovascular System; Autoimmune Disease of Gastrointestinal Tract; Autoinflammatory Syndrome; Behcet Syndrome (BD); Blau Syndrome (BLAUS); Body Mass Index Quantitative Trait Locus 11 (BMIQ11); Bone Inflammation Disease; Brain Edema; Brucellosis; Candidiasis; Cervical Adenitis; Chondrocalcinosis; Chronic Meningitis; Chronic Recurrent Multifocal Osteomyelitis (CRMO); Chronic Urticaria; Cinca Syndrome (CINCA); Conjunctival Disease; Conjunctivitis; Connective Tissue Disease; Corneal Disease; Coronavirus Infectious Disease; Covid-19; Crohn's Disease; Cryopyrin-Associated Periodic Syndrome (CAPS); Cystic Fibrosis (CF); Deafness, Autosomal Dominant 34, with or Without Inflammation (DFNA34); Dermatitis; Diabetes Mellitus; Diabetic Encephalopathy; Exanthem (RASH); Eye Disease; Familial Cold Autoinflammatory Syndrome (FCU); Familial Cold Autoinflammatory Syndrome 1 (FCAS1); Familial Cold Autoinflammatory Syndrome 2 (FCAS2); Familial Mediterranean Fever (FMF); Gout (GOUT); Hereditary Periodic Fever Syndrome; Hidradenitis; Hidradenitis Suppurativa; Hypertension, Essential (EHT); Hyperuricemia; Intellectual Developmental Disorder, Autosomal Dominant 30, with Speech Delay and Behavioral Abnormalities (MRD30); Interstitial Lung Disease 2 (ILD2); Keratitis, Hereditary (KERH); Keratoendotheliitis Fugax Hereditaria (KEFH); Lipoprotein Quantitative Trait Locus (LPAQTL); Lymph Node Disease; Macular Degeneration, Age-Related, 1 (ARMD1); Malaria (CM); Meningitis; Mevalonic Aciduria (MEVA); Muckle-Wells Syndrome (MWS); Mycobacterium Kansasii; Myocardial Infarction (MCI1); Nephrolithiasis; Nervous System Disease; Non-Alcoholic Steatohepatitis (NASH); Optic Papillitis; Osteomyelitis; Osteoporosis (OSTEOP); Otitis Media (OMS); Papilledema; Pericardial Effusion; Pericardium Disease; Pharyngitis; Polyradiculoneuropathy; Primary Bacterial Infectious Disease; Proteasome-Associated Autoinflammatory Syndrome 1 (PRAAS1); Pyoderma; Pyoderma Gangrenosum; Pyogenic Sterile Arthritis, Pyoderma Gangrenosum, and Acne (PAPAS); Respiratory Failure; Salmonellosis; Schnitzler Syndrome; Sebaceous Gland Disease; Sensorineural Hearing Loss; Serum Amyloid a Amyloidosis; Silicosis; Skin Disease; Stomatitis; Type 2 Diabetes Mellitus (T2D); Ureteral Disease; Urticaria (HIVES); Vulvovaginal Candidiasis; Wells Syndrome comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention further provides methods of treating a disease, disorder, or condition selected from: Acne (ACNE); Acrokeratoderma, Hereditary Papulotranslucent; Adult-Onset Still's Disease (AOSD); Aicardi-Goutieres Syndrome (AGS); Alzheimer Disease, Familial, 1 (AD1); Amyloidosis; Amyotrophic Lateral Sclerosis 1 (ALS1); Anthracosis; Aphthous Stomatitis; Arthritis; Arthropathy; Aseptic Meningitis; Asthma (ASTHMA); Autoimmune Disease of Cardiovascular System; Autoimmune Disease of Gastrointestinal Tract; Autoinflammatory Syndrome; Behcet Syndrome (BD); Blau Syndrome (BLAUS); Body Mass Index Quantitative Trait Locus 11 (BMIQ11); Bone Inflammation Disease; Brain Edema; Brucellosis; Candidiasis; Cervical Adenitis; Chondrocalcinosis; Chronic Meningitis; Chronic Recurrent Multifocal Osteomyelitis (CRMO); Chronic Urticaria; Cinca Syndrome (CINCA); Conjunctival Disease; Conjunctivitis; Connective Tissue Disease; Corneal Disease; Coronavirus Infectious Disease; Covid-19; Crohn's Disease; Cryopyrin-Associated Periodic Syndrome (CAPS); Cystic Fibrosis (CF); Deafness, Autosomal Dominant 34, with or Without Inflammation (DFNA34); Dermatitis; Diabetes Mellitus; Diabetic Encephalopathy; Exanthem (RASH); Eye Disease; Familial Cold Autoinflammatory Syndrome (FCU); Familial Cold Autoinflammatory Syndrome 1 (FCAS1); Familial Cold Autoinflammatory Syndrome 2 (FCAS2); Familial Mediterranean Fever (FMF); Gout (GOUT); Hereditary Periodic Fever Syndrome; Hidradenitis; Hidradenitis Suppurativa; Hypertension, Essential (EHT); Hyperuricemia; Intellectual Developmental Disorder, Autosomal Dominant 30, with Speech Delay and Behavioral Abnormalities (MRD30); Interstitial Lung Disease 2 (ILD2); Keratitis, Hereditary (KERH); Keratoendotheliitis Fugax Hereditaria (KEFH); Lipoprotein Quantitative Trait Locus (LPAQTL); Lymph Node Disease; Macular Degeneration, Age-Related, 1 (ARMD1); Malaria (CM); Meningitis; Mevalonic Aciduria (MEVA); Muckle-Wells Syndrome (MWS); Mycobacterium Kansasii; Myocardial Infarction (MCI1); Nephrolithiasis; Nervous System Disease; Non-Alcoholic Steatohepatitis (NASH); Optic Papillitis; Osteomyelitis; Osteoporosis (OSTEOP); Otitis Media (OMS); Papilledema; Pericardial Effusion; Pericardium Disease; Pharyngitis; Polyradiculoneuropathy; Primary Bacterial Infectious Disease; Proteasome-Associated Autoinflammatory Syndrome 1 (PRAAS1); Pyoderma; Pyoderma Gangrenosum; Pyogenic Sterile Arthritis, Pyoderma Gangrenosum, and Acne (PAPAS); Respiratory Failure; Salmonellosis; Schnitzler Syndrome; Sebaceous Gland Disease; Sensorineural Hearing Loss; Serum Amyloid a Amyloidosis; Silicosis; Skin Disease; Stomatitis; Type 2 Diabetes Mellitus (T2D); Ureteral Disease; Urticaria (HIVES); Vulvovaginal Candidiasis; Wells Syndrome comprising administering to a patient suffering from at least one of said diseases or disorders a compound of Formula (II), or a pharmaceutically acceptable salt, hydrate, solvate, prodrug, stereoisomer, tautomer, or pharmaceutical composition thereof.

The present invention further provides methods of treating a disease, disorder, or condition selected from: Acne (ACNE); Acrokeratoderma, Hereditary Papulotranslucent; Adult-Onset Still's Disease (AOSD); Aicardi-Goutieres Syndrome (AGS); Alzheimer Disease, Familial, 1 (AD1); Amyloidosis; Amyotrophic Lateral Sclerosis 1 (ALS1); Anthracosis; Aphthous Stomatitis; Arthritis; Arthropathy; Aseptic Meningitis; Asthma (ASTHMA); Autoimmune Disease of Cardiovascular System; Autoimmune Disease of Gastrointestinal Tract; Autoinflammatory Syndrome; Behcet Syndrome (BD); Blau Syndrome (BLAUS); Body Mass Index Quantitative Trait Locus 11 (BMIQ11); Bone Inflammation Disease; Brain Edema; Brucellosis; Candidiasis; Cervical Adenitis; Chondrocalcinosis; Chronic Meningitis; Chronic Recurrent Multifocal Osteomyelitis (CRMO); Chronic Urticaria; Cinca Syndrome (CINCA); Conjunctival Disease; Conjunctivitis; Connective Tissue Disease; Corneal Disease; Coronavirus Infectious Disease; Covid-19; Crohn's Disease; Cryopyrin-Associated Periodic Syndrome (CAPS); Cystic Fibrosis (CF); Deafness, Autosomal Dominant 34, with or Without Inflammation (DFNA34); Dermatitis; Diabetes Mellitus; Diabetic Encephalopathy; Exanthem (RASH); Eye Disease; Familial Cold Autoinflammatory Syndrome (FCU); Familial Cold Autoinflammatory Syndrome 1 (FCAS1); Familial Cold Autoinflammatory Syndrome 2 (FCAS2); Familial Mediterranean Fever (FMF); Gout (GOUT); Hereditary Periodic Fever Syndrome; Hidradenitis; Hidradenitis Suppurativa; Hypertension, Essential (EHT); Hyperuricemia; Intellectual Developmental Disorder, Autosomal Dominant 30, with Speech Delay and Behavioral Abnormalities (MRD30); Interstitial Lung Disease 2 (ILD2); Keratitis, Hereditary (KERH); Keratoendotheliitis Fugax Hereditaria (KEFH); Lipoprotein Quantitative Trait Locus (LPAQTL); Lymph Node Disease; Macular Degeneration, Age-Related, 1 (ARMD1); Malaria (CM); Meningitis; Mevalonic Aciduria (MEVA); Muckle-Wells Syndrome (MWS); Mycobacterium Kansasii; Myocardial Infarction (MCI1); Nephrolithiasis; Nervous System Disease; Non-Alcoholic Steatohepatitis (NASH); Optic Papillitis; Osteomyelitis; Osteoporosis (OSTEOP); Otitis Media (OMS); Papilledema; Pericardial Effusion; Pericardium Disease; Pharyngitis; Polyradiculoneuropathy; Primary Bacterial Infectious Disease; Proteasome-Associated Autoinflammatory Syndrome 1 (PRAAS1); Pyoderma; Pyoderma Gangrenosum; Pyogenic Sterile Arthritis, Pyoderma Gangrenosum, and Acne (PAPAS); Respiratory Failure; Salmonellosis; Schnitzler Syndrome; Sebaceous Gland Disease; Sensorineural Hearing Loss; Serum Amyloid a Amyloidosis; Silicosis; Skin Disease; Stomatitis; Type 2 Diabetes Mellitus (T2D); Ureteral Disease; Urticaria (HIVES); Vulvovaginal Candidiasis; Wells Syndrome comprising administering to a patient suffering from at least one of said diseases or disorders a compound of Formula (III), or a pharmaceutically acceptable salt, hydrate, solvate, prodrug, stereoisomer, tautomer, or pharmaceutical composition thereof.

The present invention further provides methods of treating a disease, disorder, or condition selected from: Acne (ACNE); Acrokeratoderma, Hereditary Papulotranslucent; Adult-Onset Still's Disease (AOSD); Aicardi-Goutieres Syndrome (AGS); Alzheimer Disease, Familial, 1 (AD1); Amyloidosis; Amyotrophic Lateral Sclerosis 1 (ALS1); Anthracosis; Aphthous Stomatitis; Arthritis; Arthropathy; Aseptic Meningitis; Asthma (ASTHMA); Autoimmune Disease of Cardiovascular System; Autoimmune Disease of Gastrointestinal Tract; Autoinflammatory Syndrome; Behcet Syndrome (BD); Blau Syndrome (BLAUS); Body Mass Index Quantitative Trait Locus 11 (BMIQ11); Bone Inflammation Disease; Brain Edema; Brucellosis; Candidiasis; Cervical Adenitis; Chondrocalcinosis; Chronic Meningitis; Chronic Recurrent Multifocal Osteomyelitis (CRMO); Chronic Urticaria; Cinca Syndrome (CINCA); Conjunctival Disease; Conjunctivitis; Connective Tissue Disease; Corneal Disease; Coronavirus Infectious Disease; Covid-19; Crohn's Disease; Cryopyrin-Associated Periodic Syndrome (CAPS); Cystic Fibrosis (CF); Deafness, Autosomal Dominant 34, with or Without Inflammation (DFNA34); Dermatitis; Diabetes Mellitus; Diabetic Encephalopathy; Exanthem (RASH); Eye Disease; Familial Cold Autoinflammatory Syndrome (FCU); Familial Cold Autoinflammatory Syndrome 1 (FCAS1); Familial Cold Autoinflammatory Syndrome 2 (FCAS2); Familial Mediterranean Fever (FMF); Gout (GOUT); Hereditary Periodic Fever Syndrome; Hidradenitis; Hidradenitis Suppurativa; Hypertension, Essential (EHT); Hyperuricemia; Intellectual Developmental Disorder, Autosomal Dominant 30, with Speech Delay and Behavioral Abnormalities (MRD30); Interstitial Lung Disease 2 (ILD2); Keratitis, Hereditary (KERH); Keratoendotheliitis Fugax Hereditaria (KEFH); Lipoprotein Quantitative Trait Locus (LPAQTL); Lymph Node Disease; Macular Degeneration, Age-Related, 1 (ARMD1); Malaria (CM); Meningitis; Mevalonic Aciduria (MEVA); Muckle-Wells Syndrome (MWS); Mycobacterium Kansasii; Myocardial Infarction (MCI1); Nephrolithiasis; Nervous System Disease; Non-Alcoholic Steatohepatitis (NASH); Optic Papillitis; Osteomyelitis; Osteoporosis (OSTEOP); Otitis Media (OMS); Papilledema; Pericardial Effusion; Pericardium Disease; Pharyngitis; Polyradiculoneuropathy; Primary Bacterial Infectious Disease; Proteasome-Associated Autoinflammatory Syndrome 1 (PRAAS1); Pyoderma; Pyoderma Gangrenosum; Pyogenic Sterile Arthritis, Pyoderma Gangrenosum, and Acne (PAPAS); Respiratory Failure; Salmonellosis; Schnitzler Syndrome; Sebaceous Gland Disease; Sensorineural Hearing Loss; Serum Amyloid a Amyloidosis; Silicosis; Skin Disease; Stomatitis; Type 2 Diabetes Mellitus (T2D); Ureteral Disease; Urticaria (HIVES); Vulvovaginal Candidiasis; Wells Syndrome comprising administering to a patient suffering from at least one of said diseases or disorders a compound of Formula (IV), or a pharmaceutically acceptable salt, hydrate, solvate, prodrug, stereoisomer, tautomer, or pharmaceutical composition thereof.

The present invention further provides methods of treating a disease, disorder, or condition selected from: Acne (ACNE); Acrokeratoderma, Hereditary Papulotranslucent; Adult-Onset Still's Disease (AOSD); Aicardi-Goutieres Syndrome (AGS); Alzheimer Disease, Familial, 1 (AD1); Amyloidosis; Amyotrophic Lateral Sclerosis 1 (ALS1); Anthracosis; Aphthous Stomatitis; Arthritis; Arthropathy; Aseptic Meningitis; Asthma (ASTHMA); Autoimmune Disease of Cardiovascular System; Autoimmune Disease of Gastrointestinal Tract; Autoinflammatory Syndrome; Behcet Syndrome (BD); Blau Syndrome (BLAUS); Body Mass Index Quantitative Trait Locus 11 (BMIQ11); Bone Inflammation Disease; Brain Edema; Brucellosis; Candidiasis; Cervical Adenitis; Chondrocalcinosis; Chronic Meningitis; Chronic Recurrent Multifocal Osteomyelitis (CRMO); Chronic Urticaria; Cinca Syndrome (CINCA); Conjunctival Disease; Conjunctivitis; Connective Tissue Disease; Corneal Disease; Coronavirus Infectious Disease; Covid-19; Crohn's Disease; Cryopyrin-Associated Periodic Syndrome (CAPS); Cystic Fibrosis (CF); Deafness, Autosomal Dominant 34, with or Without Inflammation (DFNA34); Dermatitis; Diabetes Mellitus; Diabetic Encephalopathy; Exanthem (RASH); Eye Disease; Familial Cold Autoinflammatory Syndrome (FCU); Familial Cold Autoinflammatory Syndrome 1 (FCAS1); Familial Cold Autoinflammatory Syndrome 2 (FCAS2); Familial Mediterranean Fever (FMF); Gout (GOUT); Hereditary Periodic Fever Syndrome; Hidradenitis; Hidradenitis Suppurativa; Hypertension, Essential (EHT); Hyperuricemia; Intellectual Developmental Disorder, Autosomal Dominant 30, with Speech Delay and Behavioral Abnormalities (MRD30); Interstitial Lung Disease 2 (ILD2); Keratitis, Hereditary (KERH); Keratoendotheliitis Fugax Hereditaria (KEFH); Lipoprotein Quantitative Trait Locus (LPAQTL); Lymph Node Disease; Macular Degeneration, Age-Related, 1 (ARMD1); Malaria (CM); Meningitis; Mevalonic Aciduria (MEVA); Muckle-Wells Syndrome (MWS); Mycobacterium Kansasii; Myocardial Infarction (MCI1); Nephrolithiasis; Nervous System Disease; Non-Alcoholic Steatohepatitis (NASH); Optic Papillitis; Osteomyelitis; Osteoporosis (OSTEOP); Otitis Media (OMS); Papilledema; Pericardial Effusion; Pericardium Disease; Pharyngitis; Polyradiculoneuropathy; Primary Bacterial Infectious Disease; Proteasome-Associated Autoinflammatory Syndrome 1 (PRAAS1); Pyoderma; Pyoderma Gangrenosum; Pyogenic Sterile Arthritis, Pyoderma Gangrenosum, and Acne (PAPAS); Respiratory Failure; Salmonellosis; Schnitzler Syndrome; Sebaceous Gland Disease; Sensorineural Hearing Loss; Serum Amyloid a Amyloidosis; Silicosis; Skin Disease; Stomatitis; Type 2 Diabetes Mellitus (T2D); Ureteral Disease; Urticaria (HIVES); Vulvovaginal Candidiasis; Wells Syndrome comprising administering to a patient suffering from at least one of said diseases or disorders a compound of Formula (V), or a pharmaceutically acceptable salt, hydrate, solvate, prodrug, stereoisomer, tautomer, or pharmaceutical composition thereof.

In some aspects, the present disclosure provides a compound obtainable by, or obtained by, a method for preparing compounds described herein (e.g., a method comprising one or more steps described in General Procedure).

In some aspects, the present disclosure provides a compound obtainable by, or obtained by, a method for preparing compounds described herein (e.g., a method comprising one or more steps described in Synthesis of the Representative Examples of the compound, examples 1-9).

In some aspects, the present disclosure provides an intermediate as described herein, being suitable for use in a method for preparing a compound as described herein (e.g., the intermediate is selected from the intermediates described in Preparative part-compounds P1-P16).

In some aspects, the present disclosure provides a method of preparing compounds of the present disclosure.

In some aspects, the present disclosure provides a method of preparing compounds of the present disclosure, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods, and examples are illustrative only and are not intended to be limiting. In the case of conflict between the chemical structures and names of the compounds disclosed herein, the chemical structures will control.

Other features and advantages of the disclosure will be apparent from the following detailed description and claims

### DETAILED DESCRIPTION

The present disclosure provides methods of treating, preventing, or ameliorating a disease or disorder associated with the NLRP3 by administering to a subject in need thereof a therapeutically effective amount of a compound as disclosed herein.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have one or more substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups but does not necessarily have any further functional groups. Suitable substituents used in the optional substitution of the described groups include, without limitation, halogen, oxo, —OH, —CN, —COOH, CH₂CN, —O—(C₁-C₆)alkyl, (C₁-C₆)alkyl, (C₁-C₆)alkoxy, (C₁-C₆) haloalkyl, (C₁-C₆) haloalkoxy, —O—(C₂-C₆)alkenyl, —O—(C₂-C₆)alkynyl, (C₂-C₆)alkenyl, (C₂-C₆)alkynyl, —OH, —OP(O)(OH)₂, —OC(O)(C₁-C₆)alkyl, —C(O)(C₁-C₆)alkyl, —OC(O)O(C₁-C₆)alkyl, —NH₂, —NH((C₁-C₆)alkyl), —N((C₁-C₆)alkyl)₂, —NHC(O) (C₁-C₆) alkyl, —C(O)NH(C₁-C₆)alkyl, —S(O)₂(C₁-C₆)alkyl, —S(O)NH(C₁-C₆)alkyl, and —S(O)N((C₁-C₆)alkyl)₂. The substituents can themselves be optionally substituted. “Optionally substituted” as used herein also refers to substituted or unsubstituted whose meaning is described below.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

Unless otherwise specifically defined, the term “aryl” refers to cyclic, aromatic hydrocarbon groups that have 1 to 3 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen, —O—(C₁-C₆)alkyl, (C₁-C₆)alkyl, —O—(C₂-C₆)alkenyl, —O—(C₂-C₆)alkynyl, (C₂-C₆)alkenyl, (C₂-C₆)alkynyl, —OH, —OP(O)(OH)₂, —OC(O)(C₁-C₆)alkyl, —C(O)(C₁-C₆)alkyl, —OC(O)O(C₁-C₆)alkyl, —NH₂, —NH((C₁-C₆)alkyl), —N((C₁-C₆)alkyl)₂, —S(O)₂—(C₁-C₆)alkyl, —S(O)NH(C₁-C₆)alkyl, and —S(O)N((C₁-C₆)alkyl)₂. The substituents can themselves be optionally substituted. Furthermore, when containing two fused rings the aryl groups herein defined may have one or more saturated or partially unsaturated ring fused with a fully unsaturated aromatic ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic or a polycyclic aromatic radical of 5 to 24 ring atoms, containing one or more ring heteroatoms selected from N, O, S, P, or B, the remaining ring atoms being C. A polycyclic aromatic radical includes two or more fused rings and may further include two or more spiro-fused rings, e.g., bicyclic, tricyclic, tetracyclic, and the like. Unless otherwise specifically defined, “fused” means two rings sharing two ring atoms. Unless otherwise specifically defined, “spiro-fused” means two rings sharing one ring atom. Heteroaryl as herein defined also means a bicyclic heteroaromatic group wherein the heteroatom is selected from N, O, S, P, or B. Heteroaryl as herein defined also means a tricyclic heteroaromatic group containing one or more ring heteroatoms selected from N, O, S, P, or B. Heteroaryl as herein defined also means a tetracyclic heteroaromatic group containing one or more ring heteroatoms selected from N, O, S, P, or B. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two or more fused rings, the heteroaryl groups defined herein may have one or more saturated or partially unsaturated ring fused with one or more fully unsaturated aromatic ring. In heteroaryl ring systems containing more than two fused rings, a saturated or partially unsaturated ring may further be fused with a saturated or partially unsaturated ring described herein. Furthermore, when containing three or more fused rings, the heteroaryl groups defined herein may have one or more saturated or partially unsaturated ring spiro-fused. Any saturated or partially unsaturated ring described herein is optionally substituted with one or more oxo. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, indolinyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizine, pyrazolo[1,5-a]pyrimidin-7 (4H)-only, 3,4-dihydropyrazino[1,2-a]indol-1 (2H)-onyl, benzo[c][1,2]oxaborol-1 (3H)-olyl, 6,6a, 7,8-tetrahydro-9H-pyrido[2,3-b]puyrrolo[1,2-d][1,4]oxazin-9-onyl, or 6a′,7′-dihydro-6′H,9′H-spiro[cyclopropane-1,8′-pyrido[2,3-b]pyrrolo[1,2-d][1,4]oxazin]-9′-onyl.

Halogen or “halo” refers to fluorine, chlorine, bromine, or iodine.

Alkyl refers to a straight or branched chain saturated hydrocarbon containing 1-12 carbon atoms. Examples of a (C₁-C₆)alkyl group include, but are not limited to, methyl, ethyl, propyl, butyl, pentyl, hexyl, isopropyl (iso-propyl), isobutyl (iso-butyl), sec-butyl, tert-butyl, isopentyl (iso-pentyl), neopentyl (neo-pentyl), and isohexyl (iso-hexyl).

“Alkoxy” refers to a straight or branched chain saturated hydrocarbon containing 1-12 carbon atoms containing a terminal “O” in the chain, i.e., —O (alkyl). Examples of alkoxy groups include without limitation, methoxy, ethoxy, propoxy, butoxy, tert-butoxy, or pentoxy groups.

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched.

“Alkynyl” refers to a straight or branched chain unsaturated hydrocarbon containing 2-12 carbon atoms. The “alkynyl” group contains at least one triple bond in the chain. Examples of alkynyl groups include ethynyl, propargyl, n-butynyl, iso-butynyl, pentynyl, or hexynyl. An alkynyl group can be unsubstituted or substituted.

The term “alkylene”, “alkylenyl” or “alkanediyl” refers to a divalent alkyl radical. Any of the above-mentioned monovalent alkyl groups may be an alkylene by abstraction of a second hydrogen atom from the alkyl. As herein defined, alkylene may also be a C₁-C₆ alkylene. An alkylene may further be a C₁-C₄ alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Cycloalkyl” means mono or polycyclic saturated carbon rings containing 3-18 carbon atoms. Polycyclic cycloalkyl may be fused bicyclic cycloalkyl, bridged bicyclic cycloalkyl, or spiro-fused bicyclic cycloalkyl. A polycyclic cycloalkyl comprises at least one non-aromatic ring. Examples of cycloalkyl groups include, without limitations, cyclopropyl, cyclobutyl, cyclopentyl, cyclohexyl, cycloheptanyl, cyclooctanyl, norbornyl, norborenyl, 1,2,3,4-tetrahydronaphthyl, 2,3-dihydro-1H-indenyl, spiro[3.5]nonyl, spiro[5.5]undecyl, bicyclo[1.1.1]pentanyl, bicyclo[2.2.2]octanyl, or bicyclo[2.2.2]octenyl.

“Heterocyclyl”, “heterocycle” or “heterocycloalkyl” mono or polycyclic rings containing 3-24 atoms which include carbon and one or more heteroatoms selected from N, O, S, P, or B and wherein the rings are not aromatic. The heterocycloalkyl ring structure may be substituted by one or more substituents. The substituents can themselves be optionally substituted. Examples of heterocyclyl rings include, but are not limited to, oxetanyl, azetidinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, and homotropanyl.

The term “aromatic” means a planar ring having 4n+2 electrons in a conjugated system. As used herein, “conjugated system” means a system of connected p-orbitals with delocalized electrons, and the system may include lone electron pairs.

The term “haloalkyl” as used herein refers to an alkyl group, as defined herein, which is substituted one or more halogen. Examples of haloalkyl groups include, but are not limited to, trifluoromethyl, difluoromethyl, pentafluoroethyl, trichloromethyl, etc.

The term “haloalkoxy” as used herein refers to an alkoxy group, as defined herein, which is substituted with one or more halogen. Examples of haloalkoxy groups include, but are not limited to, trifluoromethoxy, difluoromethoxy, pentafluoroethoxy, trichloromethoxy, etc.

The term “cyano” as used herein means a substituent having a carbon atom joined to a nitrogen atom by a triple bond, i.e., C≡N.

“Spirocycloalkyl” or “spirocyclyl” means carbogenic bicyclic ring systems with both rings connected through a single atom. The ring can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. One or more of the carbon atoms in the spirocycle can be substituted with a heteroatom (e.g., O, N, S, or P). A (C₃-C₁₂) spirocycloalkyl is a spirocycle containing between 3 and 12 carbon atoms. One or more of the carbon atoms can be substituted with a heteroatom.

The term “spiroheterocycloalkyl”, “spiroheterocycle”, or “spiroheterocyclyl” is understood to mean a spirocycle wherein at least one of the rings is a heterocycle (e.g., at least one of the rings is furanyl, morpholinyl, or piperidinyl).

The term “solvate” refers to a complex of variable stoichiometry formed by a solute and solvent. Such solvents for the purpose of the disclosure may not interfere with the biological activity of the solute. Examples of suitable solvents include, but are not limited to, water, MeOH, EtOH, DMSO, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of Formula (A) and in particular compounds of Formula (I), Formula (II), Formula (III), Formula (IV), or Formula (V) may have one or more asymmetric carbon atom and may occur as racemates, racemic mixtures and as individual enantiomers or diastereomers.

The present disclosure also contemplates isotopically-labelled compounds of Formula I (e.g., those labeled with ²H and ¹⁴C). Deuterated (i.e., ²H or D) and carbon-14 (i.e., ¹⁴C) isotopes are particularly preferred for their ease of preparation and detectability. Further, substitution with heavier isotopes such as deuterium may afford certain therapeutic advantages resulting from greater metabolic stability (e.g., increased in vivo half-life or reduced dosage requirements) and hence may be preferred in some circumstances. Isotopically labelled compounds of Formula (A) and in particular compounds of Formula (I), Formula (II), Formula (III), Formula (IV), or Formula (V) can generally be prepared by following procedures analogous to those disclosed in the Schemes and/or in the Examples herein below, by substituting an appropriate isotopically labelled reagent for a non-isotopically labelled reagent.

The disclosure also includes pharmaceutical compositions comprising a therapeutically effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, pantothenate, phosphate/diphosphate, picrate, polygalacturonate, propionate, p-toluenesulfonate, salicylate, stearate, subacetate, succinate, sulfate, sulfosalicylate, tannate, tartrate, teoclate, tosylate, triethiodide, and valerate salts.

A “patient” or “subject” is a mammal, e.g., a human, mouse, rat, guinea pig, dog, cat, horse, cow, pig, or non-human primate, such as a monkey, chimpanzee, baboon, or rhesus.

An “effective amount” when used in connection with a compound is an amount effective for treating or preventing a disease in a subject as described herein.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a disclosed compound.

The term “salt’ refers to pharmaceutically acceptable salts.

The term “pharmaceutically acceptable salt” also refers to a salt of the compositions of the present disclosure having an acidic functional group, such as a carboxylic acid functional group, and a base.

“NLRP3 inhibitor” as used herein refer to compounds of Formula A and/or compositions comprising a compound of Formula A which inhibits NLRP3 kinase.

“NLRP3 inhibitor” as used herein refer to compounds of Formula I and/or compositions comprising a compound of Formula I which inhibits NLRP3 kinase.

“NLRP3 inhibitor” as used herein refer to compounds of Formula II and/or compositions comprising a compound of Formula II which inhibits NLRP3 kinase.

“NLRP3 inhibitor” as used herein refer to compounds of Formula III and/or compositions comprising a compound of Formula III which inhibits NLRP3 kinase.

“NLRP3 inhibitor” as used herein refer to compounds of Formula IV and/or compositions comprising a compound of Formula IV which inhibits NLRP3 kinase.

“NLRP3 inhibitor” as used herein refer to compounds of Formula V and/or compositions comprising a compound of Formula V which inhibits NLRP3 kinase.

The amount of compound of composition described herein needed for achieving a therapeutic effect may be determined empirically in accordance with conventional procedures for the particular purpose. Generally, for administering therapeutic agents (e.g. compounds or compositions of Formula A, I, II, III, IV, V (and/or additional agents) described herein) for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. For example, administration of therapeutic agents to a subject suffering from cancer provides a therapeutic benefit not only when the underlying condition is eradicated or ameliorated, but also when the subject reports a decrease in the severity or duration of the symptoms associated with the disease, e.g., a decrease in tumor burden, a decrease in circulating tumor cells, an increase in progression free survival. Therapeutic benefit also includes halting or slowing the progression of the underlying disease or disorder, regardless of whether improvement is realized.

### Compounds of the Present Disclosure

In one aspect, the present disclosure provides compounds of Formula (A) and salts, stereoisomers, solvates, prodrugs, isotopic derivatives, and tautomers thereof:

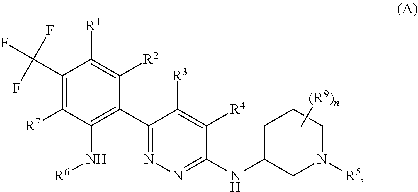

wherein R¹, R², R³, R⁴, R⁵, R⁶, R⁷, R⁹ and n are as described herein.

It is understood that, for a compound of Formula (A), R¹, R², R³, R⁴, R⁵, R⁶, R⁷, R⁹ and n can each be, where applicable, selected from the groups described herein, and any group described herein for any R¹, R², R³, R⁴, R⁵, R⁶, R⁷, R⁹ and n can be combined, where applicable, with any group described herein for one or more of the remainders of R¹, R², R³, R⁴, R⁵, R⁶, R⁷, R⁹ and n.

In some embodiments, the compound is of Formula (A*):

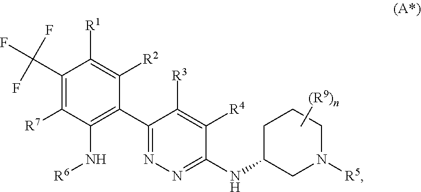

or a pharmaceutically acceptable salt, stereoisomer, solvate, prodrug, isotopic derivative, or tautomer thereof.

In some embodiments, the compound is of Formula (A**):

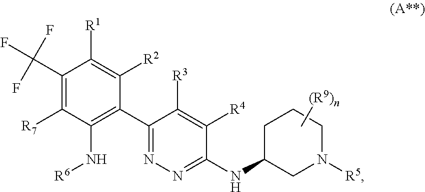

or a pharmaceutically acceptable salt, stereoisomer, solvate, prodrug, isotopic derivative, or tautomer thereof.

In some embodiments, the present disclosure provides compounds of Formula (I) and salts, stereoisomers, solvates, prodrugs, isotopic derivatives, and tautomers thereof:

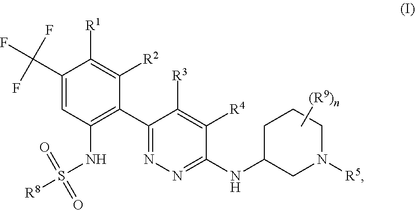

wherein R¹, R², R³, R⁴, R⁵, R⁸, R⁹ and n are as described herein.

It is understood that, for a compound of Formula (I), R¹, R², R³, R⁴, R⁵, R⁸, R⁹ and n can each be, where applicable, selected from the groups described herein, and any group described herein for any R¹, R², R³, R⁴, R⁵, R⁸, R⁹ and n can be combined, where applicable, with any group described herein for one or more of the remainders of R¹, R², R³, R⁴, R⁵, R⁸, R⁹ and n.

In some embodiments, the compound is of Formula (I*):

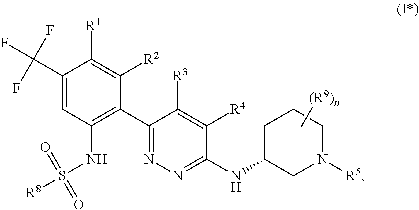

or a pharmaceutically acceptable salt, stereoisomer, solvate, prodrug, isotopic derivative, or tautomer thereof.

In some embodiments, the compound is of Formula (I**):

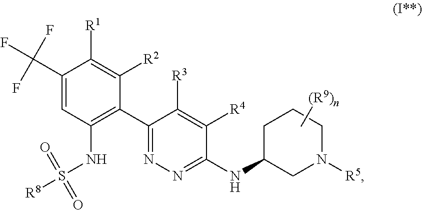

or a pharmaceutically acceptable salt, stereoisomer, solvate, prodrug, isotopic derivative, or tautomer thereof.

In some embodiments, the present disclosure provides compounds of Formula (II) and salts, stereoisomers, solvates, prodrugs, isotopic derivatives, and tautomers thereof:

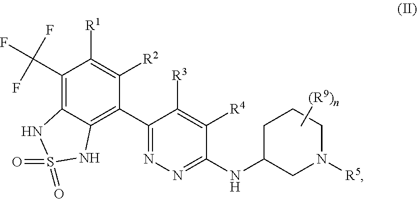

wherein R¹, R², R³, R⁴, R⁵, R⁹ and n are as described herein.

It is understood that, for a compound of Formula (II), R¹, R², R³, R⁴, R⁵, R⁹ and n can each be, where applicable, selected from the groups described herein, and any group described herein for any R¹, R², R³, R⁴, R⁵, R⁹ and n can be combined, where applicable, with any group described herein for one or more of the remainders of R¹, R², R³, R⁴, R⁵, R⁹ and n.

In some embodiments, the compound is of Formula (II*):

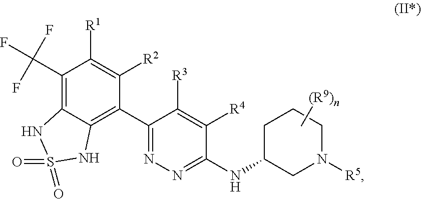

or a pharmaceutically acceptable salt, stereoisomer, solvate, prodrug, isotopic derivative, or tautomer thereof.

In some embodiments, the compound is of Formula (II**):

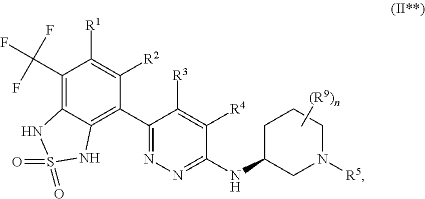

or a pharmaceutically acceptable salt, stereoisomer, solvate, prodrug, isotopic derivative, or tautomer thereof.

In some embodiments, the present disclosure provides compounds of Formula (III) and salts, stereoisomers, solvates, prodrugs, isotopic derivatives, and tautomers thereof:

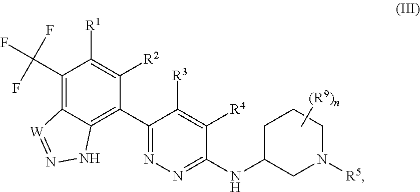

wherein R¹, R², R³, R⁴, R⁵, R⁹, W and n are as described herein.

It is understood that, for a compound of Formula (III), R¹, R², R³, R⁴, R⁵, R⁹, W and n can each be, where applicable, selected from the groups described herein, and any group described herein for any R¹, R², R³, R⁴, R⁵, R⁹, W and n can be combined, where applicable, with any group described herein for one or more of the remainders of R¹, R², R³, R⁴, R⁵, R⁹, W and n.

In some embodiments, the compound is of Formula (III*):

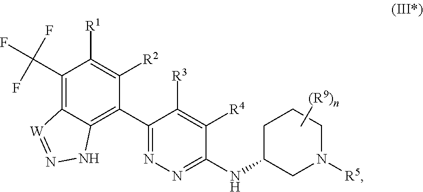

or a pharmaceutically acceptable salt, stereoisomer, solvate, prodrug, isotopic derivative, or tautomer thereof.

In some embodiments, the compound is of Formula (III**):

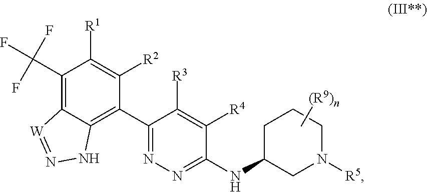

or a pharmaceutically acceptable salt, stereoisomer, solvate, prodrug, isotopic derivative, or tautomer thereof.

In some embodiments, the present disclosure provides compounds of Formula (IV) and salts, stereoisomers, solvates, prodrugs, isotopic derivatives, and tautomers thereof:

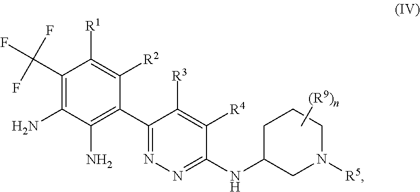

wherein R¹, R², R³, R⁴, R⁵, R⁹ and n are as described herein.

It is understood that, for a compound of Formula (IV), R¹, R², R³, R⁴, R⁵, R⁹ and n can each be, where applicable, selected from the groups described herein, and any group described herein for any R¹, R², R³, R⁴, R⁵, R⁹ and n can be combined, where applicable, with any group described herein for one or more of the remainders of R¹, R², R³, R⁴, R⁵, R⁹ and n.

In some embodiments, the compound is of Formula (IV*):

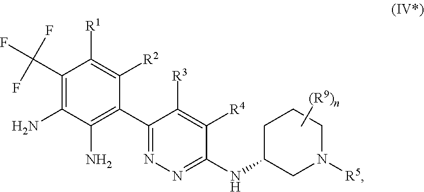

or a pharmaceutically acceptable salt, stereoisomer, solvate, prodrug, isotopic derivative, or tautomer thereof.

In some embodiments, the compound is of Formula (IV**):

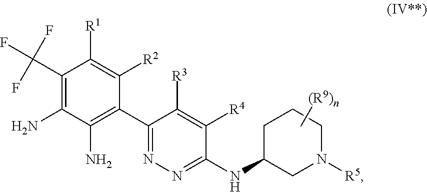

or a pharmaceutically acceptable salt, stereoisomer, solvate, prodrug, isotopic derivative, or tautomer thereof.

In some embodiments, the present disclosure provides compounds of Formula (V) and salts, stereoisomers, solvates, prodrugs, isotopic derivatives, and tautomers thereof:

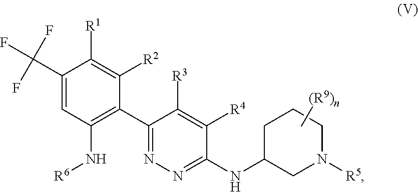

wherein R¹, R², R³, R⁴, R⁵, R⁶, R⁹ and n are as described herein.

It is understood that, for a compound of Formula (V), R¹, R², R³, R⁴, R⁵, R⁶, R⁹ and n can each be, where applicable, selected from the groups described herein, and any group described herein for any R¹, R², R³, R⁴, R⁵, R⁶, R⁹ and n can be combined, where applicable, with any group described herein for one or more of the remainders of R¹, R², R³, R⁴, R⁵, R⁶, R⁹ and n.

In some embodiments, the compound is of Formula (V*):

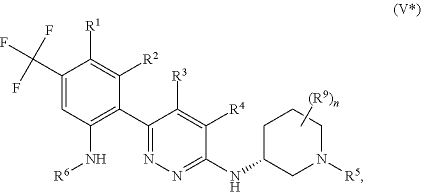

or a pharmaceutically acceptable salt, stereoisomer, solvate, prodrug, isotopic derivative, or tautomer thereof.

In some embodiments, the compound is of Formula (V**):

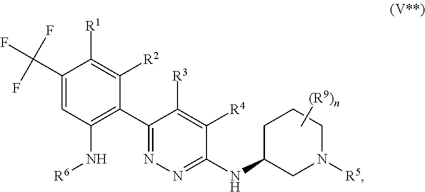

or a pharmaceutically acceptable salt, stereoisomer, solvate, prodrug, isotopic derivative, or tautomer thereof.

In some embodiments, the compound of Formula (A) is the compound of Formula (I) or a pharmaceutically acceptable salt, stereoisomer, solvate, prodrug, isotopic derivative, or tautomer thereof.

In some embodiments, the compound of Formula (A) is the compound of Formula (II) or a pharmaceutically acceptable salt, stereoisomer, solvate, prodrug, isotopic derivative, or tautomer thereof.

In some embodiments, the compound of Formula (A) is the compound of Formula (III) or a pharmaceutically acceptable salt, stereoisomer, solvate, prodrug, isotopic derivative, or tautomer thereof.

In some embodiments, the compound of Formula (A) is the compound of Formula (IV) or a pharmaceutically acceptable salt, stereoisomer, solvate, prodrug, isotopic derivative, or tautomer thereof.

In some embodiments, the compound of Formula (A) is the compound of Formula (V) or a pharmaceutically acceptable salt, stereoisomer, solvate, prodrug, isotopic derivative, or tautomer thereof.

In some embodiments, the compound is of Formula (I-A):

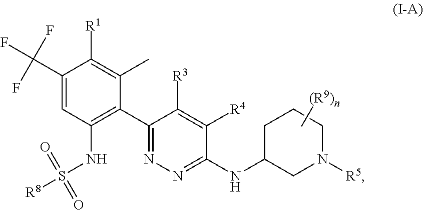

or a pharmaceutically acceptable salt, stereoisomer, solvate, prodrug, isotopic derivative, or tautomer thereof.

In some embodiments, the compound is of Formula (I-A-1):

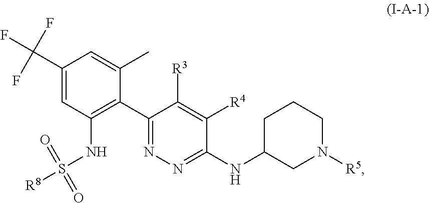

or a pharmaceutically acceptable salt, stereoisomer, solvate, prodrug, isotopic derivative, or tautomer thereof.

In some embodiments, the compound is of Formula (I-A-1-I):

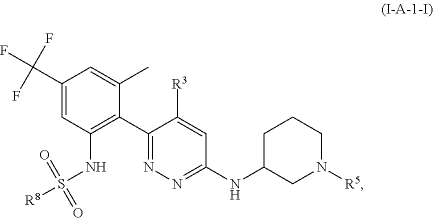

or a pharmaceutically acceptable salt, stereoisomer, solvate, prodrug, isotopic derivative, or tautomer thereof.

In some embodiments, the compound is of Formula (I-A-1-I-a):

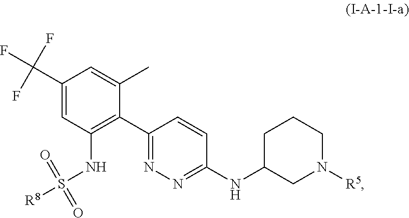

or a pharmaceutically acceptable salt, stereoisomer, solvate, prodrug, isotopic derivative, or tautomer thereof.

In some embodiments, the compound is of Formula (I-A-1-I-a*):

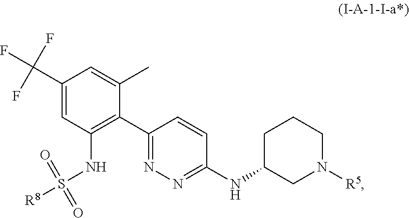

or a pharmaceutically acceptable salt, stereoisomer, solvate, prodrug, isotopic derivative, or tautomer thereof.

In some embodiments, the compound is of Formula (I-B):

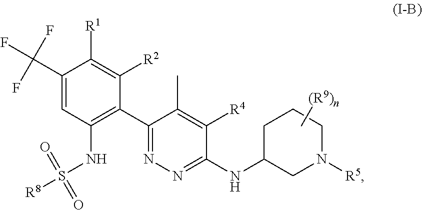

or a pharmaceutically acceptable salt, stereoisomer, solvate, prodrug, isotopic derivative, or tautomer thereof.

In some embodiments, the compound is of Formula (I-B-1):

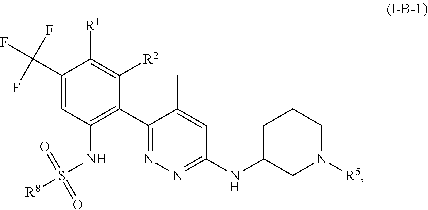

or a pharmaceutically acceptable salt, stereoisomer, solvate, prodrug, isotopic derivative, or tautomer thereof.

In some embodiments, the compound is of Formula (I-B-1-I):

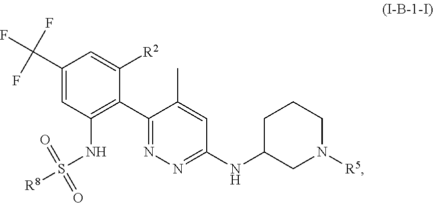

or a pharmaceutically acceptable salt, stereoisomer, solvate, prodrug, isotopic derivative, or tautomer thereof.

In some embodiments, the compound is of Formula (I-B-1-I-a):

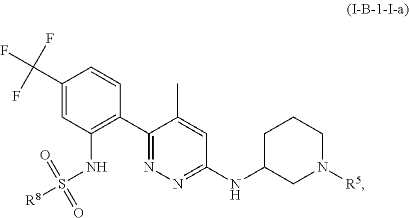

or a pharmaceutically acceptable salt, stereoisomer, solvate, prodrug, isotopic derivative, or tautomer thereof.

In some embodiments, the compound is of Formula (I-B-1-I-a*):

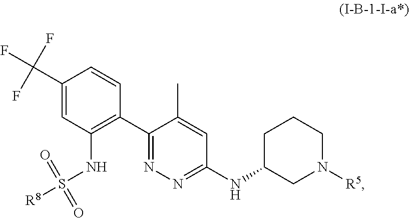

or a pharmaceutically acceptable salt, stereoisomer, solvate, prodrug, isotopic derivative, or tautomer thereof.

In some embodiments, the compound is of Formula (I-B-2):

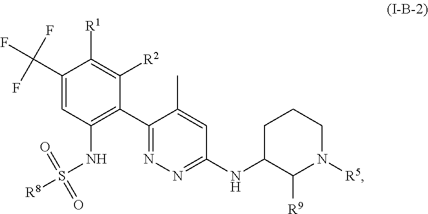

or a pharmaceutically acceptable salt, stereoisomer, solvate, prodrug, isotopic derivative, or tautomer thereof.

In some embodiments, the compound is of Formula (I-B-2-I):

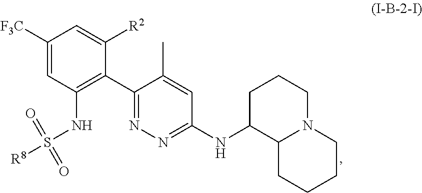

or a pharmaceutically acceptable salt, stereoisomer, solvate, prodrug, isotopic derivative, or tautomer thereof.

In some embodiments, the compound is of Formula (I-B-2-I-a):

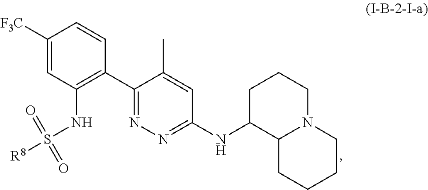

or a pharmaceutically acceptable salt, stereoisomer, solvate, prodrug, isotopic derivative, or tautomer thereof.

In some embodiments, the compound is of Formula (I-B-2-I-a*):

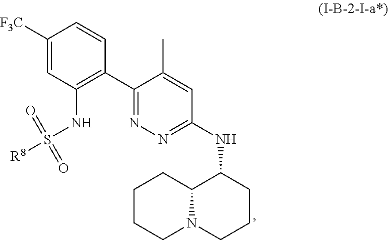

or a pharmaceutically acceptable salt, stereoisomer, solvate, prodrug, isotopic derivative, or tautomer thereof.

In some embodiments, the compound is of Formula (I-B-3):

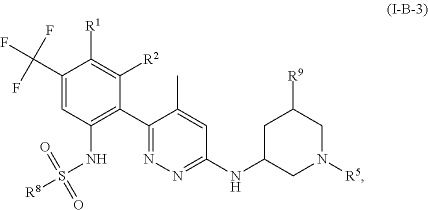

or a pharmaceutically acceptable salt, stereoisomer, solvate, prodrug, isotopic derivative, or tautomer thereof.

In some embodiments, the compound is of Formula (I-B-3-I):

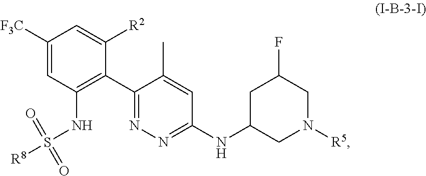

or a pharmaceutically acceptable salt, stereoisomer, solvate, prodrug, isotopic derivative, or tautomer thereof.

In some embodiments, the compound is of Formula (I-B-3-I-a):

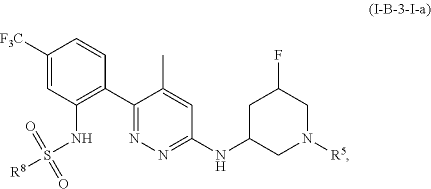

or a pharmaceutically acceptable salt, stereoisomer, solvate, prodrug, isotopic derivative, or tautomer thereof.

In some embodiments, the compound is of Formula (I-B-3-I-a*):

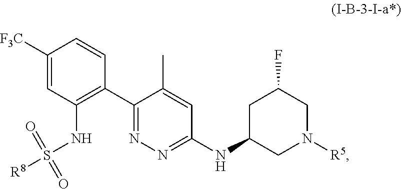

or a pharmaceutically acceptable salt, stereoisomer, solvate, prodrug, isotopic derivative, or tautomer thereof.

In some embodiments, the compound is of Formula (I-B-4):

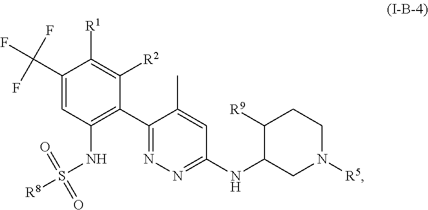

or a pharmaceutically acceptable salt, stereoisomer, solvate, prodrug, isotopic derivative, or tautomer thereof.

In some embodiments, the compound is of Formula (I-B-4-I):

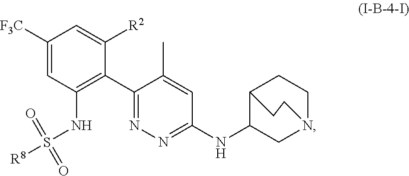

or a pharmaceutically acceptable salt, stereoisomer, solvate, prodrug, isotopic derivative, or tautomer thereof.

In some embodiments, the compound is of Formula (I-B-4-I-a):

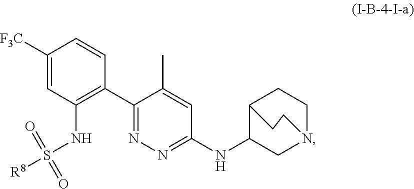

or a pharmaceutically acceptable salt, stereoisomer, solvate, prodrug, isotopic derivative, or tautomer thereof.

In some embodiments, the compound is of Formula (I-B-4-I-a*):

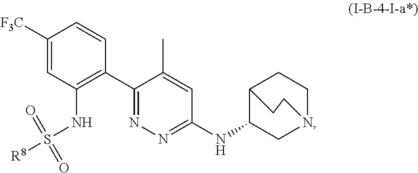

or a pharmaceutically acceptable salt, stereoisomer, solvate, prodrug, isotopic derivative, or tautomer thereof.

In some embodiments, the compound is of Formula (I-C):

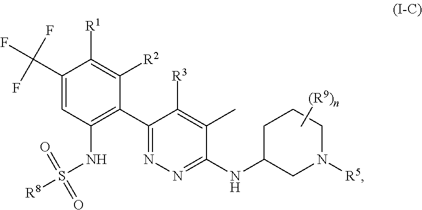

or a pharmaceutically acceptable salt, stereoisomer, solvate, prodrug, isotopic derivative, or tautomer thereof.

In some embodiments, the compound is of Formula (I-C-1):

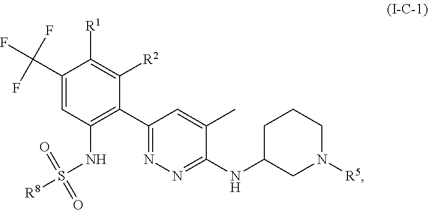

or a pharmaceutically acceptable salt, stereoisomer, solvate, prodrug, isotopic derivative, or tautomer thereof.

In some embodiments, the compound is of Formula (I-C-1-I):

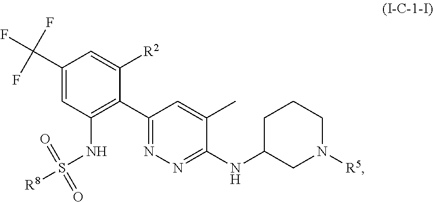

or a pharmaceutically acceptable salt, stereoisomer, solvate, prodrug, isotopic derivative, or tautomer thereof.

In some embodiments, the compound is of Formula (I-C-1-I-a):

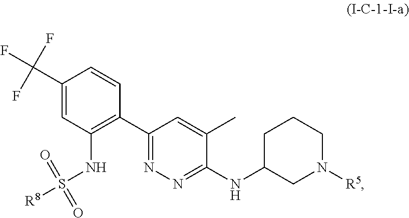

or a pharmaceutically acceptable salt, stereoisomer, solvate, prodrug, isotopic derivative, or tautomer thereof.

In some embodiments, the compound is of Formula (I-C-1-I-a*):

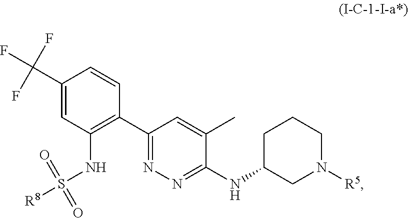

or a pharmaceutically acceptable salt, stereoisomer, solvate, prodrug, isotopic derivative, or tautomer thereof.

In some embodiments, the compound is of Formula (I-C-2):

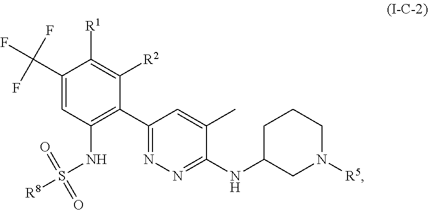

or a pharmaceutically acceptable salt, stereoisomer, solvate, prodrug, isotopic derivative, or tautomer thereof.

In some embodiments, the compound is of Formula (I-C-2-I):

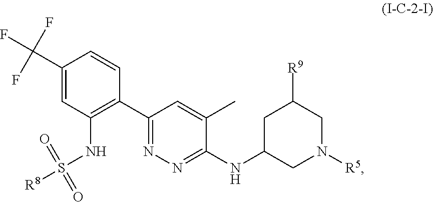

or a pharmaceutically acceptable salt, stereoisomer, solvate, prodrug, isotopic derivative, or tautomer thereof.

In some embodiments, the compound is of Formula (I-C-2-I-a):

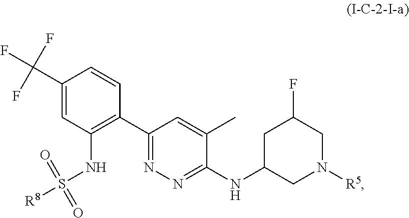

or a pharmaceutically acceptable salt, stereoisomer, solvate, prodrug, isotopic derivative, or tautomer thereof.

In some embodiments, the compound is of Formula (I-C-2-I-a*):

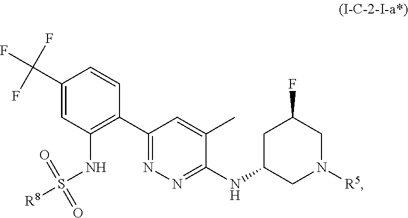

or a pharmaceutically acceptable salt, stereoisomer, solvate, prodrug, isotopic derivative, or tautomer thereof.

In some embodiments, the compound is of Formula (I-C-2-I-a**):

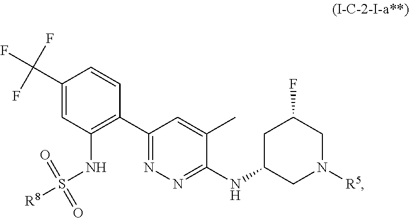

or a pharmaceutically acceptable salt, stereoisomer, solvate, prodrug, isotopic derivative, or tautomer thereof.

In some embodiments, the compound is of Formula (I-C-2-I-b):

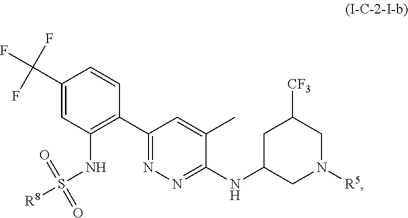

or a pharmaceutically acceptable salt, stereoisomer, solvate, prodrug, isotopic derivative, or tautomer thereof.

In some embodiments, the compound is of Formula (I-C-2-I-b*):

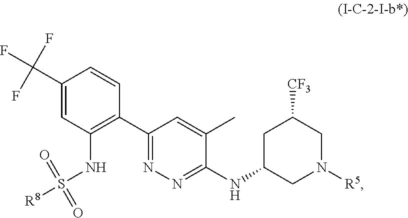

or a pharmaceutically acceptable salt, stereoisomer, solvate, prodrug, isotopic derivative, or tautomer thereof.

In some embodiments, the compound is of Formula (I-C-2-I-b**):

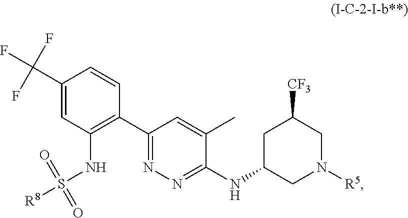

or a pharmaceutically acceptable salt, stereoisomer, solvate, prodrug, isotopic derivative, or tautomer thereof.

In some embodiments, the compound is of Formula (I-C-2-I-c):

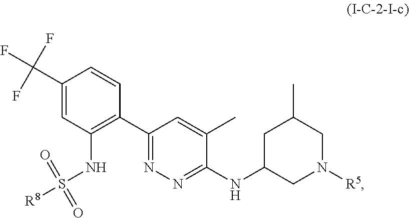

or a pharmaceutically acceptable salt, stereoisomer, solvate, prodrug, isotopic derivative, or tautomer thereof.

In some embodiments, the compound is of Formula (I-C-2-I-c*):

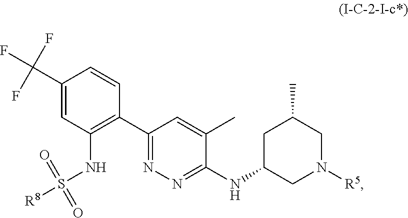

or a pharmaceutically acceptable salt, stereoisomer, solvate, prodrug, isotopic derivative, or tautomer thereof.

In some embodiments, the compound is of Formula (I-C-2-I-c**):

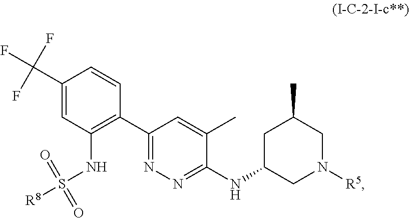

or a pharmaceutically acceptable salt, stereoisomer, solvate, prodrug, isotopic derivative, or tautomer thereof.

In some embodiments, the compound is of Formula (I-C-3):

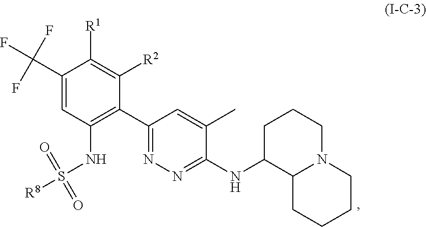

or a pharmaceutically acceptable salt, stereoisomer, solvate, prodrug, isotopic derivative, or tautomer thereof.

In some embodiments, the compound is of Formula (I-C-3-I):

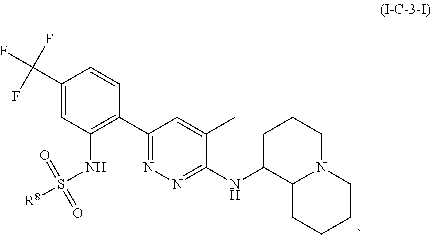

or a pharmaceutically acceptable salt, stereoisomer, solvate, prodrug, isotopic derivative, or tautomer thereof.

In some embodiments, the compound is of Formula (I-C-3-I*):

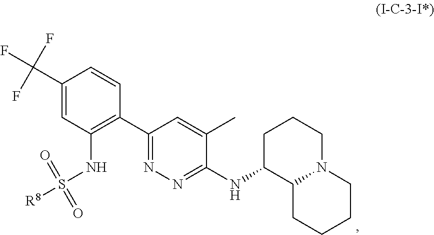

or a pharmaceutically acceptable salt, stereoisomer, solvate, prodrug, isotopic derivative, or tautomer thereof.

In some embodiments, the compound is of Formula (I-C-4):

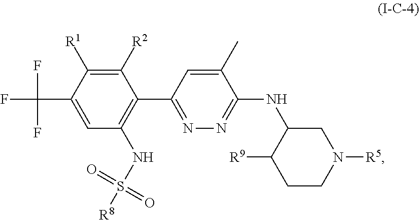

or a pharmaceutically acceptable salt, stereoisomer, solvate, prodrug, isotopic derivative, or tautomer thereof.

In some embodiments, the compound is of Formula (I-C-4-I):

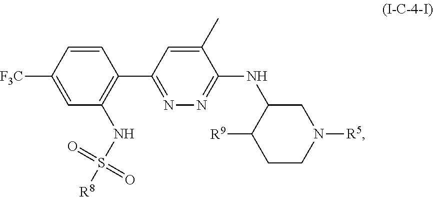

or a pharmaceutically acceptable salt, stereoisomer, solvate, prodrug, isotopic derivative, or tautomer thereof.

In some embodiments, the compound is of Formula (I-C-4-I-a):

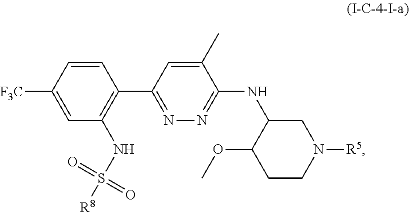

or a pharmaceutically acceptable salt, stereoisomer, solvate, prodrug, isotopic derivative, or tautomer thereof.

In some embodiments, the compound is of Formula (I-C-4-I-a*):

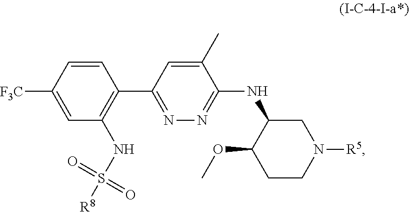

or a pharmaceutically acceptable salt, stereoisomer, solvate, prodrug, isotopic derivative, or tautomer thereof.

In some embodiments, the compound is of Formula (I-C-4-I-b):

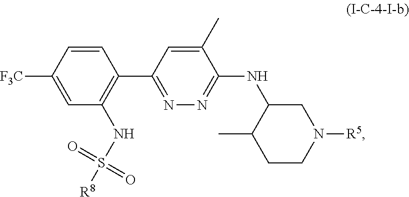

or a pharmaceutically acceptable salt, stereoisomer, solvate, prodrug, isotopic derivative, or tautomer thereof.

In some embodiments, the compound is of Formula (I-C-4-I-b*):

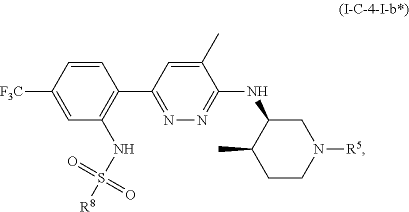

or a pharmaceutically acceptable salt, stereoisomer, solvate, prodrug, isotopic derivative, or tautomer thereof.

In some embodiments, the compound is of Formula (I-C-4-I-b**):

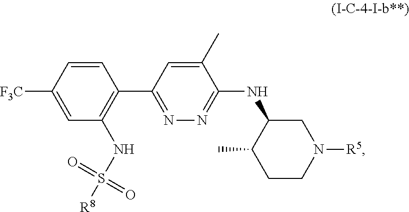

or a pharmaceutically acceptable salt, stereoisomer, solvate, prodrug, isotopic derivative, or tautomer thereof.

In some embodiments, the compound is of Formula (I-C-4-I-c):

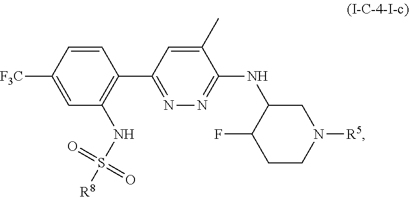

or a pharmaceutically acceptable salt, stereoisomer, solvate, prodrug, isotopic derivative, or tautomer thereof.

In some embodiments, the compound is of Formula (I-C-4-I-c*):

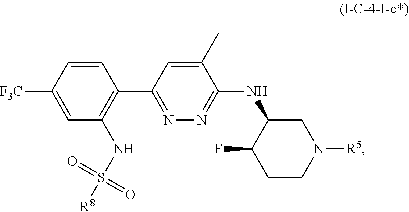

or a pharmaceutically acceptable salt, stereoisomer, solvate, prodrug, isotopic derivative, or tautomer thereof.

In some embodiments, the compound is of Formula (I-C-4-I-c**):

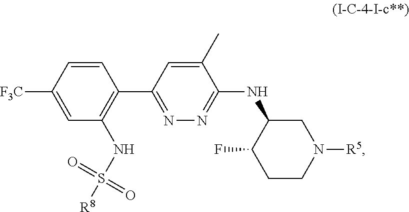

or a pharmaceutically acceptable salt, stereoisomer, solvate, prodrug, isotopic derivative, or tautomer thereof.

In some embodiments, the compound is of Formula (I-C-4-I-d):

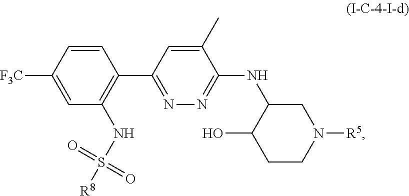

or a pharmaceutically acceptable salt, stereoisomer, solvate, prodrug, isotopic derivative, or tautomer thereof.

In some embodiments, the compound is of Formula (I-C-4-I-d*):

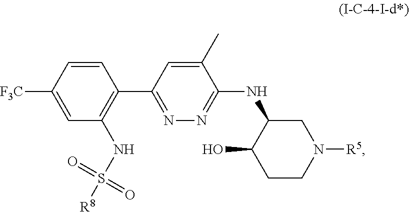

or a pharmaceutically acceptable salt, stereoisomer, solvate, prodrug, isotopic derivative, or tautomer thereof.

In some embodiments, the compound is of Formula (I-C-4-I-d**):

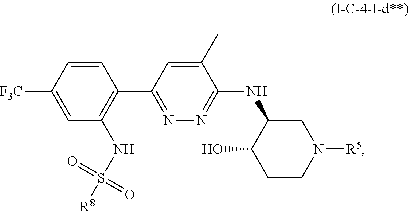

or a pharmaceutically acceptable salt, stereoisomer, solvate, prodrug, isotopic derivative, or tautomer thereof.

In some embodiments, the compound is of Formula (I-C-4-I-e):

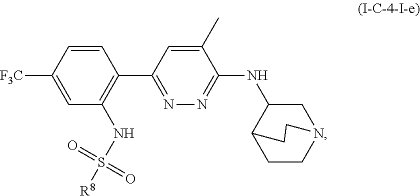

or a pharmaceutically acceptable salt, stereoisomer, solvate, prodrug, isotopic derivative, or tautomer thereof.

In some embodiments, the compound is of Formula (I-C-4-I-e*):

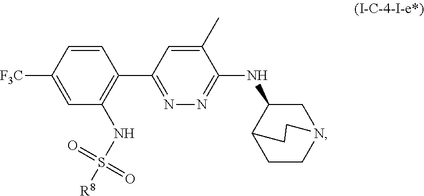

or a pharmaceutically acceptable salt, stereoisomer, solvate, prodrug, isotopic derivative, or tautomer thereof.

In some embodiments, the compound is of Formula (I-D):

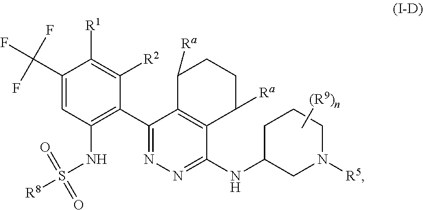

or a pharmaceutically acceptable salt, stereoisomer, solvate, prodrug, isotopic derivative, or tautomer thereof, wherein each R^{a} is H, or two R^{a} together form alkanediyl-(CH₂)ₘ-bridge, wherein m is selected from 1 and 2.

In some embodiments, the compound is of Formula (I-D-1):

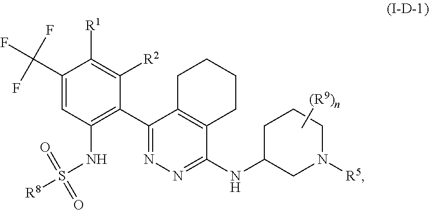

or a pharmaceutically acceptable salt, stereoisomer, solvate, prodrug, isotopic derivative, or tautomer thereof.

In some embodiments, the compound is of Formula (I-D-1-I):

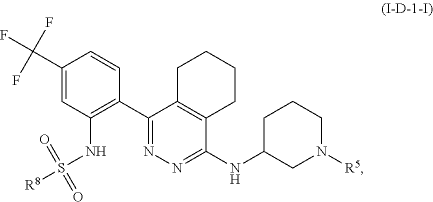

or a pharmaceutically acceptable salt, stereoisomer, solvate, prodrug, isotopic derivative, or tautomer thereof.

In some embodiments, the compound is of Formula (I-D-1-I*):

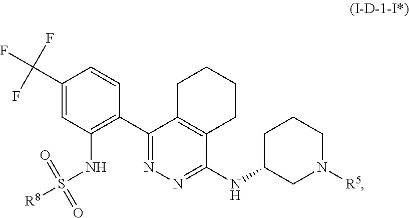

or a pharmaceutically acceptable salt, stereoisomer, solvate, prodrug, isotopic derivative, or tautomer thereof.

In some embodiments, the compound is of Formula (I-D-2):

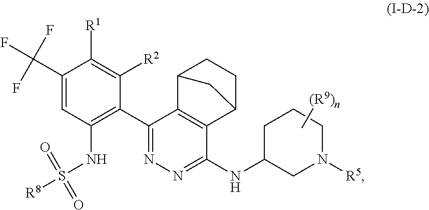

or a pharmaceutically acceptable salt, stereoisomer, solvate, prodrug, isotopic derivative, or tautomer thereof.

In some embodiments, the compound is of Formula (I-D-2-I):

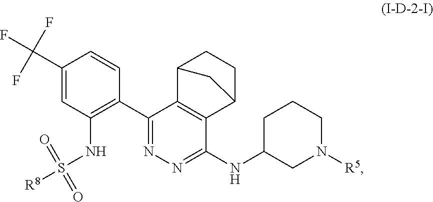

or a pharmaceutically acceptable salt, stereoisomer, solvate, prodrug, isotopic derivative, or tautomer thereof.

In some embodiments, the compound is of Formula (I-D-2-I*):

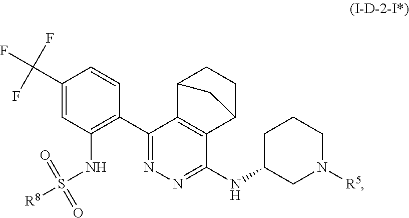

or a pharmaceutically acceptable salt, stereoisomer, solvate, prodrug, isotopic derivative, or tautomer thereof.

In some embodiments, the compound is of Formula (I-D-3):

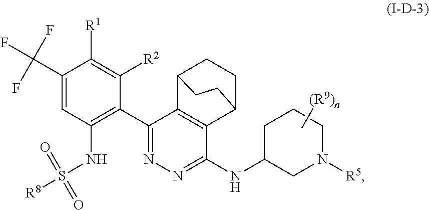

or a pharmaceutically acceptable salt, stereoisomer, solvate, prodrug, isotopic derivative, or tautomer thereof.

In some embodiments, the compound is of Formula (I-D-3-I):

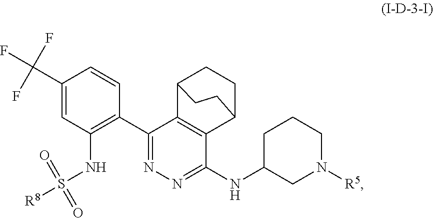

or a pharmaceutically acceptable salt, stereoisomer, solvate, prodrug, isotopic derivative, or tautomer thereof.

In some embodiments, the compound is of Formula (I-D-3-1*):

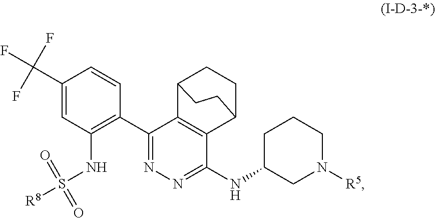

or a pharmaceutically acceptable salt, stereoisomer, solvate, prodrug, isotopic derivative, or tautomer thereof.

In some embodiments, the compound is of Formula (I-E):

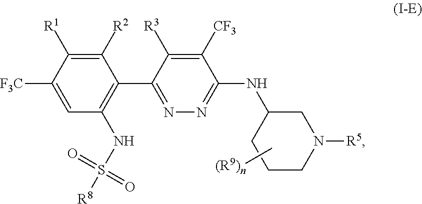

or a pharmaceutically acceptable salt, stereoisomer, solvate, prodrug, isotopic derivative, or tautomer thereof.

In some embodiments, the compound is of Formula (I-E-1):

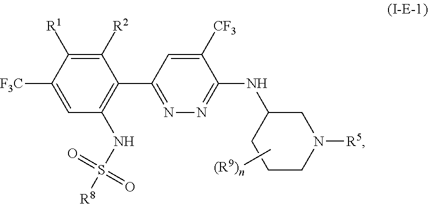

or a pharmaceutically acceptable salt, stereoisomer, solvate, prodrug, isotopic derivative, or tautomer thereof.

In some embodiments, the compound is of Formula (I-E-1-I):

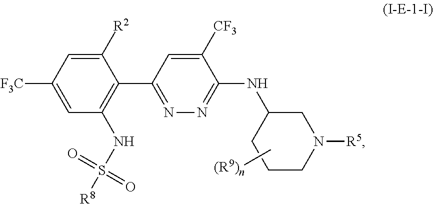

or a pharmaceutically acceptable salt, stereoisomer, solvate, prodrug, isotopic derivative, or tautomer thereof.

In some embodiments, the compound is of Formula (I-E-1-I-a):

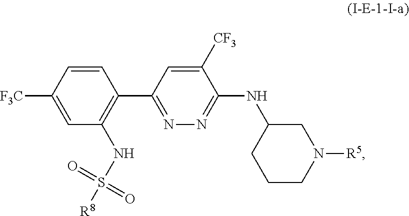

or a pharmaceutically acceptable salt, stereoisomer, solvate, prodrug, isotopic derivative, or tautomer thereof.

In some embodiments, the compound is of Formula (I-E-1-I-a*):

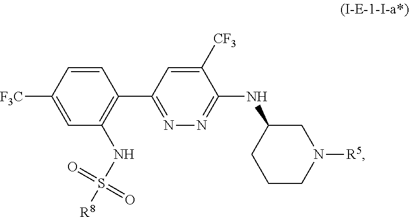

or a pharmaceutically acceptable salt, stereoisomer, solvate, prodrug, isotopic derivative, or tautomer thereof.

In some embodiments, the compound is of Formula (I-F):

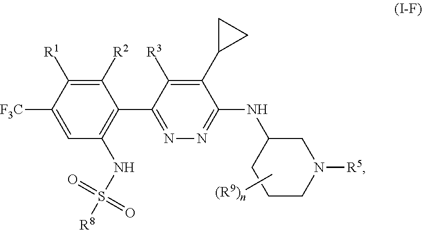

or a pharmaceutically acceptable salt, stereoisomer, solvate, prodrug, isotopic derivative, or tautomer thereof.

In some embodiments, the compound is of Formula (I-F-1):

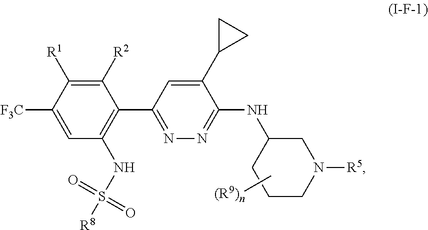

or a pharmaceutically acceptable salt, stereoisomer, solvate, prodrug, isotopic derivative, or tautomer thereof.

In some embodiments, the compound is of Formula (I-F-1-I):

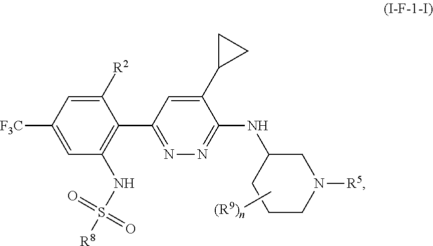

or a pharmaceutically acceptable salt, stereoisomer, solvate, prodrug, isotopic derivative, or tautomer thereof.

In some embodiments, the compound is of Formula (I-F-1-I-a):

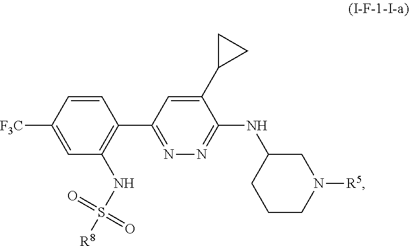

or a pharmaceutically acceptable salt, stereoisomer, solvate, prodrug, isotopic derivative, or tautomer thereof.

In some embodiments, the compound is of Formula (I-F-1-I-a*):

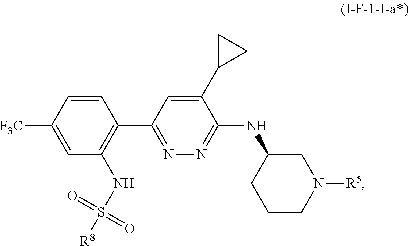

or a pharmaceutically acceptable salt, stereoisomer, solvate, prodrug, isotopic derivative, or tautomer thereof.

In some embodiments, the compound is of Formula (I-G):

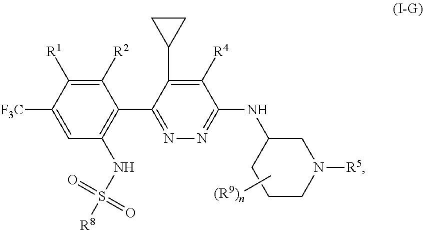

or a pharmaceutically acceptable salt, stereoisomer, solvate, prodrug, isotopic derivative, or tautomer thereof.

In some embodiments, the compound is of Formula (I-G-1):

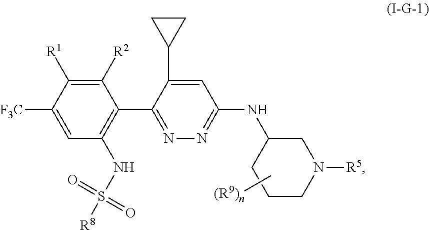

or a pharmaceutically acceptable salt, stereoisomer, solvate, prodrug, isotopic derivative, or tautomer thereof.

In some embodiments, the compound is of Formula (I-G-1-I):

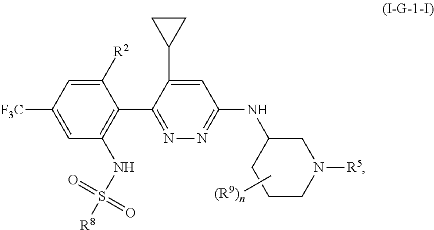

or a pharmaceutically acceptable salt, stereoisomer, solvate, prodrug, isotopic derivative, or tautomer thereof.

In some embodiments, the compound is of Formula (I-G-1-I-a):

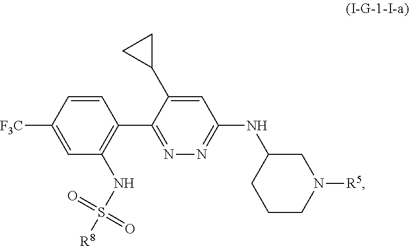

or a pharmaceutically acceptable salt, stereoisomer, solvate, prodrug, isotopic derivative, or tautomer thereof.

In some embodiments, the compound is of Formula (I-G-1-I-a*):

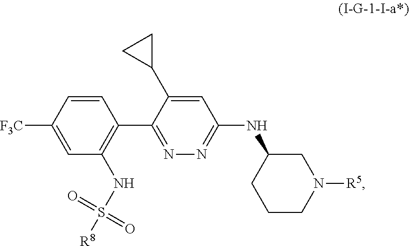

or a pharmaceutically acceptable salt, stereoisomer, solvate, prodrug, isotopic derivative, or tautomer thereof.

In some embodiments, the compound is of Formula (I-H):

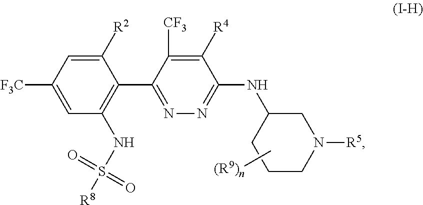

or a pharmaceutically acceptable salt, stereoisomer, solvate, prodrug, isotopic derivative, or tautomer thereof.

In some embodiments, the compound is of Formula (I-H-1):

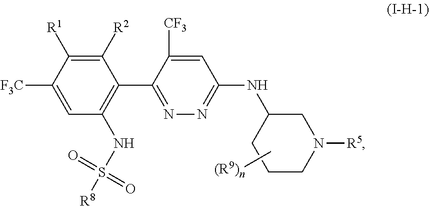

or a pharmaceutically acceptable salt, stereoisomer, solvate, prodrug, isotopic derivative, or tautomer thereof.

In some embodiments, the compound is of Formula (I-H-1-I):

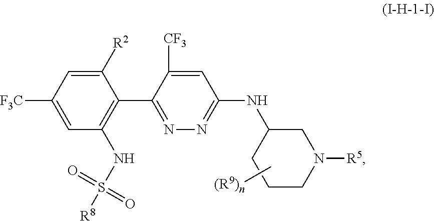

or a pharmaceutically acceptable salt, stereoisomer, solvate, prodrug, isotopic derivative, or tautomer thereof.

In some embodiments, the compound is of Formula (I-H-1-I-a):

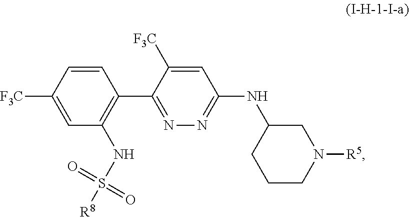

or a pharmaceutically acceptable salt, stereoisomer, solvate, prodrug, isotopic derivative, or tautomer thereof.

In some embodiments, the compound is of Formula (I-H-1-I-a*):

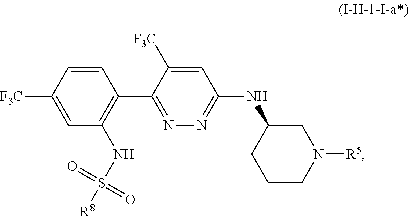

or a pharmaceutically acceptable salt, stereoisomer, solvate, prodrug, isotopic derivative, or tautomer thereof.

In some embodiments, the compound is of Formula (II-A):

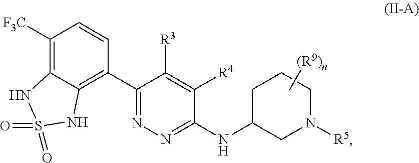

or a pharmaceutically acceptable salt, stereoisomer, solvate, prodrug, isotopic derivative, or tautomer thereof.

In some embodiments, the compound is of Formula (II-A-1):

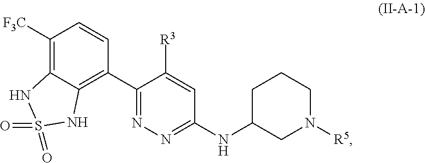

or a pharmaceutically acceptable salt, stereoisomer, solvate, prodrug, isotopic derivative, or tautomer thereof.

In some embodiments, the compound is of Formula (II-A-1-I):

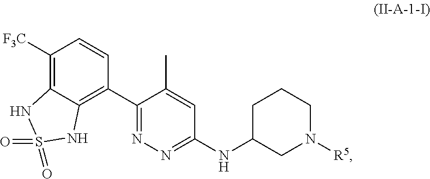

or a pharmaceutically acceptable salt, stereoisomer, solvate, prodrug, isotopic derivative, or tautomer thereof.

In some embodiments, the compound is of Formula (II-A-1-I*):

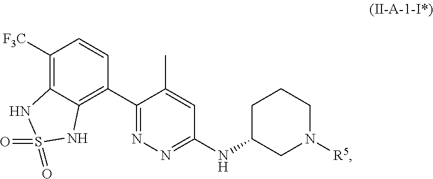

or a pharmaceutically acceptable salt, stereoisomer, solvate, prodrug, isotopic derivative, or tautomer thereof.

In some embodiments, the compound is of Formula (II-A-1-II):

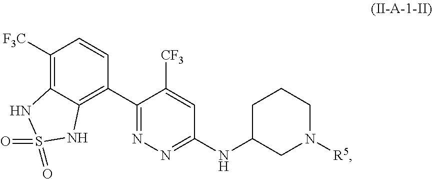

or a pharmaceutically acceptable salt, stereoisomer, solvate, prodrug, isotopic derivative, or tautomer thereof.

In some embodiments, the compound is of Formula (II-A-1-II*):

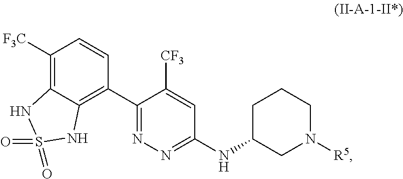

or a pharmaceutically acceptable salt, stereoisomer, solvate, prodrug, isotopic derivative, or tautomer thereof.

In some embodiments, the compound is of Formula (II-A-2):

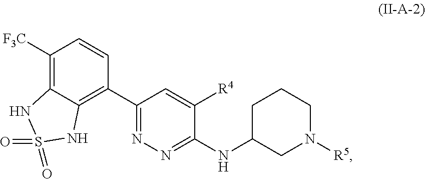

or a pharmaceutically acceptable salt, stereoisomer, solvate, prodrug, isotopic derivative, or tautomer thereof.

In some embodiments, the compound is of Formula (II-A-2-I):

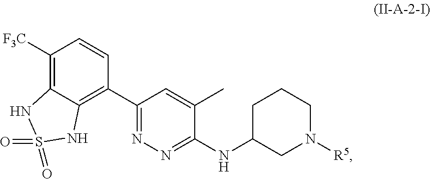

or a pharmaceutically acceptable salt, stereoisomer, solvate, prodrug, isotopic derivative, or tautomer thereof.

In some embodiments, the compound is of Formula (II-A-2-I*):

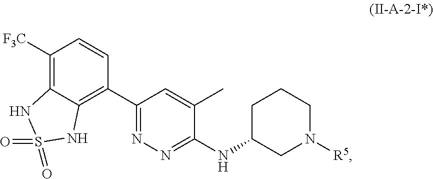

or a pharmaceutically acceptable salt, stereoisomer, solvate, prodrug, isotopic derivative, or tautomer thereof.

In some embodiments, the compound is of Formula (II-A-2-II):

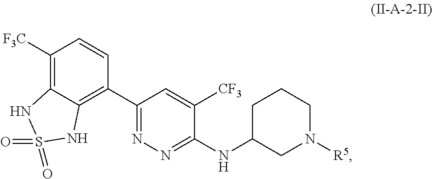

or a pharmaceutically acceptable salt, stereoisomer, solvate, prodrug, isotopic derivative, or tautomer thereof.

In some embodiments, the compound is of Formula (II-A-2-II*):

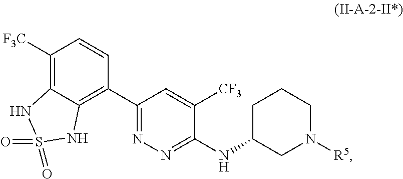

or a pharmaceutically acceptable salt, stereoisomer, solvate, prodrug, isotopic derivative, or tautomer thereof.

In some embodiments, the compound is of Formula (II-A-3):

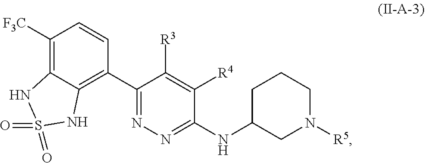

or a pharmaceutically acceptable salt, stereoisomer, solvate, prodrug, isotopic derivative, or tautomer thereof.

In some embodiments, the compound is of Formula (II-A-3-I):

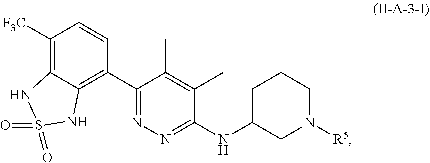

or a pharmaceutically acceptable salt, stereoisomer, solvate, prodrug, isotopic derivative, or tautomer thereof.

In some embodiments, the compound is of Formula (II-A-3-1*):

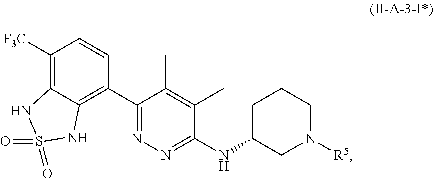

or a pharmaceutically acceptable salt, stereoisomer, solvate, prodrug, isotopic derivative, or tautomer thereof.

In some embodiments, the compound is of Formula (III-A):

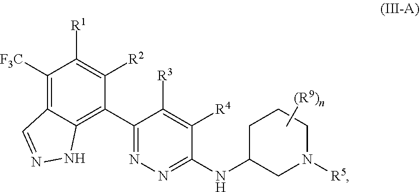

or a pharmaceutically acceptable salt, stereoisomer, solvate, prodrug, isotopic derivative, or tautomer thereof.

In some embodiments, the compound is of Formula (III-A-1):

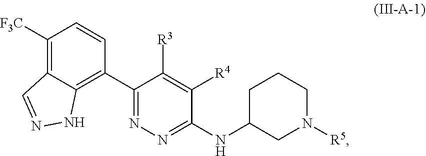

or a pharmaceutically acceptable salt, stereoisomer, solvate, prodrug, isotopic derivative, or tautomer thereof.

In some embodiments, the compound is of Formula (III-A-1-a):

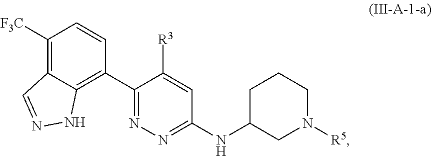

or a pharmaceutically acceptable salt, stereoisomer, solvate, prodrug, isotopic derivative, or tautomer thereof.

In some embodiments, the compound is of Formula (III-A-1-a-I):

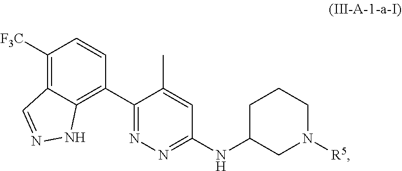

or a pharmaceutically acceptable salt, stereoisomer, solvate, prodrug, isotopic derivative, or tautomer thereof.

In some embodiments, the compound is of Formula (III-A-1-a-I*):

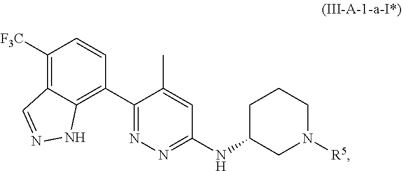

or a pharmaceutically acceptable salt, stereoisomer, solvate, prodrug, isotopic derivative, or tautomer thereof.

In some embodiments, the compound is of Formula (III-A-1-b):

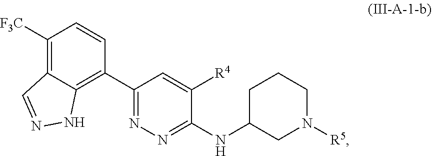

or a pharmaceutically acceptable salt, stereoisomer, solvate, prodrug, isotopic derivative, or tautomer thereof.

In some embodiments, the compound is of Formula (III-A-1-b-I):

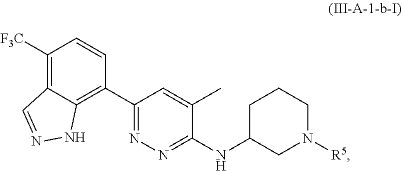

or a pharmaceutically acceptable salt, stereoisomer, solvate, prodrug, isotopic derivative, or tautomer thereof.

In some embodiments, the compound is of Formula (III-A-1-b-I*):

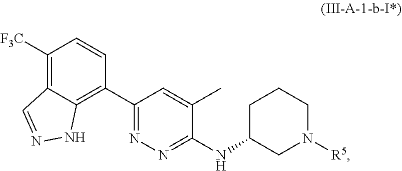

or a pharmaceutically acceptable salt, stereoisomer, solvate, prodrug, isotopic derivative, or tautomer thereof.

In some embodiments, the compound is of Formula (III-B):

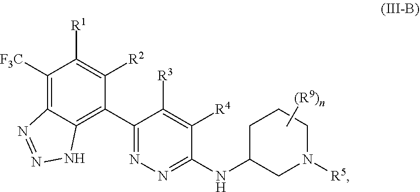

or a pharmaceutically acceptable salt, stereoisomer, solvate, prodrug, isotopic derivative, or tautomer thereof.

In some embodiments, the compound is of Formula (III-B-1):

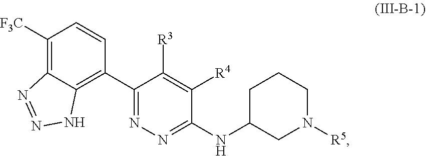

or a pharmaceutically acceptable salt, stereoisomer, solvate, prodrug, isotopic derivative, or tautomer thereof.

In some embodiments, the compound is of Formula (III-B-1-I):

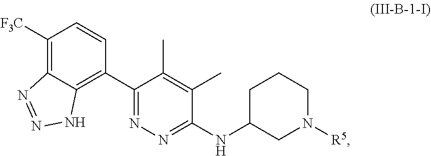

or a pharmaceutically acceptable salt, stereoisomer, solvate, prodrug, isotopic derivative, or tautomer thereof.

In some embodiments, the compound is of Formula (III-B-1-I*):

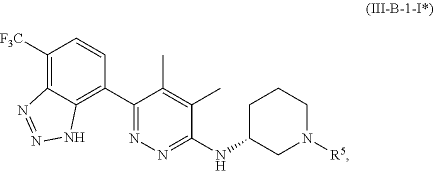

or a pharmaceutically acceptable salt, stereoisomer, solvate, prodrug, isotopic derivative, or tautomer thereof.

In some embodiments, the compound is of Formula (III-B-1-II):

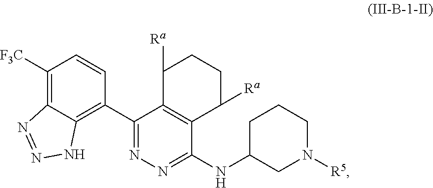

or a pharmaceutically acceptable salt, stereoisomer, solvate, prodrug, isotopic derivative, or tautomer thereof, wherein each R^{a} is H, or two R^{a} together form alkanediyl-(CH₂)ₘ-bridge, wherein m is selected from 1 and 2.

In some embodiments, the compound is of Formula (III-B-1-II-a):

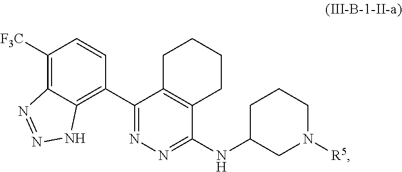

or a pharmaceutically acceptable salt, stereoisomer, solvate, prodrug, isotopic derivative, or tautomer thereof.

In some embodiments, the compound is of Formula (III-B-1-II-a*):

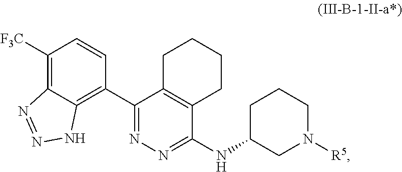

or a pharmaceutically acceptable salt, stereoisomer, solvate, prodrug, isotopic derivative, or tautomer thereof.

In some embodiments, the compound is of Formula (III-B-1-II-b):

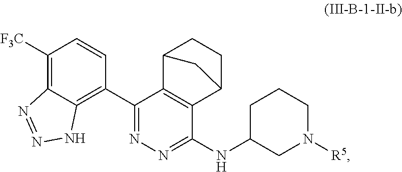

or a pharmaceutically acceptable salt, stereoisomer, solvate, prodrug, isotopic derivative, or tautomer thereof.

In some embodiments, the compound is of Formula (III-B-1-II-b*):

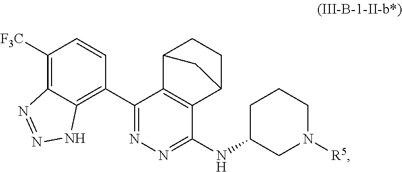

or a pharmaceutically acceptable salt, stereoisomer, solvate, prodrug, isotopic derivative, or tautomer thereof.

In some embodiments, the compound is of Formula (III-B-1-II-c):

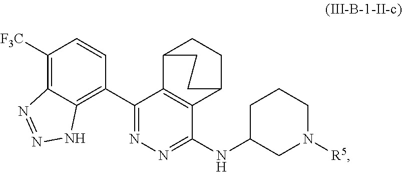

or a pharmaceutically acceptable salt, stereoisomer, solvate, prodrug, isotopic derivative, or tautomer thereof.

In some embodiments, the compound is of Formula (III-B-1-II-c*):

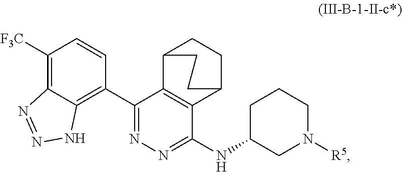

or a pharmaceutically acceptable salt, stereoisomer, solvate, prodrug, isotopic derivative, or tautomer thereof.

In some embodiments, the compound is of Formula (III-B-1-a):

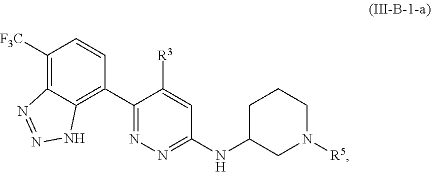

or a pharmaceutically acceptable salt, stereoisomer, solvate, prodrug, isotopic derivative, or tautomer thereof.

In some embodiments, the compound is of Formula (III-B-1-a-I):

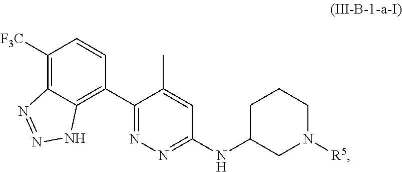

or a pharmaceutically acceptable salt, stereoisomer, solvate, prodrug, isotopic derivative, or tautomer thereof.

In some embodiments, the compound is of Formula (III-B-1-a-I*):

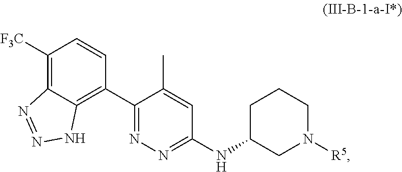

or a pharmaceutically acceptable salt, stereoisomer, solvate, prodrug, isotopic derivative, or tautomer thereof.

In some embodiments, the compound is of Formula (III-B-1-a-II):

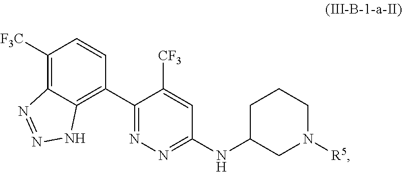

or a pharmaceutically acceptable salt, stereoisomer, solvate, prodrug, isotopic derivative, or tautomer thereof.

In some embodiments, the compound is of Formula (III-B-1-a-II*):

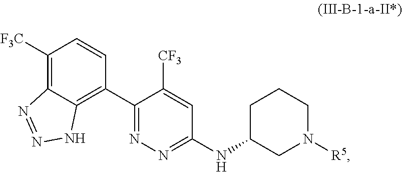

or a pharmaceutically acceptable salt, stereoisomer, solvate, prodrug, isotopic derivative, or tautomer thereof.

In some embodiments, the compound is of Formula (III-B-1-b):

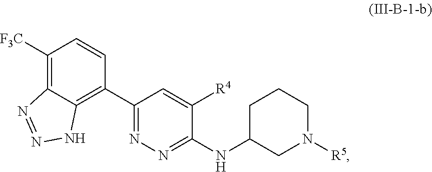

or a pharmaceutically acceptable salt, stereoisomer, solvate, prodrug, isotopic derivative, or tautomer thereof.

In some embodiments, the compound is of Formula (III-B-1-b-I):

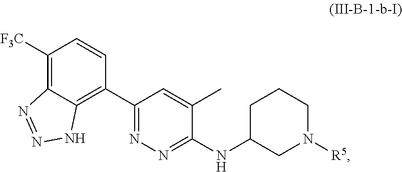

or a pharmaceutically acceptable salt, stereoisomer, solvate, prodrug, isotopic derivative, or tautomer thereof.

In some embodiments, the compound is of Formula (III-B-1-b-I*):

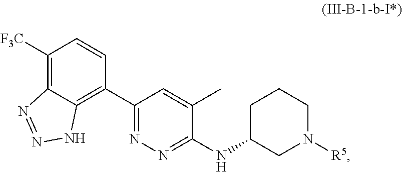

or a pharmaceutically acceptable salt, stereoisomer, solvate, prodrug, isotopic derivative, or tautomer thereof.

In some embodiments, the compound is of Formula (III-B-1-b-II):

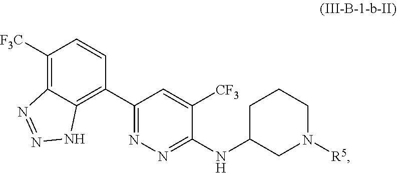

or a pharmaceutically acceptable salt, stereoisomer, solvate, prodrug, isotopic derivative, or tautomer thereof.

In some embodiments, the compound is of Formula (III-B-1-b-II*):

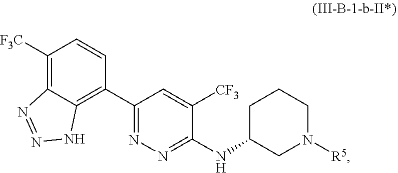

or a pharmaceutically acceptable salt, stereoisomer, solvate, prodrug, isotopic derivative, or tautomer thereof.

In some embodiments, the compound is of Formula (IV-A):

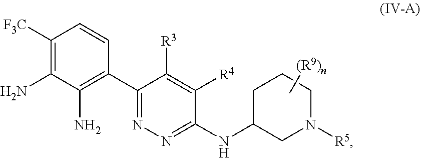

or a pharmaceutically acceptable salt, stereoisomer, solvate, prodrug, isotopic derivative, or tautomer thereof.

In some embodiments, the compound is of Formula (IV-A-1):

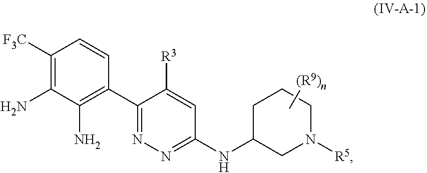

or a pharmaceutically acceptable salt, stereoisomer, solvate, prodrug, isotopic derivative, or tautomer thereof.

In some embodiments, the compound is of Formula (IV-A-1-a):

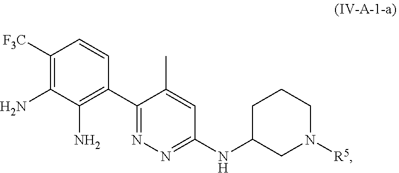

or a pharmaceutically acceptable salt, stereoisomer, solvate, prodrug, isotopic derivative, or tautomer thereof.

In some embodiments, the compound is of Formula (IV-A-1-a*):

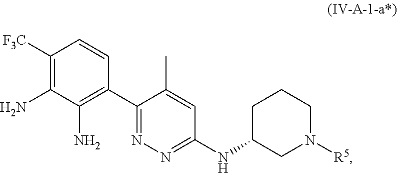

or a pharmaceutically acceptable salt, stereoisomer, solvate, prodrug, isotopic derivative, or tautomer thereof.

In some embodiments, the compound is of Formula (IV-A-1-b):

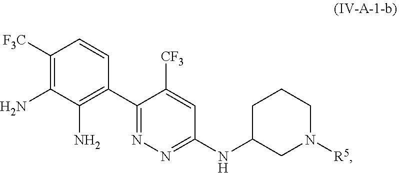

or a pharmaceutically acceptable salt, stereoisomer, solvate, prodrug, isotopic derivative, or tautomer thereof.

In some embodiments, the compound is of Formula (IV-A-1-b*):

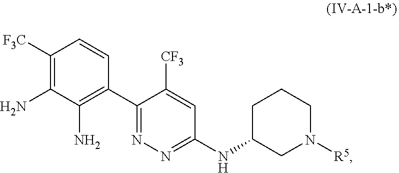

or a pharmaceutically acceptable salt, stereoisomer, solvate, prodrug, isotopic derivative, or tautomer thereof.

In some embodiments, the compound is of Formula (IV-A-2):

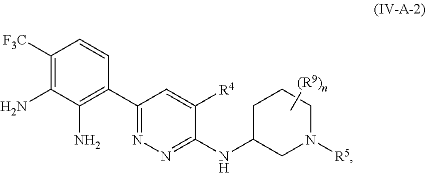

or a pharmaceutically acceptable salt, stereoisomer, solvate, prodrug, isotopic derivative, or tautomer thereof.

In some embodiments, the compound is of Formula (IV-A-2-a):

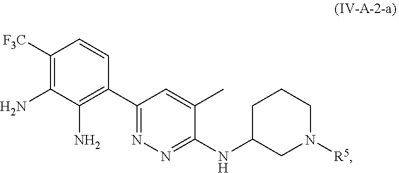

or a pharmaceutically acceptable salt, stereoisomer, solvate, prodrug, isotopic derivative, or tautomer thereof.

In some embodiments, the compound is of Formula (IV-A-2-a*):

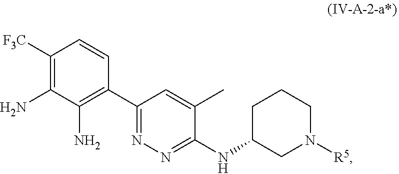

or a pharmaceutically acceptable salt, stereoisomer, solvate, prodrug, isotopic derivative, or tautomer thereof.

In some embodiments, the compound is of Formula (IV-A-2-b):

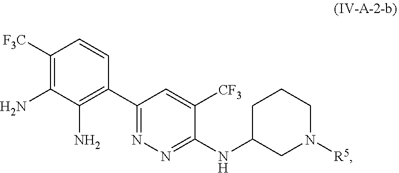

or a pharmaceutically acceptable salt, stereoisomer, solvate, prodrug, isotopic derivative, or tautomer thereof.

In some embodiments, the compound is of Formula (IV-A-2-b*):

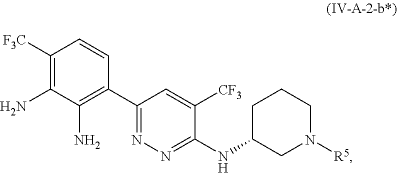

or a pharmaceutically acceptable salt, stereoisomer, solvate, prodrug, isotopic derivative, or tautomer thereof.

In some embodiments, the compound is of Formula (V-A);

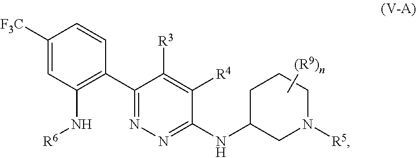

or a pharmaceutically acceptable salt, stereoisomer, solvate, prodrug, isotopic derivative, or tautomer thereof.

In some embodiments, the compound is of Formula (V-A-1);

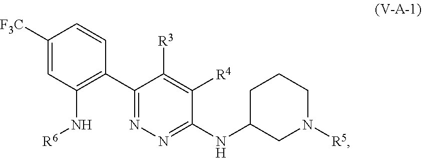

or a pharmaceutically acceptable salt, stereoisomer, solvate, prodrug, isotopic derivative, or tautomer thereof.

In some embodiments, the compound is of Formula (V-A-1-a);

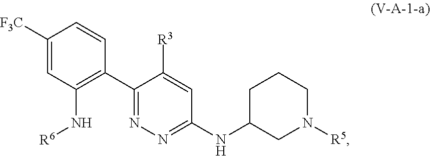

or a pharmaceutically acceptable salt, stereoisomer, solvate, prodrug, isotopic derivative, or tautomer thereof.

In some embodiments, the compound is of Formula (V-A-1-a-I);

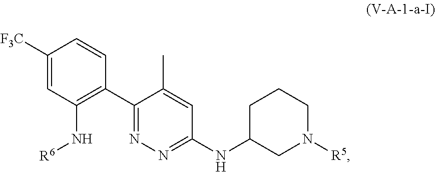

or a pharmaceutically acceptable salt, stereoisomer, solvate, prodrug, isotopic derivative, or tautomer thereof.

In some embodiments, the compound is of Formula (V-A-1-a-I*);

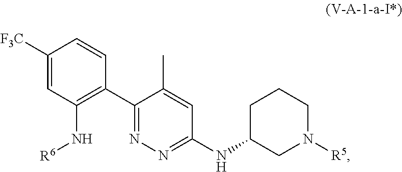

or a pharmaceutically acceptable salt, stereoisomer, solvate, prodrug, isotopic derivative, or tautomer thereof.

In some embodiments, the compound is of Formula (V-A-1-b);

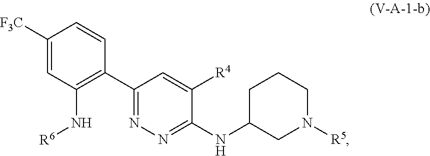

or a pharmaceutically acceptable salt, stereoisomer, solvate, prodrug, isotopic derivative, or tautomer thereof.

In some embodiments, the compound is of Formula (V-A-1-b-I);

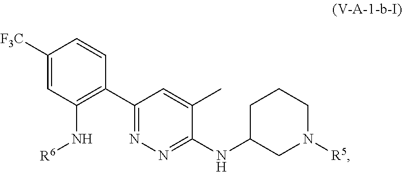

or a pharmaceutically acceptable salt, stereoisomer, solvate, prodrug, isotopic derivative, or tautomer thereof.

In some embodiments, the compound is of Formula (V-A-1-b-I*);

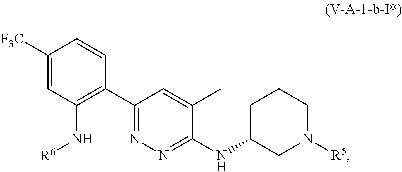

or a pharmaceutically acceptable salt, stereoisomer, solvate, prodrug, isotopic derivative, or tautomer thereof.

In some embodiments, the compound is of Formula (V-A-1-c);

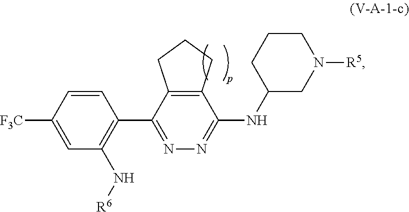

or a pharmaceutically acceptable salt, stereoisomer, solvate, prodrug, isotopic derivative, or tautomer thereof, wherein p is an integer selected from 1, 2, and 3, and all other variables are as defined herein.

In some embodiments, the compound is of Formula (V-A-1-c-I);

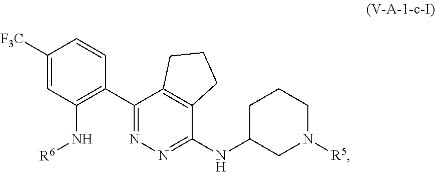

or a pharmaceutically acceptable salt, stereoisomer, solvate, prodrug, isotopic derivative, or tautomer thereof.

In some embodiments, the compound is of Formula (V-A-1-c-I*);

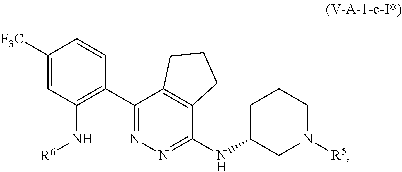

or a pharmaceutically acceptable salt, stereoisomer, solvate, prodrug, isotopic derivative, or tautomer thereof.

In some embodiments, the compound is of Formula (V-A-1-c-II);

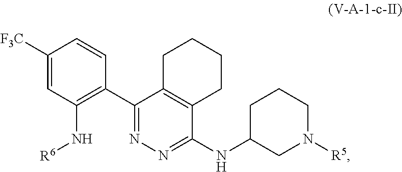

or a pharmaceutically acceptable salt, stereoisomer, solvate, prodrug, isotopic derivative, or tautomer thereof.

In some embodiments, the compound is of Formula (V-A-1-c-II*);

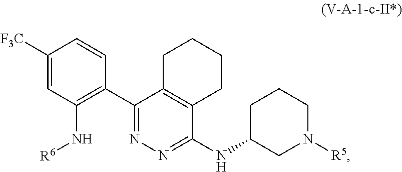

or a pharmaceutically acceptable salt, stereoisomer, solvate, prodrug, isotopic derivative, or tautomer thereof.

In some embodiments, the compound is of Formula (A). In some embodiments, the compound is of Formula (I). In some embodiments, the compound is of Formula (II). In some embodiments, the compound is of Formula (III). In some embodiments, the compound is of Formula (IV). In some embodiments, the compound is of Formula (V).

In some embodiments, the compound is of Formula (A), or a pharmaceutically acceptable salt, stereoisomer, solvate, prodrug, isotopic derivative, or tautomer thereof.

In some embodiments, the compound is of Formula (I), or a pharmaceutically acceptable salt, stereoisomer, solvate, prodrug, isotopic derivative, or tautomer thereof.

In some embodiments, the compound is of Formula (II), or a pharmaceutically acceptable salt, stereoisomer, solvate, prodrug, isotopic derivative, or tautomer thereof.

In some embodiments, the compound is of Formula (III), or a pharmaceutically acceptable salt, stereoisomer, solvate, prodrug, isotopic derivative, or tautomer thereof.

In some embodiments, the compound is of Formula (IV), or a pharmaceutically acceptable salt, stereoisomer, solvate, prodrug, isotopic derivative, or tautomer thereof.

In some embodiments, the compound is of Formula (V), or a pharmaceutically acceptable salt, stereoisomer, solvate, prodrug, isotopic derivative, or tautomer thereof.

In some embodiments, the compound is of Formula (A-0):

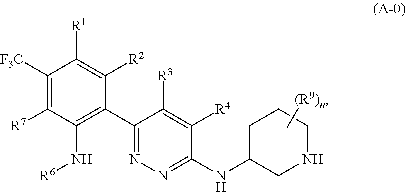

or a pharmaceutically acceptable salt, stereoisomer, solvate, prodrug, isotopic derivative, or tautomer thereof.

In some embodiments, the compound is of Formula (A-1):

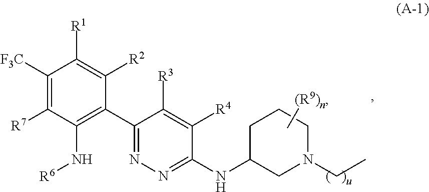

or a pharmaceutically acceptable salt, stereoisomer, solvate, prodrug, isotopic derivative, or tautomer thereof, wherein u is an integer selected from 0, 1, and all other variables are as defined herein.

In some embodiments, the compound is of Formula (A-1-a):

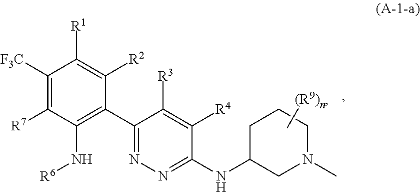

or a pharmaceutically acceptable salt, stereoisomer, solvate, prodrug, isotopic derivative, or tautomer thereof.

In some embodiments, the compound is of Formula (A-1-b):

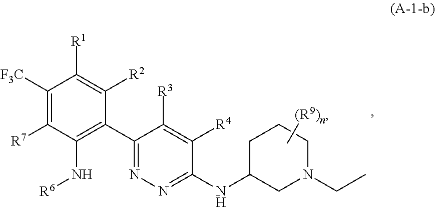

or a pharmaceutically acceptable salt, stereoisomer, solvate, prodrug, isotopic derivative, or tautomer thereof.

In some embodiments, the compound is of Formula (A-2):

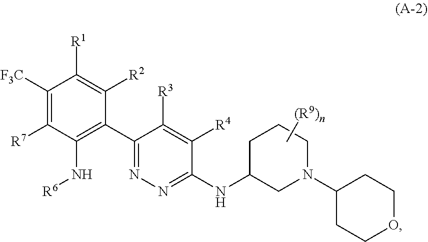

or a pharmaceutically acceptable salt, stereoisomer, solvate, prodrug, isotopic derivative, or tautomer thereof.

In some embodiments, the compound is of Formula (A-134):

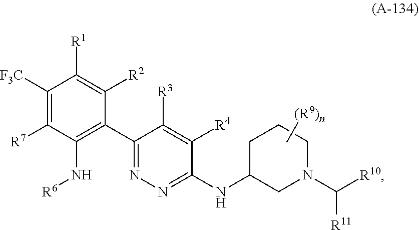

or a pharmaceutically acceptable salt, stereoisomer, solvate, prodrug, isotopic derivative, or tautomer thereof, wherein R¹⁰ is selected from —CH₃, —CH₂OH, —CH₂CH₃, —CH(CH₃)₂, —CH₂OCH₃, —C(CH₃)₂OH, —C(CH₃)₂OCH₃, —CH₂C(CH₃)₂OH, —CH₂C(CH₃)₂OCH₃; R¹¹ is selected from H, —CH₃; and all other variables are as defined herein.

In some embodiments, the compound is of Formula (A-3):

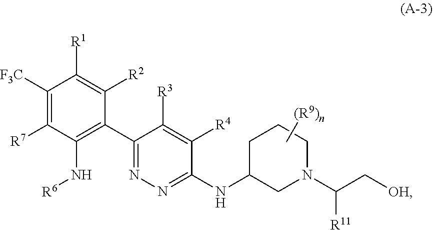

or a pharmaceutically acceptable salt, stereoisomer, solvate, prodrug, isotopic derivative, or tautomer thereof, wherein R¹¹ is selected from H, —CH₃, and all other variables are as defined herein.

In some embodiments, the compound is of Formula (A-3-a):

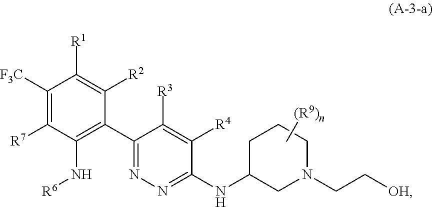

or a pharmaceutically acceptable salt, stereoisomer, solvate, prodrug, isotopic derivative, or tautomer thereof.

In some embodiments, the compound is of Formula (A-3-b):

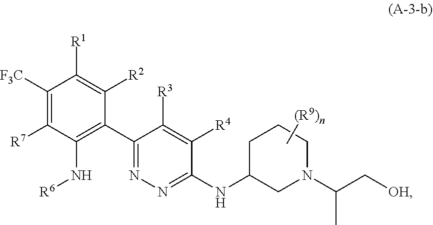

or a pharmaceutically acceptable salt, stereoisomer, solvate, prodrug, isotopic derivative, or tautomer thereof.

In some embodiments, the compound is of Formula (A-4-a):

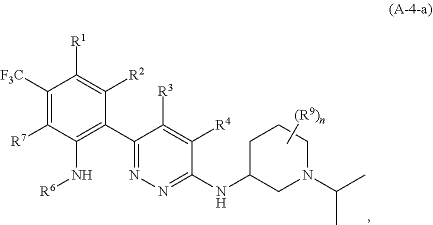

or a pharmaceutically acceptable salt, stereoisomer, solvate, prodrug, isotopic derivative, or tautomer thereof.

In some embodiments, the compound is of Formula (A-4-b):

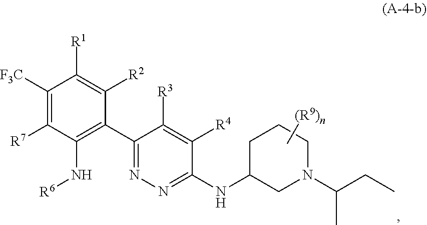

or a pharmaceutically acceptable salt, stereoisomer, solvate, prodrug, isotopic derivative, or tautomer thereof.

In some embodiments, the compound is of Formula (A-4-c):

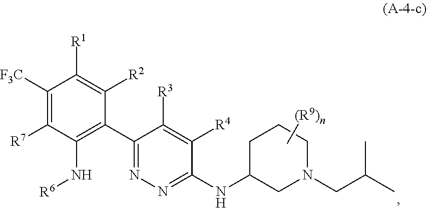

or a pharmaceutically acceptable salt, stereoisomer, solvate, prodrug, isotopic derivative, or tautomer thereof.

In some embodiments, the compound is of Formula (A-5):

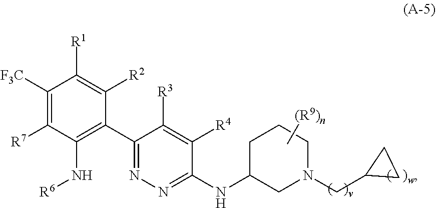

or a pharmaceutically acceptable salt, stereoisomer, solvate, prodrug, isotopic derivative, or tautomer thereof, wherein v is an integer selected from 0 and 1; w is an integer selected from 1, 2, 3, 4, and 5; and all other variables are as defined herein.

In some embodiments, the compound is of Formula (A-5-a):

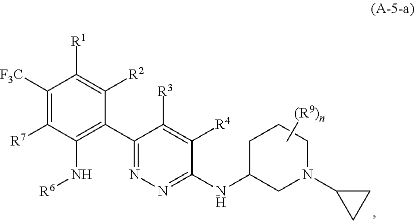

or a pharmaceutically acceptable salt, stereoisomer, solvate, prodrug, isotopic derivative, or tautomer thereof.

In some embodiments, the compound is of Formula (A-5-b):

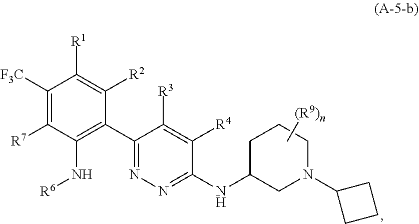

or a pharmaceutically acceptable salt, stereoisomer, solvate, prodrug, isotopic derivative, or tautomer thereof.

In some embodiments, the compound is of Formula (A-5-c):

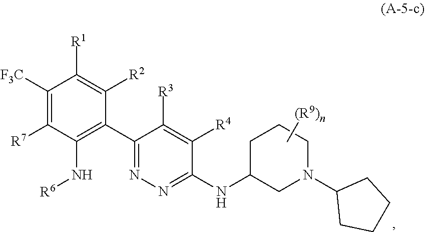

or a pharmaceutically acceptable salt, stereoisomer, solvate, prodrug, isotopic derivative, or tautomer thereof.

In some embodiments, the compound is of Formula (A-5-d):

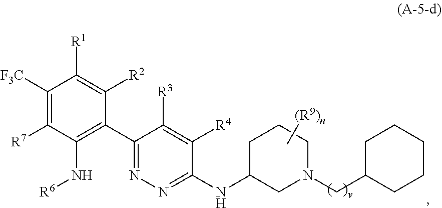

or a pharmaceutically acceptable salt, stereoisomer, solvate, prodrug, isotopic derivative, or tautomer thereof, wherein v is an integer selected from 0 and 1, and all other variables are as defined herein.

In some embodiments, the compound is of Formula (A-5-d-1):

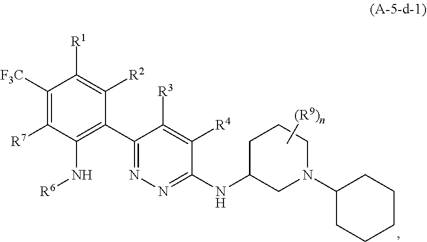

or a pharmaceutically acceptable salt, stereoisomer, solvate, prodrug, isotopic derivative, or tautomer thereof.

In some embodiments, the compound is of Formula (A-5-d-2):

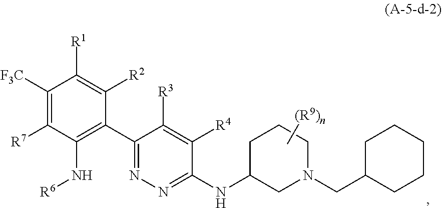

or a pharmaceutically acceptable salt, stereoisomer, solvate, prodrug, isotopic derivative, or tautomer thereof.

In some embodiments, the compound is of Formula (A-6):

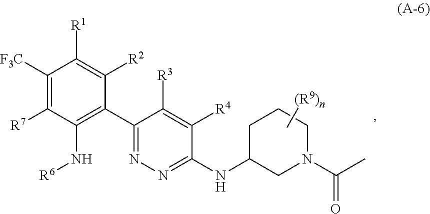

or a pharmaceutically acceptable salt, stereoisomer, solvate, prodrug, isotopic derivative, or tautomer thereof.

In some embodiments, the compound is of Formula (A-7):

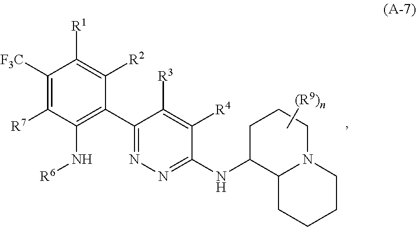

or a pharmaceutically acceptable salt, stereoisomer, solvate, prodrug, isotopic derivative, or tautomer thereof.

In some embodiments, the compound is of Formula (A-8):

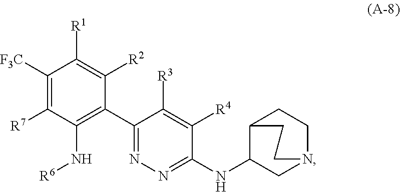

or a pharmaceutically acceptable salt, stereoisomer, solvate, prodrug, isotopic derivative, or tautomer thereof.

In some embodiments, the compound is of Formula (A-9-a):

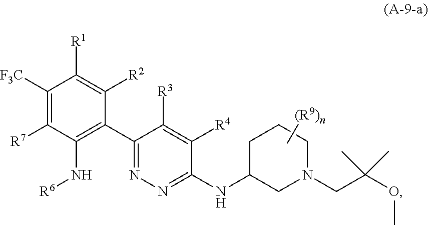

or a pharmaceutically acceptable salt, stereoisomer, solvate, prodrug, isotopic derivative, or tautomer thereof.

In some embodiments, the compound is of Formula (A-9-b):

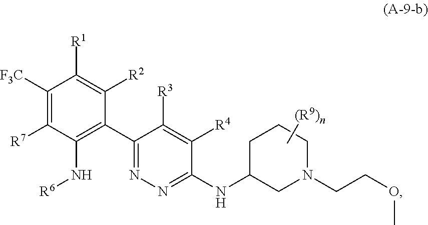

or a pharmaceutically acceptable salt, stereoisomer, solvate, prodrug, isotopic derivative, or tautomer thereof.

In some embodiments, the compound is of Formula (A-9-c):

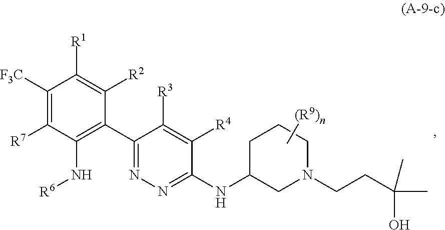

or a pharmaceutically acceptable salt, stereoisomer, solvate, prodrug, isotopic derivative, or tautomer thereof.

In some embodiments, the compound is of Formula (A-9-d):

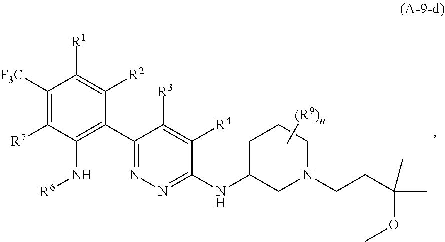

or a pharmaceutically acceptable salt, stereoisomer, solvate, prodrug, isotopic derivative, or tautomer thereof.

In some embodiments, the compound is of Formula (A-9-e):

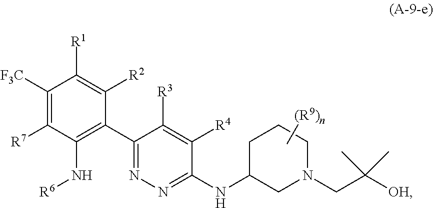

or a pharmaceutically acceptable salt, stereoisomer, solvate, prodrug, isotopic derivative, or tautomer thereof.

In some embodiments, the compound is of Formula (A-10):

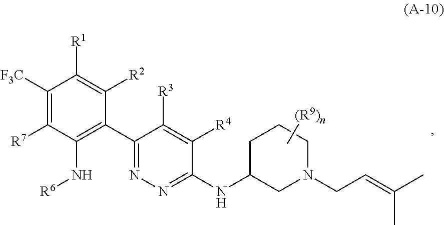

or a pharmaceutically acceptable salt, stereoisomer, solvate, prodrug, isotopic derivative, or tautomer thereof.

In some embodiments, the compound is of Formula (A-11):

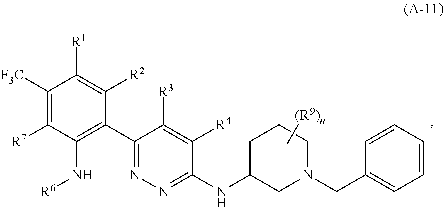

or a pharmaceutically acceptable salt, stereoisomer, solvate, prodrug, isotopic derivative, or tautomer thereof.

In some embodiments, the compound is of Formula (B):

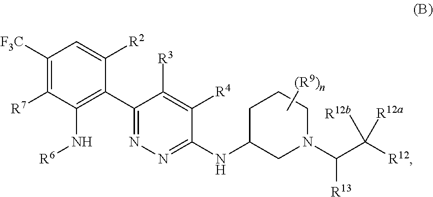

or a pharmaceutically acceptable salt, prodrug, stereoisomer, solvate, or tautomer thereof, wherein:
R² is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R³ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R⁴ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R³ and R⁴ together with the atoms to which they are attached and any intervening atoms, form a 5-8 membered cycloalkane, a 5-14 membered heterocycle, wherein the cycloalkane, heterocycle, is optionally substituted with one or more substituents independently selected from halogen, OH, CN, C₁-C₆ alkyl, C₁-C₆ alkoxy;
R⁶ is selected from H, SO₂R⁸, NH₂, C₁-C₆ alkyl, C₁-C₆ alkyl-C(O)—, aryl, wherein alkyl or aryl is optionally substituted with one or more substituents independently selected from halogen, CN, OH;
R⁷ is H, C₁-C₆ alkyl, or NH₂; or
R⁶ and R⁷ together with the atoms to which they are attached and any intervening atoms, form a 5-6 membered heterocycle or heteroarene;
R⁸ is selected from C₁-C₆ alkyl, C₃-C₈ cycloalkyl, 5-6 membered heteroaryl, wherein alkyl, cycloalkyl or heteroaryl is optionally substituted with one or more substituents independently selected from halogen, CN, OH; or each R⁹ is independently selected from halogen, OH, C₁-C₆ alkyl, C₁-C₆ alkoxy, wherein alkyl or alkoxy optionally substituted with one or more halogen, OH; or two R⁹ together with the atoms to which they are attached and any intervening atoms, form a 5-6 membered heterocycle; or
R¹² is selected from H, OH, C₁-C₆ alkyl, C₁-C₆ alkoxy, wherein alkyl is optionally substituted with one or more group selected from halogen, —OH, C₁-C₆ alkoxy;
R^{12a} is selected from H, —CH₃;
R^{12b} is selected from H, —CH₃;
R¹³ is selected from H, C₁-C₆ alkyl,
or R¹² and R¹³ together with the atoms to which they are attached and any intervening atoms, form a 3-8 membered cycloalkane, a 3-8 membered heterocycle, wherein the cycloalkane, heterocycle, is optionally substituted with one or more substituents independently selected from halogen, OH, CN, C₁-C₆ alkyl, C₁-C₆ alkoxy.

In some embodiments, R¹ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl.

In some embodiments, R¹ is H.

In some embodiments, R¹ is C₁-C₆ alkyl optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl.

In some embodiments, R¹ is C₁-C₆ alkyl optionally substituted with one or more halogen.

In some embodiments, R¹ is C₁-C₆ alkyl.

In some embodiments, R¹ is selected from methyl, ethyl, propyl, butyl, pentyl, hexyl.

In some embodiments, R¹ is selected from methyl, ethyl, n-propyl, i-propyl, n-butyl, i-butyl, sec-butyl, tert-butyl, n-pentyl, n-hexyl.

In some embodiments, R¹ is methyl.

In some embodiments, R² is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl.

In some embodiments, R² is H.

In some embodiments, R² is C₁-C₆ alkyl optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl.

In some embodiments, R² is C₁-C₆ alkyl optionally substituted with one or more halogen.

In some embodiments, R² is C₁-C₆ alkyl.

In some embodiments, R² is selected from methyl, ethyl, propyl, butyl, pentyl, hexyl.

In some embodiments, R² is selected from methyl, ethyl, n-propyl, i-propyl, n-butyl, i-butyl, sec-butyl, tert-butyl, n-pentyl, n-hexyl.

In some embodiments, R² is methyl.

In some embodiments, R¹ and R² together with the atoms to which they are attached and any intervening atoms, form a 5-8 membered cycloalkane, a 5-14 membered heterocycle, arene, or heteroarene wherein the cycloalkane, heterocycle, arene, or heteroarene is optionally substituted with one or more substituents independently selected from halogen, OH, CN, C₁-C₆ alkyl, C₁-C₆ alkoxy.

In some embodiments, R³ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl.

In some embodiments, R³ is H.

In some embodiments, R³ is C₁-C₆ alkyl optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl.

In some embodiments, R³ is C₁-C₆ alkyl optionally substituted with one or more halogen.

In some embodiments, R³ is C₁-C₆ alkyl.

In some embodiments, R³ is selected from methyl, ethyl, propyl, butyl, pentyl, hexyl.

In some embodiments, R³ is selected from methyl, ethyl, n-propyl, i-propyl, n-butyl, i-butyl, sec-butyl, tert-butyl, n-pentyl, n-hexyl.

In some embodiments, R³ is methyl.

In some embodiments, R³ is —CF₃.

In some embodiments, R³ is —CF₂CF₃.

In some embodiments, R³ is C₃-C₁₀ cycloalkyl optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl.

In some embodiments, R³ is C₃-C₁₀ cycloalkyl.

In some embodiments, R³ is cyclopropyl.

In some embodiments, R⁴ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl.

In some embodiments, R⁴ is H.

In some embodiments, R⁴ is C₁-C₆ alkyl optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl.

In some embodiments, R⁴ is C₁-C₆ alkyl optionally substituted with one or more halogen.

In some embodiments, R⁴ is C₁-C₆ alkyl.

In some embodiments, R⁴ is selected from methyl, ethyl, propyl, butyl, pentyl, hexyl.

In some embodiments, R⁴ is selected from methyl, ethyl, n-propyl, i-propyl, n-butyl, i-butyl, sec-butyl, tert-butyl, n-pentyl, n-hexyl.

In some embodiments, R⁴ is methyl.

In some embodiments, R⁴ is —CF₃.

In some embodiments, R⁴ is —CF₂CF₃.

In some embodiments, R⁴ is C₃-C₁₀ cycloalkyl optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl.

In some embodiments, R⁴ is C₃-C₁₀ cycloalkyl.

In some embodiments, R⁴ is cyclopropyl.

In some embodiments, R³ and R⁴ together with the atoms to which they are attached and any intervening atoms, form a 5-8 membered cycloalkane, a 5-14 membered heterocycle, wherein the cycloalkane, heterocycle, is optionally substituted with one or more substituents independently selected from halogen, OH, CN, C₁-C₆ alkyl, C₁-C₆ alkoxy.

In some embodiments, R³ and R⁴ together with the atoms to which they are attached and any intervening atoms, form cyclopentane.

In some embodiments, R³ and R⁴ together with the atoms to which they are attached and any intervening atoms, form

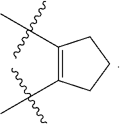 [SMILES: *C1=C(*)CCC1]

In some embodiments, R³ and R⁴ together with the atoms to which they are attached and any intervening atoms, form cyclohexane.

In some embodiments, R³ and R⁴ together with the atoms to which they are attached and any intervening atoms, form

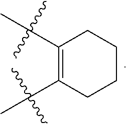 [SMILES: *C1=C(*)CCCC1]

In some embodiments, R³ and R⁴ together with the atoms to which they are attached and any intervening atoms, form norbornane.

In some embodiments, R³ and R⁴ together with the atoms to which they are attached and any intervening atoms, form

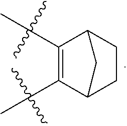 [SMILES: *C1=C(*)C2CCC1C2]

In some embodiments, R³ and R⁴ together with the atoms to which they are attached and any intervening atoms, form bicyclo[2.2.2]octane.

In some embodiments, R³ and R⁴ together with the atoms to which they are attached and any intervening atoms, form

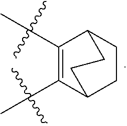 [SMILES: *C1=C(*)C2CCC1CC2]

In some embodiments, R⁵ is selected from H, C₁-C₆ alkyl, C₃-C₁₀ cycloalkyl, C₂-C₆ alkenyl, C₂-C₆ alkynyl, C₁-C₆ acyl, SO₂—C₁-C₆ alkyl, 4-10 membered heterocyclyl, wherein the alkyl, cycloalkyl, alkenyl, alkynyl, acyl, or heterocyclyl is optionally substituted with one or more substituents independently selected from halogen, oxo, OH, CN, C₁-C₆ alkoxy, C₃-C₁₀ cycloalkyl, aryl.

In some embodiments, R⁵ is H.

In some embodiments, R⁵ is C₁-C₆ alkyl.

In some embodiments, R⁵ is selected from methyl, ethyl, propyl, butyl, pentyl, hexyl.

In some embodiments, R⁵ is selected from methyl, ethyl, n-propyl, i-propyl, n-butyl, i-butyl, sec-butyl, tert-butyl, n-pentyl, n-hexyl.

In some embodiments, R⁵ is methyl.

In some embodiments, R⁵ is ethyl.

In some embodiments, R⁵ is

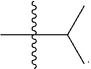 [SMILES: *C(C)C]

In some embodiments, R⁵ is

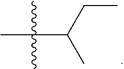 [SMILES: *C(C)CC]

In some embodiments, R⁵ is

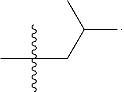 [SMILES: *CC(C)C]

In some embodiments, R⁵ is C₁-C₆ alkyl substituted with OH.

In some embodiments, R⁵ is 2-hydroxyethyl.

In some embodiments, R⁵ is

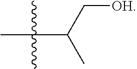 [SMILES: *C(C)CO]

In some embodiments, R⁵ is

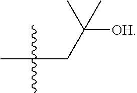 [SMILES: *CC(C)(C)O]

In some embodiments, R⁵ is

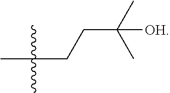 [SMILES: *CCC(C)(C)O]

In some embodiments, R⁵ is C₁-C₆ alkyl substituted with C₁-C₆ alkoxy.

In some embodiments, R⁵ is C₁-C₆ alkyl substituted with methoxy.

In some embodiments, R⁵ is

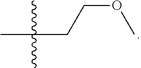 [SMILES: *CCOC]

In some embodiments, R⁵ is

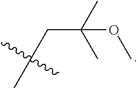 [SMILES: *CC(C)(C)OC]

In some embodiments, R⁵ is

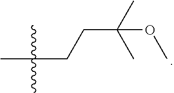 [SMILES: *CCC(C)(C)OC]

In some embodiments, R⁵ is C₁-C₆ alkyl substituted with C₃-C₁₀ cycloalkyl.

In some embodiments, R⁵ is

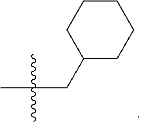 [SMILES: *CC1CCCCC1]

In some embodiments, R⁵ is C₁-C₆ alkyl substituted with aryl.

In some embodiments, R⁵ is

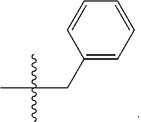 [SMILES: *Cc1ccccc1]

In some embodiments, R⁵ is C₂-C₆ alkenyl.

In some embodiments, R⁵ is

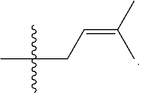 [SMILES: *CC=C(C)C]

In some embodiments, R⁵ is C₃-C₁₀ cycloalkyl.

In some embodiments, R⁵ is cyclopropyl.

In some embodiments, R⁵ is cyclobutyl.

In some embodiments, R⁵ is cyclopentyl.

In some embodiments, R⁵ is cyclohexyl.

In some embodiments, R⁵ is cycloheptyl.

In some embodiments, R⁵ is 4-10 membered heterocyclyl.

In some embodiments, R⁵ is 4-10 membered heterocyclyl comprising 1-3 heteroatoms independently selected from O, N, S.

In some embodiments, R⁵ is 4-10 membered heterocyclyl comprising 1 heteroatom selected from O, N, S.

In some embodiments, R⁵ is 4-10 membered heterocyclyl comprising 2 heteroatoms independently selected from O, N, S.

In some embodiments, R⁵ is 4 membered heterocyclyl comprising 1 heteroatom selected from O, N, S.

In some embodiments, R⁵ is 5 membered heterocyclyl comprising 1 heteroatom selected from O, N, S.

In some embodiments, R⁵ is 6 membered heterocyclyl comprising 1 heteroatom selected from O, N, S.

In some embodiments, R⁵ is

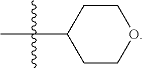 [SMILES: *C1CCOCC1]

In some embodiments, R⁵ is 7 membered heterocyclyl comprising 1 heteroatom selected from O, N, S.

In some embodiments, R⁵ is

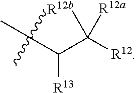

In some embodiments, R⁶ is selected from H, SO₂R⁸, NH₂, C₁-C₆ alkyl, C₁-C₆ alkyl-C(O)—, aryl, wherein alkyl or aryl is optionally substituted with one or more substituents independently selected from halogen, CN, OH.

In some embodiments, R⁶ is H.

In some embodiments, R⁶ is C₁-C₆ alkyl.

In some embodiments, R⁶ is —CH₃.

In some embodiments, R⁶ is —CH₂CH₃.

In some embodiments, R⁶ is

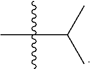 [SMILES: *C(C)C]

In some embodiments, R⁶ is C₁-C₆ alkyl optionally substituted with one or more substituents independently selected from halogen, CN, OH.

In some embodiments, R⁶ is —CF₃.

In some embodiments, R⁶ is aryl.

In some embodiments, R⁶ is

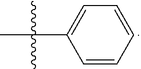 [SMILES: *c1ccccc1]

In some embodiments, R⁶ is SO₂R⁸.

In some embodiments, R⁶ is SO₂CH₃.

In some embodiments, R⁶ is SO₂CH₂CH₃.

In some embodiments, R⁶ is

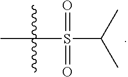 [SMILES: *S(=O)(=O)C(C)C]

In some embodiments, R⁶ is

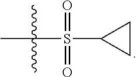 [SMILES: *S(=O)(=O)C1CC1]

In some embodiments, R⁶ is

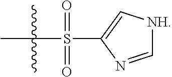 [SMILES: *S(=O)(=O)c1c[nH]cn1]

In some embodiments, R⁶ is C₁-C₆ alkyl-C(O)—, wherein alkyl is optionally substituted with one or more substituents independently selected from halogen, CN, OH.

In some embodiments, R⁶ is CH₃C(O)—.

In some embodiments, R⁶ is (CH₃)₂CHC(O)—.

In some embodiments, R⁶ is (CH₃)₃C(O)—.

In some embodiments, R⁶ is CHF₂C(O)—.

In some embodiments, R⁶ is CF₃C(O)—.

In some embodiments, R⁶ is CF₃CF₂C(O)—.

In some embodiments, R⁷ is H, C₁-C₆ alkyl, or —NH₂.

In some embodiments, R⁷ is H.

In some embodiments, R⁷ is-NH₂.

In some embodiments, R⁶ and R⁷ together with the atoms to which they are attached and any intervening atoms, form a 5-6 membered heterocycle or heteroarene.

In some embodiments, R⁶ and R⁷ together with the atoms to which they are attached and any intervening atoms, form

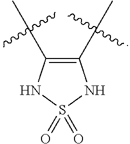 [SMILES: *C1=C(*)NS(=O)(=O)N1]

In some embodiments, cycle

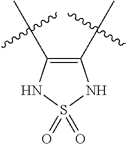 [SMILES: *C1=C(*)NS(=O)(=O)N1]

is optionally substituted with one or two substituents.

In some embodiments, R⁶ and R⁷ together with the atoms to which they are attached and any intervening atoms, form

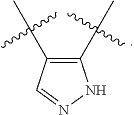 [SMILES: *c1cn[nH]c1*]

In some embodiments, cycle

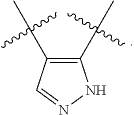 [SMILES: *c1cn[nH]c1*]

is optionally substituted with one or two substituents.

In some embodiments, R⁶ and R⁷ together with the atoms to which they are attached and any intervening atoms, form

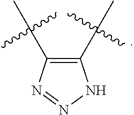 [SMILES: *c1nn[nH]c1*]

In some embodiments, cycle

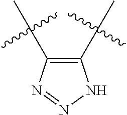 [SMILES: *c1nn[nH]c1*]

is optionally substituted.

In some embodiments, R⁸ is selected from C₁-C₆ alkyl, C₃-C₈ cycloalkyl, 5-6 membered heteroaryl, wherein alkyl, cycloalkyl or heteroaryl is optionally substituted with one or more substituents independently selected from halogen, CN, OH.

In some embodiments, R⁸ is C₁-C₆ alkyl.

In some embodiments, R⁸ is —CH₃.

In some embodiments, R⁸ is —CH₂CH₃.

In some embodiments, R⁸ is

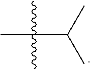 [SMILES: *C(C)C]

In some embodiments, R⁸ is C₃-C₈ cycloalkyl.

In some embodiments, R⁸ is

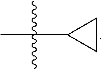 [SMILES: *C1CC1]

In some embodiments, R⁸ is 5-6 membered heteroaryl.

In some embodiments, R⁸ is

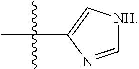 [SMILES: *c1c[nH]cn1]

In some embodiments, n is an integer selected from 0, 1, and 2.

In some embodiments, n is 0.

In some embodiments, n is 1.

In some embodiments, R⁹ is selected from halogen, OH, C₁-C₆ alkyl, C₁-C₆ alkoxy, wherein alkyl or alkoxy optionally substituted with one or more halogen, OH.

In some embodiments, n is 2.

In some embodiments, each R⁹ is independently selected from halogen, OH, C₁-C₆ alkyl, C₁-C₆ alkoxy, wherein alkyl or alkoxy optionally substituted with one or more halogen, OH.

In some embodiments, R⁹ is OH.

In some embodiments, R⁹ is halogen.

In some embodiments, R⁹ is F.

In some embodiments, R⁹ is C₁-C₆ alkyl.

In some embodiments, R⁹ is methyl.

In some embodiments, R⁹ is C₁-C₆ alkyl optionally substituted with one or more halogen.

In some embodiments, R⁹ is —CF₃.

In some embodiments, R⁹ is C₁-C₆ alkoxy.

In some embodiments, R⁹ is methoxy.

In some embodiments, two R⁹ together with the atoms to which they are attached and any intervening atoms, form a 5-6 membered heterocycle.

In some embodiments, R⁵ and R⁹ together with the atoms to which they are attached and any intervening atoms, form a 5-6 membered heterocycle.

In some embodiments, R¹ is H, R² is H, R³ is H, R⁴ is H, R⁵ is methyl, R⁶ is SO₂CH₃. R⁷ is H.

In some embodiments, R¹ is H, R² is H, R³ is H, R⁴ is H, R⁵ is ethyl, R⁶ is SO₂CH₃. R⁷ is H.

In some embodiments, R¹ is H, R² is H, R³ is H, R⁴ is H, R⁵ is 2-hydroxyethyl, R⁶ is SO₂CH₃. R⁷ is H.

In some embodiments, R¹ is H, R² is methyl, R³ is H, R⁴ is H, R⁵ is methyl, R⁶ is SO₂CH₃. R⁷ is H.

In some embodiments, R¹ is H, R² is methyl, R³ is H, R⁴ is H, R⁵ is ethyl, R⁶ is SO₂CH₃. R⁷ is H.

In some embodiments, R¹ is H, R² is methyl, R³ is H, R⁴ is H, R⁵ is 2-hydroxyethyl, R⁶ is SO₂CH₃. R⁷ is H.

In some embodiments, R¹ is H, R² is H, R³ is methyl, R⁴ is H, R⁵ is methyl, R⁶ is SO₂CH₃. R⁷ is H.

In some embodiments, R¹ is H, R² is H, R³ is methyl, R⁴ is H, R⁵ is ethyl, R⁶ is SO₂CH₃. R⁷ is H.

In some embodiments, R¹ is H, R² is H, R³ is methyl, R⁴ is H, R⁵ is 2-hydroxyethyl, R⁶ is SO₂CH₃. R⁷ is H.

In some embodiments, R¹ is H, R² is H, R³ is H, R⁴ is methyl, R⁵ is methyl, R⁶ is SO₂CH₃. R⁷ is H.

In some embodiments, R¹ is H, R² is H, R³ is H, R⁴ is methyl, R⁵ is ethyl, R⁶ is SO₂CH₃. R⁷ is H.

In some embodiments, R¹ is H, R² is H, R³ is H, R⁴ is methyl, R⁵ is 2-hydroxyethyl, R⁶ is SO₂CH₃. R⁷ is H.

In some embodiments, R¹ is H, R² is H, R³ is methyl, R⁴ is H, R⁵ is methyl, R⁶ and R⁷ together with the atoms to which they are attached and any intervening atoms, form

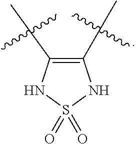 [SMILES: *C1=C(*)NS(=O)(=O)N1]

In some embodiments, R¹ is H, R² is H, R³ is methyl, R⁴ is H, R⁵ is methyl, R⁶ and R⁷ together with the atoms to which they are attached and any intervening atoms, form

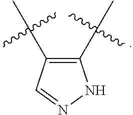 [SMILES: *c1cn[nH]c1*]

In some embodiments, R¹ is H, R² is H, R³ is methyl, R⁴ is H, R⁵ is methyl, R⁶ and R⁷ together with the atoms to which they are attached and any intervening atoms, form

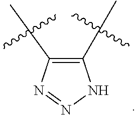 [SMILES: *c1nn[nH]c1*]

In some embodiments, R¹ is H, R² is H, R³ is methyl, R⁴ is methyl, R⁵ is methyl, R⁶ and R⁷ together with the atoms to which they are attached and any intervening atoms, form

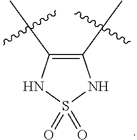 [SMILES: *C1=C(*)NS(=O)(=O)N1]

In some embodiments, R¹ is H, R² is H, R³ is methyl, R⁴ is methyl, R⁵ is methyl, R⁶ and R⁷ together with the atoms to which they are attached and any intervening atoms, form

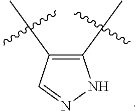 [SMILES: *c1cn[nH]c1*]

In some embodiments, R¹ is H, R² is H, R³ is methyl, R⁴ is methyl, R⁵ is methyl, R⁶ and R⁷ together with the atoms to which they are attached and any intervening atoms, form

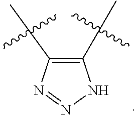 [SMILES: *c1nn[nH]c1*]

In some embodiments, R¹ is H, R² is H, R³ is —CF₃, R⁴ is H, R⁵ is methyl, R⁶ and R⁷ together with the atoms to which they are attached and any intervening atoms, form

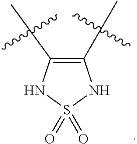 [SMILES: *C1=C(*)NS(=O)(=O)N1]

In some embodiments, R¹ is H, R² is H, R³ is —CF₃, R⁴ is H, R⁵ is methyl, R⁶ and R⁷ together with the atoms to which they are attached and any intervening atoms, form

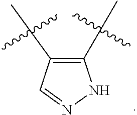 [SMILES: *c1cn[nH]c1*]

In some embodiments, R¹ is H, R² is H, R³ is —CF₃, R⁴ is H, R⁵ is methyl, R⁶ and R⁷ together with the atoms to which they are attached and any intervening atoms, form

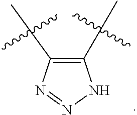 [SMILES: *c1nn[nH]c1*]

In some embodiments, R¹ is H, R² is H, R³ is H, R⁴ is methyl, R⁵ is methyl, R⁶ and R⁷ together with the atoms to which they are attached and any intervening atoms, form

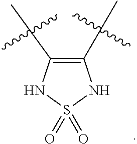 [SMILES: *C1=C(*)NS(=O)(=O)N1]

In some embodiments, R¹ is H, R² is H, R³ is H, R⁴ is methyl, R⁵ is methyl, R⁶ and R⁷ together with the atoms to which they are attached and any intervening atoms, form

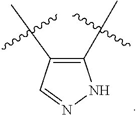 [SMILES: *c1cn[nH]c1*]

In some embodiments, R¹ is H, R² is H, R³ is H, R⁴ is methyl, R⁵ is methyl, R⁶ and R⁷ together with the atoms to which they are attached and any intervening atoms, form

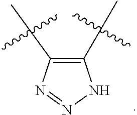 [SMILES: *c1nn[nH]c1*]

In some embodiments, R¹ is H, R² is H, R³ is H, R⁴ is —CF₃, R⁵ is methyl, R⁶ and R⁷ together with the atoms to which they are attached and any intervening atoms, form

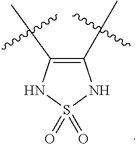 [SMILES: *C1=C(*)NS(=O)(=O)N1]

In some embodiments, R¹ is H, R² is H, R³ is H, R⁴ is —CF₃, R⁵ is methyl, R⁶ and R⁷ together with the atoms to which they are attached and any intervening atoms, form

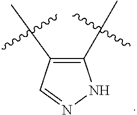 [SMILES: *c1cn[nH]c1*]

In some embodiments, R¹ is H, R² is H, R³ is H, R⁴ is —CF₃, R⁵ is methyl, R⁶ and R⁷ together with the atoms to which they are attached and any intervening atoms, form

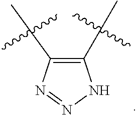 [SMILES: *c1nn[nH]c1*]

In some embodiments, R¹ is H, R² is H, R³ is methyl, R⁴ is H, R⁵ is ethyl, R⁶ and R⁷ together with the atoms to which they are attached and any intervening atoms, form

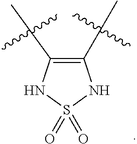 [SMILES: *C1=C(*)NS(=O)(=O)N1]

In some embodiments, R¹ is H, R² is H, R³ is methyl, R⁴ is H, R⁵ is ethyl, R⁶ and R⁷ together with the atoms to which they are attached and any intervening atoms, form

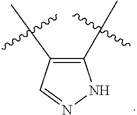 [SMILES: *c1cn[nH]c1*]

In some embodiments, R¹ is H, R² is H, R³ is methyl, R⁴ is H, R⁵ is ethyl, R⁶ and R⁷ together with the atoms to which they are attached and any intervening atoms, form

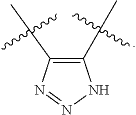 [SMILES: *c1nn[nH]c1*]

In some embodiments, R¹ is H, R² is H, R³ is methyl, R⁴ is methyl, R⁵ is ethyl, R⁶ and R⁷ together with the atoms to which they are attached and any intervening atoms, form

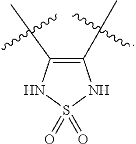 [SMILES: *C1=C(*)NS(=O)(=O)N1]

In some embodiments, R¹ is H, R² is H, R³ is methyl, R⁴ is methyl, R⁵ is ethyl, R⁶ and R⁷ together with the atoms to which they are attached and any intervening atoms, form

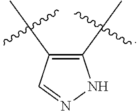 [SMILES: *c1cn[nH]c1*]

In some embodiments, R¹ is H, R² is H, R³ is methyl, R⁴ is methyl, R⁵ is ethyl, R⁶ and R⁷ together with the atoms to which they are attached and any intervening atoms, form

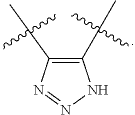 [SMILES: *c1nn[nH]c1*]

In some embodiments, R¹ is H, R² is H, R³ is —CF₃, R⁴ is H, R⁵ is ethyl, R⁶ and R⁷ together with the atoms to which they are attached and any intervening atoms, form

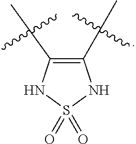 [SMILES: *C1=C(*)NS(=O)(=O)N1]

In some embodiments, R¹ is H, R² is H, R³ is —CF₃, R⁴ is H, R⁵ is ethyl, R⁶ and R⁷ together with the atoms to which they are attached and any intervening atoms, form

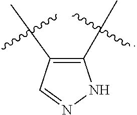 [SMILES: *c1cn[nH]c1*]

In some embodiments, R¹ is H, R² is H, R³ is —CF₃, R⁴ is H, R⁵ is ethyl, R⁶ and R⁷ together with the atoms to which they are attached and any intervening atoms, form

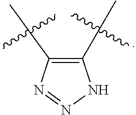 [SMILES: *c1nn[nH]c1*]

In some embodiments, R¹ is H, R² is H, R³ is H, R⁴ is methyl, R⁵ is ethyl, R⁶ and R⁷ together with the atoms to which they are attached and any intervening atoms, form

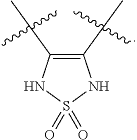 [SMILES: *C1=C(*)NS(=O)(=O)N1]

In some embodiments, R¹ is H, R² is H, R³ is H, R⁴ is methyl, R⁵ is ethyl, R⁶ and R⁷ together with the atoms to which they are attached and any intervening atoms, form

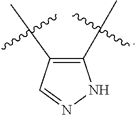 [SMILES: *c1cn[nH]c1*]

In some embodiments, R¹ is H, R² is H, R³ is H, R⁴ is methyl, R⁵ is ethyl, R⁶ and R⁷ together with the atoms to which they are attached and any intervening atoms, form

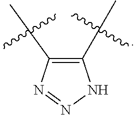 [SMILES: *c1nn[nH]c1*]

In some embodiments, R¹ is H, R² is H, R³ is H, R⁴ is —CF₃, R⁵ is ethyl, R⁶ and R⁷ together with the atoms to which they are attached and any intervening atoms, form

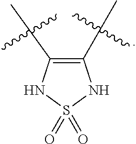 [SMILES: *C1=C(*)NS(=O)(=O)N1]

In some embodiments, R¹ is H, R² is H, R³ is H, R⁴ is —CF₃, R⁵ is ethyl, R⁶ and R⁷ together with the atoms to which they are attached and any intervening atoms, form

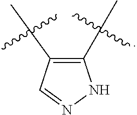 [SMILES: *c1cn[nH]c1*]

In some embodiments, R¹ is H, R² is H, R³ is H, R⁴ is —CF₃, R⁵ is ethyl, R⁶ and R⁷ together with the atoms to which they are attached and any intervening atoms, form

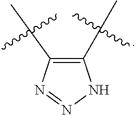 [SMILES: *c1nn[nH]c1*]

In some embodiments, R¹ is H, R² is H, R³ is methyl, R⁴ is H, R⁵ is 2-hydroxyethyl, R⁶ and R⁷ together with the atoms to which they are attached and any intervening atoms, form

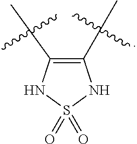 [SMILES: *C1=C(*)NS(=O)(=O)N1]

In some embodiments, R¹ is H, R² is H, R³ is methyl, R⁴ is H, R⁵ is 2-hydroxyethyl,

R⁶ and R⁷ together with the atoms to which they are attached and any intervening atoms, form

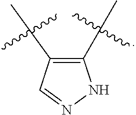 [SMILES: *c1cn[nH]c1*]

In some embodiments, R¹ is H, R² is H, R³ is methyl, R⁴ is H, R⁵ is 2-hydroxyethyl, R⁶ and R⁷ together with the atoms to which they are attached and any intervening atoms, form

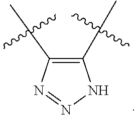 [SMILES: *c1nn[nH]c1*]

In some embodiments, R¹ is H, R² is H, R³ is methyl, R⁴ is methyl, R⁵ is 2-hydroxyethyl, R⁶ and R⁷ together with the atoms to which they are attached and any intervening atoms, form

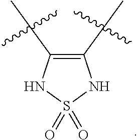 [SMILES: *C1=C(*)NS(=O)(=O)N1]

In some embodiments, R¹ is H, R² is H, R³ is methyl, R⁴ is methyl, R⁵ is 2-hydroxyethyl, R⁶ and R⁷ together with the atoms to which they are attached and any intervening atoms, form

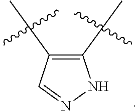 [SMILES: *c1cn[nH]c1*]

In some embodiments, R¹ is H, R² is H, R³ is methyl, R⁴ is methyl, R⁵ is 2-hydroxyethyl, R⁶ and R⁷ together with the atoms to which they are attached and any intervening atoms, form

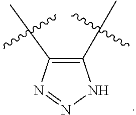 [SMILES: *c1nn[nH]c1*]

In some embodiments, R¹ is H, R² is H, R³ is —CF₃, R⁴ is H, R⁵ is 2-hydroxyethyl, R⁶ and R⁷ together with the atoms to which they are attached and any intervening atoms, form

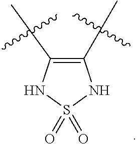 [SMILES: *C1=C(*)NS(=O)(=O)N1]

In some embodiments, R¹ is H, R² is H, R³ is —CF₃, R⁴ is H, R⁵ is 2-hydroxyethyl, R⁶ and R⁷ together with the atoms to which they are attached and any intervening atoms, form

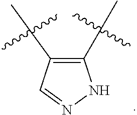 [SMILES: *c1cn[nH]c1*]

In some embodiments, R¹ is H, R² is H, R³ is —CF₃, R⁴ is H, R⁵ is 2-hydroxyethyl, R⁶ and R⁷ together with the atoms to which they are attached and any intervening atoms, form

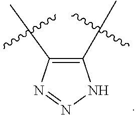 [SMILES: *c1nn[nH]c1*]

In some embodiments, R¹ is H, R² is H, R³ is H, R⁴ is methyl, R⁵ is 2-hydroxyethyl, R⁶ and R⁷ together with the atoms to which they are attached and any intervening atoms, form

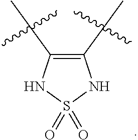 [SMILES: *C1=C(*)NS(=O)(=O)N1]

In some embodiments, R¹ is H, R² is H, R³ is H, R⁴ is methyl, R⁵ is 2-hydroxyethyl, R⁶ and R⁷ together with the atoms to which they are attached and any intervening atoms, form

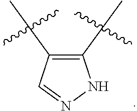 [SMILES: *c1cn[nH]c1*]

In some embodiments, R¹ is H, R² is H, R³ is H, R⁴ is methyl, R⁵ is 2-hydroxyethyl, R⁶ and R⁷ together with the atoms to which they are attached and any intervening atoms, form

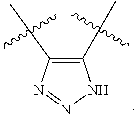 [SMILES: *c1nn[nH]c1*]

In some embodiments, R¹ is H, R² is H, R³ is H, R⁴ is —CF₃, R⁵ is 2-hydroxyethyl, R⁶ and R⁷ together with the atoms to which they are attached and any intervening atoms, form

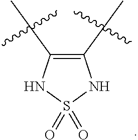 [SMILES: *C1=C(*)NS(=O)(=O)N1]

In some embodiments, R¹ is H, R² is H, R³ is H, R⁴ is —CF₃, R⁵ is 2-hydroxyethyl, R⁶ and R⁷ together with the atoms to which they are attached and any intervening atoms, form

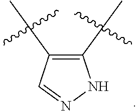 [SMILES: *c1cn[nH]c1*]

In some embodiments, R¹ is H, R² is H, R³ is H, R⁴ is —CF₃, R⁵ is 2-hydroxyethyl, R⁶ and R⁷ together with the atoms to which they are attached and any intervening atoms, form

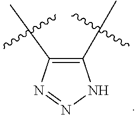 [SMILES: *c1nn[nH]c1*]

In some embodiments, the compound is selected from the compounds described in Table 1 and pharmaceutically acceptable salts, stereoisomers, solvates, prodrugs, isotopic derivatives, or tautomers thereof.

In some embodiments, the compound is selected from the compounds described in Table 1 and prodrugs and pharmaceutically acceptable salts thereof.

In some embodiments, the compound is selected from the compounds described in Table 1 and pharmaceutically acceptable salts thereof.

In some embodiments, the compound is selected from the prodrugs of the compounds described in Table 1 and pharmaceutically acceptable salts thereof.

In some embodiments, the compound is selected from the compounds described in Table 1.

TABLE 1
Certain examples of the compound of Formula A

| # | Structure | IUPAC Name |
|---|---|---|
| 1 | 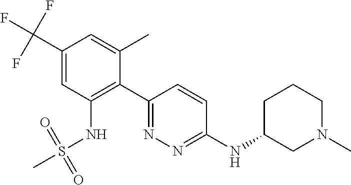 [SMILES: Cc1cc(C(F)(F)F)cc(NS(C)(=O)=O)c1-c1ccc(N[C@@H]2CCCN(C)C2)nn1] | N-[3-methyl-2-[6-[(1-methyl-3- piperidyl)amino]pyridazin-3-yl]-5- (trifluoromethyl)phenyl]methanesulfon- amide |
| 2 | 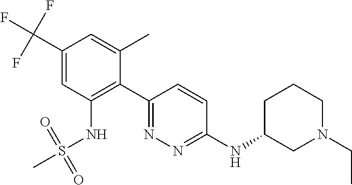 [SMILES: CCN1CCC[C@@H](Nc2ccc(-c3c(C)cc(C(F)(F)F)cc3NS(C)(=O)=O)nn2)C1] | N-[2-[6-[(1-ethyl-3- piperidyl)amino]pyridazin-3-yl]-3- methyl-5- (trifluoromethyl)phenyl]methanesulfno- amide |
| 3 | 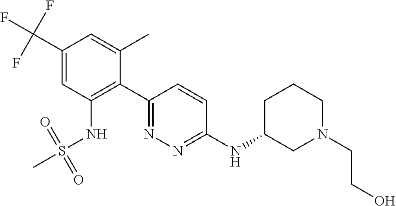 [SMILES: Cc1cc(C(F)(F)F)cc(NS(C)(=O)=O)c1-c1ccc(N[C@@H]2CCCN(CCO)C2)nn1] | N-[2-[6-[[1-(2-hydroxyethyl)-3- piperidyl]amino]pyridazin-3-yl]-3- methyl-5- (trifluoromethyl)phenyl]methanesulfon- amide |
| 4 | 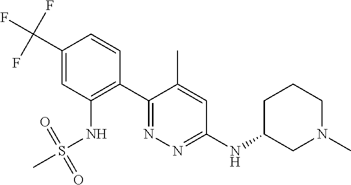 [SMILES: Cc1cc(N[C@@H]2CCCN(C)C2)nnc1-c1ccc(C(F)(F)F)cc1NS(C)(=O)=O] | N-[2-[4-methyl-6-[(1-methyl-3- piperidyl)amino]pyridazin-3-yl]-5- (trifluoromethyl)phenyl]methanesulfon- amide |
| 5 | 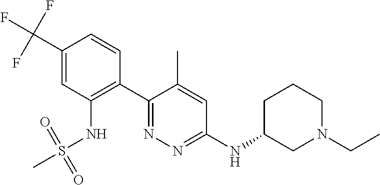 [SMILES: CCN1CCC[C@@H](Nc2cc(C)c(-c3ccc(C(F)(F)F)cc3NS(C)(=O)=O)nn2)C1] | N-[2-[6-[(1-ethyl-3-piperidyl)amino]- 4-methyl-pyridazin-3-yl]-5- (trifluoromethyl)phenyl]methanesulfon- amide |
| 6 | 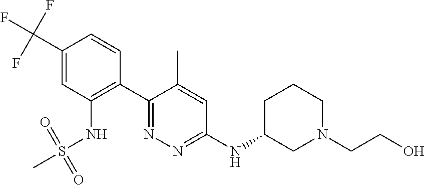 [SMILES: Cc1cc(N[C@@H]2CCCN(CCO)C2)nnc1-c1ccc(C(F)(F)F)cc1NS(C)(=O)=O] | N-[2-[6-[[1-(2-hydroxyethyl)-3- piperidyl]amino]-4-methyl-pyridazin- 3-yl]-5- (trifluoromethyl)phenyl]methanesulfon- amide |
| 7 | 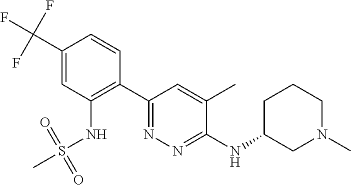 [SMILES: Cc1cc(-c2ccc(C(F)(F)F)cc2NS(C)(=O)=O)nnc1N[C@@H]1CCCN(C)C1] | N-[2-[5-methyl-6-[[(3R)-1-methyl-3- piperidyl]amino]pyridazin-3-yl]-5- (trifluoromethyl)phenyl]methanesulfon- amide |
| 8 | 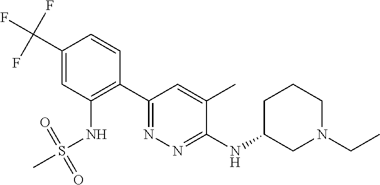 [SMILES: CCN1CCC[C@@H](Nc2nnc(-c3ccc(C(F)(F)F)cc3NS(C)(=O)=O)cc2C)C1] | N-[2-[6-[[(3R)-1-ethyl-3- piperidyl]amino]-5-methyl-pyridazin- 3-yl]-5- (trifluoromethyl)phenyl]methanesulfon- amide |
| 9 | 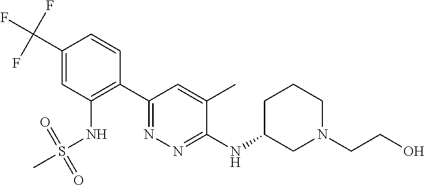 [SMILES: Cc1cc(-c2ccc(C(F)(F)F)cc2NS(C)(=O)=O)nnc1N[C@@H]1CCCN(CCO)C1] | N-[2-[6-[[(3R)-1-(2-hydroxyethyl)-3- piperidyl]amino]-5-methyl-pyridazin- 3-yl]-5- (trifluoromethyl)phenyl]methanesulfon- amide |
| 10 | 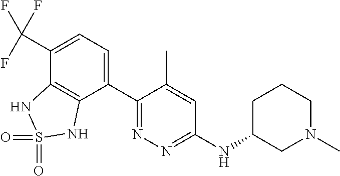 [SMILES: Cc1cc(N[C@@H]2CCCN(C)C2)nnc1-c1ccc(C(F)(F)F)c2c1NS(=O)(=O)N2] | 6-[2,2-dioxo-4-(trifluoromethyl)-1,3- dihydro-2lambda6,1,3- benzothiadiazol-7-yl]-5-methyl-N-(1- methyl-3-piperidyl)pyridazin-3- amine |
| 11 | 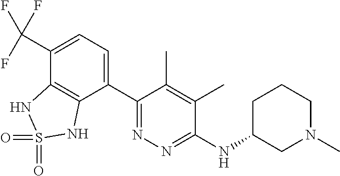 [SMILES: Cc1c(N[C@@H]2CCCN(C)C2)nnc(-c2ccc(C(F)(F)F)c3c2NS(=O)(=O)N3)c1C] | 6-[2,2-dioxo-4-(trifluoromethyl)-1,3- dihydro-2lambda6,1,3- benzothiadiazol-7-yl]-4,5-dimethyl- N-(1-methyl-3-piperidyl)pyridazin-3- amine |
| 12 | 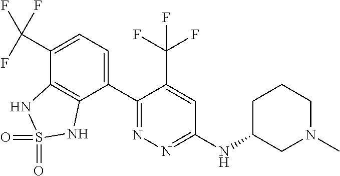 [SMILES: CN1CCC[C@@H](Nc2cc(C(F)(F)F)c(-c3ccc(C(F)(F)F)c4c3NS(=O)(=O)N4)nn2)C1] | 6-[2,2-dioxo-4-(trifluoromethyl)-1,3- dihydro-2lambda6,1,3- benzothiadiazol-7-yl]-N-[(3R)-1- methyl-3-piperidyl]-5- (trifluoromethyl)pyridazin-3-amine |
| 13 | 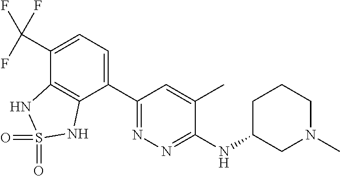 [SMILES: Cc1cc(-c2ccc(C(F)(F)F)c3c2NS(=O)(=O)N3)nnc1N[C@@H]1CCCN(C)C1] | 6-[2,2-dioxo-4-(trifluoromethyl)-1,3- dihydro-2lambda6,1,3- benzothiadiazol-7-yl]-4-methyl-N- [(3R)-1-methyl-3- piperidyl]pyridazin-3-amine |
| 14 | 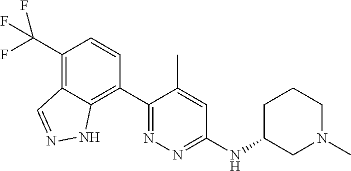 [SMILES: Cc1cc(N[C@@H]2CCCN(C)C2)nnc1-c1ccc(C(F)(F)F)c2cn[nH]c12] | 5-methyl-N-[(3R)-1-methyl-3- piperidyl]-6-[4-(trifluoromethyl)-1H- indazol-7-yl]pyridazin-3-amine |
| 15 | 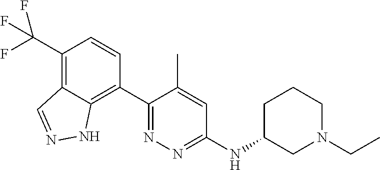 [SMILES: CCN1CCC[C@@H](Nc2cc(C)c(-c3ccc(C(F)(F)F)c4cn[nH]c34)nn2)C1] | N-[(3R)-1-ethyl-3-piperidyl]-5- methyl-6-[4-(trifluoromethyl)-1H- indazol-7-yl]pyridazin-3-amine |
| 16 | 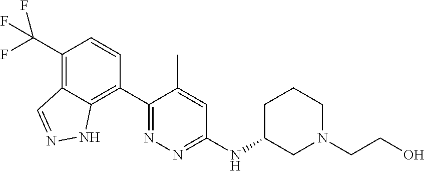 [SMILES: Cc1cc(N[C@@H]2CCCN(CCO)C2)nnc1-c1ccc(C(F)(F)F)c2cn[nH]c12] | 2-[(3R)-3-[[5-methyl-6-[4- (trifluoromethyl)-1H-indazol-7- yl]pyridazin-3-yl]amino]-1- piperidyl]ethanol |
| 17 | 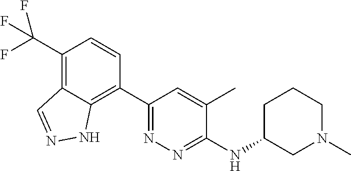 [SMILES: Cc1cc(-c2ccc(C(F)(F)F)c3cn[nH]c23)nnc1N[C@@H]1CCCN(C)C1] | 4-methyl-N-[(3R)-1-methyl-3- piperidyl]-6-[4-(trifluoromethyl)-1H- indazol-7-yl]pyridazin-3-amine |
| 18 | 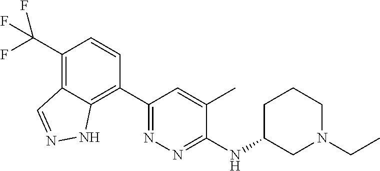 [SMILES: CCN1CCC[C@@H](Nc2nnc(-c3ccc(C(F)(F)F)c4cn[nH]c34)cc2C)C1] | N-[(3R)-1-ethyl-3-piperidyl]-4- methyl-6-[4-(trifluoromethyl)-1H- indazol-7-yl]pyridazin-3-amine |
| 19 | 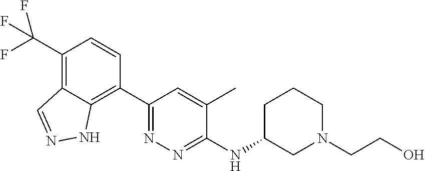 [SMILES: Cc1cc(-c2ccc(C(F)(F)F)c3cn[nH]c23)nnc1N[C@@H]1CCCN(CCO)C1] | 2-[(3R)-3-[[4-methyl-6-[4- (trifluoromethyl)-1H-indazol-7- yl]pyridazin-3-yl]amino]-1- piperidyl]ethanol |
| 20 | 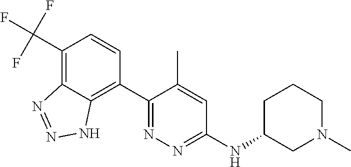 [SMILES: Cc1cc(N[C@@H]2CCCN(C)C2)nnc1-c1ccc(C(F)(F)F)c2nn[nH]c12] | 5-methyl-N-[(3R)-1-methyl-3- piperidyl]-6-[7-(trifluoromethyl)-3H- benzotriazol-4-yl]pyridazin-3-amine |
| 21 | 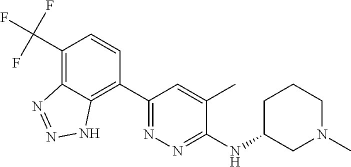 [SMILES: Cc1cc(-c2ccc(C(F)(F)F)c3nn[nH]c23)nnc1N[C@@H]1CCCN(C)C1] | 4-methyl-N-[(3R)-1-methyl-3- piperidyl]-6-[7-(trifluoromethyl)-3H- benzotriazol-4-yl]pyridazin-3-amine |
| 22 | 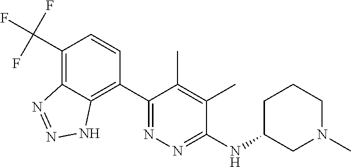 [SMILES: Cc1c(N[C@@H]2CCCN(C)C2)nnc(-c2ccc(C(F)(F)F)c3nn[nH]c23)c1C] | 4,5-dimethyl-N-[(3R)-1-methyl-3- piperidyl]-6-[7-(trifluoromethyl)-3H- benzotriazol-4-yl]pyridazin-3-amine |
| 23 | 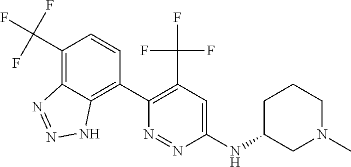 [SMILES: CN1CCC[C@@H](Nc2cc(C(F)(F)F)c(-c3ccc(C(F)(F)F)c4nn[nH]c34)nn2)C1] | N-[(3R)-1-methyl-3-piperidyl]-5- (trifluoromethyl)-6-[7- (trifluoromethyl)-3H-benzotriazol-4- yl]pyridazin-3-amine |
| 24 | 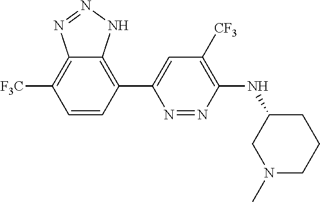 [SMILES: CN1CCC[C@@H](Nc2nnc(-c3ccc(C(F)(F)F)c4nn[nH]c34)cc2C(F)(F)F)C1] | N-[(3R)-1-methyl-3-piperidyl]-4- (trifluoromethyl)-6-[7- (trifluoromethyl)-3H-benzotriazol-4- yl]pyridazin-3-amine |
| 25 | 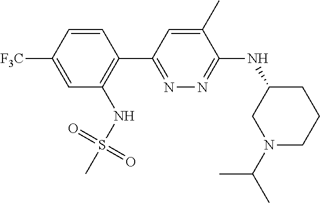 [SMILES: Cc1cc(-c2ccc(C(F)(F)F)cc2NS(C)(=O)=O)nnc1N[C@@H]1CCCN(C(C)C)C1] | N-[2-[6-[[(3R)-1-isopropyl-3- piperidyl]amino]-5-methyl-pyridazin- 3-yl]-5- (trifluoromethyl)phenyl]methanesulfon- amide |
| 26 | 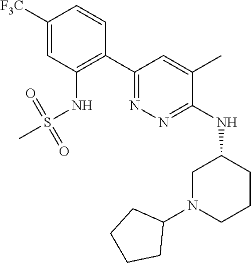 [SMILES: Cc1cc(-c2ccc(C(F)(F)F)cc2NS(C)(=O)=O)nnc1N[C@@H]1CCCN(C2CCCC2)C1] | N-[2-[6-[(3R)-1-cyclopentyl-3- piperidyl]amino]-5-methyl-pyridazin- 3-yl]-5- (trifluoromethyl)phenyl]methanesulfon- amide |
| 27 | 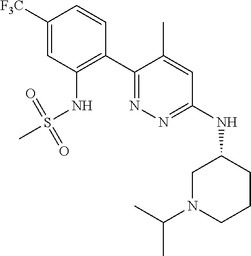 [SMILES: Cc1cc(N[C@@H]2CCCN(C(C)C)C2)nnc1-c1ccc(C(F)(F)F)cc1NS(C)(=O)=O] | N-[2-[6-[[(3R)-1-isopropyl-3- piperidyl]amino]-4-methyl-pyridazin- 3-yl-5- (trifluoromethyl)phenyl]methanesulfon- amide |
| 28 | 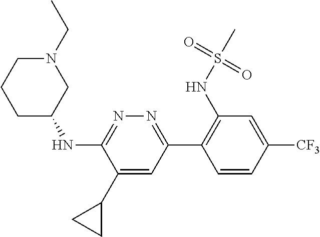 [SMILES: CCN1CCC[C@@H](Nc2nnc(-c3ccc(C(F)(F)F)cc3NS(C)(=O)=O)cc2C2CC2)C1] | N-[2-[5-cyclopropyl-6-[(3R)-1- ethyl-3-piperidyl]amino]pyridazin-3- yl]-5- (trifluoromethyl)phenyl]methanesulfon- amide |
| 29 | 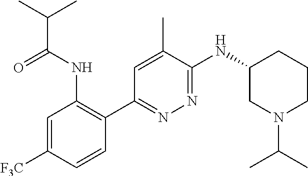 [SMILES: Cc1cc(-c2ccc(C(F)(F)F)cc2NC(=O)C(C)C)nnc1N[C@@H]1CCCN(C(C)C)C1] | N-[2-[6-[[(3R)-1-isopropyl-3- piperidyl]amino]-5-methyl-pyridazin- 3-yl]-5-(trifluoromethyl)phenyl]-2- methyl-propanamide |
| 30 | 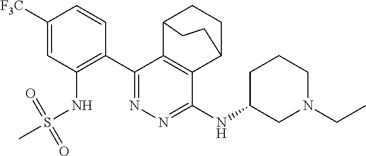 [SMILES: CCN1CCC[C@@H](Nc2nnc(-c3ccc(C(F)(F)F)cc3NS(C)(=O)=O)c3c2C2CCC3CC2)C1] | N-[2-[6-[[(3R)-1-ethyl-3- piperidyl]amino]-4,5- diazatricyclo[6.2.2.02,7]dodeca- 2(7),3,5-trien-3-yl]-5- (trifluoromethyl)phenyl]methanesulfon- amide |
| 31 | 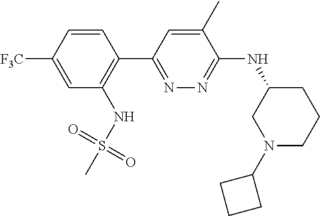 [SMILES: Cc1cc(-c2ccc(C(F)(F)F)cc2NS(C)(=O)=O)nnc1N[C@@H]1CCCN(C2CCC2)C1] | N-[2-[6-[(3R)-1-cyclobutyl-3- piperidyl]amino]-5-methyl-pyridazin- 3-yl]-5- (trifluoromethyl)phenyl]methanesulfon- amide |
| 32 | 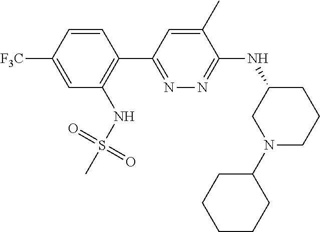 [SMILES: Cc1cc(-c2ccc(C(F)(F)F)cc2NS(C)(=O)=O)nnc1N[C@@H]1CCCN(C2CCCCC2)C1] | N-[2-[6-[(3R)-1-cyclohexyl-3- piperidyl]amino]-5-methyl-pyridazin- 3-yl]-5- (trifluoromethyl)phenyl]methanesulfon- amide |
| 33 | 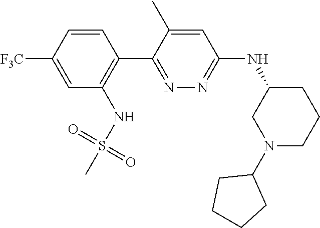 [SMILES: Cc1cc(N[C@@H]2CCCN(C3CCCC3)C2)nnc1-c1ccc(C(F)(F)F)cc1NS(C)(=O)=O] | N-[2-[6-[(3R)-1-cyclopentyl-3- piperidyl]amino]-4-methyl-pyridazin- 3-yl]-5- (trifluoromethyl)phenyl]methanesulfon- amide |
| 34 | 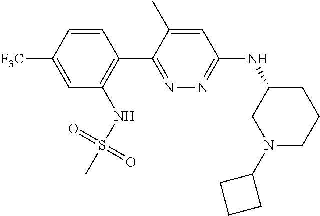 [SMILES: Cc1cc(N[C@@H]2CCCN(C3CCC3)C2)nnc1-c1ccc(C(F)(F)F)cc1NS(C)(=O)=O] | N-[2-[6-[(3R)-1-cyclobutyl-3- piperidyl]amino]-4-methyl-pyridazin- 3-yl]-5- (trifluoromethyl)phenyl]methanesulfon- amide |
| 35 | 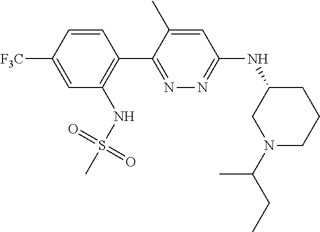 [SMILES: CCC(C)N1CCC[C@@H](Nc2cc(C)c(-c3ccc(C(F)(F)F)cc3NS(C)(=O)=O)nn2)C1] | N-[2-[4-methyl-6-[(3R)-1-sec-butyl- 3-piperidyl]amino]pyridazin-3-yl]-5- (trifluoromethyl)phenyl]methanesulfon- amide |
| 36 | 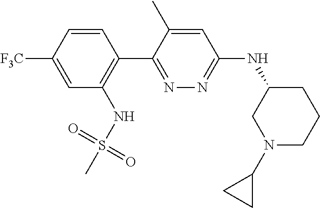 [SMILES: Cc1cc(N[C@@H]2CCCN(C3CC3)C2)nnc1-c1ccc(C(F)(F)F)cc1NS(C)(=O)=O] | N-[2-[6-[[(3R)-1-cyclopropyl-3- piperidyl]amino]-4-methyl-pyridazin- 3-yl]-5- (trifluoromethyl)phenyl]methanesulfon- amide |
| 37 | 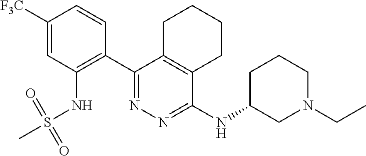 [SMILES: CCN1CCC[C@@H](Nc2nnc(-c3ccc(C(F)(F)F)cc3NS(C)(=O)=O)c3c2CCCC3)C1] | N-[2-[4-[(3R)-1-ethyl-3- piperidyl]amino]-5,6,7,8- tetrahydrophthalazin-1-yl]-5- (trifluoromethyl)phenyl]methanesulfon- amide |
| 38 | 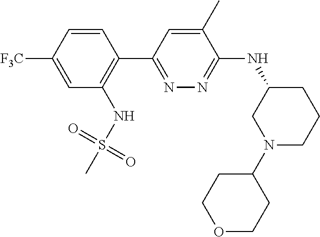 [SMILES: Cc1cc(-c2ccc(C(F)(F)F)cc2NS(C)(=O)=O)nnc1N[C@@H]1CCCN(C2CCOCC2)C1] | N-[2-[5-methyl-6-[(3R)-1- tetrahydropyran-4-yl-3- piperidyl]amino]pyridazin-3-yl]-5- (trifluoromethyl)phenyl]methanesulfon- amide |
| 39 | 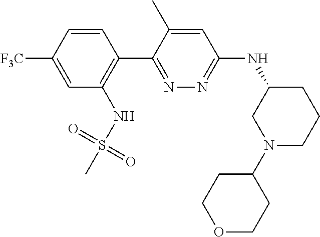 [SMILES: Cc1cc(N[C@@H]2CCCN(C3CCOCC3)C2)nnc1-c1ccc(C(F)(F)F)cc1NS(C)(=O)=O] | N-[2-[4-methyl-6-[(3R)-1- tetrahydropyran-4-yl-3- piperidyl]amino]pyridazin-3-yl]-5- (trifluoromethyl)phenyl]methanesulfon- amide |
| 40 | 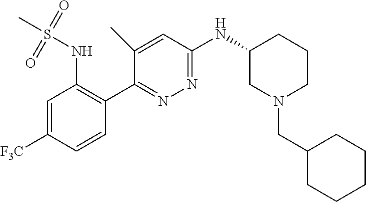 [SMILES: Cc1cc(N[C@@H]2CCCN(CC3CCCCC3)C2)nnc1-c1ccc(C(F)(F)F)cc1NS(C)(=O)=O] | N-[2-[6-[[(3R)-1-(cyclohexylmethyl)- 3-piperidyl]amino]-4-methyl- pyridazin-3-yl]-5- (trifluoromethyl)phenyl]methanesulfon- amide |
| 41 | 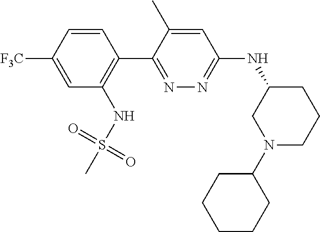 [SMILES: Cc1cc(N[C@@H]2CCCN(C3CCCCC3)C2)nnc1-c1ccc(C(F)(F)F)cc1NS(C)(=O)=O] | N-[2-[6-[[(3R)-1-cyclohexyl-3- piperidyl]amino]-4-methyl-pyridazin- 3-yl]-5- (trifluoromethyl)phenyl]methanesulfon- amide |
| 42 | 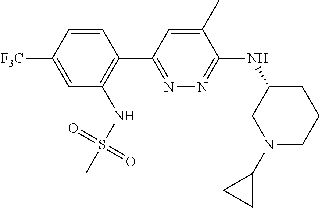 [SMILES: Cc1cc(-c2ccc(C(F)(F)F)cc2NS(C)(=O)=O)nnc1N[C@@H]1CCCN(C2CC2)C1] | N-[2-[6-[(3R)-1-cyclopropyl-3- piperidyl]amino]-5-methyl-pyridazin- 3-yl]-5- (trifluoromethyl)phenyl]methanesulfon- amide |
| 43 | 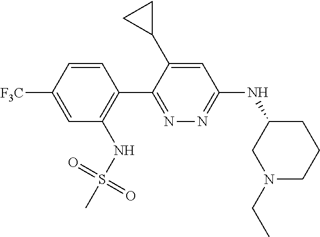 [SMILES: CCN1CCC[C@@H](Nc2cc(C3CC3)c(-c3ccc(C(F)(F)F)cc3NS(C)(=O)=O)nn2)C1] | N-[2-[4-cyclopropyl-6-[(3R)-1- ethyl-3-piperidyl]amino]pyridazin-3- yl]-5- (trifluoromethyl)phenyl]methanesulfon- amide |
| 44 | 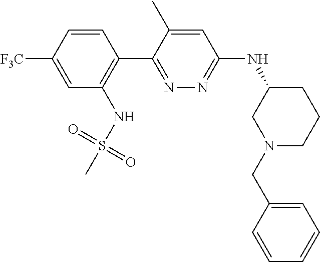 [SMILES: Cc1cc(N[C@@H]2CCCN(Cc3ccccc3)C2)nnc1-c1ccc(C(F)(F)F)cc1NS(C)(=O)=O] | N-[2-[6-[[(3R)-1-benzyl-3- piperidyl]amino]-4-methyl-pyridazin- 3-yl]-5- (trifluoromethyl)phenyl]methanesulfon- amide |
| 45 | 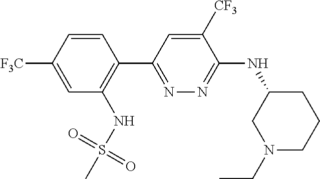 [SMILES: CCN1CCC[C@@H](Nc2nnc(-c3ccc(C(F)(F)F)cc3NS(C)(=O)=O)cc2C(F)(F)F)C1] | N-[2-[6-[[(3R)-1-ethyl-3- piperidyl]amino]-5- (trifluoromethyl)pyridazin-3-yl]-5- (trifluoromethyl)phenyl]methanesulfon- amide |
| 46 | 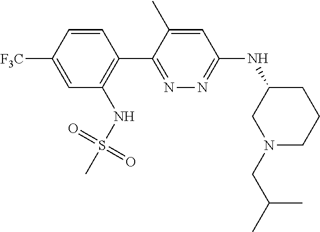 [SMILES: Cc1cc(N[C@@H]2CCCN(CC(C)C)C2)nnc1-c1ccc(C(F)(F)F)cc1NS(C)(=O)=O] | N-[2-[6-[(3R)-1-isobutyl-3- piperidyl]amino]-4-methyl-pyridazin- 3-yl]-5- (trifluoromethyl)phenyl]methanesulfon- amide |
| 47 | 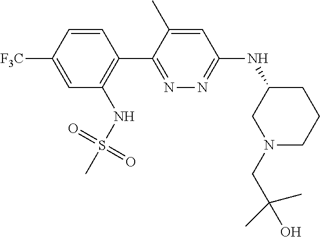 [SMILES: Cc1cc(N[C@@H]2CCCN(CC(C)(C)O)C2)nnc1-c1ccc(C(F)(F)F)cc1NS(C)(=O)=O] | N-[2-[6-[(3R)-1-(2-hydroxy-2- methyl-propyl)-3-piperidyl]amino]-4- methyl-pyridazin-3-yl]-5- (trifluoromethyl)phenyl]methanesulfon- amide |
| 48 | 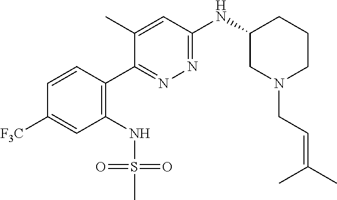 [SMILES: CC(C)=CCN1CCC[C@@H](Nc2cc(C)c(-c3ccc(C(F)(F)F)cc3NS(C)(=O)=O)nn2)C1] | N-[2-[4-methyl-6-[[(3R)-1-(3- methylbut-2-enyl)-3- piperidyl]amino]pyridazin-3-yl]-5- (trifluoromethyl)phenyl]methanesulfon- amide |
| 49 | 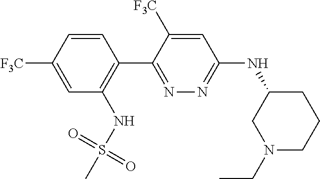 [SMILES: CCN1CCC[C@@H](Nc2cc(C(F)(F)F)c(-c3ccc(C(F)(F)F)cc3NS(C)(=O)=O)nn2)C1] | N-[2-[6-[[(3R)-1-ethyl-3- piperidyl]amino]-4- (trifluoromethyl)pyridazin-3-yl]-5- (trifluoromethyl)phenyl]methanesulfon- amide |
| 50 | 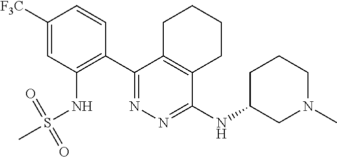 [SMILES: CN1CCC[C@@H](Nc2nnc(-c3ccc(C(F)(F)F)cc3NS(C)(=O)=O)c3c2CCCC3)C1] | N-[2-[4-[[(3R)-1-methyl-3- piperidyl]amino]-5,6,7,8- tetrahydrophthalazin-1-yl]-5- (trifluoromethyl)phenyl]methanesulfon- amide |
| 51 | 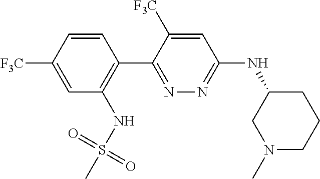 [SMILES: CN1CCC[C@@H](Nc2cc(C(F)(F)F)c(-c3ccc(C(F)(F)F)cc3NS(C)(=O)=O)nn2)C1] | N-[2-[6-[[(3R)-1-methyl-3- piperidyl]amino]-4- (trifluoromethyl)pyridazin-3-yl]-5- (trifluoromethyl)phenyl]methanesulfon- amide |
| 52 | 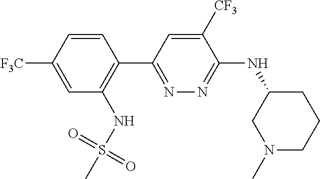 [SMILES: CN1CCC[C@@H](Nc2nnc(-c3ccc(C(F)(F)F)cc3NS(C)(=O)=O)cc2C(F)(F)F)C1] | N-[2-[6-[[(3R)-1-methyl-3- piperidyl]amino]-5- (trifluoromethyl)pyridazin-3-yl]-5- (trifluoromethyl)phenyl]methanesulfon- amide |
| 53 | 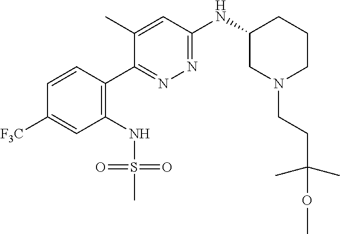 [SMILES: COC(C)(C)CCN1CCC[C@@H](Nc2cc(C)c(-c3ccc(C(F)(F)F)cc3NS(C)(=O)=O)nn2)C1] | N-[2-[6-[[(3R)-1-(3-methoxy-3- methyl-butyl)-3-piperidyl]amino]-4- methyl-pyridazin-3-yl]-5- (trifluoromethyl)phenyl]methanesulfon- amide |
| 54 | 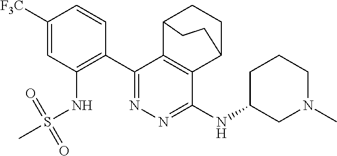 [SMILES: CN1CCC[C@@H](Nc2nnc(-c3ccc(C(F)(F)F)cc3NS(C)(=O)=O)c3c2C2CCC3CC2)C1] | N-[2-[6-[[(3R)-1-methyl-3- piperidyl]amino]-4,5- diazatricyclo[6.2.2.02,7]dodeca- 2(7),3,5-trien-3-yl]-5- (trifluoromethyl)phenyl]methanesulfon- amide |
| 55 | 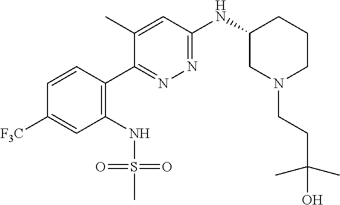 [SMILES: Cc1cc(N[C@@H]2CCCN(CCC(C)(C)O)C2)nnc1-c1ccc(C(F)(F)F)cc1NS(C)(=O)=O] | N-[2-[6-[[(3R)-1-(3-hydroxy-3- methyl-butyl)-3-piperidyl]amino]-4- methyl-pyridazin-3-yl]-5- (trifluoromethyl)phenyl]methanesulfon- amide |
| 56 | 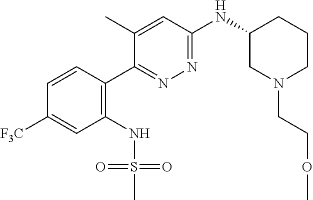 [SMILES: COCCN1CCC[C@@H](Nc2cc(C)c(-c3ccc(C(F)(F)F)cc3NS(C)(=O)=O)nn2)C1] | N-[2-[6-[[(3R)-1-(2-methoxyethyl)-3- piperidyl]amino]-4-methyl-pyridazin- 3-yl]-5- (trifluoromethyl)phenyl]methanesulfon- amide |
| 57 | 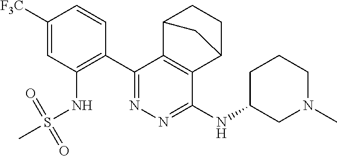 [SMILES: CN1CCC[C@@H](Nc2nnc(-c3ccc(C(F)(F)F)cc3NS(C)(=O)=O)c3c2C2CCC3C2)C1] | N-[2-[6-[[(3R)-1-methyl-3- piperidyl]amino]-4,5- diazatricyclo[6.2.1.02,7]undeca- 2(7), 3,5-trien-3-yl]-5- (trifluoromethyl)phenyl]methanesulfon- amide |
| 58 | 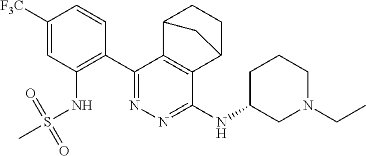 [SMILES: CCN1CCC[C@@H](Nc2nnc(-c3ccc(C(F)(F)F)cc3NS(C)(=O)=O)c3c2C2CCC3C2)C1] | N-[2-[6-[[(3R)-1-ethyl-3- piperidyl]amino]-4,5- diazatricyclo[6.2.1.02,7]undeca- 2(7),3,5-trien-3-yl]-5- (trifluoromethyl)phenyl]methanesulfon- amide |
| 59 | 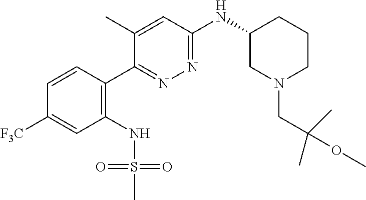 [SMILES: COC(C)(C)CN1CCC[C@@H](Nc2cc(C)c(-c3ccc(C(F)(F)F)cc3NS(C)(=O)=O)nn2)C1] | N-[2-[6-[(3R)-1-(2-methoxy-2- methyl-propyl)-3-piperidyl]amino]-4- methyl-pyridazin-3-yl]-5- (trifluoromethyl)phenyl]methanesulfon- amide |
| 60 | 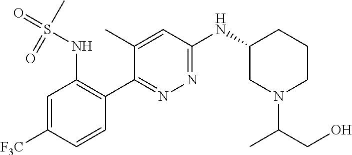 [SMILES: Cc1cc(N[C@@H]2CCCN(C(C)CO)C2)nnc1-c1ccc(C(F)(F)F)cc1NS(C)(=O)=O] | N-[2-[6-[[(3R)-1-(2-hydroxy-1- methyl-ethyl)-3-piperidyl]amino]-4- methyl-pyridazin-3-yl]-5- (trifluoromethyl)phenyl]methanesulfon- amide |
| 61 | 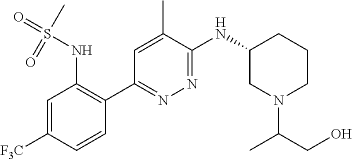 [SMILES: Cc1cc(-c2ccc(C(F)(F)F)cc2NS(C)(=O)=O)nnc1N[C@@H]1CCCN(C(C)CO)C1] | N-[2-[6-[[(3R)-1-(2-hydroxy-1- methyl-ethyl)-3-piperidyl]amino]-5- methyl-pyridazin-3-yl]-5- (trifluoromethyl)phenyl]methanesulfon- amide |
| 62 | 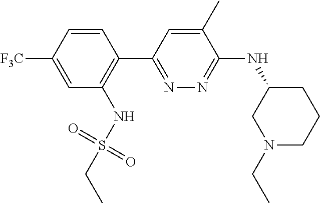 [SMILES: CCN1CCC[C@@H](Nc2nnc(-c3ccc(C(F)(F)F)cc3NS(=O)(=O)CC)cc2C)C1] | N-[2-[6-[[(3R)-1-ethyl-3- piperidyl]amino]-5-methyl-pyridazin- 3-yl]-5- (trifluoromethyl)phenyl]ethanesulfon- amide |
| 63 | 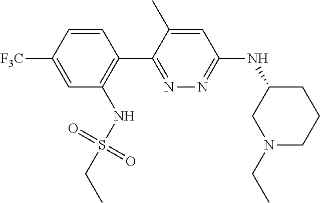 [SMILES: CCN1CCC[C@@H](Nc2cc(C)c(-c3ccc(C(F)(F)F)cc3NS(=O)(=O)CC)nn2)C1] | N-[2-[6-[[(3R)-1-ethyl-3- piperidyl]amino]-4-methyl-pyridazin- 3-yl]-5- (trifluoromethyl)phenyl]ethanesulfon- amide |
| 64 | 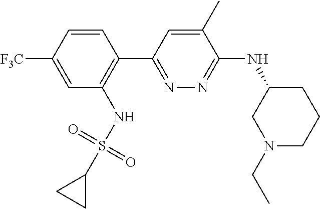 [SMILES: CCN1CCC[C@@H](Nc2nnc(-c3ccc(C(F)(F)F)cc3NS(=O)(=O)C3CC3)cc2C)C1] | N-[2-[6-[(3R)-1-ethyl-3- piperidyl]amino]-5-methyl-pyridazin- 3-yl]-5- (trifluoromethyl)phenyl]cyclopropane- sulfonamide |
| 65 | 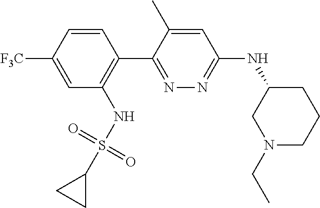 [SMILES: CCN1CCC[C@@H](Nc2cc(C)c(-c3ccc(C(F)(F)F)cc3NS(=O)(=O)C3CC3)nn2)C1] | N-[2-[6-[[(3R)-1-ethyl-3- piperidyl]amino]-4-methyl-pyridazin- 3-yl]-5- (trifluoromethyl)phenyl]cyclopropane- sulfonamide |
| 66 | 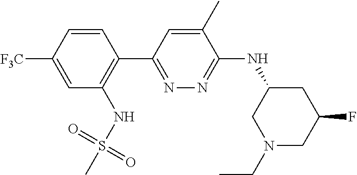 [SMILES: CCN1C[C@H](F)C[C@@H](Nc2nnc(-c3ccc(C(F)(F)F)cc3NS(C)(=O)=O)cc2C)C1] | N-[2-[6-[[(3R,5R)-1-ethyl-5-fluoro- 3-piperidyl]amino]-5-methyl- pyridazin-3-yl]-5- (trifluoromethyl)phenyl]methanesulfon- amide |
| 67 | 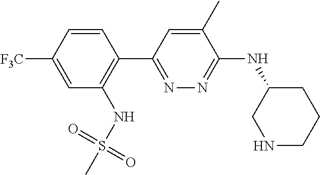 [SMILES: Cc1cc(-c2ccc(C(F)(F)F)cc2NS(C)(=O)=O)nnc1N[C@@H]1CCCNC1] | N-[2-[5-methyl-6-[[(3R)-3- piperidyl]amino]pyridazin-3-yl]-5- (trifluoromethyl)phenyl]methanesulfon- amide |
| 68 | 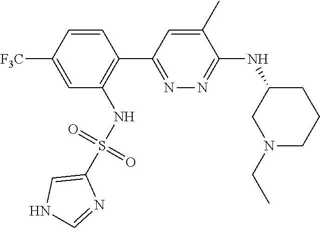 [SMILES: CCN1CCC[C@@H](Nc2nnc(-c3ccc(C(F)(F)F)cc3NS(=O)(=O)c3c[nH]cn3)cc2C)C1] | N-[2-[6-[[(3R)-1-ethyl-3- piperidyl]amino]-5-methyl-pyridazin- 3-yl]-5-(trifluoromethyl)phenyl]-1H- imidazole-4-sulfonamide |
| 69 | 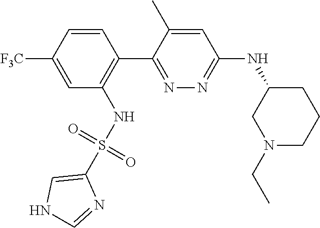 [SMILES: CCN1CCC[C@@H](Nc2cc(C)c(-c3ccc(C(F)(F)F)cc3NS(=O)(=O)c3c[nH]cn3)nn2)C1] | N-[2-[6-[[(3R)-1-ethyl-3- piperidyl]amino]-4-methyl-pyridazin- 3-yl]-5-(trifluoromethyl)phenyl]-1H- imidazole-4-sulfonamide |
| 70 | 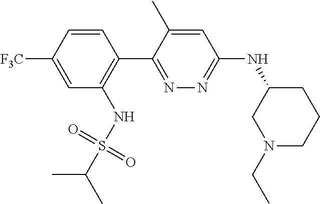 [SMILES: CCN1CCC[C@@H](Nc2cc(C)c(-c3ccc(C(F)(F)F)cc3NS(=O)(=O)C(C)C)nn2)C1] | N-[2-[6-[[(3R)-1-ethyl-3- piperidyl]amino]-4-methyl-pyridazin- 3-yl]-5- (trifluoromethyl)phenyl]propane-2- sulfonamide |
| 71 | 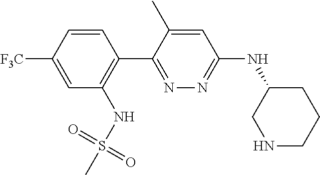 [SMILES: Cc1cc(N[C@@H]2CCCNC2)nnc1-c1ccc(C(F)(F)F)cc1NS(C)(=O)=O] | N-[2-[4-methyl-6-[[(3R)-3- piperidyl]amino]pyridazin-3-yl]-5- (trifluoromethyl)phenyl]methanesulfon- amide |
| 72 | 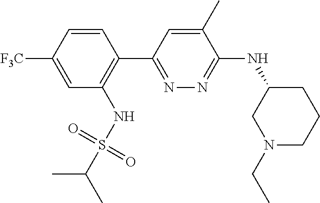 [SMILES: CCN1CCC[C@@H](Nc2nnc(-c3ccc(C(F)(F)F)cc3NS(=O)(=O)C(C)C)cc2C)C1] | N-[2-[6-[[(3R)-1-ethyl-3- piperidyl]amino]-5-methyl-pyridazin- 3-yl]-5- (trifluoromethyl)phenyl]propane-2- sulfonamide |
| 73 | 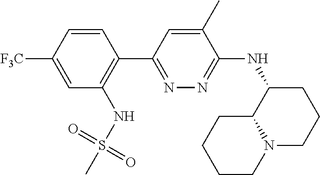 [SMILES: Cc1cc(-c2ccc(C(F)(F)F)cc2NS(C)(=O)=O)nnc1N[C@@H]1CCCN2CCCC[C@H]12] | N-[2-[6-[[(1R,9aR)-2,3,4,6,7,8,9,9a- octahydro-1H-quinolizin-1- yl]amino]-5-methyl-pyridazin-3-yl]- 5- (trifluoromethyl)phenyl]methanesulfon- amide |
| 74 | 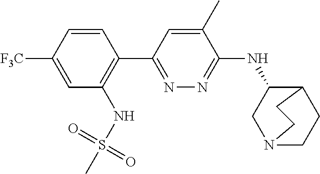 [SMILES: Cc1cc(-c2ccc(C(F)(F)F)cc2NS(C)(=O)=O)nnc1N[C@H]1CN2CCC1CC2] | N-[2-[5-methyl-6-[[(3R)-quinuclidin- 3-yl]amino]pyridazin-3-yl]-5- (trifluoromethyl)phenyl]methanesulfon- amide |
| 75 | 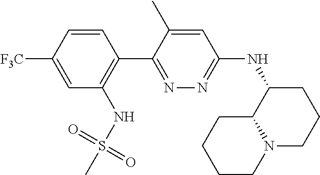 [SMILES: Cc1cc(N[C@@H]2CCCN3CCCC[C@H]23)nnc1-c1ccc(C(F)(F)F)cc1NS(C)(=O)=O] | N-[2-[6-[[(1R,9aR)-2,3,4,6,7,8,9,9a- octahydro-1H-quinolizin-1- yl]amino]-4-methyl-pyridazin-3-yl]- 5- (trifluoromethyl)phenyl]methanesulfon- amide |
| 76 | 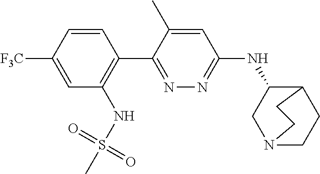 [SMILES: Cc1cc(N[C@H]2CN3CCC2CC3)nnc1-c1ccc(C(F)(F)F)cc1NS(C)(=O)=O] | N-[2-[4-methyl-6-[[(3R)-quinuclidin- 3-yl]amino]pyridazin-3-yl]-5- (trifluoromethyl)phenyl]methanesulfon- amide |
| 77 | 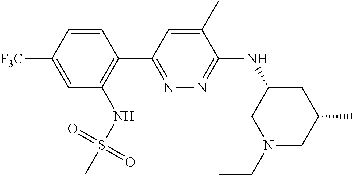 [SMILES: CCN1C[C@@H](C)C[C@@H](Nc2nnc(-c3ccc(C(F)(F)F)cc3NS(C)(=O)=O)cc2C)C1] | N-[2-[6-[[(3R,5S)-1-ethyl-5-methyl- 3-piperidyl]amino]-5-methyl- pyridazin-3-yl]-5- (trifluoromethyl)phenyl]methanesulfon- amide |
| 78 | 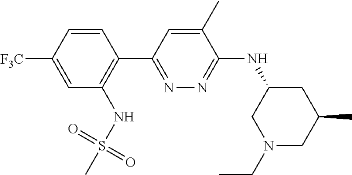 [SMILES: CCN1C[C@H](C)C[C@@H](Nc2nnc(-c3ccc(C(F)(F)F)cc3NS(C)(=O)=O)cc2C)C1] | N-[2-[6-[[(3R,5R)-1-ethyl-5-methyl- 3-piperidyl]amino]-5-methyl- pyridazin-3-yl]-5- (trifluoromethyl)phenyl]methanesulfon- amide |
| 79 | 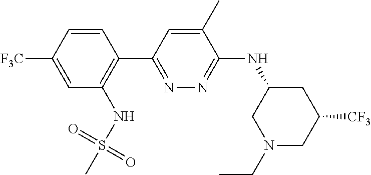 [SMILES: CCN1C[C@H](Nc2nnc(-c3ccc(C(F)(F)F)cc3NS(C)(=O)=O)cc2C)C[C@H](C(F)(F)F)C1] | N-[2-[6-[[(3R,5S)-1-ethyl-5- (trifluoromethyl)-3-piperidyl]amino]- 5-methyl-pyridazin-3-yl]-5- (trifluoromethyl)phenyl]methanesulfon- amide |
| 80 | 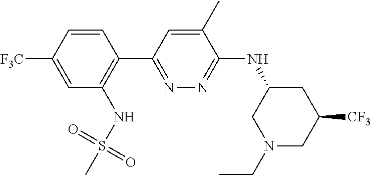 [SMILES: CCN1C[C@H](Nc2nnc(-c3ccc(C(F)(F)F)cc3NS(C)(=O)=O)cc2C)C[C@@H](C(F)(F)F)C1] | N-[2-[6-[[(3R,5R)-1-ethyl-5- (trifluoromethyl)-3-piperidyl]amino]- 5-methyl-pyridazin-3-yl]-5- (trifluoromethyl)phenyl]methanesulfon- amide |
| 81 | 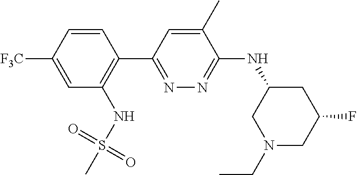 [SMILES: CCN1C[C@@H](F)C[C@@H](Nc2nnc(-c3ccc(C(F)(F)F)cc3NS(C)(=O)=O)cc2C)C1] | N-[2-[6-[[(3R,5S)-1-ethyl-5-fluoro-3- piperidyl]amino]-5-methyl-pyridazin- 3-yl]-5- (trifluoromethyl)phenyl]methanesulfon- amide |
| 82 | 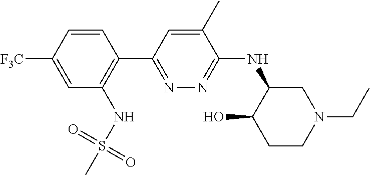 [SMILES: CCN1CC[C@@H](O)[C@@H](Nc2nnc(-c3ccc(C(F)(F)F)cc3NS(C)(=O)=O)cc2C)C1] | N-[2-[6-[[(3S,4R)-1-ethyl-4-hydroxy- 3-piperidyl]amino]-5-methyl- pyridazin-3-yl]-5- (trifluoromethyl)phenyl]methanesulfon- amide |
| 83 | 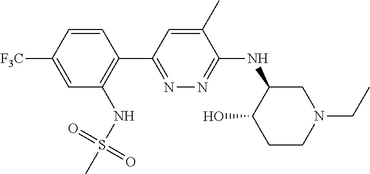 [SMILES: CCN1CC[C@H](O)[C@@H](Nc2nnc(-c3ccc(C(F)(F)F)cc3NS(C)(=O)=O)cc2C)C1] | N-[2-[6-[[(3S,4S)-1-ethyl-4-hydroxy- 3-piperidyl]amino]-5-methyl- pyridazin-3-yl]-5- (trifluoromethyl)phenyl]methanesulfon- amide |
| 84 | 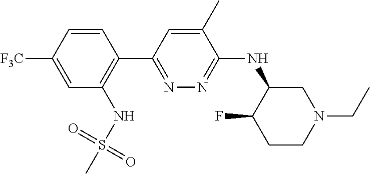 [SMILES: CCN1CC[C@@H](F)[C@@H](Nc2nnc(-c3ccc(C(F)(F)F)cc3NS(C)(=O)=O)cc2C)C1] | N-[2-[6-[[(3S,4R)-1-ethyl-4-fluoro-3- piperidyl]amino]-5-methyl-pyridazin- 3-yl]-5- (trifluoromethyl)phenyl]methanesulfon- amide |
| 85 | 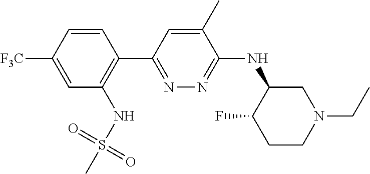 [SMILES: CCN1CC[C@H](F)[C@@H](Nc2nnc(-c3ccc(C(F)(F)F)cc3NS(C)(=O)=O)cc2C)C1] | N-[2-[6-[[(3S,4S)-1-ethyl-4-fluoro-3- piperidyl]amino]-5-methyl-pyridazin- 3-yl]-5- (trifluoromethyl)phenyl]methanesulfon- amide |
| 86 | 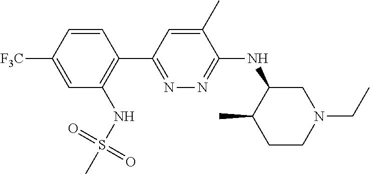 [SMILES: CCN1CC[C@@H](C)[C@@H](Nc2nnc(-c3ccc(C(F)(F)F)cc3NS(C)(=O)=O)cc2C)C1] | N-[2-[6-[[(3R,4R)-1-ethyl-4-methyl- 3-piperidyl]amino]-5-methyl- pyridazin-3-yl]-5- (trifluoromethyl)phenyl]methanesulfon- amide |
| 87 | 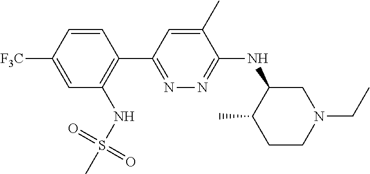 [SMILES: CCN1CC[C@H](C)[C@@H](Nc2nnc(-c3ccc(C(F)(F)F)cc3NS(C)(=O)=O)cc2C)C1] | N-[2-[6-[(3R,4S)-1-ethyl-4-methyl- 3-piperidyl]amino]-5-methyl- pyridazin-3-yl]-5- (trifluoromethyl)phenyl]methanesulfon- amide |
| 88 | 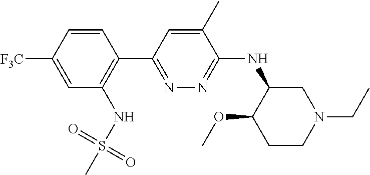 [SMILES: CCN1CC[C@@H](OC)[C@@H](Nc2nnc(-c3ccc(C(F)(F)F)cc3NS(C)(=O)=O)cc2C)C1] | N-[2-[6-[[(3S,4R)-1-ethyl-4- methoxy-3-piperidyl]amino]-5- methyl-pyridazin-3-yl]-5- (trifluoromethyl)phenyl]methanesulfon- amide |
| 89 | 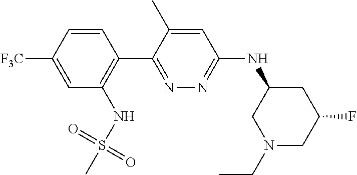 [SMILES: CCN1C[C@@H](F)C[C@H](Nc2cc(C)c(-c3ccc(C(F)(F)F)cc3NS(C)(=O)=O)nn2)C1] | N-[2-[6-[[(3S,5S)-1-ethyl-5-fluoro-3- piperidyl]amino]-4-methyl-pyridazin- 3-yl]-5- (trifluoromethyl)phenyl]methanesulfon- amide |
| 90 | 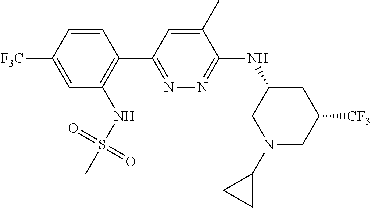 [SMILES: Cc1cc(-c2ccc(C(F)(F)F)cc2NS(C)(=O)=O)nnc1N[C@@H]1C[C@H](C(F)(F)F)CN(C2CC2)C1] | N-[2-[6-[[(3R,5S)-1-cyclopropyl-5- (trifluoromethyl)-3-piperidyl]amino]- 5-methyl-pyridazin-3-yl]-5- (trifluoromethyl)phenyl]methanesulfon- amide |
| 91 | 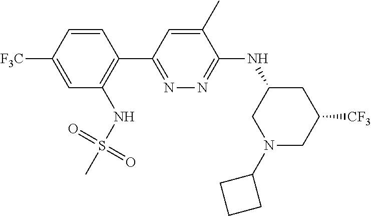 [SMILES: Cc1cc(-c2ccc(C(F)(F)F)cc2NS(C)(=O)=O)nnc1N[C@@H]1C[C@H](C(F)(F)F)CN(C2CCC2)C1] | N-[2-[6-[(3R,5S)-1-cyclobutyl-5- (trifluoromethyl)-3-piperidyl]amino]- 5-methyl-pyridazin-3-yl]-5- (trifluoromethyl)phenyl]methanesulfon- amide |
| 92 | 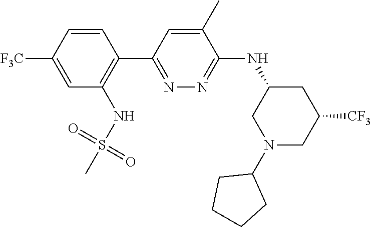 [SMILES: Cc1cc(-c2ccc(C(F)(F)F)cc2NS(C)(=O)=O)nnc1N[C@@H]1C[C@H](C(F)(F)F)CN(C2CCCC2)C1] | N-[2-[6-[[(3R,5S)-1-cyclopentyl-5- (trifluoromethyl)-3-piperidyl]amino]- 5-methyl-pyridazin-3-yl]-5- (trifluoromethyl)phenyl]methanesulfon- amide |
| 93 | 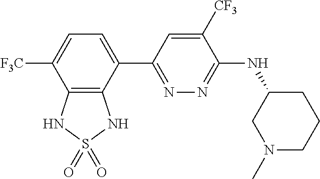 [SMILES: CN1CCC[C@@H](Nc2nnc(-c3ccc(C(F)(F)F)c4c3NS(=O)(=O)N4)cc2C(F)(F)F)C1] | 6-[2,2-dioxo-4-(trifluoromethyl)-1,3- dihydro-2lambda6,1,3- benzothiadiazol-7-yl]-N-[(3R)-1- methyl-3-piperidyl]-4- (trifluoromethyl)pyridazin-3-amine |
| 94 | 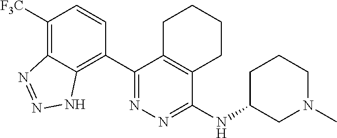 [SMILES: CN1CCC[C@@H](Nc2nnc(-c3ccc(C(F)(F)F)c4nn[nH]c34)c3c2CCCC3)C1] | N-[(3R)-1-methyl-3-piperidyl]-4-[7- (trifluoromethyl)-3H-benzotriazol-4- yl]-5,6,7,8-tetrahydrophthalazin-1- amine |
| 95 | 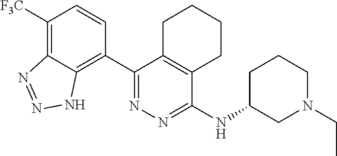 [SMILES: CCN1CCC[C@@H](Nc2nnc(-c3ccc(C(F)(F)F)c4nn[nH]c34)c3c2CCCC3)C1] | N-[(3R)-1-ethyl-3-piperidyl]-4-[7- (trifluoromethyl)-3H-benzotriazol-4- yl]-5,6,7,8-tetrahydrophthalazin-1- amine |
| 96 | 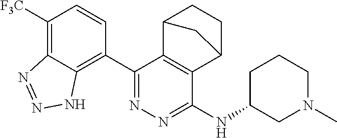 [SMILES: CN1CCC[C@@H](Nc2nnc(-c3ccc(C(F)(F)F)c4nn[nH]c34)c3c2C2CCC3C2)C1] | N-[(3R)-1-methyl-3-piperidyl]-6-[7- (trifluoromethyl)-3H-benzotriazol-4- yl]-4,5- diazatricyclo[6.2.1.02,7]undeca- 2(7), 3,5-trien-3-amine |
| 97 | 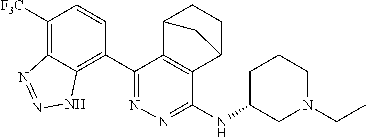 [SMILES: CCN1CCC[C@@H](Nc2nnc(-c3ccc(C(F)(F)F)c4nn[nH]c34)c3c2C2CCC3C2)C1] | N-[(3R)-1-ethyl-3-piperidyl]-6-[7- (trifluoromethyl)-3H-benzotriazol-4- yl]-4,5- diazatricyclo[6.2.1.02,7]undeca- 2(7),3,5-trien-3-amine |
| 98 | 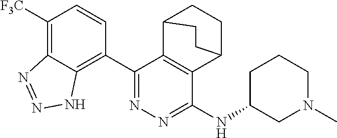 [SMILES: CN1CCC[C@@H](Nc2nnc(-c3ccc(C(F)(F)F)c4nn[nH]c34)c3c2C2CCC3CC2)C1] | N-[(3R)-1-methyl-3-piperidyl]-6-[7- (trifluoromethyl)-3H-benzotriazol-4- yl]-4,5- diazatricyclo[6.2.2.02,7]dodeca- 2(7),3,5-trien-3-amine |
| 99 | 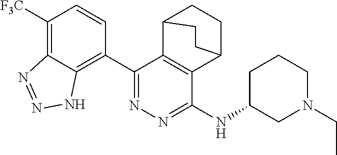 [SMILES: CCN1CCC[C@@H](Nc2nnc(-c3ccc(C(F)(F)F)c4nn[nH]c34)c3c2C2CCC3CC2)C1] | N-[(3R)-1-ethyl-3-piperidyl]-6-[7- (trifluoromethyl)-3H-benzotriazol-4- yl]-4,5- diazatricyclo[6.2.2.02,7]dodeca- 2(7), 3,5-trien-3-amine |
| 100 | 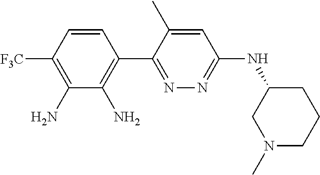 [SMILES: Cc1cc(N[C@@H]2CCCN(C)C2)nnc1-c1ccc(C(F)(F)F)c(N)c1N] | 3-[4-methyl-6-[(3R)-1-methyl-3- piperidyl]amino]pyridazin-3-yl]-6- (trifluoromethyl)benzene-1,2-diamine |
| 101 | 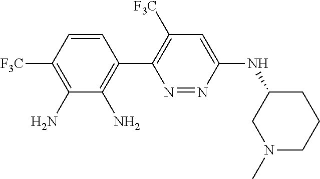 [SMILES: CN1CCC[C@@H](Nc2cc(C(F)(F)F)c(-c3ccc(C(F)(F)F)c(N)c3N)nn2)C1] | 3-[6-[[(3R)-1-methyl-3- piperidyl]amino]-4- (trifluoromethyl)pyridazin-3-yl]-6- (trifluoromethyl)benzene-1,2-diamine |
| 102 | 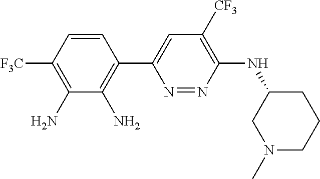 [SMILES: CN1CCC[C@@H](Nc2nnc(-c3ccc(C(F)(F)F)c(N)c3N)cc2C(F)(F)F)C1] | 3-[6-[[(3R)-1-methyl-3- piperidyl]amino]-5- (trifluoromethyl)pyridazin-3-yl]-6- (trifluoromethyl)benzene-1,2-diamine |
| 103 | 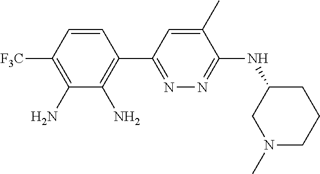 [SMILES: Cc1cc(-c2ccc(C(F)(F)F)c(N)c2N)nnc1N[C@@H]1CCCN(C)C1] | 3-[5-methyl-6-[[(3R)-1-methyl-3- piperidyl]amino]pyridazin-3-yl]-6- (trifluoromethyl)benzene-1,2-diamine |
| 104 | 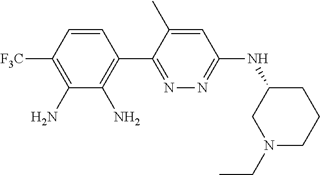 [SMILES: CCN1CCC[C@@H](Nc2cc(C)c(-c3ccc(C(F)(F)F)c(N)c3N)nn2)C1] | 3-[6-[[(3R)-1-ethyl-3- piperidyl]amino]-4-methyl-pyridazin- 3-yl]-6-(trifluoromethyl)benzene-1,2- diamine |
| 105 | 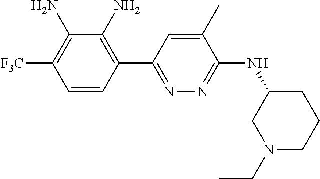 [SMILES: CCN1CCC[C@@H](Nc2nnc(-c3ccc(C(F)(F)F)c(N)c3N)cc2C)C1] | 3-[6-[[(3R)-1-ethyl-3- piperidyl]amino]-5-methyl-pyridazin- 3-yl]-6-(trifluoromethyl)benzene-1,2- diamine |
| 106 | 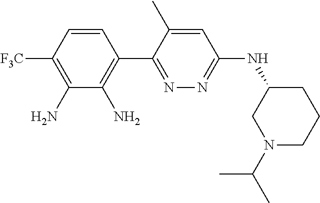 [SMILES: Cc1cc(N[C@@H]2CCCN(C(C)C)C2)nnc1-c1ccc(C(F)(F)F)c(N)c1N] | 3-[6-[[(3R)-1-isopropyl-3- piperidyl]amino]-4-methyl-pyridazin- 3-yl]-6-(trifluoromethyl)benzene-1,2- diamine |
| 107 | 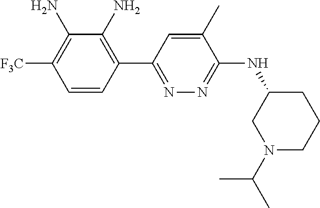 [SMILES: Cc1cc(-c2ccc(C(F)(F)F)c(N)c2N)nnc1N[C@@H]1CCCN(C(C)C)C1] | 3-[6-[[(3R)-1-isopropyl-3- piperidyl]amino]-5-methyl-pyridazin- 3-yl]-6-(trifluoromethyl)benzene-1,2- diamine |
| 108 | 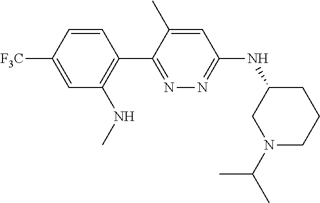 [SMILES: CNc1cc(C(F)(F)F)ccc1-c1nnc(N[C@@H]2CCCN(C(C)C)C2)cc1C] | N-[(3R)-1-isopropyl-3-piperidyl]-5- methyl-6-[2-(methylamino)-4- (trifluoromethyl)phenyl]pyridazin-3- amine |
| 109 | 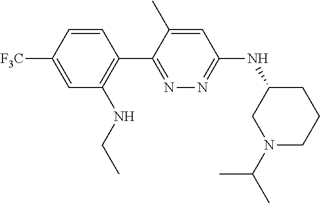 [SMILES: CCNc1cc(C(F)(F)F)ccc1-c1nnc(N[C@@H]2CCCN(C(C)C)C2)cc1C] | 6-[2-(ethylamino)-4- (trifluoromethyl)phenyl]-N-[(3R)-1- isopropyl-3-piperidyl]-5-methyl- pyridazin-3-amine |
| 110 | 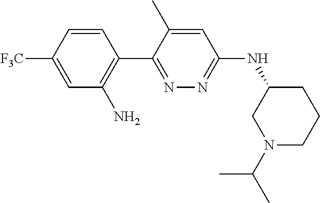 [SMILES: Cc1cc(N[C@@H]2CCCN(C(C)C)C2)nnc1-c1ccc(C(F)(F)F)cc1N] | 6-[2-amino-4- (trifluoromethyl)phenyl]-N-[(3R)-1- isopropyl-3-piperidyl]-5-methyl- pyridazin-3-amine |
| 111 | 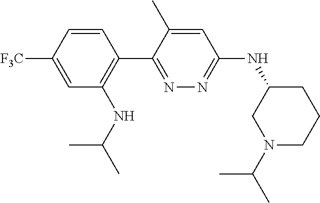 [SMILES: Cc1cc(N[C@@H]2CCCN(C(C)C)C2)nnc1-c1ccc(C(F)(F)F)cc1NC(C)C] | 6-[2-(isopropylamino)-4- (trifluoromethyl)phenyl]-N-[(3R)-1- isopropyl-3-piperidyl]-5-methyl- pyridazin-3-amine |
| 112 | 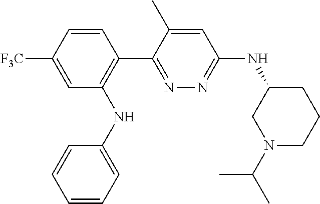 [SMILES: Cc1cc(N[C@@H]2CCCN(C(C)C)C2)nnc1-c1ccc(C(F)(F)F)cc1Nc1ccccc1] | 6-[2-anilino-4- (trifluoromethyl)phenyl]-N-[(3R)-1- isopropyl-3-piperidyl]-5-methyl- pyridazin-3-amine |
| 113 | 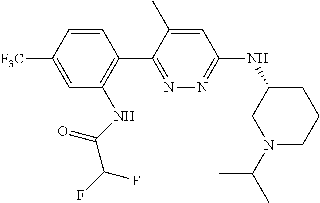 [SMILES: Cc1cc(N[C@@H]2CCCN(C(C)C)C2)nnc1-c1ccc(C(F)(F)F)cc1NC(=O)C(F)F] | 2,2-difluoro-N-[2-[6-[(3R)-1- isopropyl-3-piperidyl]amino]-4- methyl-pyridazin-3-yl]-5- (trifluoromethyl)phenyl]acetamide |
| 114 | 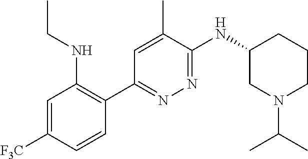 [SMILES: CCNc1cc(C(F)(F)F)ccc1-c1cc(C)c(N[C@@H]2CCCN(C(C)C)C2)nn1] | 6-[2-(ethylamino)-4- (trifluoromethyl)phenyl]-N-[(3R)-1- isopropyl-3-piperidyl]-4-methyl- pyridazin-3-amine |
| 115 | 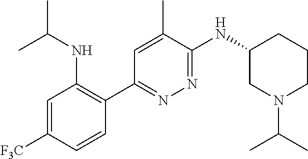 [SMILES: Cc1cc(-c2ccc(C(F)(F)F)cc2NC(C)C)nnc1N[C@@H]1CCCN(C(C)C)C1] | 6-[2-(isopropylamino)-4- (trifluoromethyl)phenyl]-N-[(3R)-1- isopropyl-3-piperidyl]-4-methyl- pyridazin-3-amine |
| 116 | 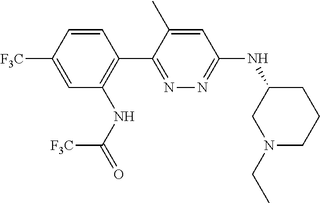 [SMILES: CCN1CCC[C@@H](Nc2cc(C)c(-c3ccc(C(F)(F)F)cc3NC(=O)C(F)(F)F)nn2)C1] | N-[2-[6-[[(3R)-1-ethyl-3- piperidyl]amino]-4-methyl-pyridazin- 3-yl]-5-(trifluoromethyl)phenyl]- 2,2,2-trifluoro-acetamide |
| 117 | 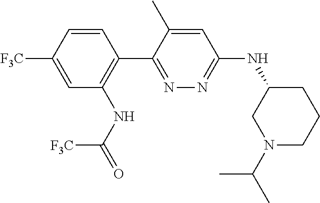 [SMILES: Cc1cc(N[C@@H]2CCCN(C(C)C)C2)nnc1-c1ccc(C(F)(F)F)cc1NC(=O)C(F)(F)F] | 2,2,2-trifluoro-N-[2-[6-[[(3R)-1- isopropyl-3-piperidyl]amino]-4- methyl-pyridazin-3-yl]-5- (trifluoromethyl)phenyl]acetamide |
| 118 | 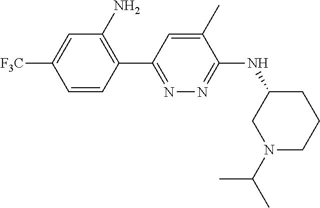 [SMILES: Cc1cc(-c2ccc(C(F)(F)F)cc2N)nnc1N[C@@H]1CCCN(C(C)C)C1] | 6-[2-amino-4- (trifluoromethyl)phenyl]-N-[(3R)-1- isopropyl-3-piperidyl]-4-methyl- pyridazin-3-amine |
| 119 | 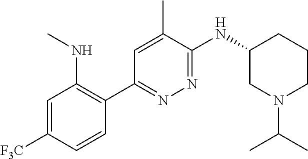 [SMILES: CNc1cc(C(F)(F)F)ccc1-c1cc(C)c(N[C@@H]2CCCN(C(C)C)C2)nn1] | N-[(3R)-1-isopropyl-3-piperidyl]-4- methyl-6-[2-(methylamino)-4- (trifluoromethyl)phenyl]pyridazin-3- amine |
| 120 | 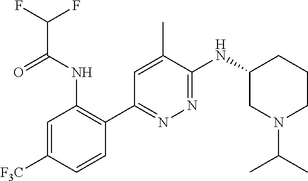 [SMILES: Cc1cc(-c2ccc(C(F)(F)F)cc2NC(=O)C(F)F)nnc1N[C@@H]1CCCN(C(C)C)C1] | 2,2-difluoro-N-[2-[6-[(3R)-1- isopropyl-3-piperidyl]amino]-5- methyl-pyridazin-3-yl]-5- (trifluoromethyl)phenyl]acetamide |
| 121 | 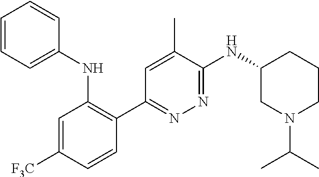 [SMILES: Cc1cc(-c2ccc(C(F)(F)F)cc2Nc2ccccc2)nnc1N[C@@H]1CCCN(C(C)C)C1] | 6-[2-anilino-4- (trifluoromethyl)phenyl]-N-[(3R)-1- isopropyl-3-piperidyl]-4-methyl- pyridazin-3-amine |
| 122 | 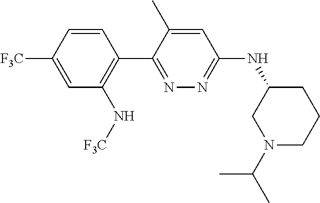 [SMILES: Cc1cc(N[C@@H]2CCCN(C(C)C)C2)nnc1-c1ccc(C(F)(F)F)cc1NC(F)(F)F] | N-[(3R)-1-isopropyl-3-piperidyl]-5- methyl-6-[4-(trifluoromethyl)-2- (trifluoromethylamino)phenyl]- pyridazin-3-amine |
| 123 | 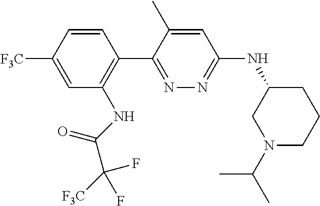 [SMILES: Cc1cc(N[C@@H]2CCCN(C(C)C)C2)nnc1-c1ccc(C(F)(F)F)cc1NC(=O)C(F)(F)C(F)(F)F] | 2,2,3,3,3-pentafluoro-N-[2-[6-[[(3R)- 1-isopropyl-3-piperidyl]amino]-4- methyl-pyridazin-3-yl]-5- (trifluoromethyl)phenyl]propanamide |
| 124 | 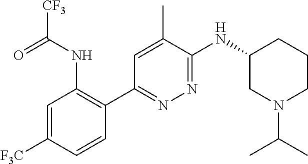 [SMILES: Cc1cc(-c2ccc(C(F)(F)F)cc2NC(=O)C(F)(F)F)nnc1N[C@@H]1CCCN(C(C)C)C1] | N-[2-[6-[[(3R)-1-ethyl-3- piperidyl]amino]-5-methyl-pyridazin- 3-yl]-5-(trifluoromethyl)phenyl]- 2,2,2-trifluoro-acetamide |
| 125 | 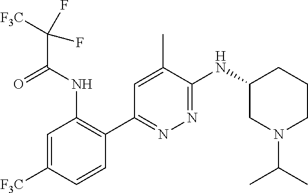 [SMILES: Cc1cc(-c2ccc(C(F)(F)F)cc2NC(=O)C(F)(F)C(F)(F)F)nnc1N[C@@H]1CCCN(C(C)C)C1] | 2,2,3,3,3-pentafluoro-N-[2-[6-[[(3R)- 1-isopropyl-3-piperidyl]amino]-5- methyl-pyridazin-3-yl]-5- (trifluoromethyl)phenyl]propanamide |
| 126 | 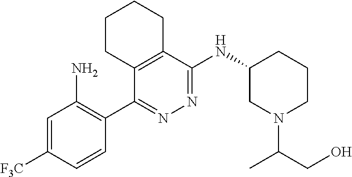 [SMILES: CC(CO)N1CCC[C@@H](Nc2nnc(-c3ccc(C(F)(F)F)cc3N)c3c2CCCC3)C1] | 2-[(3R)-3-[[4-[2-amino-4- (trifluoromethyl)phenyl]-5,6,7,8- tetrahydrophthalazin-1-yl]amino]-1- piperidyl]propan-1-ol |
| 127 | 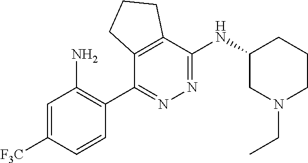 [SMILES: CCN1CCC[C@@H](Nc2nnc(-c3ccc(C(F)(F)F)cc3N)c3c2CCC3)C1] | 1-[2-amino-4- (trifluoromethyl)phenyl]-N-[(3R)-1- ethyl-3-piperidyl]-6,7-dihydro-5H- cyclopenta[d]pyridazin-4-amine |
| 128 | 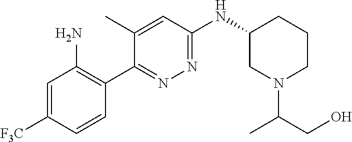 [SMILES: Cc1cc(N[C@@H]2CCCN(C(C)CO)C2)nnc1-c1ccc(C(F)(F)F)cc1N] | 2-[(3R)-3-[[6-[2-amino-4- (trifluoromethyl)phenyl]-5-methyl- pyridazin-3-yl]amino]-1- piperidyl]propan-1-ol |
| 129 | 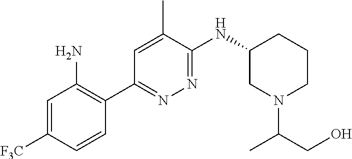 [SMILES: Cc1cc(-c2ccc(C(F)(F)F)cc2N)nnc1N[C@@H]1CCCN(C(C)CO)C1] | 2-[(3R)-3-[[6-[2-amino-4- (trifluoromethyl)phenyl]-4-methyl- pyridazin-3-yl]amino]-1- piperidyl]propan-1-ol |
| 130 | 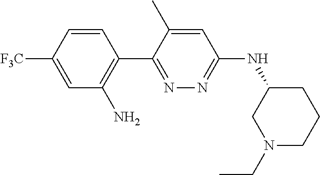 [SMILES: CCN1CCC[C@@H](Nc2cc(C)c(-c3ccc(C(F)(F)F)cc3N)nn2)C1] | 6-[2-amino-4- (trifluoromethyl)phenyl]-N-[(3R)-1- ethyl-3-piperidy1]-5-methyl- pyridazin-3-amine |
| 131 | 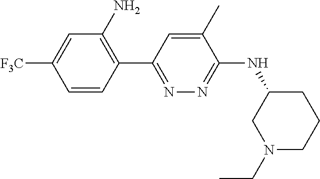 [SMILES: CCN1CCC[C@@H](Nc2nnc(-c3ccc(C(F)(F)F)cc3N)cc2C)C1] | 6-[2-amino-4- (trifluoromethyl)phenyl]-N-[(3R)-1- ethyl-3-piperidyl]-4-methyl- pyridazin-3-amine |
| 132 | 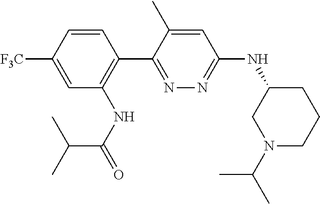 [SMILES: Cc1cc(N[C@@H]2CCCN(C(C)C)C2)nnc1-c1ccc(C(F)(F)F)cc1NC(=O)C(C)C] | N-[2-[6-[[(3R)-1-isopropyl-3- piperidyl]amino]-4-methyl-pyridazin- 3-yl]-5-(trifluoromethyl)phenyl]-2- methyl-propanamide |
| 133 | 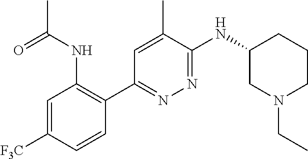 [SMILES: CCN1CCC[C@@H](Nc2nnc(-c3ccc(C(F)(F)F)cc3NC(C)=O)cc2C)C1] | N-[2-[6-[[(3R)-1-ethyl-3- piperidyl]amino]-5-methyl-pyridazin- 3-yl]-5- (trifluoromethyl)phenyl]acetamide |
| 134 | 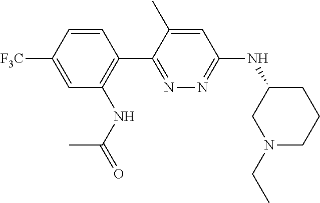 [SMILES: CCN1CCC[C@@H](Nc2cc(C)c(-c3ccc(C(F)(F)F)cc3NC(C)=O)nn2)C1] | N-[2-[6-[[(3R)-1-ethyl-3- piperidyl]amino]-4-methyl-pyridazin- 3-yl]-5- (trifluoromethyl)phenyl]acetamide |
| 135 | 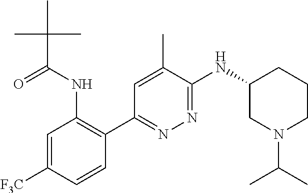 [SMILES: Cc1cc(-c2ccc(C(F)(F)F)cc2NC(=O)C(C)(C)C)nnc1N[C@@H]1CCCN(C(C)C)C1] | N-[2-[6-[[(3R)-1-isopropyl-3- piperidyl]amino]-5-methyl-pyridazin- 3-yl]-5-(trifluoromethyl)phenyl]-2,2- dimethyl-propanamide |
| 136 | 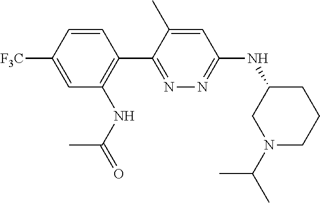 [SMILES: CC(=O)Nc1cc(C(F)(F)F)ccc1-c1nnc(N[C@@H]2CCCN(C(C)C)C2)cc1C] | N-[2-[6-[[(3R)-1-isopropyl-3- piperidyl]amino]-4-methyl-pyridazin- 3-yl]-5- (trifluoromethyl)phenyl]acetamide |
| 137 | 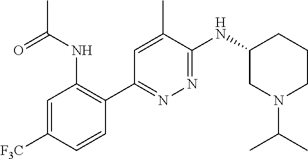 [SMILES: CC(=O)Nc1cc(C(F)(F)F)ccc1-c1cc(C)c(N[C@@H]2CCCN(C(C)C)C2)nn1] | N-[2-[6-[[(3R)-1-isopropyl-3- piperidyl]amino]-5-methyl-pyridazin- 3-yl]-5- (trifluoromethyl)phenyl]acetamide |
| 138 | 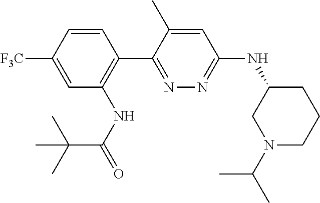 [SMILES: Cc1cc(N[C@@H]2CCCN(C(C)C)C2)nnc1-c1ccc(C(F)(F)F)cc1NC(=O)C(C)(C)C] | N-[2-[6-[[(3R)-1-isopropyl-3- piperidyl]amino]-4-methyl-pyridazin- 3-yl]-5-(trifluoromethyl)phenyl]-2,2- dimethyl-propanamide |
| 139 | 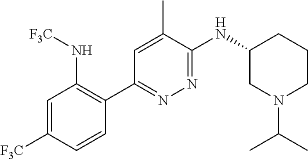 [SMILES: Cc1cc(-c2ccc(C(F)(F)F)cc2NC(F)(F)F)nnc1N[C@@H]1CCCN(C(C)C)C1] | N-[(3R)-1-isopropyl-3-piperidyl]-4- methyl-6-[4-(trifluoromethyl)-2- (trifluoromethylamino)phenyl]pyrida- zin-3-amine |
| 140 | 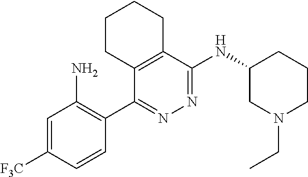 [SMILES: CCN1CCC[C@@H](Nc2nnc(-c3ccc(C(F)(F)F)cc3N)c3c2CCCC3)C1] | 4-[2-amino-4- (trifluoromethyl)phenyl]-N-[(3R)-1- ethyl-3-piperidyl]-5,6,7,8- tetrahydrophthalazin-1-amine |
| 141 | 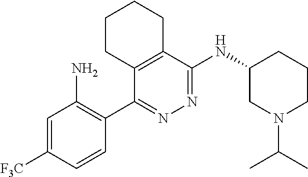 [SMILES: CC(C)N1CCC[C@@H](Nc2nnc(-c3ccc(C(F)(F)F)cc3N)c3c2CCCC3)C1] | 4-[2-amino-4- (trifluoromethyl)phenyl]-N-[(3R)-1- isopropyl-3-piperidyl]-5,6,7,8- tetrahydrophthalazin-1-amine |

In some embodiments, the compound is a pharmaceutically acceptable salt of any one of the compounds described in Table 1.

In some embodiments, the compound is a lithium salt, sodium salt, potassium salt, calcium salt, or magnesium salt of any one of the compounds described in Table 1.

In some embodiments, the compound is a salt of any one of acid described in the Table 2 and any one of the compounds described in Table 1.

TABLE 2
Pharmaceutical acceptable acid forming salts
with the Compound of Formula (A), (I)-(V).

| | | |
|---|---|---|
| 1-hydroxy-2-naphthoic acid | ethanesulfonic acid | methanesulfonic acid |
| 2,2-dichloroacetic acid | formic acid | naphthalene-1,5-disulfonic acid |
| 2-hydroxyethanesulfonic acid | fumaric acid | naphthalene-2-sulfonic acid |
| 2-oxoglutaric acid | galactaric acid | nicotinic acid |
| 4-acetamidobenzoic acid | gentisic acid | nitric acid |
| 4-aminosalicylic acid | glucoheptonic acid (D) | oleic acid |
| acetic acid | gluconic acid (D) | oxalic acid |
| adipic acid | glucuronic acid (D) | palmitic acid |
| ascorbic acid (L) | glutamic acid | pamoic acid |
| aspartic acid (L) | glutaric acid | phosphoric acid |
| benzenesulfonic acid | glycerophosphoric acid | proprionic acid |
| benzoic acid | glycolic acid | pyroglutamic acid (−L) |
| camphoric acid (+) | hippuric acid | salicylic acid |
| camphor-10-sulfonic acid (+) | hydrobromic acid | sebacic acid |
| capric acid (decanoic acid) | hydrochloric acid | stearic acid |
| caproic acid (hexanoic acid) | isobutyric acid | succinic acid |
| caprylic acid (octanoic acid) | lactic acid (DL) | sulfuric acid |
| carbonic acid | lactobionic acid | tartaric acid (+L) |
| cinnamic acid | lauric acid | thiocyanic acid |
| citric acid | maleic acid | toluenesulfonic acid (p) |
| cyclamic acid | malic acid (−L) | undecylenic acid |
| dodecylsulfuric acid | malonic acid | |
| ethane-1,2-disulfonic acid | mandelic acid (DL) | |

In some embodiments, the compound is a salt of acetic acid and any one of the compounds described in Table 1.

In some embodiments, the compound is a salt of adipic acid and any one of the compounds described in Table 1.

In some embodiments, the compound is a salt of ascorbic acid (L) and any one of the compounds described in Table 1.

In some embodiments, the compound is a salt of hydrobromic acid and any one of the compounds described in Table 1.

In some embodiments, the compound is a salt of hydrochloric acid and any one of the compounds described in Table 1.

In some embodiments, the compound is a salt of citric acid and any one of the compounds described in Table 1.

In some embodiments, the compound is a salt of glutamic acid and any one of the compounds described in Table 1.

In some embodiments, the compound is a salt of oxalic acid and any one of the compounds described in Table 1.

In some embodiments, the compound is a salt of formic acid and any one of the compounds described in Table 1.

In some embodiments, the compound is a salt of sulfuric acid and any one of the compounds described in Table 1.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1 and prodrugs and pharmaceutically acceptable salts thereof.

In some embodiments, the compound is an isotopic derivative of any one of the compounds described in Table 1 and pharmaceutically acceptable salts thereof.

In some embodiments, the compound is an isotopic derivative of any one of prodrugs of the compounds described in Table 1 and pharmaceutically acceptable salts thereof.

In some embodiments, the compound is an isotopic derivative of any one of the compounds described in Table 1.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (I)-(V) is isotopically enriched with regard to, or labelled with, one or more isotopes as compared to the corresponding compound of Formula (I)-(V). In some embodiments, the isotopic derivative is enriched with regard to, or labelled with, one or more atoms selected from ²H, ¹³C, ¹⁴C, ¹⁵N, ¹⁸O, ²⁹Si, 31P, and ³⁴S.

In some embodiments, the isotopic derivative is a deuterium labeled compound (i.e., being enriched with ²H with regard to one or more atoms thereof).

In some embodiments, the compound is a deuterium labeled compound of any one of the compounds described in Table 1 and prodrugs and pharmaceutically acceptable salts thereof.

In some embodiments, the compound is a deuterium labeled compound of any one of the compounds described in Table 1 and pharmaceutically acceptable salts thereof.

In some embodiments, the compound is a deuterium labeled compound of any one of the prodrugs of the compounds described in Table 1 and pharmaceutically acceptable salts thereof.

In some embodiments, the compound is a deuterium labeled compound of any one of the compounds described in Table 1.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the disclosure or a pharmaceutically acceptable salt or solvate thereof that contains the aforementioned deuterium atom(s) is within the scope of the disclosure. Further, substitution with deuterium (i.e., ²H) may afford certain therapeutic advantages resulting from greater metabolic stability, e.g., increased in vivo half-life or reduced dosage requirements.

In some embodiments, the compound is a ¹⁸F labeled compound.

In some embodiments, the compound is a ¹²³I labeled compound, a ¹²⁴I labeled compound, a ¹²⁵I labeled compound, a ¹²⁹I labeled compound, a ¹³¹I labeled compound, a ¹³⁵I labeled compound, or any combination thereof.

In some embodiments, the compound is a ³³S labeled compound, a ³⁴S labeled compound, a ³⁵S labeled compound, a ³⁶S labeled compound, or any combination thereof.

It is understood that the ¹⁸F, ¹²³I, ¹²⁴I, ¹²⁵I, ¹²⁹I, ¹³¹I, ¹³⁵I, ³³S, ³⁴S, ³⁵S, and/or ³⁶S labeled compound, can be prepared using any of a variety of art-recognized techniques. For example, the deuterium labeled compound can generally be prepared by carrying out the procedures disclosed in the Schemes and/or in the Examples described herein, by substituting a ¹⁸F, ¹²³I, ¹²⁴I, ¹²⁵I, ¹²⁹I, ¹³¹I, ¹³⁵I, ³³S, ³⁴S, ³⁵S, and/or ³⁶S labeled reagent for a non-isotope labeled reagent.

A compound of the disclosure or a pharmaceutically acceptable salt or solvate thereof that contains one or more of the aforementioned ¹⁸F, ¹²³I, ¹²⁴I, ¹²⁵I, ¹²⁹I, ¹³¹I, ¹³⁵I, ³³S, ³⁴S, ³⁵S, and ³⁶S atom(s) is within the scope of the disclosure. Further, substitution with isotope (e.g., ¹⁸F, ¹²³I, ¹²⁴I, ¹²⁵I, ¹²⁹I, ¹³¹I, ¹³⁵I, ³³S, ³⁴S, ³⁵S, and/or ³⁶S) may afford certain therapeutic advantages resulting from greater metabolic stability, e.g., increased in vivo half-life or reduced dosage requirements.

For the avoidance of doubt, it is to be understood that, where in this specification a group is qualified by “described herein”, the said group encompasses the first occurring and broadest definition as well as each and all of the particular definitions for that group.

The various functional groups and substituents making up the compounds of the Formula (A) are typically chosen such that the molecular weight of the compound does not exceed 1000 Daltons. More usually, the molecular weight of the compound will be less than 900, for example less than 800, or less than 750, or less than 700, or less than 650

Daltons. More conveniently, the molecular weight is less than 600 and, for example, is 550 Daltons or less, for example 500 Daltons or less, for example 450 Daltons or less.

A suitable pharmaceutically acceptable salt of a compound of the disclosure is, for example, an acid-addition salt of a compound of the disclosure, which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl) amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent, and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centres; such compounds can therefore be produced as individual (R)- or(S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess inflammasome inhibitory activity.

It is also to be understood that certain compounds of any one of the Formulae disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compounds of Formula (A), (I), (II) or (III) include all such forms. For the avoidance of doubt, where a compound can exist in one of several tautomeric forms, and only one is specifically described or shown, all others are nevertheless embraced by Formula (A), (I), (II) or (III). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

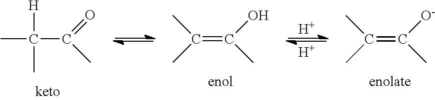

Compounds of any one of the Formulae disclosed herein containing an amine function may also form N-oxides. A reference herein to a compound of Formula (I) or (II) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (m-CPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl) piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁-4alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl) piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

### Method of Synthesizing the Compounds

The compounds of the present invention may be made by a variety of methods, including standard chemistry. Suitable synthetic routes are depicted in the Schemes given below.

The compounds of Formula (A), in particular Formula (I), Formula (II), Formula (III), Formula (IV) or Formula (V) may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of those skilled in the art will recognize if a stereocenter exists in the compounds of Formula (A), (I)-(III). Accordingly, the present invention includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

### Preparation of Compounds

The compounds of the present invention can be prepared in a number of ways well known to those skilled in the art of organic synthesis. By way of example, compounds of the present invention can be synthesized using the methods described below, together with synthetic methods known in the art of synthetic organic chemistry, or variations thereon as appreciated by those skilled in the art. Suitable methods include but are not limited to those methods described below. Compounds of the present invention can be synthesized by following the steps outlined in General Procedures which comprise different sequences of assembling intermediates or compounds. Starting materials are either commercially available or made by known procedures in the reported literature or as illustrated below.

### General Procedure

In general, the compound of the Formula (A) can be prepared according to sequence of reactions presented at the Scheme 1.

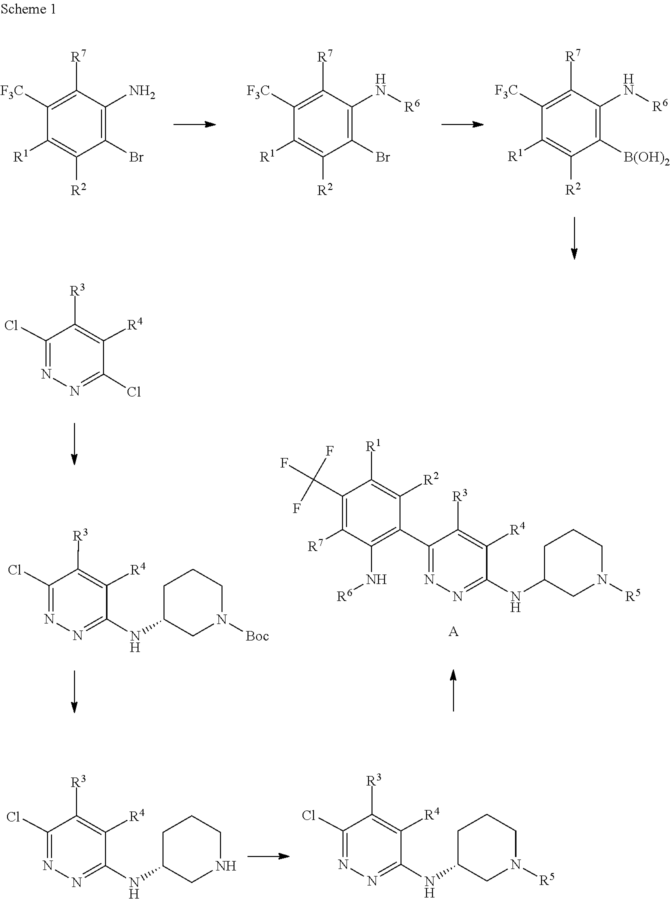

It must be understood that any of the compounds at the Scheme 1 any of substituents in these compounds can be further modified at any step of presented sequences to provide modified derivatives of these compounds useful for preparation of compounds of Formula (A), in particular compounds of Formula (I), Formula (II), Formula (III), Formula (IV), or Formula (V).

All reagents may be commercially available compounds itself or products of synthesis from commercially available reagents. For preparation these reagents may be used one step or multistep synthetic procedures, including but not limited procedures described herein in preparative part.

It should be obvious for specialist in this field that any of compound of Formula (A), (I), (II), (III), (IV) or (V) obtained according to the procedures described above may be a subject for further transformation and modification that will led to obtain other compound of Formula (A), (I), (II), (III), (IV) or (V).

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected from Table 1.

As used herein, the term “composition” is intended to encompass a product comprising the specified ingredients in the specified amounts, as well as any product which results, directly or indirectly, from combination of the specified ingredients in the specified amounts.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well-known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

The compounds of present disclosure can be formulated for oral administration in forms such as tablets, capsules (each of which includes sustained release or timed-release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio) propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

In some embodiments, examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof. In some embodiments, the tonicity agent is selected from the group consisting of a glycol (such as propylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols-such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ε-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

In some embodiments, a pharmaceutical composition described herein may further comprise one or more additional pharmaceutically active agents.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

A therapeutically effective amount of a compound of the present disclosure for use in therapy is an amount sufficient to treat or prevent a NLRP3 related condition referred to herein, slow its progression and/or reduce the symptoms associated with the condition.

A therapeutically effective amount of a compound of the present disclosure for use in therapy is an amount sufficient to treat an NLRP3 related condition referred to herein, slow its progression and/or reduce the symptoms associated with the condition.

The size of the dose for therapeutic or prophylactic purposes of a compound of Formula (A), (I), (II), (III), (IV), (V) will naturally vary according to the nature and severity of the conditions, the age and sex of the animal or subject and the route of administration, according to well-known principles of medicine.

### Methods of Use

In some aspects, the present disclosure provides a method of inhibiting of NLRP3 (e.g., in vitro or in vivo), comprising contacting a cell with a therapeutically effective amount of a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some embodiments, the disease or disorder is associated with NLRP3. In some embodiments, the disease or disorder is a disease or disorder in which NLRP3 is implicated.

The compounds of the invention are also useful in treating diseases associated with NLRP3. For example, diseases and conditions treatable according to the methods of the invention include Acne (ACNE); Acrokeratoderma, Hereditary Papulotranslucent; Adult-Onset Still's Disease (AOSD); Aicardi-Goutieres Syndrome (AGS); Alzheimer Disease, Familial, 1 (AD1); Amyloidosis; Amyotrophic Lateral Sclerosis 1 (ALS1); Anthracosis; Aphthous Stomatitis; Arthritis; Arthropathy; Aseptic Meningitis; Asthma (ASTHMA); Autoimmune Disease of Cardiovascular System; Autoimmune Disease of Gastrointestinal Tract; Autoinflammatory Syndrome; Behcet Syndrome (BD); Blau Syndrome (BLAUS); Body Mass Index Quantitative Trait Locus 11 (BMIQ11); Bone Inflammation Disease; Brain Edema; Brucellosis; Candidiasis; Cervical Adenitis; Chondrocalcinosis; Chronic Meningitis; Chronic Recurrent Multifocal Osteomyelitis (CRMO); Chronic Urticaria; Cinca Syndrome (CINCA); Conjunctival Disease; Conjunctivitis; Connective Tissue Disease; Corneal Disease; Coronavirus Infectious Disease; Covid-19; Crohn's Disease; Cryopyrin-Associated Periodic Syndrome (CAPS); Cystic Fibrosis (CF); Deafness, Autosomal Dominant 34, with or Without Inflammation (DFNA34); Dermatitis; Diabetes Mellitus; Diabetic Encephalopathy; Exanthem (RASH); Eye Disease; Familial Cold Autoinflammatory Syndrome (FCU); Familial Cold Autoinflammatory Syndrome 1 (FCAS1); Familial Cold Autoinflammatory Syndrome 2 (FCAS2); Familial Mediterranean Fever (FMF); Gout (GOUT); Hereditary Periodic Fever Syndrome; Hidradenitis; Hidradenitis Suppurativa; Hypertension, Essential (EHT); Hyperuricemia; Intellectual Developmental Disorder, Autosomal Dominant 30, with Speech Delay and Behavioral Abnormalities (MRD30); Interstitial Lung Disease 2 (ILD2); Keratitis, Hereditary (KERH); Keratoendotheliitis Fugax Hereditaria (KEFH); Lipoprotein Quantitative Trait Locus (LPAQTL); Lymph Node Disease; Macular Degeneration, Age-Related, 1 (ARMD1); Malaria (CM); Meningitis; Mevalonic Aciduria (MEVA); Muckle-Wells Syndrome (MWS); Mycobacterium Kansasii; Myocardial Infarction (MCI1); Nephrolithiasis; Nervous System Disease; Non-Alcoholic Steatohepatitis (NASH); Optic Papillitis; Osteomyelitis; Osteoporosis (OSTEOP); Otitis Media (OMS); Papilledema; Pericardial Effusion; Pericardium Disease; Pharyngitis; Polyradiculoneuropathy; Primary Bacterial Infectious Disease; Proteasome-Associated Autoinflammatory Syndrome 1 (PRAAS1); Pyoderma; Pyoderma Gangrenosum; Pyogenic Sterile Arthritis, Pyoderma Gangrenosum, and Acne (PAPAS); Respiratory Failure; Salmonellosis; Schnitzler Syndrome; Sebaceous Gland Disease; Sensorineural Hearing Loss; Serum Amyloid a Amyloidosis; Silicosis; Skin Disease; Stomatitis; Type 2 Diabetes Mellitus (T2D); Ureteral Disease; Urticaria (HIVES); Vulvovaginal Candidiasis; Wells Syndrome.

The method of treatment comprises administering to a patient suffering from at least one of said diseases or disorders a compound of Formula (A), or a pharmaceutically acceptable salt, hydrate, solvate, prodrug, stereoisomer, tautomer, or pharmaceutical composition thereof.

In some embodiments, the method of treatment comprises administering to a patient suffering from at least one of said diseases or disorders a compound of Formula (I), or a pharmaceutically acceptable salt, hydrate, solvate, prodrug, stereoisomer, tautomer, or pharmaceutical composition thereof.

In some embodiments, the method of treatment comprises administering to a patient suffering from at least one of said diseases or disorders a compound of Formula (II), or a pharmaceutically acceptable salt, hydrate, solvate, prodrug, stereoisomer, tautomer, or pharmaceutical composition thereof.

In some embodiments, the method of treatment comprises administering to a patient suffering from at least one of said diseases or disorders a compound of Formula (III), or a pharmaceutically acceptable salt, hydrate, solvate, prodrug, stereoisomer, tautomer, or pharmaceutical composition thereof.

In some embodiments, the method of treatment comprises administering to a patient suffering from at least one of said diseases or disorders a compound of Formula (IV), or a pharmaceutically acceptable salt, hydrate, solvate, prodrug, stereoisomer, tautomer, or pharmaceutical composition thereof.

In some embodiments, the method of treatment comprises administering to a patient suffering from at least one of said diseases or disorders a compound of Formula (V), or a pharmaceutically acceptable salt, hydrate, solvate, prodrug, stereoisomer, tautomer, or pharmaceutical composition thereof.

In some embodiments, the disease or disorder is an Autoimmune Disease.

In some embodiments, the disease or disorder is Acne (ACNE).

In some embodiments, the disease or disorder is Acrokeratoderma, Hereditary Papulotranslucent.

In some embodiments, the disease or disorder is Adult-Onset Still's Disease (AOSD).

In some embodiments, the disease or disorder is Aicardi-Goutieres Syndrome (AGS).

In some embodiments, the disease or disorder is Alzheimer Disease, Familial, 1 (AD1).

In some embodiments, the disease or disorder is Amyloidosis.

In some embodiments, the disease or disorder is Amyotrophic Lateral Sclerosis 1 (ALS1).

In some embodiments, the disease or disorder is Anthracosis.

In some embodiments, the disease or disorder is Aphthous Stomatitis.

In some embodiments, the disease or disorder is Arthritis.

In some embodiments, the disease or disorder is Arthropathy.

In some embodiments, the disease or disorder is Aseptic Meningitis.

In some embodiments, the disease or disorder is Asthma (ASTHMA).

In some embodiments, the disease or disorder is Autoimmune Disease of Cardiovascular System.

In some embodiments, the disease or disorder is Autoimmune Disease of Gastrointestinal Tract.

In some embodiments, the disease or disorder is Autoinflammatory Syndrome.

In some embodiments, the disease or disorder is Behcet Syndrome (BD).

In some embodiments, the disease or disorder is Blau Syndrome (BLAUS).

In some embodiments, the disease or disorder is Body Mass Index Quantitative Trait Locus 11 (BMIQ11).

In some embodiments, the disease or disorder is Bone Inflammation Disease.

In some embodiments, the disease or disorder is Brain Edema.

In some embodiments, the disease or disorder is Brucellosis.

In some embodiments, the disease or disorder is Candidiasis.

In some embodiments, the disease or disorder is Cervical Adenitis.

In some embodiments, the disease or disorder is Chondrocalcinosis.

In some embodiments, the disease or disorder is Chronic Meningitis.

In some embodiments, the disease or disorder is Chronic Recurrent Multifocal Osteomyelitis (CRMO).

In some embodiments, the disease or disorder is Chronic Urticaria.

In some embodiments, the disease or disorder is Cinca Syndrome (CINCA).

In some embodiments, the disease or disorder is Conjunctival Disease.

In some embodiments, the disease or disorder is Conjunctivitis.

In some embodiments, the disease or disorder is Connective Tissue Disease.

In some embodiments, the disease or disorder is Corneal Disease.

In some embodiments, the disease or disorder is Coronavirus Infectious Disease.

In some embodiments, the disease or disorder is Covid-19.

In some embodiments, the disease or disorder is Crohn's Disease.

In some embodiments, the disease or disorder is Cryopyrin-Associated Periodic Syndrome (CAPS).

In some embodiments, the disease or disorder is Cystic Fibrosis (CF).

In some embodiments, the disease or disorder is Deafness, Autosomal Dominant 34, with or Without Inflammation (DFNA34).

In some embodiments, the disease or disorder is Dermatitis.

In some embodiments, the disease or disorder is Diabetes Mellitus.

In some embodiments, the disease or disorder is Diabetic Encephalopathy.

In some embodiments, the disease or disorder is Exanthem (RASH).

In some embodiments, the disease or disorder is Eye Disease.

In some embodiments, the disease or disorder is Familial Cold Autoinflammatory Syndrome (FCU).

In some embodiments, the disease or disorder is Familial Cold Autoinflammatory Syndrome 1 (FCAS1).

In some embodiments, the disease or disorder is Familial Cold Autoinflammatory Syndrome 2 (FCAS2).

In some embodiments, the disease or disorder is Familial Mediterranean Fever (FMF).

In some embodiments, the disease or disorder is Gout (GOUT).

In some embodiments, the disease or disorder is Hereditary Periodic Fever Syndrome.

In some embodiments, the disease or disorder is Hidradenitis.

In some embodiments, the disease or disorder is Hidradenitis Suppurativa.

In some embodiments, the disease or disorder is Hypertension, Essential (EHT).

In some embodiments, the disease or disorder is Hyperuricemia.

In some embodiments, the disease or disorder is Intellectual Developmental Disorder, Autosomal Dominant 30, with Speech Delay and Behavioral Abnormalities (MRD30).

In some embodiments, the disease or disorder is Interstitial Lung Disease 2 (ILD2).

In some embodiments, the disease or disorder is Keratitis, Hereditary (KERH).

In some embodiments, the disease or disorder is Keratoendotheliitis Fugax Hereditaria (KEFH).

In some embodiments, the disease or disorder is Lipoprotein Quantitative Trait Locus (LPAQTL).

In some embodiments, the disease or disorder is Lymph Node Disease.

In some embodiments, the disease or disorder is Macular Degeneration, Age-Related, 1 (ARMD1).

In some embodiments, the disease or disorder is Malaria (CM).

In some embodiments, the disease or disorder is Meningitis.

In some embodiments, the disease or disorder is Mevalonic Aciduria (MEVA).

In some embodiments, the disease or disorder is Muckle-Wells Syndrome (MWS).

In some embodiments, the disease or disorder is Mycobacterium Kansasii.

In some embodiments, the disease or disorder is Myocardial Infarction (MCI1).

In some embodiments, the disease or disorder is Nephrolithiasis.

In some embodiments, the disease or disorder is Nervous System Disease.

In some embodiments, the disease or disorder is Non-Alcoholic Steatohepatitis (NASH).

In some embodiments, the disease or disorder is Optic Papillitis.

In some embodiments, the disease or disorder is Osteomyelitis.

In some embodiments, the disease or disorder is Osteoporosis (OSTEOP).

In some embodiments, the disease or disorder is Otitis Media (OMS).

In some embodiments, the disease or disorder is Papilledema.

In some embodiments, the disease or disorder is Pericardial Effusion.

In some embodiments, the disease or disorder is Pericardium Disease.

In some embodiments, the disease or disorder is Pharyngitis.

In some embodiments, the disease or disorder is Polyradiculoneuropathy.

In some embodiments, the disease or disorder is Primary Bacterial Infectious Disease.

In some embodiments, the disease or disorder is Proteasome-Associated Autoinflammatory Syndrome 1 (PRAAS1).

In some embodiments, the disease or disorder is Pyoderma.

In some embodiments, the disease or disorder is Pyoderma Gangrenosum.

In some embodiments, the disease or disorder is Pyogenic Sterile Arthritis, Pyoderma Gangrenosum, and Acne (PAPAS).

In some embodiments, the disease or disorder is Respiratory Failure.

In some embodiments, the disease or disorder is Salmonellosis.

In some embodiments, the disease or disorder is Schnitzler Syndrome.

In some embodiments, the disease or disorder is Sebaceous Gland Disease.

In some embodiments, the disease or disorder is Sensorineural Hearing Loss.

In some embodiments, the disease or disorder is Serum Amyloid a Amyloidosis.

In some embodiments, the disease or disorder is Silicosis.

In some embodiments, the disease or disorder is Skin Disease.

In some embodiments, the disease or disorder is Stomatitis.

In some embodiments, the disease or disorder is Type 2 Diabetes Mellitus (T2D).

In some embodiments, the disease or disorder is Ureteral Disease.

In some embodiments, the disease or disorder is Urticaria (HIVES).

In some embodiments, the disease or disorder is Vulvovaginal Candidiasis.

In some embodiments, the disease or disorder is Wells Syndrome.

In some aspects, the present disclosure provides a method of treating or preventing an Autoimmune Disease in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating an Autoimmune Disease in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in inhibiting of NLRP3 (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in treating or preventing a Autoimmune Disease in a subject in need thereof.

In some aspects, the present disclosure provides a compound of the present disclosure or a pharmaceutically acceptable salt thereof for use in treating or preventing a Coronavirus Infectious Disease in a subject in need thereof.

In some aspects, the present disclosure provides a compound of the present disclosure or a pharmaceutically acceptable salt thereof for use in treating Autoimmune Disease in a subject in need thereof.

In some aspects, the present disclosure provides a compound of the present disclosure or a pharmaceutically acceptable salt thereof for use in treating Coronavirus Infectious Disease in a subject in need thereof.

In some aspects, the present disclosure provides use of a compound of the present disclosure or a pharmaceutically acceptable salt thereof in the manufacture of a medicament for inhibiting of NLRP3 (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for treating or preventing Autoimmune Disease in a subject in need thereof.

In some aspects, the present disclosure provides use of a compound of the present disclosure or a pharmaceutically acceptable salt thereof in the manufacture of a medicament for treating or preventing Coronavirus Infectious Disease in a subject in need thereof.

In some aspects, the present disclosure provides use of a compound of the present disclosure or a pharmaceutically acceptable salt thereof in the manufacture of a medicament for treating Autoimmune Disease in a subject in need thereof.

In some aspects, the present disclosure provides use of a compound of the present disclosure or a pharmaceutically acceptable salt thereof in the manufacture of a medicament for treating Coronavirus Infectious Disease in a subject in need thereof.

The present disclosure provides compounds that function as inhibitors of NLRP3 (e.g., in vitro or in vivo). The present disclosure therefore provides a method of inhibiting of NLRP3 in vitro or in vivo, said method comprising contacting a cell with a therapeutically effective amount of a compound, or a pharmaceutically acceptable salt thereof, as defined herein.

In some embodiments, the inhibitor of NLRP3 is a compound of the present disclosure.

Effectiveness of compounds of the disclosure can be determined by industry-accepted assays/disease models according to standard practices of elucidating the same as described in the art and are found in the current general knowledge.

The present disclosure also provides a method of treating a disease or disorder in which NLRP3 is implicated in a subject in need of such treatment, said method comprising administering to said subject a therapeutically effective amount of a compound, or a pharmaceutically acceptable salt thereof, or a pharmaceutical composition as defined herein.

In some embodiments, the subject is a mammal. In some embodiments, the subject is a human.

### Routes of Administration

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### EXAMPLES

### General Synthetical Procedures and Examples of the Compound's Preparation

All reagents were commercial and were used without further purification. Yields refer to purified and spectroscopically pure compounds. Thin layer chromatography (TLC) was performed using Merck TLC Aluminum sheets silica gel 60 F₂₅₄ plates and visualized by fluorescence quenching under UV light. Flash chromatography was performed using silica gel (Chromatorex, MB 70-40/75, 40-75 μm) purchased by Fuji Silysia Chemicals. NMR spectra were recorded on a Varian-400MR operating at 400 MHz for ¹H. Chemical shifts are reported in ppm with the solvent resonance as the internal standard. Data is reported as follows: s=singlet, br.=broad, d=doublet, t=triplet, q=quartet, m=multiplet, dd=doublet of doublets; coupling constants in Hz; integration. The purities were recorded on Waters e2695 separations Module/2998 PDA Detector HPLC system (Column: XBridge C18, 5 μm, 4.6 mm (ID)×150 mm (L), Eluent: the mixture of mobile phase A and B, mobile phase A: 100% acetonitrile; mobile phase B: pure water containing 0.1% formic acid and 10 mM NH₄OAc, Flow rate: 0.5 mL/min. detection: UV, 254 nm) Abbreviations used in the following examples and elsewhere herein are:
AA acetic acid
ACN acetonitrile
aq. aqueous
anh. anhydrous
br. broad
BSA bovine serum albumin
BTMA·ICl₂ benzyltrimethylammonium dichloroiodide
d duplet
DCM dichloromethane
DIPA diisopropylamine
DIPEA N,N-diisopropylethylamine
DMAP dimethyl aminopyridine
DMF N,N-dimethyl formamide
DMSO dimethyl sulfoxide
EA ethyl acetate
eq equivalent
FA formic acid
FBS fetal bovine serum
h hour(s)
HPLC high pressure (or performance) liquid chromatography
LCMS liquid chromatography mass spectrometry
m multiplet
M molar
MHz megahertz
min minutes
NBS N-bromosuccinimide
NMR nuclear magnetic resonance
Pd₂(dba)₃ tris(dibenzylideneacetone)dipalladium(0)
Pd(dppf)Cl₂×DCM [1,1′-Bis(diphenylphosphino)ferrocene]dichloropalladium(II), complex with dichloromethane
q quadruplet
rt room temperature
s singlet
s solid
sat. saturated
t temperature, triplet
TEA triethylamine
THF tetrahydrofuran
TLC thin layer chromatography

### Synthesis of Building Blocks

### Synthesis of (2-(methyl sulfonamido)-4-(trifluoromethyl)phenyl) boronic acid (P2)

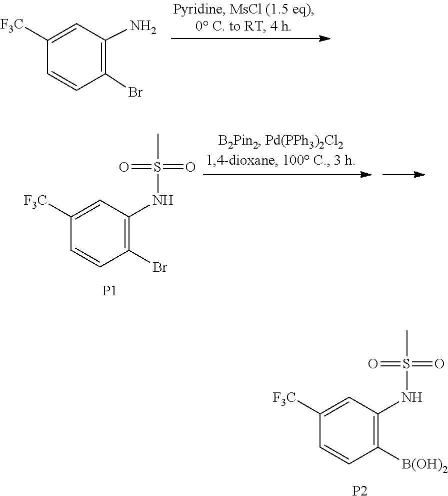

Preparation 1. N-(2-Bromo-5-(trifluoromethyl)phenyl) methanesulfonamide (P1). To a stirred solution of 2-bromo-5-(trifluoromethyl) aniline (5.0 g, 20.83 mmol, 1.0 eq) in pyridine (20 mL) was added MsCl (2.5 mL, 31.24 mmol, 1.5 eq) at 0° C. The resulting reaction mixture was stirred at 0° C. to ambient temperature for 4 h. The progress of reaction was monitored by TLC and LCMS. After completion the reaction mixture was concentrated under reduced pressure and crude reaction mass was diluted with chloroform and extracted with water (3×100 mL). The combined organic layers were washed with 1N HCl (100 mL) and dried over anh. Na₂SO₄, filtered and concentrated under reduced pressure. The residue was subjected to combi-flash chromatography using mixture of EtOAc (10→13%) and hexanes as an eluent to afford 2.5 g (37%) of the title compound as an off-white solid.

Analytical data: ¹H NMR (400 MHz, CDCl₃), δ: 7.92 (d, J=1.6 Hz, 1H), 7.73 (d, J=8.0 Hz, 1H), 7.32 (dd, J=8.4, 1.6 Hz, 1H), 6.92 (br. s, 1H), 3.07 (s, 3H). LCMS (ESI): calc. for C₈H₇BrF₃NO₂S [M+H]⁺ 317.9, found 318.1.

### Preparation 2. (2-(Methyl sulfonamido)-4-(trifluoromethyl)phenyl) boronic acid (P2)

To a stirred solution of P2 (2.0 g, 6.28 mmol, 1.0 eq) in 1,4-dioxane (25.0 mL) were added KOAc (2.1 g, 22.00 mmol, 3.5 eq) and bis(pinacolato)diboron (2.3 g, 9.43 mmol, 1.5 eq) at ambient temperature. The resulting reaction mixture was degassed with argon for 15 min and Pd(PPh₃)₂Cl₂ (440 mg, 0.629 mmol, 0.1 eq) was added at ambient temperature and reaction mixture was stirred at 100° C. for 3 h. The progress of reaction was monitored by TLC and LCMS. After completion of starting material, reaction mixture was diluted with DCM (150 mL) filtered through celite bed, filtrate was concentrated under reduced pressure to afford 2.0 g (crude) of the title compound as a brown gummy liquid. Note: Crude itself proceeded to next step without further purification.

Analytical data: LCMS (ESI): calc, for C₂₁H₂₃BFN₃O₃ [M−H]⁺ 282.1, found 282.1.

### Synthesis of (R)-6-chloro-N-(1-ethylpiperidin-3-yl)-5-methylpyridazin-3-amine (P5)

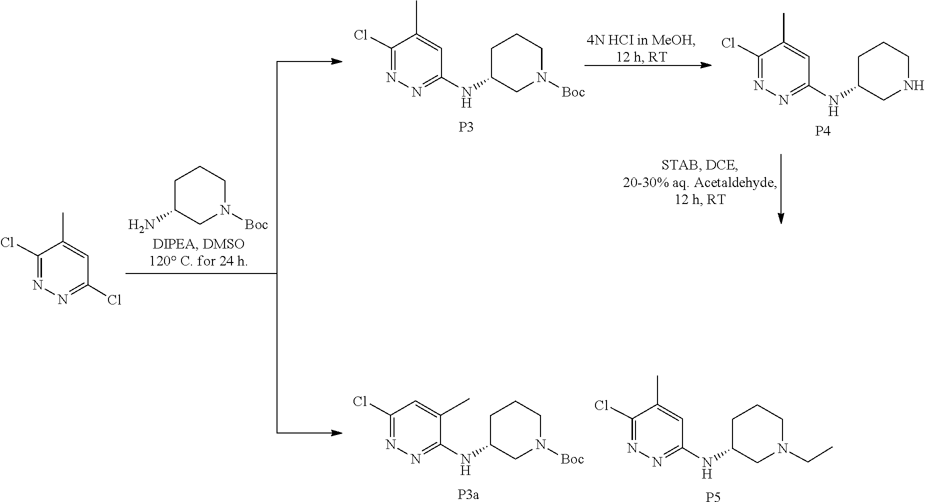

### Preparation 3. tert-Butyl (R)-3-((6-chloro-5-methylpyridazin-3-yl) amino)piperidine-1-carboxylate (P3) and tert-butyl (R)-3-((6-chloro-4-methylpyridazin-3-yl)amino)-piperidine-1-carboxylate (P3a)

To a stirred solution of 3,6-dichloro-4-methylpyridazine (5.0 g, 30.673 mmol, 1.0 eq) in DMSO (20 mL) was added DIPEA (27.3 mL, 153.36 mmol, 5.0 eq) followed by tert-butyl (R)-3-aminopiperidine-1-carboxylate (7.97 g, 39.87 mmol, 1.3 eq) and the resulting reaction mixture was stirred at 140° C. to ambient temperature for 24 h. The progress of reaction was monitored by TLC and LCMS. After completion of starting material, reaction mixture was quenched with water (100 mL) and extracted with EtOAc (3×100 mL). The combined organic layers were dried over anh. Na₂SO₄, filtered and concentrated under reduced pressure. The residue was subjected to flash chromatography using 15-20% EA in pet ether as an eluent to afford P3 and P3a.

Analytical data of P3: Yield of P3, 2.5 g (24.2%) of the title compound as a light brown solid. ¹H NMR (400 MHz, DMSO-d₆), δ: 6.88 (d, J=7.2 Hz, 1H), 6.81 (br. s, 1H), 4.03-3.60 (m, 3H), 3.24-2.92 (m, 2H), 2.19 (s, 3H), 1.98 (br. s, 1H), 1.74 (br. s, 1H), 1.43-1.15 (m, 11H). LCMS (ESI): calc, for C₁₅H₂₃ClN₄O₂ [M+H]⁺ 327.1, found 327.3.

Analytical data of P3a: Yield of P3a, 1.14 (14%) of the title compound as a light brown solid. ¹H NMR (400 MHz, DMSO-d₆), δ: 7.33 (s, 1H), 6.03 (br. s, 1H), 4.02-3.94 (m, 1H), 3.82-3.65 (m, 1H), 3.30-2.80 (m, 2H), 2.10 (s, 3H), 1.98-1.94 (m, 1H), 1.74-1.72 (br. s, 2H), 1.46-1.15 (m, 11H). LCMS (ESI): calc, for C₁₅H₂₃ClN₄O₂ [M+H]⁺ 327.1, found 327.3.

### Preparation 4. (R)-6-Chloro-5-methyl-N-(piperidin-3-yl)pyridazin-3-amine (P4)

To a stirred solution of tert-butyl (R)-3-((6-chloro-5-methylpyridazin-3-yl) amino)piperidine-1-carboxylate (P3, 2.5 g, 7.669 mmol, 1.0 eq) in methanol (5.0 mL) was added 4N HCl in MeOH (10 mL) at ambient temperature. The resulting reaction mixture was stirred at ambient temperature for 16 h. The progress of reaction was monitored by TLC and LCMS. After completion of starting material, volatiles were concentrated under reduced pressure. The residue was triturated with diethyl ether and dried to afford 1.7 g (97.7%) of the title compound as an off white solid.

Note: Crude itself proceeded to next step without further purification.

Analytical data: ¹H NMR (400 MHz, DMSO-d₆), δ: 9.54 (s, 1H), 9.21 (br. s, 1H), 8.98 (br. s, 1H), 7.32 (s, 1H), 4.26 (br. s, 1H), 3.38-3.12 (m, 2H), 2.94-2.90 (m, 2H), 2.33 (s, 3H), 2.01-1.90 (m, 2H), 1.80-1.71 (m, 1H), 1.66-1.58 (m, 1H). LCMS (ESI): calc, for C₁₀H₁₅ClN₄ [M+H]⁺ 227.1, found 227.3.

### Preparation 5. (R)-6-Chloro-N-(1-ethylpiperidin-3-yl)-5-methylpyridazin-3-amine (P5)

To a stirred solution of (R)-6-Chloro-5-methyl-N-(piperidin-3-yl)pyridazin-3-amine (P4, 500 mg, 2.212 mmol, 1.0 eq) in DCE (5.0 mL) were added AcOH (27 mg, 0.442 mmol, 0.2 eq) and 30% acetaldehyde solution (5.0 mL) at ambient temperature. The resulting reaction mixture was stirred at same temperature for 16 h. The progress of reaction was monitored by TLC and LCMS. After completion of starting material, reaction mixture was quenched with sat. NaHCO₃ (50 mL) and extracted with EtOAc (3×50 mL). The combined organic layers were dried over anh. Na₂SO₄, filtered and concentrated under reduced pressure. The residue was subjected to flash chromatography using mixture of MeOH (5→10%) in DCM as an eluent to afford 450 mg (95.7%) of the title compound as an off white solid.

Analytical data: ¹H NMR (400 MHz, DMSO-d₆), δ: 6.80 (s, 1H), 6.77 (d, J=8.0 Hz, 1H), 3.95-3.88 (m, 1H), 2.89-2.97 (m, 1H), 2.63-2.61 (m, 1H), 2.32 (q, J=4.0 Hz, 2H), 2.17 (s, 3H), 1.99 (t, J=9.6 Hz, 1H), 1.89-1.80 (m, 2H), 1.70-1.65 (m, 1H), 1.54-1.44 (m, 1H), 1.31-1.28 (m, 1H), 1.03 (t, J=7.0 Hz, 3H). LCMS (ESI): calc, for C₁₂H₁₉ClN₄ [M+H]⁺ 255.1, found 255.3.

### Synthesis of (R)-2-(3-((6-Chloro-5-methylpyridazin-3-yl)amino) piperidin-1-yl) ethan-1-ol (P6)

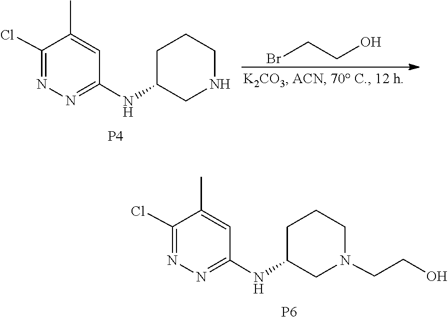

### Preparation 6. (R)-2-(3-((6-Chloro-5-methylpyridazin-3-yl)amino) piperidin-1-yl) ethan-1-ol (P6)

To a stirred solution of (R)-6-chloro-5-methyl-N-(piperidin-3-yl)pyridazin-3-amine (P4, 500 mg, 2.212 mmol, 1.0 eq) in ACN (5.0 mL) were added potassium carbonate (612 mg, 4.425 mmol, 2.0 eq) and 2-bromoethan-1-ol (412 mg, 3.31 mmol, 1.5 eq) at ambient temperature. The resulting reaction mixture stirred at 70° C. for 12 h. The progress of reaction was monitored by TLC and LCMS. After completion of starting material, reaction mixture quenched with water (50 mL) extracted with EA (3×50 mL). The combined organic layers were dried over anh. Na₂SO₄, filtered and concentrated under reduced pressure. The residue was subjected to flash chromatography using mixture of (5→25%) MeOH in DCM as an eluent to afford 400 mg (66.0%) of the title compound as off-white solid.

Analytical data: ¹H NMR (400 MHz, DMSO-d₆), δ: 6.80 (br. s, 1H), 6.77 (br. s, 1H), 4.34 (t, J=5.2 Hz, 1H), 4.09 (dd, J=10.4 Hz, 5.2 Hz, 1H), 3.93 (t, J=4.0 Hz, 1H), 3.48 (dd, J=11.2 Hz, 5.6 Hz, 2H), 3.15 (d, J=4.8 Hz, 2H), 2.86 (d, J=11.2 Hz, 1H), 2.67-2.61 (m, 1H), 2.41-2.32 (m, 2H), 2.18 (s, 3H), 2.15-1.99 (m, 1H), 1.79-1.77 (m, 1H), 1.76-1.67 (m, 1H). LCMS (ESI): calc, for C₁₂H₁₉ClN₄O [M+H]⁺ 271.1, found 271.3.

### Synthesis of (R)-6-chloro-N-(1-ethylpiperidin-3-yl)-4-methylpyridazin-3-amine (P8)

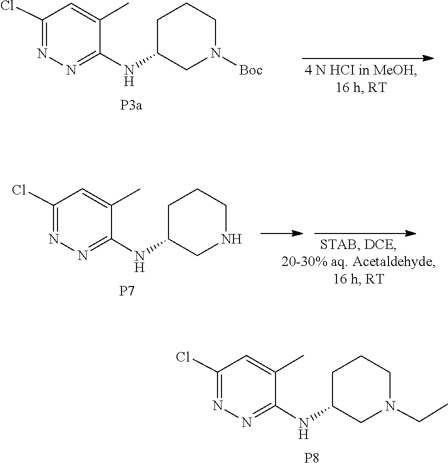

### Preparation 7. (R)-6-Chloro-4-methyl-N-(piperidin-3-yl)pyridazin-3-amine (P7)

To a stirred solution of tert-butyl (R)-3-((6-chloro-4-methylpyridazin-3-yl)amino)piperidine-1-carboxylate (P3a, 1.3 g, 3.988 mmol, 1.0 eq) in methanol (5.0 mL) was added 4N HCl in MeOH (5 mL) at ambient temperature. The resulting reaction mixture was stirred at ambient temperature for 16 h. The progress of reaction was monitored by TLC and LCMS. After completion of starting material, volatiles were concentrated under reduced pressure. The residue was triturated with diethyl ether and dried to afford 900 mg crude of the title compound as an off-white solid.

Note: Crude itself proceeded to next step without further purification.

Analytical data: ¹H NMR (400 MHz, DMSO-d₆), δ: 9.46 (br. s, 1H), 7.52 (s, 1H), 6.82 (br. s, 1H), 4.41 (br. s, 1H), 3.41-3.33 (m, 1H), 3.20-3.14 (m, 1H), 2.97-2.84 (m, 2H), 2.20 (s, 3H), 1.98-1.91 (m, 2H), 1.75-1.65 (m, 2H). LCMS (ESI): calc, for C₁₀H₁₅ClN₄ [M+H]⁺ 227.1, found 227.2.

### Preparation 8. (R)-6-Chloro-N-(1-ethylpiperidin-3-yl)-4-methylpyridazin-3-amine (P8)

To a stirred solution of (R)-6-Chloro-4-methyl-N-(piperidin-3-yl)pyridazin-3-amine (P7, 500 mg, 2.205 mmol, 1.0 eq) in DCE (5.0 mL) were added AcOH (27 mg, 0.442 mmol, 0.2 eq) and 30% aq. acetaldehyde solution (5.0 mL) at ambient temperature. The resulting reaction mixture was stirred at ambient temperature for 16 h. The progress of reaction was monitored by TLC and LCMS. After completion of starting material, reaction mixture was quenched with sat. NaHCO₃ (20 mL) and extracted with EtOAc (3×50 mL). The combined organic layers were dried over anh. Na₂SO₄, filtered and concentrated under reduced pressure. The residue was subjected to flash chromatography using mixture of MeOH (5→10%) in DCM as an eluent to afford 450 mg (80%) of the title compound as an off white solid.

Analytical data: ¹H NMR (400 MHz, DMSO-d₆), δ: 7.29 (s, 1H), 5.95 (d, J=8.0 Hz, 1H), 4.16-4.09 (m, 1H), 2.96 (d, J=8.4 Hz, 1H), 2.70 (d, J=11.2 Hz, 1H), 2.34 (q, J=7.2 Hz, 2H), 2.09 (s, 3H), 1.95-1.83 (m, 3H), 1.56-1.33 (m, 3H), 0.97 (t, J=9.6 Hz, 3H). LCMS (ESI): calc, for C₁₂H₁₉ClN₄ [M+H]⁺ 255.1, found 255.3.

### Synthesis of (R)-2-(3-((6-chloro-4-methylpyridazin-3-yl)amino) piperidin-1-yl) ethan-1-ol (P9)

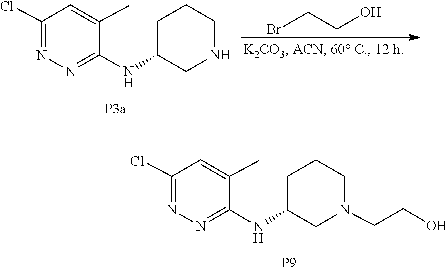

### Preparation 9. (R)-2-(3-((6-Chloro-4-methylpyridazin-3-yl)amino) piperidin-1-yl) ethan-1-ol (P9)

To a stirred solution of (R)-6-chloro-4-methyl-N-(piperidin-3-yl)pyridazin-3-amine (P3a, 500 mg, 2.212 mmol, 1.0 eq) in ACN (5.0 mL) were added potassium carbonate (612 mg, 4.425 mmol, 2.0 eq) and 2-bromoethan-1-ol (412 mg, 3.31 mmol, 1.5 eq) at ambient temperature. The resulting reaction mixture stirred at 60° C. for 12 h. The progress of reaction was monitored by TLC and LCMS. After completion of starting material, reaction mixture quenched with water (50 mL) extracted with EA (3× 50 mL). The combined organic layers were dried over anh. Na₂SO₄, filtered and concentrated under reduced pressure. The residue was subjected to flash chromatography using mixture (5→25%) MeOH in DCM as an eluent to afford 350 mg (58.0%) of the title compound as off-white solid.

Analytical data: ¹H NMR (400 MHz, DMSO-d₆), δ: 7.29 (s, 1H), 5.95 (d, J=7.6 Hz, 1H), 4.36 (br. s, 1H), 4.14-4.10 (m, 1H), 3.49 (br. s, 2H), 2.93 (d, J=8.8 Hz, 1H), 2.70-2.68 (m, 1H), 2.40 (t, J=6.4 Hz, 2H), 2.09 (s, 3H), 2.07-2.02 (m, 1H), 1.83-1.80 (m, 1H), 1.68-1.64 (m, 1H), 1.53-1.39 (m, 1H). LCMS (ESI): calc, for C₁₂H₁₉ClN₄O [M+H]⁺ 271.1, found 271.2.

### Synthesis of (R)-6-Chloro-5-Methyl-N-(1-methylpiperidin-3-yl)pyridazin-3-amine (P10) and (R)-6-chloro-4-Methyl-N-(1-methylpiperidin-3-yl)pyridazin-3-amine (P10a)

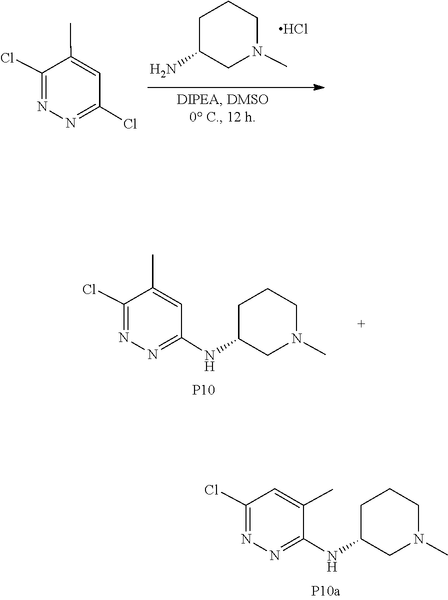

### Preparation 10. (R)-2-(3-((6-Chloro-4-methylpyridazin-3-yl)amino) piperidin-1-yl) ethan-1-ol (P10)

To a stirred solution of 3,6-dichloro-4-methylpyridazine (1.0 g, 6.13 mmol, 1.0 eq) in DMSO (10.0 mL) in a sealed tube were added DIPEA (15.8 g, 122.6 mmol, 20 eq) and (R)-1-methylpiperidin-3-amine (2.3 g, 12.26 mmol, 2.0 eq) at ambient temperature. The resulting reaction mixture stirred for 48 h at 165° C. The progress of the reaction was monitored by TLC and LCMS. After completion of the starting material, reaction mixture was quenched with ice-water (30 mL) and adjusted to pH ~10-12 using NaHCO₃ then extracted with EtOAc (3×50 mL). The combined organic layers were dried over anh. Na₂SO₄ filtered and concentrated under reduced pressure. The residue was subjected to combi-flash chromatography using mixture of 0-10% MeOH-DCM as an eluent to afford 700 mg (47%) as the mixture of regioisomers P10 and P10a which was further purified by SFC purification to afford P10 (180 mg) (12.2%) and P10a (130 mg) (8.8%) as an off white solid.

Analytical data: P10: 1H NMR (400 MHz, CDCl₃), δ: 6.53 (s, 1H), 5.2 (br. s, 1H), 4.05 (br. s, 1H), 2.54-2.42 (m, 3H), 2.26 (s, 7H), 1.8-1.7 (m, 7H), 1.7 (s, 3H). LCMS (ESI): calc. for: C₁₁H₁₇ClN₄ [M+H]⁺ 241.11, found 241.35. P10a: ¹H NMR (400 MHz, DMSO-d₆), δ: 7.30 (d, J=0.8 Hz, 1H), 5.95 (d, J=8.0 Hz, 1H), 4.16-4.09 (m, 1H), 2.88 (d, J=8.0 Hz, 1H), 2.60 (d, J=10.4 Hz, 1H), 2.16 (s, 3H), 2.09 (s, 3H), 1.88-1.65 (m, 8H). LCMS (ESI): calc, for C₁₁H₁₇ClN₄ [M+H]⁺ 241.11, found 241.36.

### Synthesis of 7-(4,4,5,5-tetramethyl-1,3,2-dioxaborolan-2-yl)-4-(trifluoromethyl)-1H-indazole (P13)

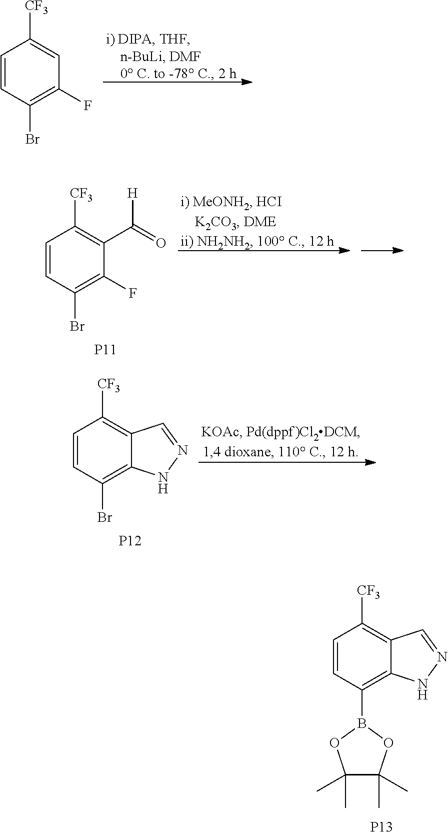

### Preparation 11. 3-Bromo-2-fluoro-6-(trifluoromethyl)benzaldehyde (P11)

To a stirred solution of DIPA (2.39 g, 23.66 mmol, 1.1 eq) in dry THF (50 mL), n-BuLi (1.6 M, 13.5 mL, 21.6 mmol, 1.0 eq) was added at 0° C. The resulting reaction mixture was stirred for 30 min at 0° C. After that a solution of 1-bromo-2-fluoro-4-(difluoromethyl)benzene (5.0 g, 20.57 mmol, 1.0 eq) in THF (50 mL) was added at −78° C. and stirred for 1 h at −78° C. then DMF (1.9 mL, 24.69 mmol, 1.2 eq) was added at −78° C. and reaction mixture was stirred for 30 min at 0° C. The progress of the reaction was monitored by TLC and LCMS. After completion of starting material, reaction mixture was quenched with AcOH (50 mL) followed by ice water (100 mL) was added, extracted with diethyl ether (2×100 mL). The combined organic layers were washed successively with dilute HCl solution (0.2 M, 100 mL), water (100 mL), brine solution (100 mL) and dried over anh. Na₂SO₄ filtered and concentrated under reduced pressure to afford (5.0 g crude) (100%) as a pale-yellow liquid.

Note: The crude itself proceed for the next step without further purification.

Analytical data: ¹H NMR (400 MHz, CDCl₃), δ: 10.38 (s, 1H), 7.92-7.88 (m, 1H), 7.5 (d, J=8.4 Hz, 1H).

### Preparation 12. 7-Bromo-4-(trifluoromethyl)-1H-indazole (P12)

To a stirred solution of 3-bromo-2-fluoro-6-(difluoromethyl)benzaldehyde (P11, 5.0 g, 18.4 mmol, 1.0 eq) in DME (50 mL) were added N-methylhydroxylamine hydrochloride (1.54 g, 18.4 mmol, 1.0 eq) and K₂CO₃ (2.8 g, 20.29 mmol, 1.1 eq) at ambient temperature. The reaction mixture was stirred for 4-5 h at 40° C. The progress of the reaction was monitored by TLC and LCMS. After completion of the starting material, reaction mixture was filtered and washed with DME (40 mL). The filtrate was concentrated under reduced pressure to afford the intermediate. Hydrazine monohydrate (50 mL, 92.24 mmol, 5.0 eq) was added to that intermediate at ambient temperature. The resulting reaction mixture was refluxed for 12 h at 100° C. The progress of reaction was monitored by TLC and LCMS. After completion of starting material, all volatiles evaporated under reduced pressure and extracted with EtOAc (3×50 mL). The combined organic layers were dried over anh. Na₂SO₄, filtered and concentrated under reduced pressure. The residue was subjected to combi-flash chromatography using 10-20% EtOAc in pet ether as eluent to afford 1.5 g of the title compound (P12, 31%) as a pale-yellow solid.

Analytical data: ¹H NMR (400 MHz, DMSO-d₆), δ: 14.11 (s, 1H), 8.34 (s, 1H), 7.81 (d, J=7.6 Hz, 1H), 7.48 (d, J=7.6 Hz, 1H). LCMS (ESI): calc, for C₈H₄BrF₃N₂ [M+H]⁺ 264.95, found 265.07.

### Preparation 13. 7-(4,4,5,5-Tetramethyl-1,3,2-dioxaborolan-2-yl)-4-(trifluoromethyl)-1H-indazole (P13)

To a stirred a solution of 7-bromo-4-(trifluoromethyl)-1H-indazole (P12, 1.5 g, 5.68 mmol, 1.0 eq) in 1,4-dioxane (30.0 mL) were added bis(pinacolato)diboron (2.88 g, 11.36 mmol, 2.0 eq), KOAc (2.78 g, 28.4 mmol, 5.0 eq) at ambient temperature. The reaction mixture was degassed with Ar for 10 min and PdCl₂(dppf)×DCM (0.416 g, 0.56 mmol, 0.1 eq) was added at ambient temperature. The reaction mixture was stirred for 12 h at 110° C. The progress of reaction was monitored by TLC and LCMS. After consuming of the starting material, reaction mixture was cooled to rt, filtered through celite pad and washed with diethyl ether (2×50 mL). The filtrate was washed with water (100 mL) and extracted with diethyl ether (3× 50 mL). The combined organic layers were dried over anh. Na₂SO₄, filtered and concentrated under reduced pressure. The residue was triturated with n-hexane at −78° C. filtered off to afford 1.2 g (P13, 79%) of the title compound as a pale-yellow solid.

Analytical data: ¹H NMR (400 MHz, DMSO-d₆), δ: 13.1 (s, 1H), 8.22 (br. s, 1H), 7.81 (d, J=6.8 Hz, 1H), 7.54 (d, J=7.2 Hz, 1H), 1.38 (s, 12H). LCMS (ESI): calc. for C₁₄H₁₆BF₃N₂O₂ [M+H]⁺ 231.05, found 231.06.

### Synthesis of 7-(4,4,5,5-tetramethyl-1,3,2-dioxaborolan-2-yl)-4-(trifluoromethyl)-1H-indazole (P16)

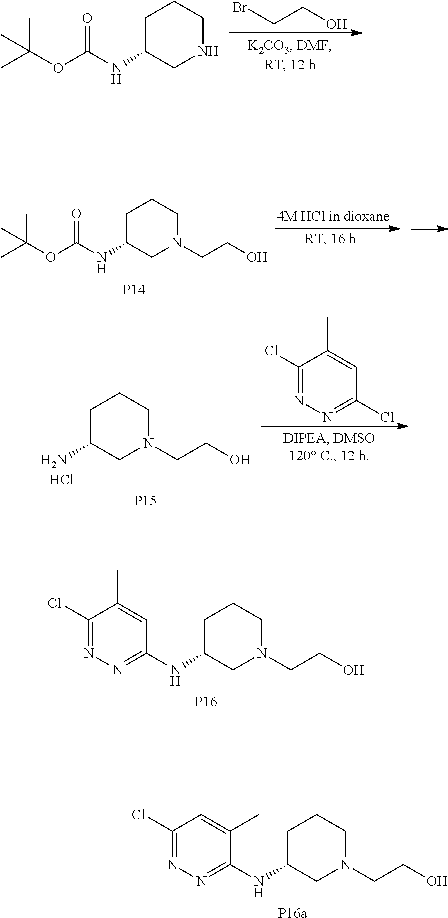

### Preparation 14. tert-Butyl (R)-(1-(2-hydroxyethyl) piperidin-3-yl) carbamate (P14)

To a stirred solution of tert-butyl-(R)-piperidin-3-ylcarbamate (10.0 g, 50.0 mmol, 1.0 eq) in ACN (150 mL) were added Na₂CO₃ (10.5 g, 100.0 mmol, 2.0 eq), 2-bromoethanol (9.3 g, 75.0 mmol, 1.5 eq) at 0° C. The resulting reaction mixture was stirred at ambient temperature for 12 h. The progress of the reaction was monitored by TLC and LCMS. After completion of starting material, reaction mixture was filtered through celite bed and washed with EtOAc (100 mL). The filtrate was concentrated under reduced pressure. The residue was subjected to combi-flash chromatography using mixture of 1-2% MeOH in DCM as an eluent to afford 8.0 g (65%) of the title compound P14 as an off-white solid.

Analytical data: ¹H NMR (400 MHz, DMSO-d₆), δ: 6.68 (d, J=8.0 Hz, 1H), 4.36 (bs, 1H), 4.09-4.08 (m, 1H), 3.44 (qt, J=5.6 Hz, 2H), 3.31 (bs, 1H), 3.16 (d, J=6.0 Hz, 1H), 2.74-2.72 (m, 1H), 2.60 (bs, 1H), 2.35 (t, J=5.6 Hz, 1H), 1.94-1.81 (m, 1H), 1.64-1.56 (m, 2H), 1.37 (s, 9H), 1.15-1.12 (m, 1H). LCMS (ESI): calc. for: C₁₂H₂₄N₂O₃ [M+H]⁺ 245.18, found 245.38.

### Preparation 15. (R)-2-(3-Aminopiperidin-1-yl) ethan-1-ol, hydrochloride (P15)

To a stirred solution of tert-butyl (R)-(1-(2-hydroxyethyl) piperidin-3-yl) carbamate (P14, 8.0 g, 32.62 mmol, 1.0 eq) in 1,4-dioxane (40 mL) was added 4N HCl in 1,4-dioxane (160 mL) at 0° C. The resulting reaction mixture was stirred at ambient temperature for 24 h. The progress of the reaction was monitored by TLC. After completion of starting material, all volatiles evaporated under reduced pressure. The residue was triturated with diethyl ether to afford 4.0 g (85%) of the title compound P15 as an off white solid.

Analytical data: ¹H NMR (400 MHz, DMSO-d₆), δ: 3.81-3.75 (m, 2H), 3.74-3.71 (m, 1H), 3.56 (s, 3H), 3.39-3.34 (m, 2H), 3.22 (bs, 1H), 3.07-2.98 (m, 2H), 2.96-2.89 (m, 2H), 2.09-2.00 (m, 1H), 1.91-1.86 (m, 2H). LCMS (ESI): calc. for: C₇H₁₆N₂O [M+H]⁺ 145.13, found 145.28.

### Preparation 16. (R)-6-chloro-N-(1-ethylpiperidin-3-yl)-5-methylpyridazin-3-amine (P16) and (R)-6-chloro-N-(1-ethylpiperidin-3-yl)-4-methylpyridazin-3-amine (P16A)

To a stirred solution of 3,6-dichloro-4-methylpyridazine (P15, 4.0 g, 24.5 mmol, 1.0 eq) in DMSO (40.0 mL), DIPEA (63.43 g, 490.76 mmol, 20 eq) and (R)-2-(3-aminopiperidin-1-yl) ethan-1-ol (6.3 g, 29.4 mmol, 1.2 eq) were added at ambient temperature in a sealed tube. The resulting reaction mixture stirred for 48 h at 165° C. The progress of the reaction was monitored by TLC and LCMS. After completion of the starting material, reaction mixture was quenched with ice-water (150 mL) and pH adjusted to ~10-12 with NaHCO₃ solution then extracted with EtOAc (3× 100 mL). The combined organic layers were dried over anh. Na₂SO₄, filtered and concentrated under reduced pressure. The residue was purified by flash chromatography using 0-10% MeOH in DCM as an eluent to afford 1.5 g (40%) of the title compound as the mixture of regioisomers. Further SFC purification afford P16 (200 mg, 3.0%) and P16A (180 mg, 2.7%) as an off-white solid.

Analytical data: P16: 1H NMR (400 MHz, CDCl₃), δ: 6.55 (s, 1H), 5.17 (br. s, 1H), 4.04 (br. s, 1H), 3.67-3.61 (m, 1H), 2.76-2.74 (m, 1H), 2.58-2.32 (m, 5H), 2.27 (s, 5H), 1.76 (br. s, 2H), 1.62 (br. s, 2H). LCMS (ESI): calc. for: C₁₂H₁₉ClN₄O [M+H]⁺ 271.12, found 271.19. P16A: ¹H NMR (400 MHz, CDCl₃), δ: 7.01 (s, 1H), 4.71 (br. s, 1H), 4.48 (br. s, 1H), 3.67-3.61 (m, 2H), 2.78-2.60 (m, 1H), 2.60-2.52 (m, 5H), 2.06 (s, 5H), 1.74-1.63 (m, 3H). LCMS (ESI): calc. for: C₁₂H₁₉ClN₄O [M+H]⁺ 271.12, found 271.35.

### Synthesis of N-(3-methyl-2-(4,4,5,5-tetramethyl-1,3,2-dioxaborolan-2-yl)-5-(trifluoromethyl)phenyl) methanesulfonamide (P19)

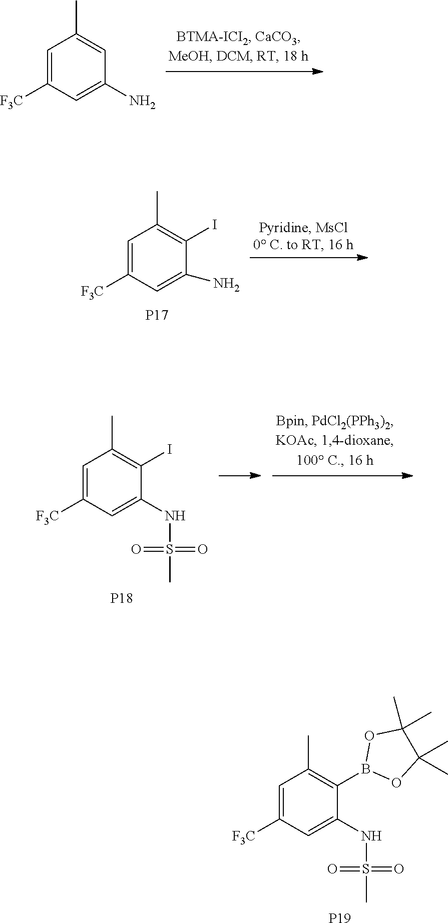

### Preparation 17. 2-Iodo-3-methyl-5-(trifluoromethyl) aniline (P17)

To a stirred solution of 3-methyl-5-(trifluoromethyl) aniline (2.5 g, 14.27 mmol, 1.0 eq) in DCM (25 mL) and MeOH (5 mL) was added BTMA·ICl₂ (9.93 g, 28.54 mmol, 2.0 eq) and CaCO₃ (3.57 g, 35.68 mmol, 2.5 eq) at ambient temperature. The resulting reaction mixture was stirred for 18 h at this temperature. The progress of reaction was monitored by TLC & LCMS. After completion of the reaction, the reaction mixture was filtered and washed with DCM (80 mL). The filtrate was washed with sat. Na₂S₂O₃ solution (2×60 mL), water (40 mL), and dried over anh. Na₂SO₄, filtered, evaporated under reduced pressure at 45° C. The residue was subjected to flash chromatography over silica gel (230-400 mesh) using mixture of EtOAc (10→20%) and hexanes as an eluent to afford 1.5 g (35%) of the title compound P17 as a yellow liquid.

Analytical data: ¹H NMR (400 MHz, CDCl₃), δ: 6.86-6.85 (m, 1H), 6.78-6.77 (m, 1H), 4.37 (br. s, 2H), 2.46 (s, 3H); LCMS (ESI): calc, for C₈H₈F₃IN [M+H]⁺ 301.9, found 301.9.

### Preparation 18. N-(2-iodo-3-methyl-5-(trifluoromethyl)phenyl) methanesulfonamide (P18)

To a stirred solution of P17 (1.8 g, 5.979 mmol, 1.0 eq) in pyridine (20 mL) was added MsCl (2.3 mL, 29.89 mmol, 5.0 eq) at 0° C. The resulting reaction mixture was stirred at ambient temperature for 16 h. The progress of reaction was monitored by TLC & LCMS. After completion of the reaction, the reaction mixture was quenched with water (30 mL) and extracted with EtOAc (2×60 mL). The combined organic layer was washed with brine (40 mL), dried over anh. Na₂SO₄, filtered, evaporated under reduced pressure at 45° C. The residue was subjected to flash chromatography over silica gel (230-400 mesh) using mixture of EtOAc (15→20%) and hexanes as an eluent to afford 1.7 g (75%) of the title compound P18 as an off white solid.

Analytical data: ¹H NMR (400 MHz, CDCl₃), δ: 7.50 (br. s, 1H), 7.12 (br. s, 1H), 2.92 (s, 3H), 2.48 (s, 3H); LCMS (ESI): calc, for C₉H₁₀F₃INO₂S [M+H]⁺ 379.9, found 380.2.

### Preparation 19. N-(3-methyl-2-(4,4,5,5-tetramethyl-1,3,2-dioxaborolan-2-yl)-5-(trifluoromethyl)phenyl) methanesulfonamide (P19)

To a stirred solution of P18 (0.5 g, 1.31 mmol, 1.0 eq), bis(pinacolato)diboron (670 mg, 2.63 mmol, 2.0 eq) in 1,4-dioxane (10 mL) was added KOAc (388 mg, 3.95 mmol, 3.0 eq) at ambient temperature. The reaction mixture was degassed with a stream of nitrogen for 15 min. Thereafter, Pd(dppf)Cl₂·DCM (46 mg, 0.066 mmol, 0.05 eq) was added at this temperature. The reaction mixture was heated to 100° C., stirred for 16 h at 100° C. The progress of reaction was monitored by TLC and LCMS. The reaction mixture was cooled to ambient temperature and filtered through celite pad; the pad was washed with 10% MeOH in DCM (40 mL). The filtrate was evaporated under reduced pressure at 40° C. The residue was triturated with hexane (2×30 mL) and decanted. The decanted hexane layer was concentrated under reduced pressure to afford 0.8 g (crude) of the title compound P19 as a yellow liquid.

Analytical data: LCMS (ESI): calc, for C₁₅H₂₁BF₃NO₄S [M+H]⁺ 380.12, found 380.18.

### Synthesis of (R)-6-chloro-N-(1-methylpiperidin-3-yl)pyridazin-3-amine (P20)

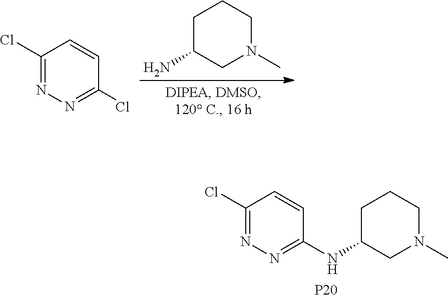

### Preparation 20. (R)-6-Chloro-N-(1-methylpiperidin-3-yl)pyridazin-3-amine (P20)

To a stirred solution of 3,6-dichloropyridazine (0.5 g, 3.35 mmol, 1.0 eq) in DMSO (10.0 mL) was added DIPEA (1.78 mL, 10.06 mmol, 3.0 eq), followed by (R)-1-methylpiperidin-3-amine (0.38 g, 3.35 mmol, 1.0 eq) at ambient temperature. The resulting reaction mixture stirred at 110° C. for 16 h. The progress of reaction was monitor by TLC and LCMS. After completion of the starting material, the reaction mixture was quenched with water (30 mL) and extracted with 10% MeOH in DCM (2× 40 mL). The combined organic layers were dried over anh. Na₂SO₄, filtered and concentrated under reduced pressure at 45° C. to give residue. The residue was subjected to flash chromatography over silica gel (230-400 mesh) using mixture of 5% Et₃N in MeOH (10→20%) and DCM as an eluent to afford 220 mg (28%) of the title compound P20 as an off white solid.

Analytical data: ¹H NMR (400 MHz, CD₃OD), δ: 8.53 (s, 1H), 7.33 (d, J=9.6 Hz, 1H), 6.93 (d, J=9.6 Hz, 1H), 4.20-4.15 (m, 1H), 3.24-3.17 (m, 2H), 2.64 (s, 3H), 2.05-1.95 (m, 2H), 1.82-1.77 (m, 1H), 1.59-1.52 (m, 1H), 1.30 (t, J=7.2 Hz, 4H); LCMS (ESI): calc. for: C₁₀H₁₅ClN₄ [M+H]⁺ 227.1, found 227.3.

### Synthesis of (R)-6-chloro-N-(1-ethylpiperidin-3-yl)pyridazin-3-amine (P23)

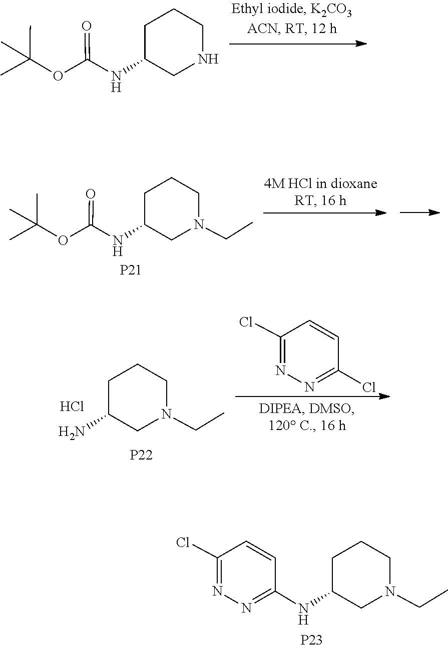

### Preparation 21. tert-Butyl (R)-(1-ethylpiperidin-3-yl) carbamate (P21)

To a stirred solution of tert-butyl (R)-piperidin-3-ylcarbamate (5.0 g, 25.0 mmol, 1.0 eq) in ACN (75 mL) were added K₂CO₃ (4.14 g, 30.0 mmol, 1.2 eq), followed by ethyl iodide (4.28 g, 27.5 mmol, 1.1 eq) at 0° C. The resulting reaction mixture was stirred at ambient temperature for 12 h. The progress of the reaction was monitored by TLC and LCMS. After completion of starting material, the reaction mixture was filtered through celite bed and washed with DCM (30 mL). The filtrate was evaporated under reduced pressure at 40° C. to give residue. The residue was subjected to flash chromatography over silica gel (230-400 mesh) using mixture of MeOH (1→2%) and DCM as an eluent to afford 2.5 g (43%) of the title compound P21 as a pale-yellow liquid.

Analytical data: ¹H NMR (400 MHz, CDCl₃), δ: 5.06 (br. s, 1H), 3.79 (s, 1H), 2.78-2.25 (m, 6H), 1.77-1.55 (m, 3H), 1.44 (s, 9H), 1.12 (t, J=6.8 Hz, 3H); LCMS (ELSD) (ESI): calc. for: C₁₂H₂₄N₂O₂ [M+H]⁺ 229.18, found 229.25.

### Preparation 22. (R)-1-Ethylpiperidin-3-amine hydrochloride (P22)

To a stirred solution of tert-butyl (R)-(1-ethylpiperidin-3-yl) carbamate (P21, 2.5 g, 10.95 mmol, 1.0 eq) in 1,4-dioxane (12.5 mL) was added 4M HCl in 1,4-dioxane (25 mL) at 0° C. The resulting reaction mixture was stirred at ambient temperature for 16 h. The progress of the reaction was monitored by TLC and LCMS. After completion of starting material, the reaction mixture was concentrated under reduced pressure at 40° C. to give residue. The residue was triturate with diethyl ether (20 mL) and dried to afford 2.0 g (crude) of the title compound P22 as a yellow solid.

Analytical data: ¹H NMR (400 MHz, DMSO-d₆), δ: 8.71 (br. s, 3H), 3.60-3.56 (m, 2H), 3.45-3.42 (m, 1H), 3.21-3.02 (m, 2H), 2.98-2.66 (m, 2H), 2.17-1.75 (m, 3H), 1.62-1.48 (m, 1H), 1.25 (t, J=7.2 Hz, 3H); LCMS (ESI): calc. for: C₇H₁₇ClN₂ [M−HCl+H]⁺ 129.13, found 129.18.

### Preparation 23. (R)-6-chloro-N-(1-ethylpiperidin-3-yl)pyridazin-3-amine (P23)

To a stirred solution of 3,6-dichloropyridazine (0.5 g, 3.35 mmol, 1.0 eq) in DMSO (5.0 mL) was added DIPEA (1.75 mL, 10.06 mmol, 3.0 eq), followed by P22 (607 mg, 3.69 mmol, 1.1 eq) at ambient temperature. The resulting reaction mixture stirred at 120° C. for 16 h. The progress of reaction was monitor by TLC and LCMS. After completion of the starting material, the reaction mixture was quenched with water (30 mL) and extracted with 10% MeOH in DCM (2× 40 mL). The combined organic layers were dried over anh. Na₂SO₄, filtered and concentrated under reduced pressure at 45° C. to give residue. The residue was subjected to flash chromatography over silica gel (230-400 mesh) using mixture of 5% Et₃N in MeOH (10→20%) and DCM as an eluent to afford 300 mg (37%) of the title compound P23 as a light brown solid.

Analytical data: ¹H NMR (400 MHz, CD₃OD), δ: 7.37 (d, J=9.6 Hz, 1H), 6.96 (d, J=9.6 Hz, 1H), 4.28-4.21 (m, 1H), 3.72-3.54 (m, 1H), 3.16-3.07 (m, 2H), 2.95-2.72 (m, 2H), 2.17-2.03 (m, 2H), 1.93-1.84 (m, 1H), 1.76-1.61 (m, 1H), 1.31 (t, J=7.6 Hz, 4H). LCMS (ESI): calc. for: C₁₁H₁₇ClN₄ [M+H]⁺ 241.1, found 241.36.

### Synthesis of (R)-6-Chloro-N-(1-ethylpiperidin-3-yl)-5-methylpyridazin-3-amine and (R)-6-chloro-N-(1-ethylpiperidin-3-yl)-4-methylpyridazin-3-amine (P24 and P24A)

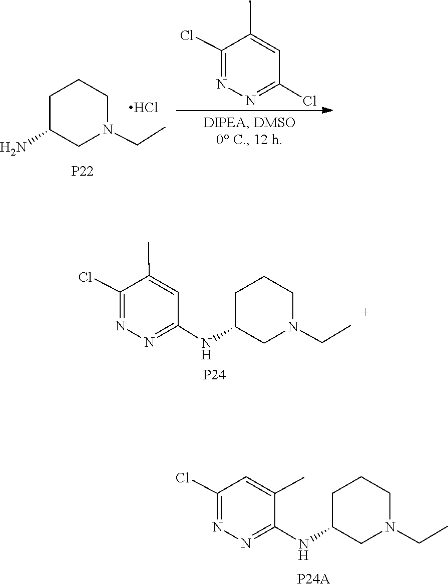

### Preparation 24. (R)-6-chloro-N-(1-ethylpiperidin-3-yl)-5-methylpyridazin-3-amine and (R)-6-chloro-N-(1-ethylpiperidin-3-yl)-4-methylpyridazin-3-amine (P24 and P24A)

To a stirred solution of 3,6-dichloro-4-methylpyridazine (2.5 g, 15.33 mmol, 1.0 eq) in DMSO (25.0 mL) were added DIPEA (39.5 g, 306.7 mmol, 20 eq) and P22 (3.6 g, 18.0 mmol, 1.2 eq) at ambient temperature. The resulting reaction mixture was stirred for 48 h at 165° C. The progress of the reaction was monitored by TLC and LCMS. After completion of the starting material, reaction mixture was quenched with ice-water (30 mL), adjusted pH~10-12 with NaHCO₃ solution and extracted with EtOAc (3×100 mL). The combined organic layers were dried over anh. Na₂SO₄, filtered and concentrated under reduced pressure. The residue was subjected to combi-flash chromatography using 0-10% MeOH in DCM as an eluent to afford 1.5 g (40%) of the title compound as a mixture of regioisomers, which was further purified by SFC to afford peak-1 (P24, 500 mg, 8.0%), peak-2 (P24A, 400 mg, 6.4%) as an off white solid.

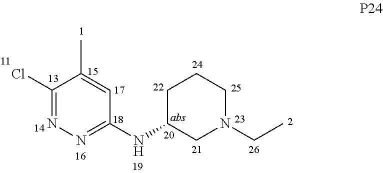 [SMILES: CCN1CCC[C@@H](Nc2cc(C)c(Cl)nn2)C1]

In NOE experiment data, on irradiation of H1 Proton at 2.18 ppm giving enhancement of H17 Proton at 6.81 ppm.

Analytical data: ¹H NMR (400 MHz, DMSO-d₆), δ: 6.81 (s, 1H), 6.78 (d, J=8.0 Hz, 1H), 3.95-3.89 (m, 1H), 2.89-2.83 (m, 1H), 2.67-2.61 (m, 1H), 2.35 (qt, J=6.4 Hz, 2H), 2.18 (s, 3H), 2.07-1.80 (m, 3H), 1.70-1.66 (m, 1H), 1.55-1.44 (1H), 1.32-1.10 (m, 1H), 1.0 (t, J=8.0 Hz, 3H). LCMS (ESI): calc. for: C₁₂H₁₉ClN₄ [M+H]⁺ 255.13, found 255.32.

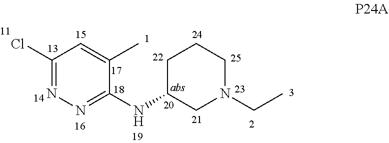 [SMILES: CCN1CCC[C@@H](Nc2nnc(Cl)cc2C)C1]

In NOE experiment data, on irradiation of H19 Proton at 5.95 ppm giving enhancement of H20 Proton at 4.11 ppm and H1 Proton at 2.96 ppm

Analytical data: ¹H NMR (400 MHz, DMSO-d₆), δ: 7.29 (s, 1H), 5.95 (d, J=7.6 Hz, 1H), 4.16-4.09 (m, 1H), 2.97-2.91 (m, 1H), 2.72-2.54 (m, 2H), 2.35 (qt, J=7.2 Hz, 2H), 2.09 (s, 3H), 1.95-1.75 (m, 3H), 1.65-1.55 (1H), 1.52-1.49 (m, 1H), 1.45-1.35 (m, 1H), 1.02 (t, J=7.2 Hz, 3H). LCMS (ESI): calc, for C₁₂H₁₉ClN₄ [M+H]⁺ 255.13, found 255.28.

### Synthesis of (R)-3-(4-Methyl-6-((1-methylpiperidin-3-yl)amino)pyridazin-3-yl)-6-(trifluoro methyl)benzene-1,2-diamine (P25)

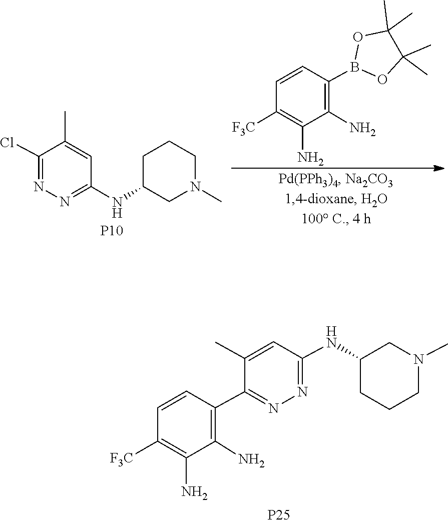

### Preparation 25. (R)-3-(4-Methyl-6-((1-methylpiperidin-3-yl)amino)pyridazin-3-yl)-6-(trifluoro methyl)benzene-1,2-diamine (P25)

To a stirred solution of 3-(4,4,5,5-tetramethyl-1,3,2-dioxaborolan-2-yl)-6-(trifluoromethyl)benzene-1,2-diamine (0.50 g, 2.27 mmol, 1.0 eq), P10 (0.54 g, 2.27 mmol, 1.0 eq) in 1,4-dioxane (6.0 mL) and water (2.0 mL) was added sodium carbonate (0.48 g, 4.54 mmol, 2.0 eq) at ambient temperature. The reaction mixture was degassed with a stream of nitrogen for 15 min. Thereafter, tetrakis (triphenylphosphine) palladium (0) (0.13 g, 0.114 mmol, 0.05 eq) was added at this temperature. The mixture was stirred for 4 h at 100° C. The progress of reaction was monitored by TLC and LCMS. After completion of starting material, the reaction mixture was cooled to ambient temperature and concentrated under reduced pressure. The residue was subjected to reverse phase column chromatography using mixture of ACN (0 to 70%) and water as eluent to afford 0.210 g (24%) of the title compound as a brown solid.

Analytical data: ¹H NMR (400 MHz, DMSO-d₆), δ: 8.42 (s, 2H), 7.82 (br. s, 1H), 7.04 (d, J=8.4 Hz, 1H), 6.98-6.92 (m, 1H), 6.68 (d, J=8.4 Hz, 1H), 4.79-4.57 (m, 1H), 3.10-2.95 (m, 2H), 2.80-2.62 (m, 5H), 2.45-2.30 (m, 1H), 2.20-2.02 (m, 4H), 1.89-1.80 (m, 2H). LCMS (ESI): calc, for C₁₈H₂₃F₃N₆ [M+H]⁺ 381.19, found 381.54.

### Synthesis of 7-(6-Chloro-4,5-dimethylpyridazin-3-yl)-4-(trifluoromethyl)-1H-benzo[d][1,2,3]triazole (P27)

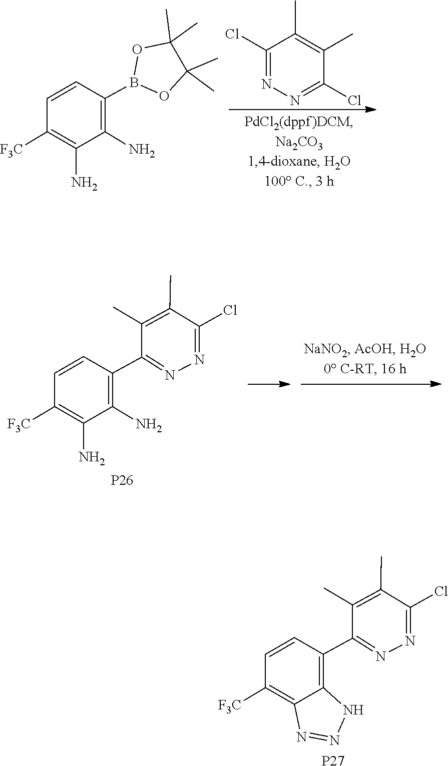

### Preparation 26. 3-(6-Chloro-4,5-dimethylpyridazine-3-yl)-6-(trifluoromethyl)benzene-1,2-diamine (P26)

To a stirred solution of 3,6-dichloro-4,5-dimethylpyridazine (0.58 g, 1.0 eq, 3.31 mmol) in a mixture of 1,4-dioxane (3.0 mL) and water (1.0 mL) were added 3-(4,4,5,5-tetramethyl-1,3,2-dioxaborolan-2-yl)-6-(trifluoromethyl)benzene-1,2-diamine (1.0 g, 1.0 eq, 3.31 mmol) and sodium carbonate (0.70 g, 2.0 eq, 6.62 mmol) at rt. The resulting mixture was purged with argon for 10 min. Thereafter, [1,1′-bis(diphenylphosphino) ferrocene]dichloro palladium (II) complex with dichloromethane (0.121 g, 0.05 eq, 0.16 mmol) was added at rt. The resulting reaction mixture was stirred for 3 h at 100° C. The progress of reaction was monitored by TLC. The reaction mixture was diluted with 10% MeOH in DCM (20 mL) and filtered through a celite pad. The celite pad was washed with 10% MeOH in DCM (10 mL) and the combined filtrate was concentrated under reduced pressure. The crude compound was purified by reverse phase column chromatography over C18 column using a mixture of ACN (0→60%) and water as an eluent to afford 0.3 g (28%) of the title compound 3 as a brown solid.

Analytical data: LCMS (ESI): calc, for C₁₃H₁₂ClF₃N₄ [M+H]⁺ 317.07, found 317.07.

### Preparation 27. 7-(6-Chloro-4,5-dimethylpyridazin-3-yl)-4-(trifluoromethyl)-1Hbenzo[d][1,2,3]triazole (P27)

To a stirred solution of P26 (0.30 g, 1.0 eq, 0.949 mmol) in AA (0.11 mL, 2.0 eq, 1.89 mmol) was added a solution of sodium nitrite (0.131 g, 2.0 eq, 1.899 mmol) in water (1.5 mL) at 0° C. and the resulting reaction mixture was stirred for 16 h at rt. The progress of the reaction is monitored by TLC. After completion of the reaction, the reaction mixture concentrated under reduced pressure and diluted with water (20 ml) and extracted with EA (2×30 mL). The combined organic layer was dried over sodium sulphate and concentrated under reduced pressure to afford 310 mg (crude) of the title compound P27 as a brown solid.

Analytical data: LCMS (ESI): calc, for C₁₃H₁₀ClF₃N₅ [M+H]⁺ 328.05, found 328.14.

### Synthesis of (R)-3-(6-((1-Methylpiperidin-3-yl)amino)-4-(trifluoromethyl)pyridazin-3-yl)-6-(trifluoromethyl)benzene-1,2-diamine (P29)

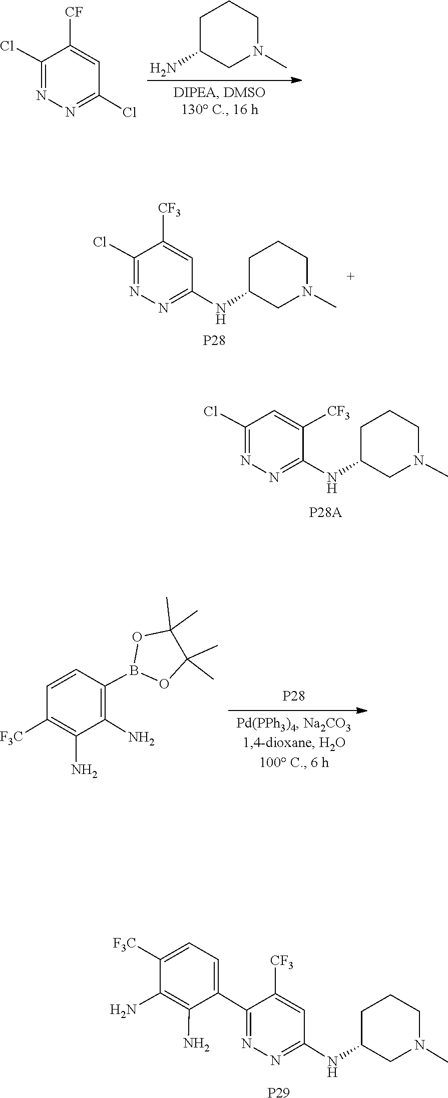

### Preparation 28. (R)-6-Chloro-N-(1-methylpiperidin-3-yl)-5-(trifluoromethyl)pyridazin-3-amine (P28)

To a stirred solution of 3,6-dichloro-4-(trifluoromethyl)pyridazine (0.50 g, 2.30 mmol, 1.0 eq) in DMSO (3.0 mL) were added (R)-1-methylpiperidin-3-amine (0.211 g, 1.84 mmol, 0.8 eq) and N,N-diisopropylethylamine (2.0 mL, 11.52 mmol, 5.0 eq) at ambient temperature and stirred at 130° C. for 16 h. The progress of reaction was monitored by TLC and LCMS. After completion of starting material, reaction mixture was concentrated under reduced pressure. Following the above procedure, another 0.50 g of 3,6-dichloro-4-(trifluoromethyl)pyridazine was treated with (R)-1-methylpiperidin-3-amine and both these batches were mixed. The combined residue was subjected to reverse phase column purification using mixture of water (0-90%) and ACN (10%) as eluent to afford 0.43 g (32%) of the title compound P28 and 0.42 g (31%) of the compound P28A as brown solids.

Analytical data for compound P28: 1H NMR (400 MHz, DMSO-d₆), δ: 7.55 (d, J=7.6 Hz, 1H), 7.39 (s, 1H), 4.15-4.05 (m, 1H), 2.85-2.80 (m, 1H), 2.52-2.50 (m, 1H), 2.35-2.30 (m, 1H), 2.25 (s, 3H), 2.20-2.15 (m, 1H), 1.80-1.70 (m, 2H), 1.60-1.50 (m, 1H), 1.45-1.35 (m, 1H). LCMS (ESI): calc, for C₁₁H₁₄ClF₃N₄ [M+H] 295.09, found 295.23.

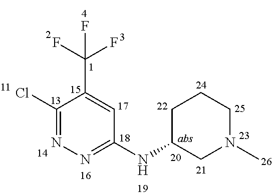 [SMILES: CN1CCC[C@@H](Nc2cc(C(F)(F)F)c(Cl)nn2)C1]

The structure of compound P28 was confirmed by NOE: On irradiation of H17 (7.39 ppm) proton showing enhancement of H19 (—NH, doublet, 7.55 ppm) proton.

Analytical data for P28A: LCMS (ESI): calc, for C₁₁H14ClF₃N₄ [M+H]⁺ 295.09, found 295.23.

### Preparation 29. (R)-3-(6-((1-Methylpiperidin-3-yl)amino)-4-(trifluoromethyl)pyridazin-3-yl)-6-(trifluoromethyl)benzene-1,2-diamine (P29)

To a stirred solution of 3-(4,4,5,5-tetramethyl-1,3,2-dioxaborolan-2-yl)-6-(trifluoromethyl)benzene-1,2-diamine (0.40 g, 1.32 mmol, 1.0 eq), P28 (0.39 g, 1.32 mmol, 1.0 eq) in a mixture of 1,4-dioxane (3.0 mL) and water (1.0 mL) was added sodium carbonate (0.28 g, 2.64 mmol, 2.0 eq) at ambient temperature. The reaction mixture was degassed with a stream of nitrogen for 15 min. Thereafter, tetrakis (triphenylphosphine) palladium (0) (76 mg, 0.06 mmol, 0.05 eq) was added at this temperature. The mixture was heated to 100° C. and stirred for 6 h. The progress of reaction was monitored by TLC and LCMS. After completion of starting material, the reaction mixture was cooled to ambient temperature and concentrated under reduced pressure. The residue was subjected to reverse column chromatography using mixture of ACN (0 to 70%) and water as an eluent to afford 0.270 g (47%) of the title compound as a brown solid.

Analytical data: LCMS (ESI): calc, for C₁₈H₂₀F₆N₆ [M+H]⁺ 435.17, found 435.66.

### Synthesis of (R)-3-(6-((1-Methylpiperidin-3-yl)amino)-4-(trifluoromethyl)pyridazin-3-yl)-6-(trifluoromethyl)benzene-1,2-diamine (P30)

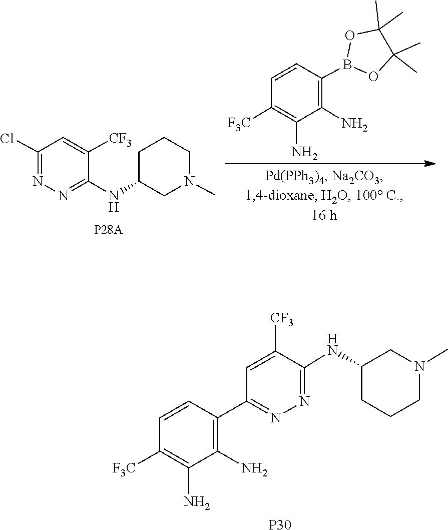

### Preparation 30. (S)-3-(6-((1-Methylpiperidine-3-yl)amino)-4-(trifluoromethyl)pyridazin-3-yl)-6-(trifluoromethyl)benzene-1,2-diamine (P30)

To a stirred solution of P28A (0.35 g, 1.18 mmol, 1.0 eq) in a mixture of 1,4-dioxane (6 mL) and water (0.5 mL) were added sodium carbonate (0.25 g, 2.36 mmol, 2.0 eq) and 3-(4,4,5,5-tetramethyl-1,3,2-dioxaborolan-2-yl)-6-(trifluoromethyl)benzene-1,2-diamine (0.53 g, 1.77 mmol, 1.5 eq) at ambient temperature. The reaction mixture was degassed with a stream of nitrogen for 10 min. Thereafter tetrakis(triphenylphosphine) palladium (0) (0.127 g, 0.11 mmol, 0.1 eq) was added at ambient temperature. The reaction mixture was heated to 100° C. and stirred for 16 h at 100° C. The progress of reaction was monitored by TLC and LCMS. The reaction mixture was cooled to ambient temperature and filtered through a celite bed and washed the celite bed with 1,4-dioxane (2×25 mL). The combined filtrate was concentrated under reduced pressure. The crude compound was subjected to flash chromatography over silica gel (230-400 mesh) using mixture of MeOH (0→10%) and DCM as an eluant to afford 0.20 g (39%) of the title compound P30 as a brown solid.

Analytical data: LCMS (ESI): calc, for C₁₈H₂₀F₆N₆ [M+H]⁺ 435.17, found 435.59.

### Synthesis of (R)-4-Chloro-N-(1-methylpiperidin-3-yl)-5,6,7,8-tetrahydrophthalazin-1-amine (P33)

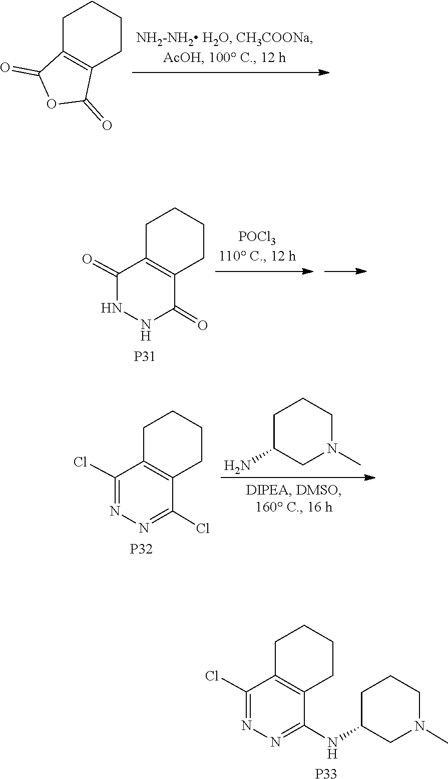

### Preparation 31. 2,3,5,6,7,8-Hexahydrophthalazine-1,4-dione (P31)

To a stirred solution of 4,5,6,7-tetrahydroisobenzofuran-1,3-dione (9.0 g, 59.21 mmol, 1.0 eq) in AA (270 mL) were added sodium acetate (38.85 g, 473.68 mmol, 8.0 eq) and hydrazine monohydrate (5.5 mL, 177.63 mmol, 3.0 eq) at ambient temperature. The resulting reaction mixture was stirred for 12 h at 100° C. The progress of reaction mixture monitored by TLC and LCMS. After completion of starting material, reaction mixture was quenched into cold water (200 mL) and the precipitated solid was collected by filtration and washed with cold water and dried in vacuo. This solid was triturated with n-hexane and dried to get to afford 6.3 g (64%) of the title compound as a colorless solid.

Analytical data: ¹H NMR (400 MHz, DMSO-d₆), δ: 11.34-11.30 (m, 2H), 2.38-2.30 (m, 4H), 1.68-1.61 (m, 4H). LCMS (ESI): calc. for: C₈H₁₀N₂O₂ [M+H]⁺ 167.07, found 167.09.

### Preparation 32. 1,4-Dichloro-5,6,7,8-tetrahydrophthalazine (P32)

POCl₃ (31.5 mL) was added to P31 (6.3 g, 37.91 mmol, 1.0 eq) at ambient temperature. The resulting reaction mixture stirred for 12 h at 110° C. The progress of reaction mixture monitored by TLC and LCMS. After completion of the starting material, the reaction mixture was concentrated under reduced pressure. The residue was quenched into cold water (100 mL) and the precipitated solid was collected by filtration and washed with cold water and dried in vacuo. This solid was triturated with n-hexane and dried to get to afford 5.0 g (65%) of the title compound P32 as a brown solid.

Analytical data: ¹H NMR (400 MHz, DMSO-d₆), δ: 2.76-2.73 (m, 4H), 1.89-1.85 (m, 4H). LCMS (ESI): calc. for: C₈H₈Cl₂N₂ [M+H]⁺ 203.01, found 203.10.

### Preparation 33. (R)-4-Chloro-N-(1-methylpiperidin-3-yl)-5,6,7,8-tetrahydrophthalazin-1-amine (P33)

To a stirred solution of P32 (1.0 g, 4.92 mmol, 1.0 eq) in DMSO (10 mL) were added DIPEA (6.35 g, 49.24 mmol, 10 eq) and (R)-1-methylpiperidin-3-amine (1.12 g, 9.84 mmol, 2.0 eq) at ambient temperature in a sealed tube. The resulting reaction mixture stirred for 16 h at 160° C. The progress of reaction was monitored by TLC and LCMS. After completion of the starting material, the reaction mixture was quenched with sat. NaHCO₃ solution (10 mL) and extracted with EtOAc (3×25 mL). The combined organic layers were dried over anh. Na₂SO₄, filtered and concentrated under reduced pressure. The residue was subjected to flash column chromatography using mixture of MeOH (0→10%) and DCM as an eluent to afford 0.30 g (23%) of the title compound as a brown liquid.

Analytical data: LCMS (ESI): calc. for: C₁₄H₂₁ClN₄ [M+H]⁺ 281.15, found 281.17.

### Synthesis of (R)-4-Chloro-N-(1-ethylpiperidin-3-yl)-5,6,7,8-tetrahydrophthalazin-1-amine (P34)

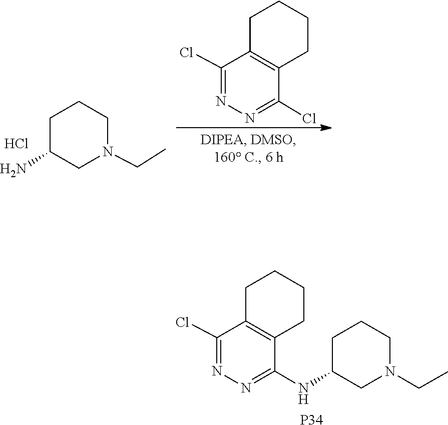

### Preparation 34. (R)-4-Chloro-N-(1-ethylpiperidin-3-yl)-5,6,7,8-tetrahydrophthalazin-1-amine (P34)

To a stirred solution of 1,4-dichloro-5,6,7,8-tetrahydrophthalazine (P-32, 1.0 g, 4.924 mmol, 1.0 eq) in DMSO (10 mL) were added DIPEA (6.352 g, 49.244 mmol, 10 eq) and (R)-1-ethylpiperidin-3-amine (1.25 g, 9.849 mmol, 2.0 eq) at ambient temperature. The resulting reaction mixture stirred for 6 h at 160° C. in a sealed tube. The progress of the reaction was monitored by TLC and LCMS. After completion of the starting material, all volatiles were removed in vacuo. The residue was purified by reverse phase column chromatography using mixture of ACN (0→30%) and 0.1% FA in water to afford 0.5 g (34%) of the title compound P34 as a brown liquid.

Analytical data: ¹H NMR (400 MHz, CDCl₃), δ: 8.37 (br. s, exch. with D₂O, 1H), 6.23 (br. s, 1H), 4.85 (s, 1H), 3.74-3.55 (m, 1H), 3.10-2.95 (m, 2H), 2.63 (s, 8H), 2.32-2.18 (m, 1H), 1.93-1.68 (m, 3H), 1.56 (merged with water, 3H), 1.36 (t, J=7.2 Hz, 2H). LCMS (ESI): calc. for: C₁₅H₂₃ClN₄ [M+H]⁺ 295.16, found 295.32.

### Synthesis of N-(2-(4,4,5,5-tetramethyl-1,3,2-dioxaborolan-2-yl)-5-(trifluoromethyl)phenyl) methane sulfonamide (P37)

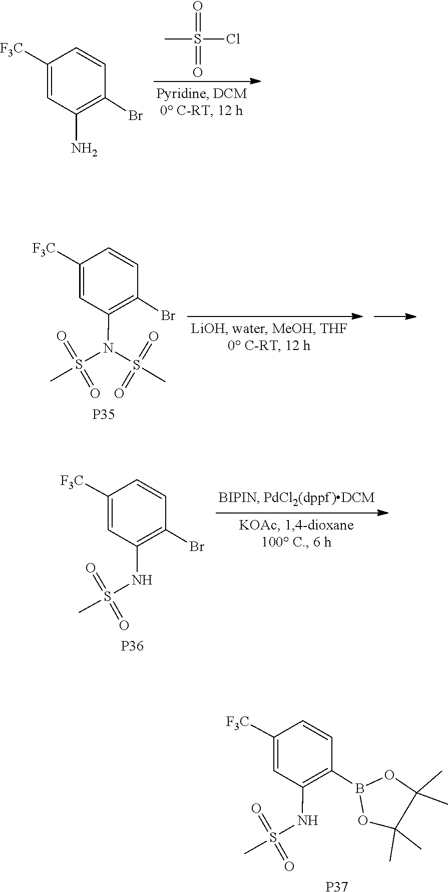

### Preparation 35. N-(2-Bromo-5-(trifluoromethyl)phenyl)-N-(methylsulfonyl) methane sulfonamide (P35)

To a stirred solution of 2-bromo-5-(trifluoromethyl) aniline (10.0 g, 41.60 mmol, 1.0 eq) and pyridine (16.81 mL, 208.316 mmol, 5.0 eq) in DCM (200 mL) was added methane sulfonyl chloride (16.12 mL, 208.31 mmol, 5.0 eq) at 0° C. The resulting reaction mixture was stirred for 12 h at ambient temperature. The progress of the reaction was monitored by TLC and LCMS. After completion of the starting material, reaction mixture was quenched with water (250 mL) and extracted with EtOAc (3× 150 mL). The combined organic layers were dried over anh. Na₂SO₄, filtered and concentrated under reduced pressure to afford 10 g (crude) of the title compound P37 as a brown liquid. Crude itself proceeded for the next step without any purification.

Analytical data: LCMS (ESI): calc. for: C₉H₉BrF₃NO₄S₂ [M+H]⁺ 366.19, found 366.85.

### Preparation 36. N-(2-Bromo-5-(trifluoromethyl)phenyl) methane sulfonamide (P36)

To a stirred solution of P35 (10.0 g, 25.38 mmol, 1 eq) in a mixture of THF/MeOH (1:1, 200 mL) was added a solution of LiOH monohydrate (31.94 g, 761.42 mmol, 30 eq) in water (100 mL) at 0° C. The resulting reaction mixture was stirred for 12 h at ambient temperature. The progress of the reaction was monitored by TLC and LCMS. After completion of the starting material, the reaction mixture was concentrated under reduced pressure. The residue was quenched with cold water (50 mL) and extracted with EtOAc (3× 100 mL). The combined organic layers were dried over anh. Na₂SO₄, filtered and concentrated under reduced pressure. The residue was subjected to flash chromatography over silica gel (230-400 mesh) using mixture of EtOAc (0→20%) and hexanes to afford 7.0 g (87%) of the title compound P36 as a colorless solid.

Analytical data: ¹H NMR (400 MHz, CDCl₃), δ: 7.92 (d, J=1.6 Hz, 1H), 7.73, 7.72 (dd, J=0.4 Hz, 1H), 7.34-7.33 (m, 1H), 6.94 (br. s, 1H), 3.06 (s, 3H). LCMS (ESI): calc. for C₈H₇BrF₃NO₂S [M−H]⁻ 315.93, found 315.99.

### Preparation 37. N-(2-(4,4,5,5-Tetramethyl-1,3,2-dioxaborolan-2-yl)-5-(trifluoromethyl)phenyl) methane sulfonamide (P37)

To a stirred solution of P36 (5.0 g, 15.718 mmol, 1 eq), bis(pinacolato)diboron (5.98 g, 23.577 mmol, 1.5 eq) in 1,4-dioxane (75 mL) was added KOAc (4.628 g, 47.154 mmol, 3.0 eq) at ambient temperature. The reaction mixture was degassed with a stream of nitrogen for 15 min, then added PdCl₂ (dppf)·DCM (0.552 g, 0.786 mmol, 0.05 eq) at rt. The resulting reaction mixture was stirred for 6 h at 100° C. The progress of reaction was monitored by TLC and LCMS. After completion of starting material, reaction mixture filtered through a celite pad and the celite pad was washed with EtOAc (50 mL). The filtrate was concentrated under reduced pressure at 40° C. The residue was subjected to flash chromatography over silica gel (230-400 mesh) using mixture of EtOAc (0→10%) and hexanes as an eluent to afford 5.0 g (crude) of the title compound P37 as a brown solid. Crude itself proceeded for next step.

Analytical data: LCMS (ESI): calc, for corresponding boronic acid C₈H₉BF₃NO₄S [M−H]⁻ 282.03, found 282.01.

### Synthesis of (R)-4-Chloro-N-(1-ethylpiperidin-3-yl)-5,6,7,8-tetrahydro-5,8-ethanophthalazin-1-amine (P38)

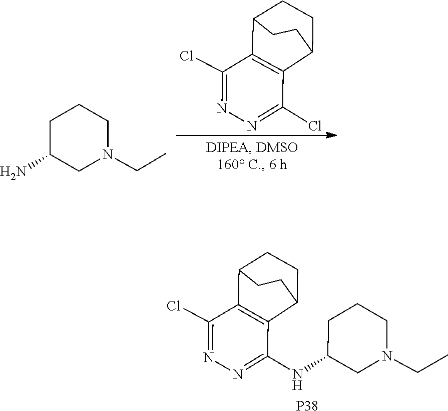

### Preparation 38. (R)-4-Chloro-N-(1-ethylpiperidin-3-yl)-5,6,7,8-tetrahydro-5,8-ethanophthalazin-1-amine (P38)

To a stirred solution of 1,4-dichloro-5,6,7,8-tetrahydro-5,8-ethanophthalazine (0.6 g, 2.619 mmol, 1.0 eq) in DMSO (10.0 mL) was added N,N-diisopropylethylamine (2.346 mL, 13.094 mmol, 5.0 eq) and (R)-1-ethylpiperidin-3-amine (1.0 g, 7.86 mmol, 3.0 eq) at ambient temperature and stirred for 6 h at 160° C. The progress of the reaction was monitored by TLC and LCMS. After completion of the starting material, all volatiles were removed in vacuo. The residue was purified by reverse phase column chromatography using ACN and 0.1% FA in water to afford 0.4 g (47%) of the title compound P38 as a brown liquid.

Analytical data: LCMS (ESI): calc. for: C₁₇H₂₅ClN₄ [M+H]⁺ 321.18, found 321.34.

### Synthesis of N-(2-(4,4,5,5-tetramethyl-1,3,2-dioxaborolan-2-yl)-5-(trifluoromethyl)phenyl) ethane sulfonamide (P40)

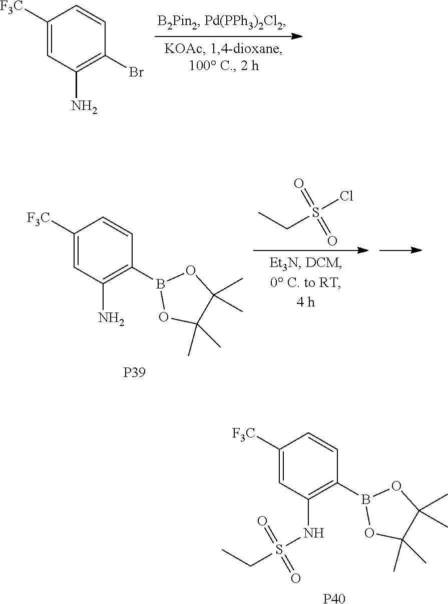

### Preparation 39. 2-(4,4,5,5-Tetramethyl-1,3,2-dioxaborolan-2-yl)-5-(trifluoromethyl) aniline (P39)

To a stirred solution of 2-bromo-5-(trifluoromethyl) aniline (5 g, 20.83 mmol, 1.0 eq), bis(pinacolato)diboron (7.93 g, 31.24 mmol, 1.5 eq) in 1,4-dioxane (150 mL) was added KOAc (6.1 g, 62.49 mmol, 3.0 eq) at ambient temperature. The mixture was degassed with a stream of argon for 10 min. Thereafter, Pd(PPh₃)₂Cl₂ (0.73 g, 1.04 mmol, 0.05 eq) was added. The resulting reaction mixture was stirred for 2 h at 100° C. The progress of reaction was monitored by TLC and LCMS. The reaction mixture was filtered through celite pad, the pad was washed with 1,4-dioxane (50 mL). The filtrate was concentrated completely under reduced pressure to give residue. The residue was subjected to flash chromatography over silica gel (80 g, 230-400 mesh) using mixture of EtOAc (0→20%) and hexanes as an eluent to afford 4.1 g (69%) of the title compound P39 as an off-white solid.

Analytical data: ¹H NMR (400 MHz, CDCl₃), δ: 7.68 (d, J=7.6 Hz, 1H), 6.86 (dd, J=7.6 Hz, 0.8 Hz, 1H), 6.79 (d, J=0.8 Hz, 1H), 4.91 (br. s, 2H), 1.34 (s, 12H); LCMS (ESI): calc. for: C₁₃H₁₈BF₃NO₂ [M+H]⁺ 288.13, found 288.27.

### Preparation 40. N-(2-(4,4,5,5-Tetramethyl-1,3,2-dioxaborolan-2-yl)-5-(trifluoromethyl)phenyl) ethane sulfonamide (P40)

To a stirred solution of P39 (1.0 g, 3.48 mmol, 1.0 eq) in DCM (8 mL) was added TEA (2.1 g, 20.90 mmol, 6.0 eq) followed by ethanesulfonyl chloride (0.67 g, 5.22 mmol, 1.5 eq) at 0° C. The resulting reaction mixture was stirred for 4 h at ambient temperature. The progress of reaction was monitored by TLC and LCMS. The reaction mixture was washed with water (2×10 mL), brine (20 mL). The organic layer was dried over anh. Na₂SO₄, filtered and concentrated under reduced pressure at 40° C. to give residue. The residue was subjected to flash chromatography over silica gel (40 g, 230-400 mesh) using a mixture of EtOAc (0→30%) and hexanes as an eluent to afford 0.8 g (60%) of the title compound P40 as a pale brown gummy compound.

Analytical data: ¹H NMR (400 MHz, CDCl₃), δ: 8.63 (s, 1H), 7.89-7.88 (m, 2H), 7.33-7.31 (m, 1H), 3.14 (q, J=7.2 Hz, 2H), 1.38-1.32 (m, 15H); LCMS (ESI): calc. for: C₁₅H₂₂BF₃NO₄S [M+H]⁺ 380.12, found 380.48.

### Synthesis of N-(2-(4,4,5,5-tetramethyl-1,3,2-dioxaborolan-2-yl)-5-(trifluoromethyl)phenyl) propane-2-sulfonamide (P43)

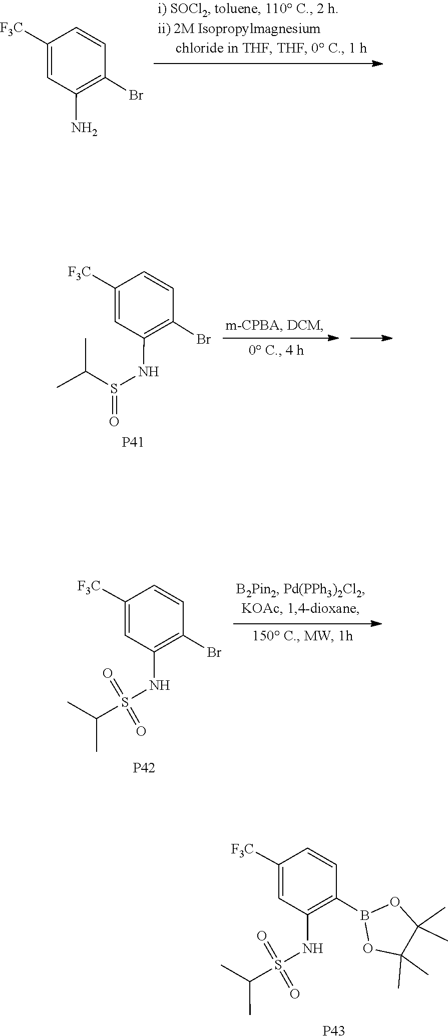

### Preparation 41. N-(2-Bromo-5-(trifluoromethyl)phenyl) propane-2-sulfinamide (P41)

To a stirred solution of 2-bromo-5-(trifluoromethyl) aniline (2.0 g, 8.33 mmol, 1.0 eq) in toluene (40.0 mL) was added SOCl₂ (0.8 mL, 10.83 mmol, 1.3 eq) at ambient temperature. The resulting reaction mixture was stirred for 2 h at 110° C. The progress of reaction was monitored by TLC. The reaction mixture was concentrated under reduced pressure at 45° C. to give residue. The residue was dissolved in dry THF (40.0 mL) and cooled to 0° C. Thereafter, 2M Isopropyl magnesium chloride in THF (6.24 mL, 12.49 mmol, 1.5 eq) was added at 0° C. The resulting reaction mixture was stirred for 1 h at 0° C. The progress of reaction was monitored by TLC and LCMS. The reaction mixture was quenched with NH₄Cl solution (40 mL) and extracted with EtOAc (2×20 mL). The combined organic layer was washed with brine (20 mL), dried over anh. Na₂SO₄, filtered and concentrated under reduced pressure at 45° C. to give residue. The residue was subjected to flash chromatography over silica (40 g, 230-400 mesh) using mixture of EtOAc (0→10%) in hexanes as an eluent to afford 2.1 g (76%) of the title compound P41 as a brown gummy compound.

Analytical data: ¹H NMR (400 MHz, CDCl₃), δ: 6.86 (dd, J=7.64 Hz, 0.8 Hz, 1H), 7.55 (d, J=1.6 Hz, 1H), 7.15-7.13 (m, 1H), 6.41 (s, 1H), 3.11-3.08 (m, 1H), 1.43-1.41 (m, 6H); LCMS (ESI): calc. for: C₁₀H₁₁BrF₃NOS [M+H]⁺ 329.97, found 330.14.

### Preparation 42. N-(2-Bromo-5-(trifluoromethyl)phenyl) propane-2-sulfonamide (P42)

To a stirred solution of P41 (1.6 g, 4.84 mmol, 1.0 eq) in DCM (48.0 mL) was added m-CPBA (65% in moisture) (1.92 g, 7.26 mmol, 1.5 eq) at 0° C. The resulting reaction mixture was stirred for 4 h at 0° C. The progress of reaction was monitored by TLC and LCMS. The reaction mixture was washed with sat. NaHCO₃ solution (2×60 mL), brine (30 mL). The organic layer was dried over anh. Na₂SO₄, filtered, and concentrated under reduced pressure at 45° C. to give residue. The residue was subjected to flash chromatography over silica gel (230-400 mesh) using mixture of EtOAc (0→20%) and hexanes as an eluent to afford 1.25 g (75%) of the title compound P42 as an off white solid.

Analytical data: ¹H NMR (400 MHz, CDCl₃), δ: 8.00 (d, J=1.6 Hz, 1H), 7.69 (d, J=8.4 Hz, 1H), 7.25 (dd, J=8.4 Hz, 1.6 Hz, 1H), 6.82 (s, 1H), 3.31 (sep, J=6.8 Hz, 1H), 1.42 (d, J=6.8 Hz, 1H); LCMS (ESI): calc. for: C₁₀H₁₁BrF₃NO₂S [M+H]⁺ 345.96, found 346.28.

### Preparation 43. N-(2-(4,4,5,5-Tetramethyl-1,3,2-dioxaborolan-2-yl)-5-(trifluoromethyl)phenyl) propane-2-sulfonamide (P43)

To a stirred solution of P42 (600 mg, 1.73 mmol, 1.0 eq), bis(pinacolato)diboron (660 mg, 2.60 mmol, 1.5 eq) in 1,4-dioxane (12 mL) was added KOAc (510 mg, 5.20 mmol, 3.0 eq) at ambient temperature in a microwave vial. The mixture was degassed with a stream of argon for 10 min. Thereafter, Pd(PPh₃)₂Cl₂ (61 mg, 0.087 mmol, 0.05 eq) was added.

The mixture was stirred for 1 h at 150° C. in microwave synthesizer. The reaction mixture was filtered through celite pad, the pad was washed with 1,4-dioxane (10 mL). The filtrate was concentrated under reduced pressure at 45° C. to afford of the title compound P43 (850 mg, crude) as a brown gummy compound.

Analytical data: LCMS (ESI): calc. for: C₁₆H₂₃BF₃NO₄S [M−H]⁻ 392.14, found 392.26.

### Synthesis of (R)-6-(2-amino-4-(trifluoromethyl)phenyl)-N-(1-ethylpiperidin-3-yl)-4-methylpyridazin-3-amine (P44)

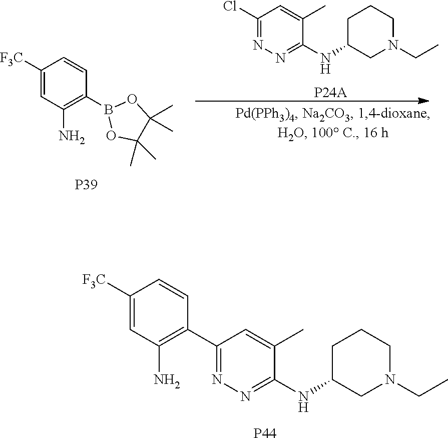

### Preparation 44. (R)-6-(2-Amino-4-(trifluoromethyl)phenyl)-N-(1-ethylpiperidin-3-yl)-4-methylpyridazin-3-amine (P44)

To a stirred solution of P24A (300 mg, 1.17 mmol, 1.0 eq), P39 (1.0 g, 3.53 mmol, 3.0 eq) in mixture of 1,4-dioxane (24.0 mL) and H₂O (6.0 mL) was added Na₂CO₃ (374 mg, 3.53 mmol, 3.0 eq) at ambient temperature. The reaction mixture was degassed with a stream of nitrogen for 5 min. Thereafter, Pd(PPh₃)₄ (68 mg, 0.059 mmol, 0.05 eq) was added. The reaction mixture was stirred for 16 h at 100° C. The progress of reaction was monitored by TLC and LCMS. The reaction mixture was filtered through celite pad, the pad was washed with 1,4-dioxane (10 mL). The filtrate was concentrated under reduced pressure to give residue. The residue was subjected to flash chromatography over silica gel (40 g, 230-400 mesh) using a mixture of MeOH (0→20%) and DCM as an eluent to afford 360 mg (81%) of the title compound P44 as a brown gummy compound.

Analytical data: LCMS (ESI): calc. for: C₁₉H₂₄F₃N₅ [M+H]⁺ 380.20, found 380.41.

### Synthesis of tert-butyl (R)-3-((4-methyl-6-(2-(methylsulfonamido)-4-(trifluoromethyl)phenyl) pyridazin-3-yl)amino)piperidine-1-carboxylate (P45)

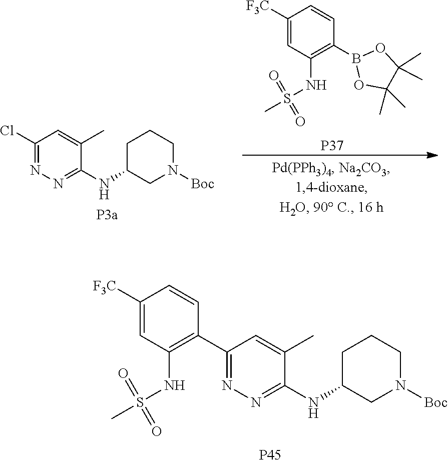

### Preparation 45. tert-Butyl (R)-3-((4-methyl-6-(2-(methylsulfonamido)-4-(trifluoromethyl)phenyl) pyridazin-3-yl)amino)piperidine-1-carboxylate (P45)

To a stirred solution of P37 (1.14 g, 3.122 mmol, 1 eq) in dioxane/water (20 mL, 9:1) was degassed with argon for 5 min. Thereafter, P3a (1.02 g, 3.122 mmol, 1 eq), sodium carbonate (0.66 g, 6.24 mmol, 2.0 eq) and Pd(PPh₃)₄ (0.18 g, 0.156 mmol, 0.05 eq) were added under argon atmosphere. Resulting reaction mixture was stirred for 16 h at 90° C. Progress of the reaction was monitored by TLC and LCMS. Reaction mixture was filtered through celite pad, washed with dioxane (20 mL). Filtrate was concentrated under reduced pressure. The crude compound was subjected to flash column chromatography using a mixture of EtOAc (0→100%) and hexane as an eluent to afford 0.44 g (26%) of the title compound as a pale brown gummy liquid.

Analytical data: LCMS (ESI): calc, for C₂₃H₃₀F₃N₅O₄S [M+H]⁺ 530.20, found 530.41.

### Synthesis of N-(2-(6-chloro-4-methylpyridazin-3-yl)-5-(trifluoromethyl)phenyl) methanesulfonamide (P46a) and N-(2-(6-chloro-5-methylpyridazin-3-yl)-5-(trifluoromethyl)phenyl) methanesulfonamide (P46b)

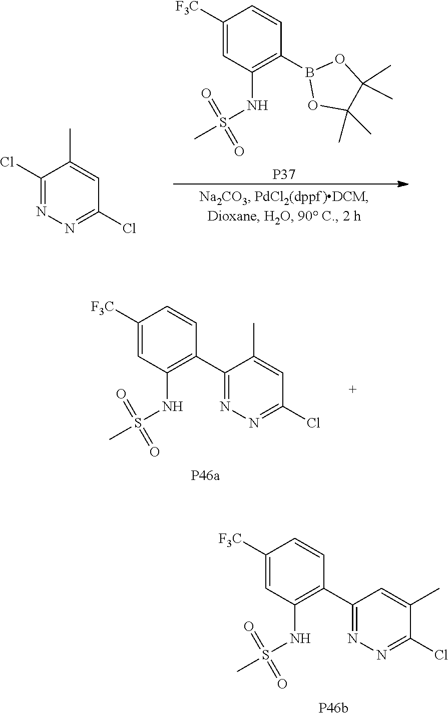

Preparation 46. N-(2-(6-chloro-4-methylpyridazin-3-yl)-5-(trifluoromethyl)phenyl)methanesulfonamide (P46a) and N-(2-(6-chloro-5-methylpyridazin-3-yl)-5-(trifluoromethyl)phenyl) methanesulfonamide (P46b).

To a stirred solution of 3,6-dichloro-4-methylpyridazine (200 mg, 1.22 mmol, 1.0 eq), N-(2-(4,4,5,5-tetramethyl-1,3,2-dioxaborolan-2-yl)-5-(trifluoromethyl)phenyl) methane-sulfonamide (448 mg, 1.22 mmol, 1.0 eq) in 1,4-dioxane (8.0 mL), water (2.0 mL) was added Na₂CO₃ (390 mg, 3.68 mmol, 3.0 eq) at ambient temperature. The reaction mixture was degassed with a stream of nitrogen for 15 min. Thereafter, PdCl₂ (dppf)·DCM (50 mg, 0.06 mmol, 0.05 eq) was added at this temperature. The resulting reaction mixture was stirred for 2 h at 90° C. The progress of reaction was monitored by TLC and LCMS. The reaction mixture was filtered through a celite pad and the pad was washed with 10% MeOH in DCM (30 mL). The filtrate was washed with water (20 mL), brine (15 mL), dried over anh. Na₂SO₄, filtered and concentrated under reduced pressure at 45° C. The residue was subjected to revers phase C18 (12 g) flash chromatography using mixture of 0.1% FA in H₂O (0→70%) and ACN as an eluent to afford 60 mg (13%) of the title compound P46a as a light pink solid and 50 mg (11%) of the title compound P46b as a light brown solid.

### Structure Confirmation of P46a:

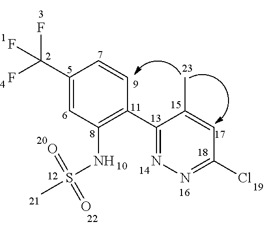 [SMILES: Cc1cc(Cl)nnc1-c1ccc(C(F)(F)F)cc1NS(C)(=O)=O]

In one experiment, on irradiation of H23 proton at 2.16 ppm showing enhancement for H17 proton at 8.00 ppm and H9 proton at 7.65 ppm.

Analytical data: ¹H NMR (400 MHz, DMSO-d₆), δ: 9.47 (s, 1H), 7.99 (s, 1H), 7.87 (s, 1H), 7.69 (d, J=8.0 Hz, 1H), 7.64 (d, J=8.0 Hz, 1H), 3.02 (s, 3H), 2.15 (s, 3H); LCMS (ESI): calc, for C₁₃H₁₁ClF₃N₃O₂S [M+H]⁺ 366.02, found 366.22.

### Structure Confirmation of P46b:

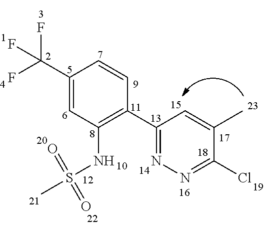 [SMILES: Cc1cc(-c2ccc(C(F)(F)F)cc2NS(C)(=O)=O)nnc1Cl]

In one experiment, on irradiation of H23 proton at 2.47 ppm showing enhancement for H15 proton at 8.23 ppm

Analytical data: ¹H NMR (400 MHz, DMSO-d₆), δ: 10.41 (s, 1H), 8.23 (s, 1H), 7.97 (d, J=8.0 Hz, 1H), 7.90 (s, 1H), 7.72 (d, J=7.6 Hz, 1H), 3.10 (s, 3H), 2.47 (s, 3H); LCMS (ESI): calc, for C₁₃H₁₁ClF₃N₃O₂S [M+H]⁺ 366.02, found 366.22.

### Synthesis of (1R,9aR)-octahydro-2H-quinolizin-1-amine hydrochloride (P49)

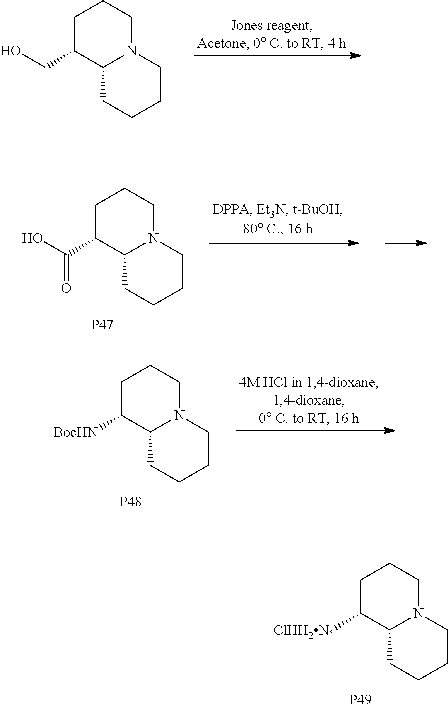

### Preparation 47. (1R,9aR)-Octahydro-2H-quinolizine-1-carboxylic acid (P47)

To a stirred solution of ((1R,9aR)-octahydro-2H-quinolizin-1-yl) methanol (5.0 g, 29.53 mmol, 1.0 eq) in acetone (150 mL) was added Jones reagent (2M CrO₃ in aq. H₂SO₄) (44.308 mL, 88.61 mmol, 3.0 eq) at 0° C. The resulting reaction mixture was stirred at ambient temperature for 4 h. The progress of reaction was monitored by TLC & LCMS. After completion of the reaction, the reaction mixture was diluted with DCM (80 mL), followed by silica gel (100-200 mesh, 15 g), IPA (30 mL) and NH₄OH (20 mL) was added, stirred vigorously for 20 min. The reaction mixture was filtered through a celite pad and the pad was washed with 5% NH₄OH in MeOH (200 mL). The filtrate was evaporated under reduced pressure at 45° C. The residue was subjected to flash chromatography over silica gel (120 g, 230-400 mesh) using mixture of 1% NH₄OH in MeOH (0→20%) and DCM as an eluent to afford 2.3 g (42%) of the title compound P47 as a brown gummy solid.

Analytical data: ¹H NMR (400 MHz, CD₃OD), δ: 3.42-3.34 (m, 2H), 3.06-3.02 (m, 1H), 2.95-2.88 (m, 1H), 2.84-2.77 (m, 1H), 2.58 (br. s, 1H), 2.12-2.04 (m, 2H), 1.97-1.93 (m, 1H), 1.91-1.71 (m, 7H), 1.57-1.53 (m, 1H); LCMS (ELSD) (ESI): calc, for C₁₀H₁₇NO₂ [M+H]⁺ 184.13, found 184.34.

### Preparation 48. tert-Butyl ((1R,9aR)-octahydro-2H-quinolizin-1-yl) carbamate (P48)

To a stirred solution of P47 (2.0 g, 10.91 mmol, 1.0 eq) in t-BuOH (100.0 mL) was added DPPA (4.69 mL, 21.83 mmol, 2.0 eq) followed by Et₃N (4.6 mL, 32.74 mmol, 3.0 eq) at ambient temperature. The mixture was heated to 80° C., stirred for 16 h at 80° C. The progress of reaction was monitored by TLC & LCMS. After completion of the reaction, the reaction mixture was evaporated under reduced pressure at 45° C. The residue was subjected to flash chromatography over silica gel (80 g, 230-400 mesh) using mixture of MeOH (0→20%) and DCM as an eluent to afford 3.0 g (crude) of the title compound 3 as a yellow oil.

Analytical data: LCMS (ELSD) (ESI): calc, for C₁₄H₂₆N₂O₂ [M+H]⁺ 255.20, found 255.43.

### Preparation 49. (1R,9aR)-octahydro-2H-quinolizin-1-amine hydrochloride (P49)

To a stirred solution of P48 (3.0 g, 11.79 mmol, 1.0 eq) in 1,4-dioxane (15.0 mL) was added 4M HCl in 1,4-dioxane (15.0 mL) at 0° C. The resulting reaction mixture was stirred at ambient temperature for 16 h. The progress of reaction was monitored by TLC & LCMS. After completion of the reaction, the reaction mixture was concentrated under reduced pressure to get residue. The residue was triturated with EtOAc (10 mL) and dried under reduced pressure to afford 500 mg (crude) of the title compound P49 as an off-white solid. The crude compound was carried to next step without further purification.

Analytical data: LCMS (ESI): calc, for C₉H₁₈N₂ [M+H]⁺ 155.15, found 155.24.

### Synthesis of (2-(Cyclopropanesulfonamido)-4-(trifluoromethyl)phenyl) boronic acid (P51)

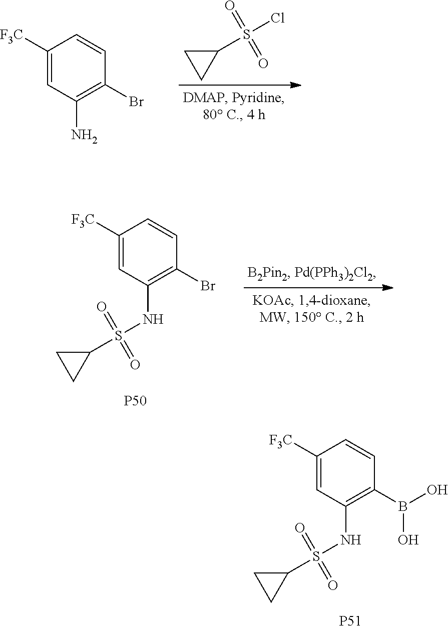 [SMILES: Nc1cc(C(F)(F)F)ccc1Br ; O=S(=O)(Cl)C1CC1 ; "P50" ; O=S(=O)(Nc1cc(C(F)(F)F)ccc1Br)C1CC1 ; "P51" ; O=S(=O)(Nc1cc(C(F)(F)F)ccc1B(O)O)C1CC1]

### Preparation 50. N-(2-Bromo-5-(trifluoromethyl)phenyl) cyclopropanesulfonamide (P50)

To a stirred solution of 2-bromo-5-(trifluoromethyl) aniline (2.0 g, 8.33 mmol, 1.0 eq) in pyridine (5 mL) were added 4-dimethylaminopyridine (204 mg, 1.67 mmol, 0.2 eq) followed by cyclopropanesulfonyl chloride (1.5 mL, 16.66 mmol, 2.0 eq) at ambient temperature. The resulting reaction mixture was stirred for 4 h at 80° C. The progress of reaction was monitored by TLC and LCMS. The reaction mixture was diluted with H₂O (30 mL) and extracted with EtOAc (2×40 mL). The combined organic layer was washed with H₂O (40 mL), brine (20 mL), dried over anh. Na₂SO₄, filtered and concentrated under reduced pressure at 45° C. to give residue. The residue was subjected to flash chromatography over silica gel (230-400 mesh) using mixture of EtOAc (0→20%) and hexanes as an eluent to afford 1.8 g (62%) of the title compound P50 as a yellow solid.

Analytical data: ¹H NMR (400 MHz, CDCl₃), δ: 7.96 (d, J=2.0 Hz, 1H), 7.72 (d, J=8.4 Hz, 1H), 7.32-7.26 (m, 1H), 6.87 (br. s, 1H), 2.53-2.47 (m, 1H), 1.26-1.21 (m, 2H), 1.08-1.04 (m, 2H); LCMS (ESI): calc. for: C₁₀H₉BrF₃NO₂S [M−H]⁺ 341.95, found 342.21.

### Preparation 51. (2-(Cyclopropanesulfonamido)-4-(trifluoromethyl)phenyl) boronic acid (P51)

To a stirred solution of P50 (500 mg, 1.45 mmol, 1.0 eq), bis(pinacolato)diboron (1.8 g, 7.26 mmol, 5.0 eq) in 1,4-dioxane (10.0 mL) was added KOAc (285 mg, 2.90 mmol, 2.0 eq) at ambient temperature. The resulting reaction mixture was degassed under stream of argon for 10 min. Thereafter, Pd(PPh₃)₂Cl₂ (51 mg, 0.07 mmol, 0.05 eq) was added. The resulting reaction mixture was irradiated under microwave at 150° C. for 2 h. The progress of reaction was monitored by TLC and LCMS. The reaction mixture was filtered through celite pad, and the pad was washed with 10% MeOH:DCM (20 mL). The filtrate was evaporated under reduced pressure at 45° C. to afford 1.2 g (crude) of the title compound P51 as a pale brown gummy compound. The crude residue was used for next step without further purification.

Analytical data: LCMS (ESI): calc, for C₁₀H₁₁BF₃NO₄S [M−H]⁺ 308.05, found 308.04.

### Synthesis of (3R,5R)-1-ethyl-5-fluoropiperidin-3-amine (P53)

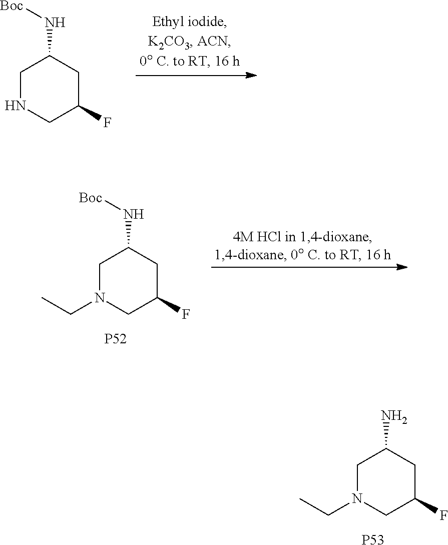

### Preparation 52. tert-Butyl ((3R,5R)-1-ethyl-5-fluoropiperidin-3-yl) carbamate (P52)

To a stirred solution of tert-butyl ((3R,5R)-5-fluoropiperidin-3-yl) carbamate (780 mg, 3.57 mmol, 1.0 eq) in ACN (10 mL) were added K₂CO₃ (740 mg, 5.36 mmol, 1.5 eq) followed by ethyl iodide (0.32 mL, 3.93 mmol, 1.1 eq) at ambient temperature. The resulting reaction mixture was stirred for 16 h at ambient temperature. The progress of reaction was monitored by TLC & LCMS. After completion of the reaction, the reaction mixture was filtered and washed with DCM (80 mL). The filtrate was evaporated under reduced pressure at 45° C. The residue was subjected to flash chromatography over silica gel (40 g, 230-400 mesh) using mixture of MeOH (0→10%) and DCM as an eluent to afford 600 mg (68%) of the title compound P52 as an off-white solid.

Analytical data: ¹H NMR (400 MHz, CDCl₃), δ: 4.89-4.69 (m, 2H), 3.98 (br. s, 1H), 2.86 (br. s, 1H), 2.48-2.45 (m, 4H), 2.32 (br. s, 1H), 2.06 (br. s, 1H), 1.74 (br. s, 1H), 1.45 (s, 9H), 1.06 (t, J=7.2 Hz, 3H); LCMS (ESI): calc, for C₁₂H₂₃FN₂O₂ [M+H]⁺ 247.17, found 247.42.

### Preparation 53. (3R,5R)-1-Ethyl-5-fluoropiperidin-3-amine (P53)

To a stirred solution of P52 (600 mg, 2.43 mmol, 1.0 eq) in 1,4-dioxane (5.0 mL) was added 4M HCl in 1,4-dioxane (5.0 mL) at 0° C. The resulting reaction mixture was stirred for 6 h at ambient temperature. The progress of reaction was monitored by TLC & LCMS. After completion of the reaction, the reaction mixture was concentrated under reduced pressure to give residue. The residue was diluted with water (10 mL), then neutralized (pH=7) with sat. NaHCO₃ solution and extracted with 10% MeOH in DCM (2× 50 mL). The combined organic layer was washed with brine (10 mL), dried over anh. Na₂SO₄, filtered, evaporated under reduced pressure to afford 280 mg (78%) of the title compound P53 as a pale-yellow gummy compound.

Analytical data: ¹H NMR (400 MHz, DMSO-d₆), δ: 4.81 (d, J=54.4 Hz, 3H), 3.92-3.86 (m, 1H), 2.79-2.71 (m, 2H), 2.37-2.28 (m, 2H), 2.09 (dd, J=35.2, 12.4 Hz, 1H), 1.91 (t, J=11.6 Hz, 1H), 1.66 (t, J=9.6 Hz, 1H), 1.48 (br. s, exch. with D₂O, 2H), 1.34-1.30 (m, 1H), 1.28-1.23 (m, 1H), 0.99 (t, J=7.2 Hz, 3H); LCMS (ESI): calc, for C₇H₁₅FN₂ [M+H]⁺ 147.12, found 147.36.

### Synthesis of (R)-6-Chloro-5-cyclopropyl-N-(1-ethylpiperidin-3-yl)pyridazin-3-amine (P57A) and (R)-6-chloro-4-cyclopropyl-N-(1-ethylpiperidin-3-yl)pyridazin-3-amine (P57B)

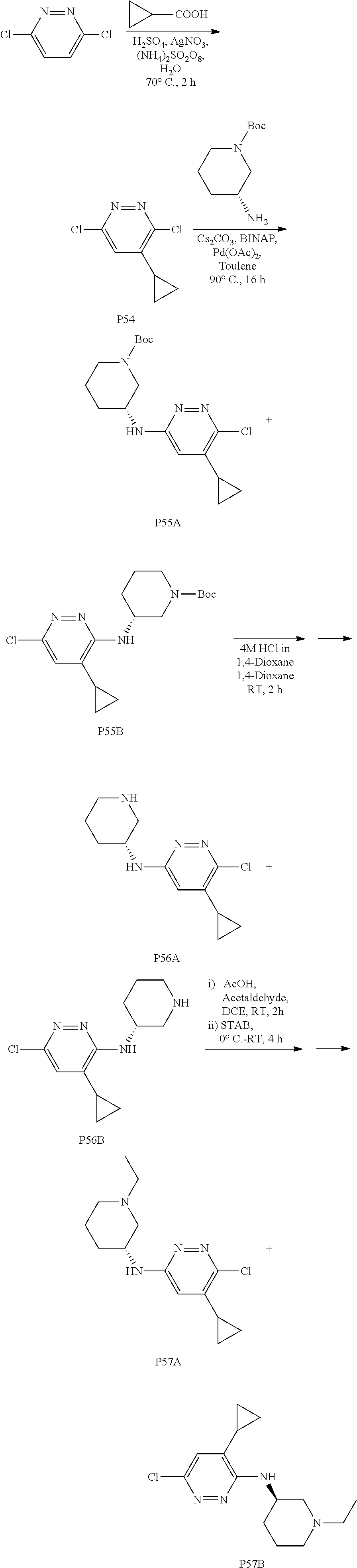

### Preparation 54. 3,6-Dichloro-4-cyclopropylpyridazine (P54)

To a stirred solution of 3,6-dichloropyridazine (10.0 g, 67.128 mmol, 1.0 eq) in H₂O (200 mL), was added H₂SO₄ (10 mL) followed by cyclopropanecarboxylic acid (6.935 g, 80.553 mmol, 1.2 eq) at ambient temperature. The reaction mixture was then heated to 70° C. and added a solution of AgNO₃ (11.345 g, 67.128 mmol, 1.0 eq) in H₂O (10.0 mL) dropwise over a period of 10 min at 70° C. To the reaction mixture was then added solution of (NH₄)₂S₂O₈ (87.266 g, 671.276 mmol, 10 eq) in H₂O (150.0 mL). The resulting reaction mixture was stirred for 2 h at 70° C. The progress of the reaction was monitored by TLC and LCMS. After completion of reaction, the reaction mixture was cooled to ambient temperature, added NH₄OH solution to adjust the pH~9, extracted with EtOAc (2×100 mL). The combined organic layer was washed with brine, dried over anh. Na₂SO₄, and concentrated under reduced pressure. The residue was subjected to flash column chromatography using mixture of EtOAc (10%→15%) and hexane as an eluent to afford 2.8 g (22.07%) of the title compound P54 as a brown liquid.

Analytical data: LCMS (ESI): calc. for: C₇H₆Cl₂N₂ [M+H]⁺ 188.99, found 188.88.

### Preparation 55. tert-Butyl (R)-3-((6-chloro-5-cyclopropylpyridazin-3-yl)amino)piperidine-1-carboxylate (P55A) and tert-butyl (R)-3-((6-chloro-4-cyclopropylpyridazin-3-yl)amino)piperidine-1-carboxylate (P55B)

To a stirred solution of P54 (1.0 g, 5.290 mmol, 1.0 eq), tert-butyl (R)-3-aminopiperidine-1-carboxylate (1.589 g, 7.935 mmol, 1.5 eq) in toluene (30 mL) at ambient temperature was added Cs₂CO₃ (4.020 g, 10.580 mmol, 2.0 eq). The reaction mixture was purged with a stream of N₂ for 10 min, followed by the addition of BINAP (0.658 g, 1.058 mmol, 0.2 eq) and Pd(OAc)₂ (0.178 g, 0.264 mmol, 0.05 eq) at ambient temperature. The reaction was then heated to 90° C. and stirred for 16 h. The progress of the reaction was monitored by TLC and LCMS. After completion of reaction, the reaction mixture was filtered through a pad of celite, the celite pad was washed with EtOAc (2×10 mL) and concentrated under reduced pressure. The residue was subjected to flash column chromatography using mixture of MeOH (0→10%) and DCM as an eluent to afford 0.820 g (43.92%) of mixture of title compounds P55A and P55B as a brown solid.

Analytical data: LCMS (ESI): calc. for: C₁₇H₂₅ClN₄O₂ [M+H]⁺ 353.17 found 353.23.

### Preparation 56. (R)-6-Chloro-5-cyclopropyl-N-(piperidin-3-yl)pyridazin-3-amine (P56A) and (R)-6-chloro-4-cyclopropyl-N-(piperidin-3-yl)pyridazin-3-amine (P56B)

To a stirred solution of the mixture P55 (0.800 g, 2.267 mmol, 1.0 eq) in 1,4 dioxane (20 mL) was added 4N—HCl in 1,4-dioxane (10 mL) at 0° C. The reaction was stirred at ambient temperature for 2 h. The progress of the reaction was monitored by TLC and LCMS. After completion of reaction, reaction mixture was concentrated under reduced pressure to afford 0.600 g (91.51%) of mixture of title compounds (P56A) and (P56B) as a brown solid.

Analytical data: LCMS (ESI): calc. for: C₁₂H₁₇ClN₄ [M+H]⁺ 253.11, found 253.07.

### Preparation 57. (R)-6-Chloro-5-cyclopropyl-N-(1-ethylpiperidin-3-yl)pyridazin-3-amine (P57A) and (R)-6-chloro-4-cyclopropyl-N-(1-ethylpiperidin-3-yl)pyridazin-3-amine (P57B)

To a stirred solution of the mixture of P56 (0.600 g, 2.075 mmol, 1.0 eq) in DCE (12.00 mL) was added AA (0.025 g, 0.415 mmol, 0.2 eq) followed by acetaldehyde (0.137 g, 3.112 mmol, 1.5 eq) at ambient temperature and stirred for 2 h. Thereafter, sodium triacetoxyborohydride (0.660 g, 3.112 mmol, 1.5 eq) was added portion wise at 0° C. The reaction mixture was then stirred at ambient temperature for 4 h. The progress of the reaction was monitored by TLC and LCMS. After completion of reaction, the reaction mixture was concentrated under reduced pressure. The crude compound was then quenched with sat. NaHCO₃ solution (20 mL) and extracted with 10% MeOH in DCM (2×50 mL). The combined organic layer was dried over anhydrous Na₂SO₄, and concentrated under reduced pressure. The residue was then subjected to flash column chromatography using mixture MeOH (0→10%) and DCM as an eluent to afford 0.5 g (85.83%) of mixture of title compounds (P57A) and (P57B) as brown liquid.

Analytical data: LCMS (ESI): calc. for: C₁₄H₂₁ClN₄ [M+H]⁺ 281.15, found 281.17.

### Synthesis of (R)-6-chloro-N-(1-isopropylpiperidin-3-yl)-5-methylpyridazin-3-amine (P58)

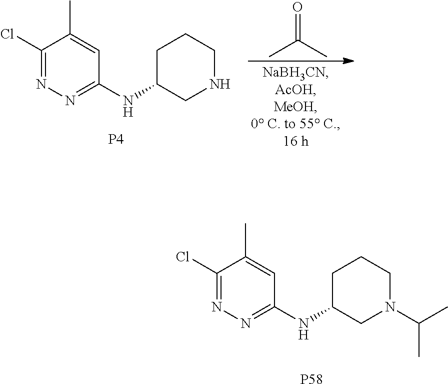

### Preparation 58. (R)-6-chloro-N-(1-isopropylpiperidin-3-yl)-5-methylpyridazin-3-amine (P58)

To a stirred solution of (R)-6-chloro-5-methyl-N-(piperidin-3-yl)pyridazin-3-amine (P4, 5.0 g, 18.9 mmol, 1.0 eq) in MeOH (100.0 mL) was added AA (1.0 mL), acetone (14.15 mL, 190 mmol, 10.0 eq.) at ambient temperature, followed by NaBH₃CN (2.4 g, 37.99 mmol, 2 eq) at 0° C. The resulting reaction mixture was stirred at 55° C. for 16 h. The progress of reaction was monitored by TLC and LCMS. After completion of reaction, the reaction mixture was quenched with sat. NaHCO₃ solution (50 mL) and extracted with 10% MeOH in DCM (2×70 mL). The combined organic layer was dried over anh. Na₂SO₄, filtered and evaporated under reduced pressure to give residue. The residue was subjected to flash chromatography over silica gel (80 g, 230-400 mesh) using a mixture of MeOH (0→20%) in DCM as an eluent to afford 3.5 g (68%) of the title compound P58 as a pale yellow solid.

Analytical data: ¹H NMR (400 MHz, CD₃OD), δ: 6.80 (d, J=1.2 Hz, 1H), 4.85 (s, 3H), 4.13-4.08 (m, 1H), 3.06-2.96 (m, 2H), 2.60-2.54 (m, 1H), 2.44-2.42 (m, 1H), 2.27 (s, 3H), 2.04-2.00 (m, 1H), 1.93-1.88 (m, 1H), 1.78-1.72 (m, 1H), 1.48-1.45 (m, 1H), 1.18-1.12 (m, 6H). LCMS (ESI): calc, for C₁₃H₂₁ClN₄ [M+H]⁺ 269.15, found 269.25.

### Synthesis of (3R,5S)-1-cyclopropyl-5-(trifluoromethyl) piperidin-3-amine hydrochloride

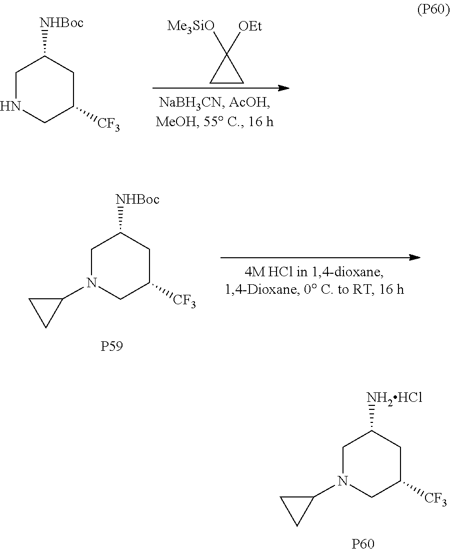

### Preparation 59. tert-Butyl ((3R,5S)-1-cyclopropyl-5-(trifluoromethyl) piperidin-3-yl) carbamate (P59)

To a stirred solution of tert-butyl ((3R,5S)-5-(trifluoromethyl) piperidin-3-yl) carbamate (500 mg, 1.86 mmol, 1.0 eq) in MeOH (15 mL) were added (1-ethoxycyclopropoxy) trimethylsilane (1.89 mL, 9.32 mmol, 5.0 eq), AA (0.5 mL) and sodium cyanoborohydride (234 mg, 3.73 mmol, 2.0 eq) at ambient temperature. The resulting reaction mixture was stirred for 16 h at 55° C. The progress of reaction was monitored by TLC and LCMS. After completion of the reaction, the reaction mixture was diluted with sat. NaHCO₃ solution (20 mL) and extracted with 10% MeOH in DCM (2× 30 mL). The combined organic layer was washed with brine (10 mL), dried over anh. Na₂SO₄, filtered, evaporated under reduced pressure at 45° C. The residue was subjected to flash chromatography over silica gel (40 g, 230-400 mesh) using mixture of MeOH (0→20%) and DCM as an eluent to afford 510 mg (88%) of the title compound P59 as an off-white solid.

Analytical data: ¹H NMR (400 MHz, CDCl₃), δ: 4.36 (br. s, 1H), 3.60 (br. s, 1H), 3.24-3.20 (m, 1H), 3.15-3.12 (m, 1H), 2.38-2.30 (m, 1H), 2.22-2.19 (m, 1H), 2.10 (t, J=11.2 Hz, 1H), 1.86 (t, J=10.8 Hz, 1H), 1.70-1.60 (m, 1H), 1.44 (s, 9H), 1.10 (q, J=12.0 Hz, 1H), 0.47-0.38 (m, 4H); LCMS (ESI): calc, for C₁₄H₂₃F₃N₂O₂ [M+H]⁺ 309.17, found 309.39.

### Preparation 60. (3R,5S)-1-Cyclopropyl-5-(trifluoromethyl) piperidin-3-amine hydrochloride (P60)

To a stirred solution of P59 (500 mg, 1.62 mmol, 1.0 eq) in 1,4-dioxane (5.0 mL) was added 4M HCl in 1,4-dioxane (5.0 mL) at 0° C. The resulting reaction mixture was stirred for 16 h at ambient temperature. The progress of reaction was monitored by TLC and LCMS. After completion of the reaction, the reaction mixture was concentrated under reduced pressure to afford 400 mg (crude) of the title compound P60 as an off-white solid. The crude compound was carried to next step without further purification.

Analytical data: ¹H NMR (400 MHz, CD₃OD), δ: 3.92-3.79 (m, 3H), 3.41-3.21 (m, 3H), 3.03-2.97 (m, 1H), 2.46-2.43 (m, 1H), 1.82 (q, J=12.4 Hz, 1H), 1.30-1.23 (m, 2H), 1.03-0.97 (m, 2H); LCMS (ESI): calc, for C₉H₁₅F₃N₂ [M+H]⁺ 209.12, found 209.31.

### Synthesis of 2-((R)-3-((6-chloro-5-methylpyridazin-3-yl)amino) piperidin-1-yl) propan-1-ol (P61)

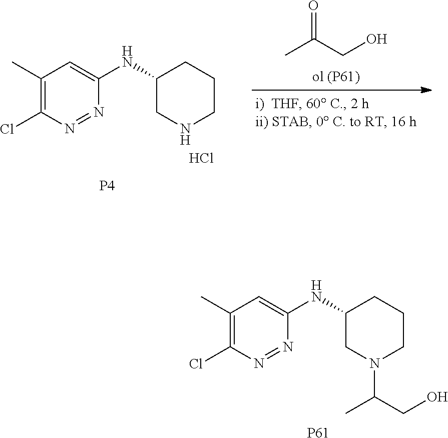

### Preparation 61. 2-((R)-3-((6-Chloro-5-methylpyridazin-3-yl)amino) piperidin-1-yl) propan-1-ol (P61)

To a stirred solution of P4 (1.6 g, 6.08 mmol, 1.0 eq) in THF (32 mL) was added 1-hydroxypropan-2-one (0.90 g, 12.16 mmol, 2.0 eq) at ambient temperature. The resulting reaction mixture was stirred at 60° C. for 2 h. The reaction mixture was cooled to 0° C. and STAB (3.85 g, 18.23 mmol, 3.0 eq) was added at same temperature. The resulting reaction mixture was stirred for 16 h at ambient temperature. The progress of reaction was monitored by TLC and LCMS. After completion of reaction, the reaction mixture was quenched with sat. NaHCO₃ solution and extracted with EtOAc (2×50 mL). The combined organic layer was dried over Na₂SO₄, filtered, and concentrated under reduced pressure at 40° C. to give residue. The residue was subjected to flash chromatography over silica gel (230-400 mesh) using mixture of MeOH (0→20%) and hexanes as an eluent to afford 1.1 g (63%) of the title compound P61 as a pale-yellow gummy liquid.

Analytical data: LCMS (ESI): calc, for C₁₃H₂₁ClN₄O [M+H]⁺ 285.14, found 285.33.

### Synthesis of (R)—N-(2-(4-Methyl-6-(piperidin-3-ylamino)pyridazin-3-yl)-5-(trifluoromethyl)phenyl) methanesulfonamide hydrochloride (P65)

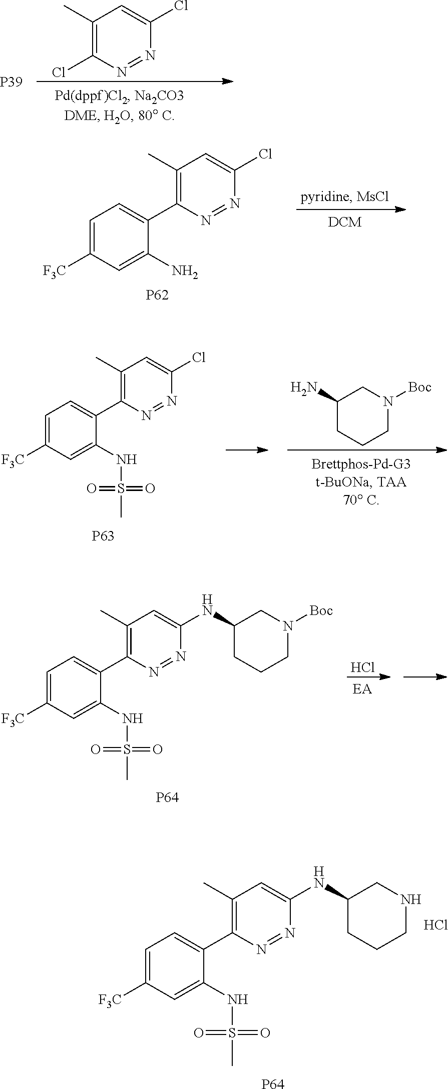

### Preparation 62. 2-(6-Chloro-4-methylpyridazin-3-yl)-5-(trifluoromethyl) aniline (P62)

The solution of P39 (5.5 g, 13.42 mmol), 3,6-dichloro-4-methylpyridazine (2.81 g, 17.24 mmol), sodium carbonate (3.05 g, 28.74 mmol) in 1,2-dimethoxyethane (50 mL) and water (10 ml) was degassed with nitrogen for 10 min and added [1,1′-Bis (diphenylphosphino) ferrocene]dichloropalladium (II) (980 mg, 1.34 mmol) at 25° C. The reaction mixture was stirred at 80° C. for 12 h and then concentrated. The residue was purified by silica gel column chromatography (petroleum ether:EA=50:0 to 20:1) to give the title compound P62 (3.0 g, 90% purity from ¹HNMR, 70% yield) as white solids. LCMS (ESI): RT=1.780 min, mass calc, for Cl₂H₉ClF₃N₃ 287.0, m/z found 288.1 [M+H]⁺. ¹H NMR (400 MHz, CDCl₃) § 7.50-7.49 (m, 1H), 7.21-7.19 (m, 1H), 7.09-7.07 (m, 2H), 2.29 (s, 3H).

### Preparation 63. N-(2-(6-Chloro-4-methylpyridazin-3-yl)-5-(trifluoromethyl)phenyl) methanesulfonamide (P63)

To a solution of P62 (3.0 g, 9.39 mmol) and pyridine (891 mg, 11.26 mmol) in DCM (30 mL) was added methanesulfonyl chloride (1.1 mg, 9.39 mmol) at 0° C. After stirring at 25° C. for 16 h, the reaction mixture diluted with water (30 mL) and extracted with DCM (30 mL) three times. The combined organic layers were washed with brine (30 mL), dried over Na₂SO₄ (S), filtered and concentrated. The residue was purified by silica gel column chromatography (petroleum ether:EA=50:0 to 20:1) to give P63 (1.5 g, 90% purity from ¹H NMR, 39% yield) as yellow oil. LCMS (ESI): R_{T}=1.611 min, mass calc. for C₁₃H₁₁ClF₃N₃O₂S 365.0, m/z found 366.1 [M+H]⁺. ¹H NMR (400 MHz, CDCl₃), δ: 8.09 (s, 1H), 7.96 (s, 1H), 7.54-7.52 (m, 2H), 7.42-7.40 (m, 1H), 3.01 (s, 3H), 2.30 (s, 3H).

### Preparation 64. tert-Butyl (R)-3-((5-methyl-6-(2-(methylsulfonamido)-4-(trifluoromethyl)phenyl) pyridazin-3-yl)amino)piperidine-1-carboxylate (P64)

The solution of P63 (1.5 g, 3.69 mmol), tert-butyl (R)-3-aminopiperidine-1-carboxylate (1.48 g, 7.38 mmol) and sodium tert-butoxide (709 mg, 7.38 mmol) in tert-amyl alcohol (10 mL) was degassed with nitrogen for 10 minutes and added methanesulfonato (2-dicyclohexylphosphino-3,6-dimethoxy-2′,4′,6′-tri-i-propyl-1,1′-biphenyl) (2′-amino-1,1′-biphenyl-2-yl) palladium (359 mg, 0.369 mmol) at 25° C. The reaction mixture was stirred at 70° C. for 12 h under nitrogen atmosphere. The reaction mixture was diluted with water (15 mL) and extracted with EA (15 mL) for three times. The combined organic layers were washed with brine (20 mL), dried over Na₂SO₄ (S), filtered and concentrated. The residue was purified by C18 column (ACN: water=5% to 95%) to give the desired product P64 (1.1 g, 90% purity from ¹H NMR, 51% yield) as yellow solids. LCMS (ESI): R_{T}=1.720 min, mass calc, for C₂₃H₃₀F₃N₅O₄S 529.2, m/z found 530.2 [M+H]⁺. ¹H NMR (400 MHz, CDCl₃), δ: 8.67 (s, 1H), 7.95 (s, 1H), 7.45-7.36 (m, 2H), 6.65 (s, 1H), 3.92 (s, 1H), 3.79-3.76 (m, 1H), 3.53-3.48 (m, 1H), 3.39-3.34 (m, 2H), 3.02 (s, 3H), 2.21 (s, 3H), 2.02-1.98 (m, 2H), 1.80-1.75 (m, 2H), 1.45 (s, 9H)

### Preparation 65. (R)—N-(2-(4-Methyl-6-(piperidin-3-ylamino)pyridazin-3-yl)-5-(trifluoromethyl)phenyl) methanesulfonamide hydrochloride (P65)

To a solution of P64 (1.1 g, 1.87 mmol) in EA (10 mL) was added 4 M hydrochloride in dioxane (10 mL) at 0° C. The reaction mixture was stirred at 25° C. for 3 h. The resulting mixture was concentrated under reduced pressure to give the desired product P65 (891 mg, 92% purity from LCMS, 94% yield) as white solids. LCMS (ESI): R_{T}=1.352 min, mass calc, for C₁₈H₂₃ClF₃N₅O₂S 465.1, m/z found 430.2 [M+H−HCl]⁺.

### Synthesis of 3-Hydroxy-3-methylbutanal (P66)

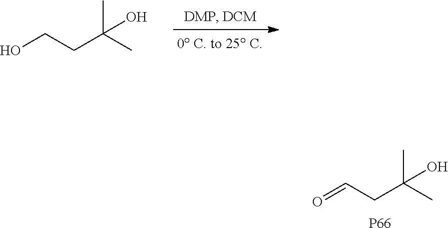

### Preparation 66. 3-Hydroxy-3-methylbutanal (P66)

To a solution of 3-methylbutane-1,3-diol (2.0 g, 19.20 mmol) in DCM (20 mL) was added Dess-Martin periodinane (9.7 g, 23.04 mmol) at 0° C. After stirred at 0° C. for 30 min, the reaction mixture was warmed to 25° C. and stirred for another 1.5 h. The reaction mixture was filtered. The filtrate was concentrated to one-third volume and diluted with diethyl ether (15 mL). Sodium carbonate (10 g) was added to the solution and stirred it at 25° C. for 2 h. The solution was filtered and washed with diethyl ether (30 mL). The filtrate was concentrated to give the target compound (1.9 g) as colorless oil, which was used next step directly without further purification.

### Examples of the Final Compound

In the Table 3 presented certain non-limiting examples of the compound of Formula (A).

TABLE 3
Selected examples of the compound of Formula (A) and LCMS (ESI) data

| | | [MH]⁺ | [MH]⁺ |
|---|---|---|---|
| # | Structure | Calc. | Found |
| 1 | 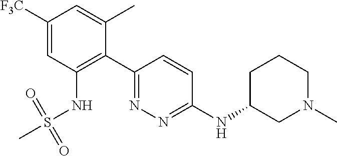 [SMILES: Cc1cc(C(F)(F)F)cc(NS(C)(=O)=O)c1-c1ccc(N[C@@H]2CCCN(C)C2)nn1] | 444.1681 | 444 |
| 2 | 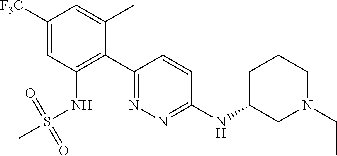 [SMILES: CCN1CCC[C@@H](Nc2ccc(-c3c(C)cc(C(F)(F)F)cc3NS(C)(=O)=O)nn2)C1] | 458.1837 | 458 |
| 3 | 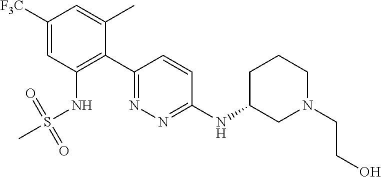 [SMILES: Cc1cc(C(F)(F)F)cc(NS(C)(=O)=O)c1-c1ccc(N[C@@H]2CCCN(CCO)C2)nn1] | 474.1786 | 474 |
| 4 | 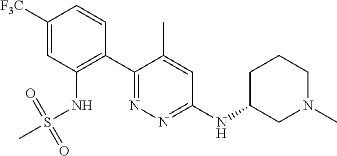 [SMILES: Cc1cc(N[C@@H]2CCCN(C)C2)nnc1-c1ccc(C(F)(F)F)cc1NS(C)(=O)=O] | 444.1681 | 444 |
| 5 | 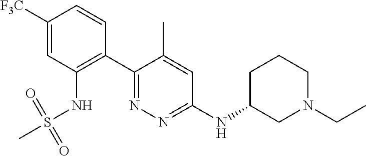 [SMILES: CCN1CCC[C@@H](Nc2cc(C)c(-c3ccc(C(F)(F)F)cc3NS(C)(=O)=O)nn2)C1] | 458.1837 | 458 |
| 6 | 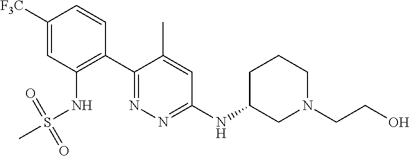 [SMILES: Cc1cc(N[C@@H]2CCCN(CCO)C2)nnc1-c1ccc(C(F)(F)F)cc1NS(C)(=O)=O] | 474.1786 | 474 |
| 7 | 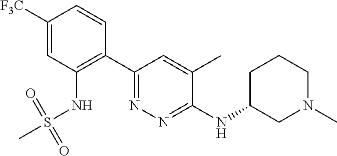 [SMILES: Cc1cc(-c2ccc(C(F)(F)F)cc2NS(C)(=O)=O)nnc1N[C@@H]1CCCN(C)C1] | 444.1681 | 444 |
| 8 | 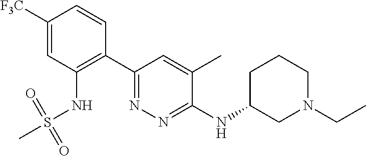 [SMILES: CCN1CCC[C@@H](Nc2nnc(-c3ccc(C(F)(F)F)cc3NS(C)(=O)=O)cc2C)C1] | 458.1837 | 458 |
| 9 | 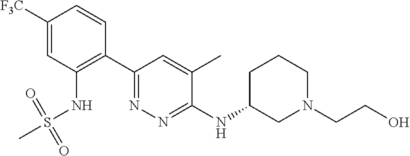 [SMILES: Cc1cc(-c2ccc(C(F)(F)F)cc2NS(C)(=O)=O)nnc1N[C@@H]1CCCN(CCO)C1] | 474.1786 | 474 |
| 10 | 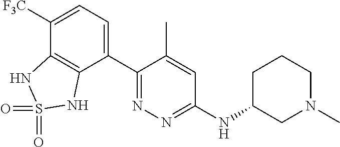 [SMILES: Cc1cc(N[C@@H]2CCCN(C)C2)nnc1-c1ccc(C(F)(F)F)c2c1NS(=O)(=O)N2] | 443.1477 | 443 |
| 11 | 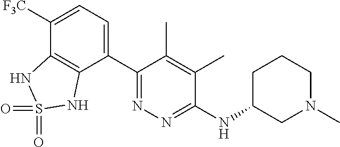 [SMILES: Cc1c(N[C@@H]2CCCN(C)C2)nnc(-c2ccc(C(F)(F)F)c3c2NS(=O)(=O)N3)c1C] | 457.1633 | 457 |
| 12 | 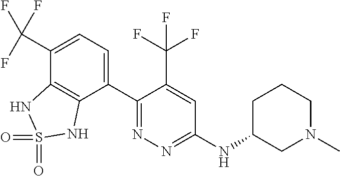 [SMILES: CN1CCC[C@@H](Nc2cc(C(F)(F)F)c(-c3ccc(C(F)(F)F)c4c3NS(=O)(=O)N4)nn2)C1] | 497.1194 | 497 |
| 13 | 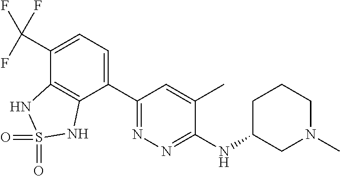 [SMILES: Cc1cc(-c2ccc(C(F)(F)F)c3c2NS(=O)(=O)N3)nnc1N[C@@H]1CCCN(C)C1] | 443.1477 | 443 |
| 14 | 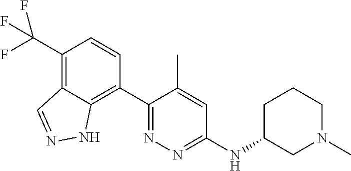 [SMILES: Cc1cc(N[C@@H]2CCCN(C)C2)nnc1-c1ccc(C(F)(F)F)c2cn[nH]c12] | 391.1858 | 391 |
| 15 | 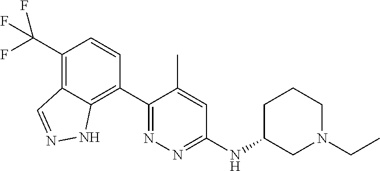 [SMILES: CCN1CCC[C@@H](Nc2cc(C)c(-c3ccc(C(F)(F)F)c4cn[nH]c34)nn2)C1] | 405.2014 | 405 |
| 16 | 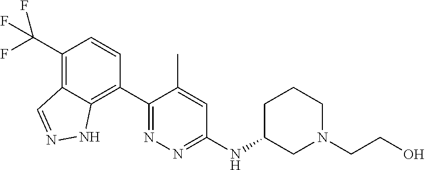 [SMILES: Cc1cc(N[C@@H]2CCCN(CCO)C2)nnc1-c1ccc(C(F)(F)F)c2cn[nH]c12] | 421.1963 | 421 |
| 17 | 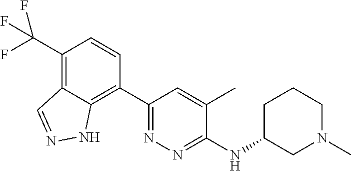 [SMILES: Cc1cc(-c2ccc(C(F)(F)F)c3cn[nH]c23)nnc1N[C@@H]1CCCN(C)C1] | 391.1858 | 391 |
| 18 | 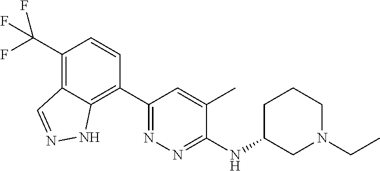 [SMILES: CCN1CCC[C@@H](Nc2nnc(-c3ccc(C(F)(F)F)c4cn[nH]c34)cc2C)C1] | 405.2014 | 405 |
| 19 | 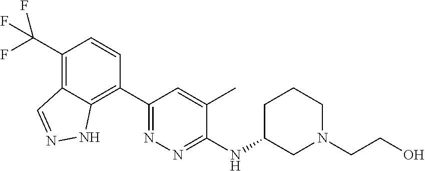 [SMILES: Cc1cc(-c2ccc(C(F)(F)F)c3cn[nH]c23)nnc1N[C@@H]1CCCN(CCO)C1] | 421.1963 | 421 |
| 20 | 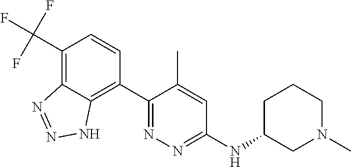 [SMILES: Cc1cc(N[C@@H]2CCCN(C)C2)nnc1-c1ccc(C(F)(F)F)c2nn[nH]c12] | 392.181 | 392 |
| 21 | 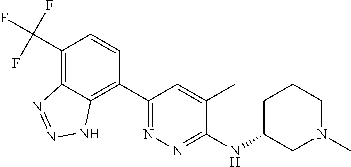 [SMILES: Cc1cc(-c2ccc(C(F)(F)F)c3nn[nH]c23)nnc1N[C@@H]1CCCN(C)C1] | 392.181 | 392 |
| 22 | 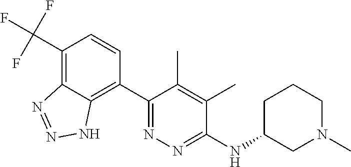 [SMILES: Cc1c(N[C@@H]2CCCN(C)C2)nnc(-c2ccc(C(F)(F)F)c3nn[nH]c23)c1C] | 406.1967 | 406 |
| 23 | 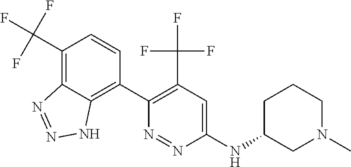 [SMILES: CN1CCC[C@@H](Nc2cc(C(F)(F)F)c(-c3ccc(C(F)(F)F)c4nn[nH]c34)nn2)C1] | 446.1528 | 446 |
| 24 | 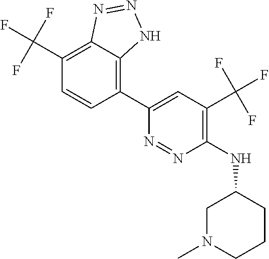 [SMILES: CN1CCC[C@@H](Nc2nnc(-c3ccc(C(F)(F)F)c4nn[nH]c34)cc2C(F)(F)F)C1] | 446.1528 | 446 |
| 25 | 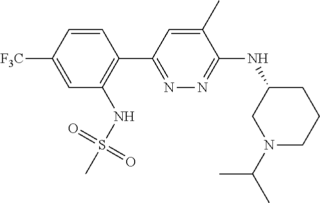 [SMILES: Cc1cc(-c2ccc(C(F)(F)F)cc2NS(C)(=O)=O)nnc1N[C@@H]1CCCN(C(C)C)C1] | 472.1994 | 472 |
| 26 | 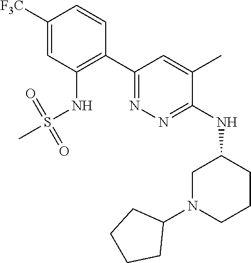 [SMILES: Cc1cc(-c2ccc(C(F)(F)F)cc2NS(C)(=O)=O)nnc1N[C@@H]1CCCN(C2CCCC2)C1] | 498.215 | 498 |
| 27 | 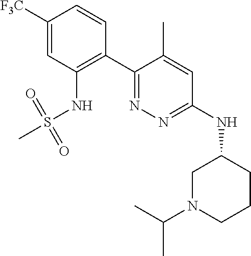 [SMILES: Cc1cc(N[C@@H]2CCCN(C(C)C)C2)nnc1-c1ccc(C(F)(F)F)cc1NS(C)(=O)=O] | 472.1994 | 472 |
| 28 | 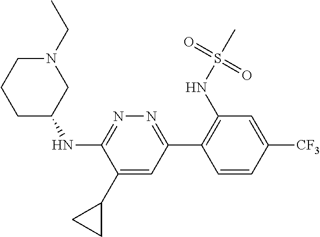 [SMILES: CCN1CCC[C@@H](Nc2nnc(-c3ccc(C(F)(F)F)cc3NS(C)(=O)=O)cc2C2CC2)C1] | 484.1994 | 484 |
| 29 | 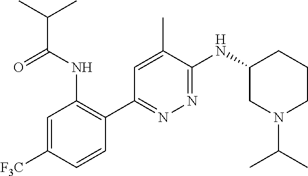 [SMILES: Cc1cc(-c2ccc(C(F)(F)F)cc2NC(=O)C(C)C)nnc1N[C@@H]1CCCN(C(C)C)C1] | 464.2637 | 464 |
| 30 | 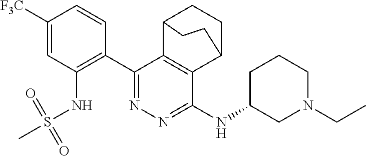 [SMILES: CCN1CCC[C@@H](Nc2nnc(-c3ccc(C(F)(F)F)cc3NS(C)(=O)=O)c3c2C2CCC3CC2)C1] | 524.2307 | 524 |
| 31 | 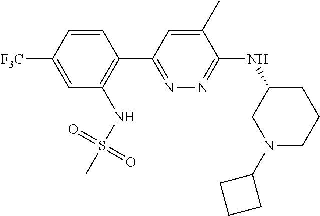 [SMILES: Cc1cc(-c2ccc(C(F)(F)F)cc2NS(C)(=O)=O)nnc1N[C@@H]1CCCN(C2CCC2)C1] | 484.1994 | 484 |
| 32 | 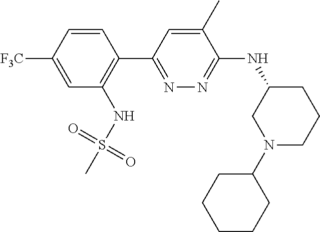 [SMILES: Cc1cc(-c2ccc(C(F)(F)F)cc2NS(C)(=O)=O)nnc1N[C@@H]1CCCN(C2CCCCC2)C1] | 512.2307 | 512 |
| 33 | 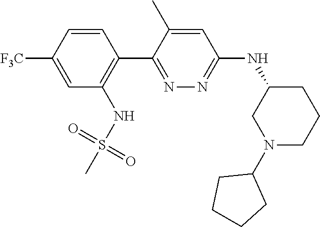 [SMILES: Cc1cc(N[C@@H]2CCCN(C3CCCC3)C2)nnc1-c1ccc(C(F)(F)F)cc1NS(C)(=O)=O] | 498.215 | 498 |
| 34 | 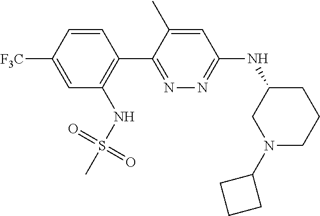 [SMILES: Cc1cc(N[C@@H]2CCCN(C3CCC3)C2)nnc1-c1ccc(C(F)(F)F)cc1NS(C)(=O)=O] | 484.1994 | 484 |
| 35 | 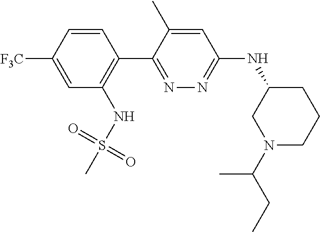 [SMILES: CCC(C)N1CCC[C@@H](Nc2cc(C)c(-c3ccc(C(F)(F)F)cc3NS(C)(=O)=O)nn2)C1] | 486.215 | 486 |
| 36 | 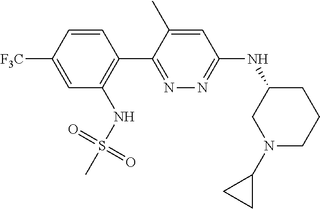 [SMILES: Cc1cc(N[C@@H]2CCCN(C3CC3)C2)nnc1-c1ccc(C(F)(F)F)cc1NS(C)(=O)=O] | 470.1837 | 470 |
| 37 | 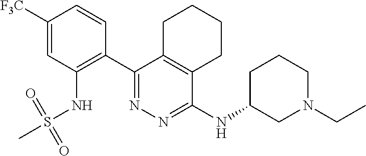 [SMILES: CCN1CCC[C@@H](Nc2nnc(-c3ccc(C(F)(F)F)cc3NS(C)(=O)=O)c3c2CCCC3)C1] | 498.215 | 498 |
| 38 | 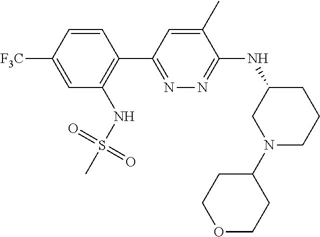 [SMILES: Cc1cc(-c2ccc(C(F)(F)F)cc2NS(C)(=O)=O)nnc1N[C@@H]1CCCN(C2CCOCC2)C1] | 514.2099 | 514 |
| 39 | 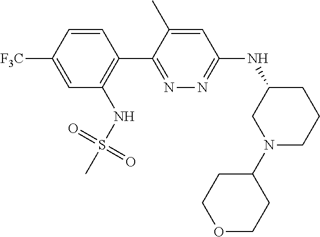 [SMILES: Cc1cc(N[C@@H]2CCCN(C3CCOCC3)C2)nnc1-c1ccc(C(F)(F)F)cc1NS(C)(=O)=O] | 514.2099 | 514 |
| 40 | 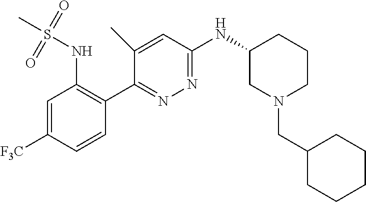 [SMILES: Cc1cc(N[C@@H]2CCCN(CC3CCCCC3)C2)nnc1-c1ccc(C(F)(F)F)cc1NS(C)(=O)=O] | 526.2463 | 526 |
| 41 | 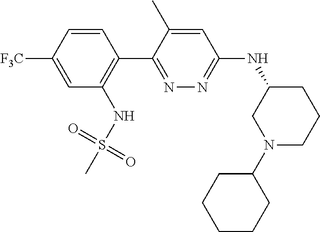 [SMILES: Cc1cc(N[C@@H]2CCCN(C3CCCCC3)C2)nnc1-c1ccc(C(F)(F)F)cc1NS(C)(=O)=O] | 512.2307 | 512 |
| 42 | 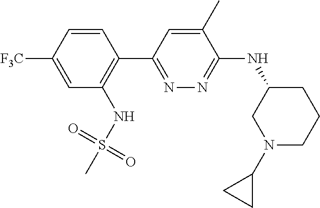 [SMILES: Cc1cc(-c2ccc(C(F)(F)F)cc2NS(C)(=O)=O)nnc1N[C@@H]1CCCN(C2CC2)C1] | 470.1837 | 470 |
| 43 | 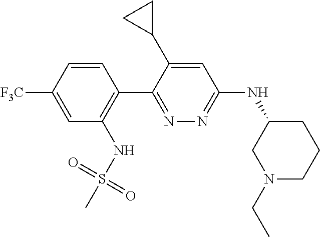 [SMILES: CCN1CCC[C@@H](Nc2cc(C3CC3)c(-c3ccc(C(F)(F)F)cc3NS(C)(=O)=O)nn2)C1] | 484.1994 | 484 |
| 44 | 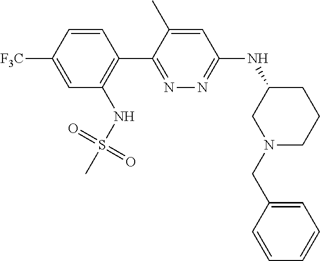 [SMILES: Cc1cc(N[C@@H]2CCCN(Cc3ccccc3)C2)nnc1-c1ccc(C(F)(F)F)cc1NS(C)(=O)=O] | 520.1994 | 520 |
| 45 | 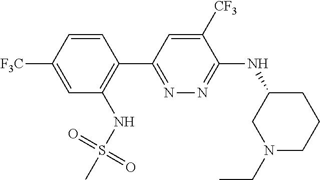 [SMILES: CCN1CCC[C@@H](Nc2nnc(-c3ccc(C(F)(F)F)cc3NS(C)(=O)=O)cc2C(F)(F)F)C1] | 512.1555 | 512 |
| 46 | 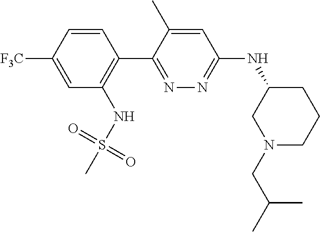 [SMILES: Cc1cc(N[C@@H]2CCCN(CC(C)C)C2)nnc1-c1ccc(C(F)(F)F)cc1NS(C)(=O)=O] | 486.215 | 486 |
| 47 | 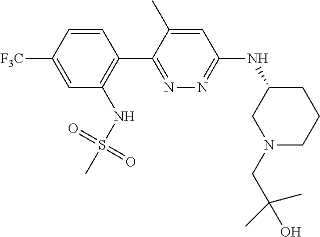 [SMILES: Cc1cc(N[C@@H]2CCCN(CC(C)(C)O)C2)nnc1-c1ccc(C(F)(F)F)cc1NS(C)(=O)=O] | 502.2099 | 502 |
| 48 | 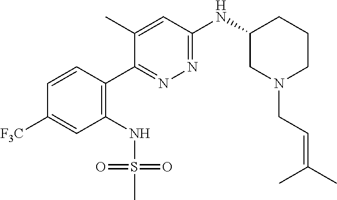 [SMILES: CC(C)=CCN1CCC[C@@H](Nc2cc(C)c(-c3ccc(C(F)(F)F)cc3NS(C)(=O)=O)nn2)C1] | 498.215 | 498 |
| 49 | 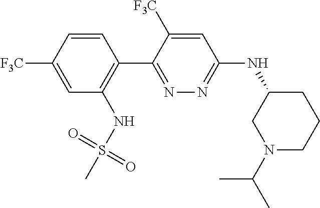 [SMILES: CC(C)N1CCC[C@@H](Nc2cc(C(F)(F)F)c(-c3ccc(C(F)(F)F)cc3NS(C)(=O)=O)nn2)C1] | 512.1555 | 512 |
| 50 | 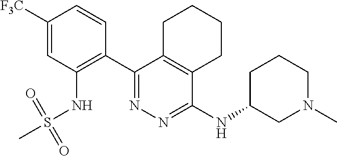 [SMILES: CN1CCC[C@@H](Nc2nnc(-c3ccc(C(F)(F)F)cc3NS(C)(=O)=O)c3c2CCCC3)C1] | 484.1994 | 484 |
| 51 | 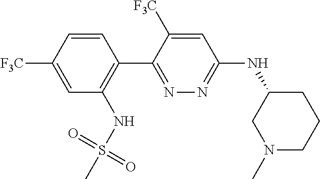 [SMILES: CN1CCC[C@@H](Nc2cc(C(F)(F)F)c(-c3ccc(C(F)(F)F)cc3NS(C)(=O)=O)nn2)C1] | 498.1398 | 498 |
| 52 | 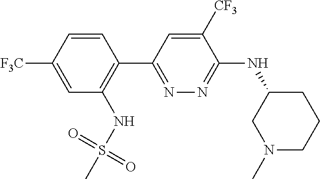 [SMILES: CN1CCC[C@@H](Nc2nnc(-c3ccc(C(F)(F)F)cc3NS(C)(=O)=O)cc2C(F)(F)F)C1] | 498.1398 | 498 |
| 53 | 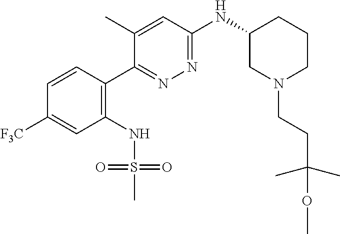 [SMILES: COC(C)(C)CCN1CCC[C@@H](Nc2cc(C)c(-c3ccc(C(F)(F)F)cc3NS(C)(=O)=O)nn2)C1] | 530.2413 | 530 |
| 54 | 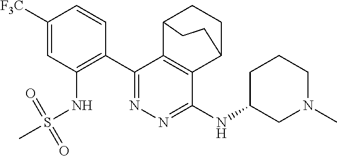 [SMILES: CN1CCC[C@@H](Nc2nnc(-c3ccc(C(F)(F)F)cc3NS(C)(=O)=O)c3c2C2CCC3CC2)C1] | 510.215 | 510 |
| 55 | 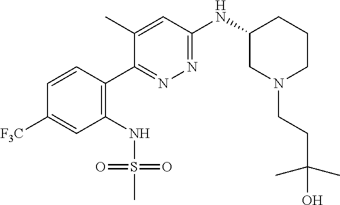 [SMILES: Cc1cc(N[C@@H]2CCCN(CCC(C)(C)O)C2)nnc1-c1ccc(C(F)(F)F)cc1NS(C)(=O)=O] | 516.2256 | 516 |
| 56 | 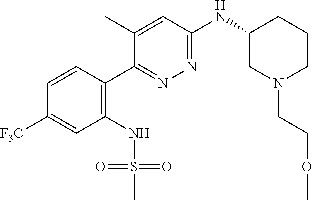 [SMILES: COCCN1CCC[C@@H](Nc2cc(C)c(-c3ccc(C(F)(F)F)cc3NS(C)(=O)=O)nn2)C1] | 488.1943 | 488 |
| 57 | 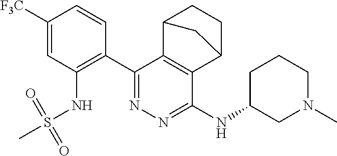 [SMILES: CN1CCC[C@@H](Nc2nnc(-c3ccc(C(F)(F)F)cc3NS(C)(=O)=O)c3c2C2CCC3C2)C1] | 496.1994 | 496 |
| 58 | 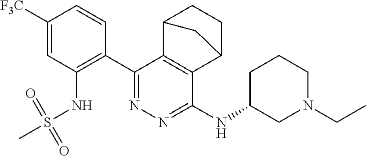 [SMILES: CCN1CCC[C@@H](Nc2nnc(-c3ccc(C(F)(F)F)cc3NS(C)(=O)=O)c3c2C2CCC3C2)C1] | 510.215 | 510 |
| 59 | 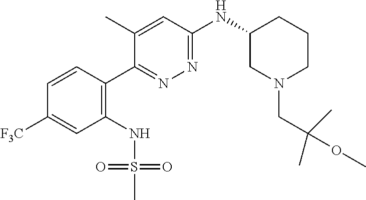 [SMILES: COC(C)(C)CN1CCC[C@@H](Nc2cc(C)c(-c3ccc(C(F)(F)F)cc3NS(C)(=O)=O)nn2)C1] | 516.2256 | 516 |
| 60 | 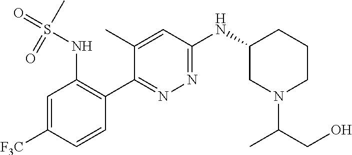 [SMILES: Cc1cc(N[C@@H]2CCCN(C(C)CO)C2)nnc1-c1ccc(C(F)(F)F)cc1NS(C)(=O)=O] | 488.1943 | 488 |
| 61 | 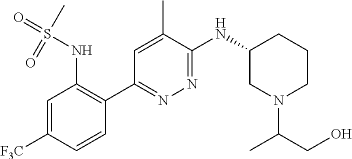 [SMILES: Cc1cc(-c2ccc(C(F)(F)F)cc2NS(C)(=O)=O)nnc1N[C@@H]1CCCN(C(C)CO)C1] | 488.1943 | 488 |
| 62 | 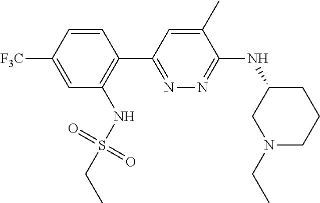 [SMILES: CCN1CCC[C@@H](Nc2nnc(-c3ccc(C(F)(F)F)cc3NS(=O)(=O)CC)cc2C)C1] | 472.1994 | 472 |
| 63 | 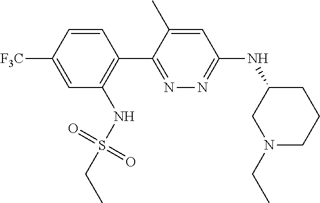 [SMILES: CCN1CCC[C@@H](Nc2cc(C)c(-c3ccc(C(F)(F)F)cc3NS(=O)(=O)CC)nn2)C1] | 472.1994 | 472 |
| 64 | 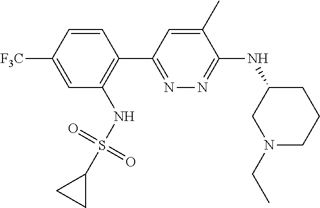 [SMILES: CCN1CCC[C@@H](Nc2nnc(-c3ccc(C(F)(F)F)cc3NS(=O)(=O)C3CC3)cc2C)C1] | 484.1994 | 484 |
| 65 | 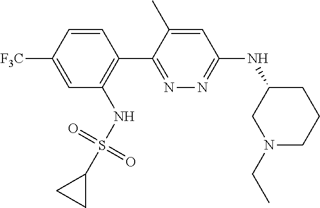 [SMILES: CCN1CCC[C@@H](Nc2cc(C)c(-c3ccc(C(F)(F)F)cc3NS(=O)(=O)C3CC3)nn2)C1] | 484.1994 | 484 |
| 66 | 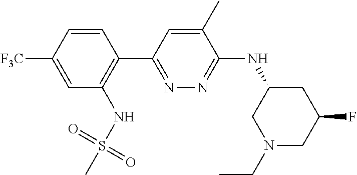 [SMILES: CCN1C[C@H](F)C[C@@H](Nc2nnc(-c3ccc(C(F)(F)F)cc3NS(C)(=O)=O)cc2C)C1] | 476.1743 | 476 |
| 67 | 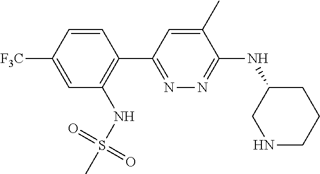 [SMILES: Cc1cc(-c2ccc(C(F)(F)F)cc2NS(C)(=O)=O)nnc1N[C@@H]1CCCNC1] | 430.1524 | 430 |
| 68 | 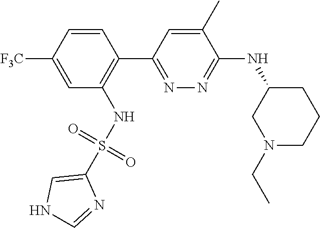 [SMILES: CCN1CCC[C@@H](Nc2nnc(-c3ccc(C(F)(F)F)cc3NS(=O)(=O)c3c[nH]cn3)cc2C)C1] | 510.1899 | 510 |
| 69 | 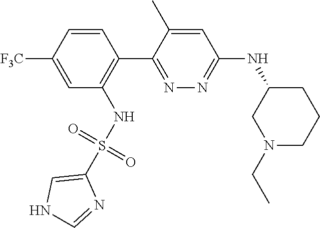 [SMILES: CCN1CCC[C@@H](Nc2cc(C)c(-c3ccc(C(F)(F)F)cc3NS(=O)(=O)c3c[nH]cn3)nn2)C1] | 510.1899 | 510 |
| 70 | 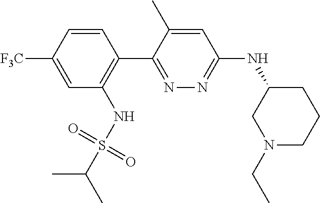 [SMILES: CCN1CCC[C@@H](Nc2cc(C)c(-c3ccc(C(F)(F)F)cc3NS(=O)(=O)C(C)C)nn2)C1] | 486.215 | 486 |
| 71 | 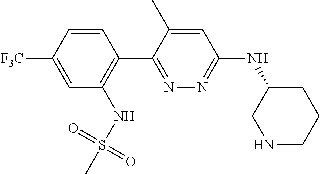 [SMILES: Cc1cc(N[C@@H]2CCCNC2)nnc1-c1ccc(C(F)(F)F)cc1NS(C)(=O)=O] | 430.1524 | 430 |
| 72 | 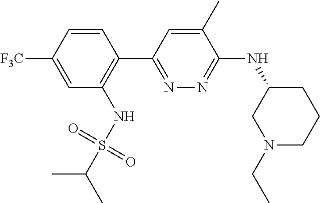 [SMILES: CCN1CCC[C@@H](Nc2nnc(-c3ccc(C(F)(F)F)cc3NS(=O)(=O)C(C)C)cc2C)C1] | 486.215 | 486 |
| 73 | 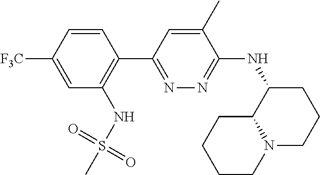 [SMILES: Cc1cc(-c2ccc(C(F)(F)F)cc2NS(C)(=O)=O)nnc1N[C@@H]1CCCN2CCCC[C@H]12] | 484.1994 | 484 |
| 74 | 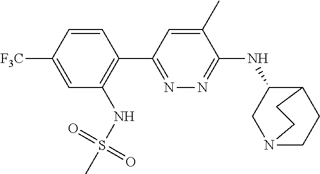 [SMILES: Cc1cc(-c2ccc(C(F)(F)F)cc2NS(C)(=O)=O)nnc1N[C@H]1CN2CCC1CC2] | 456.1681 | 456 |
| 75 | 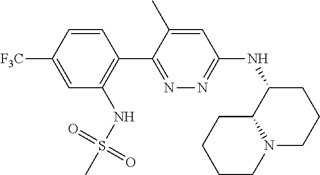 [SMILES: Cc1cc(N[C@@H]2CCCN3CCCC[C@H]23)nnc1-c1ccc(C(F)(F)F)cc1NS(C)(=O)=O] | 484.1994 | 484 |
| 76 | 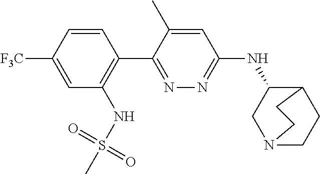 [SMILES: Cc1cc(N[C@H]2CN3CCC2CC3)nnc1-c1ccc(C(F)(F)F)cc1NS(C)(=O)=O] | 456.1681 | 456 |
| 77 | 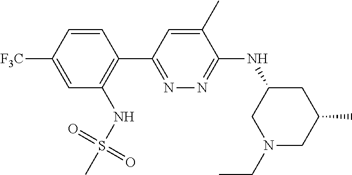 [SMILES: CCN1C[C@@H](C)C[C@@H](Nc2nnc(-c3ccc(C(F)(F)F)cc3NS(C)(=O)=O)cc2C)C1] | 472.1994 | 472 |
| 78 | 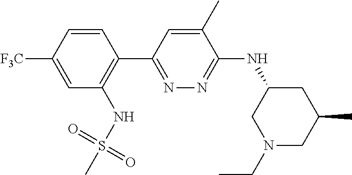 [SMILES: CCN1C[C@H](C)C[C@@H](Nc2nnc(-c3ccc(C(F)(F)F)cc3NS(C)(=O)=O)cc2C)C1] | 472.1994 | 472 |
| 79 | 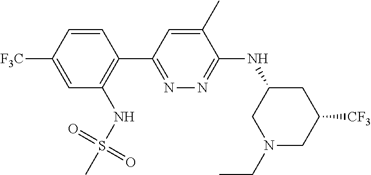 [SMILES: CCN1C[C@H](Nc2nnc(-c3ccc(C(F)(F)F)cc3NS(C)(=O)=O)cc2C)C[C@H](C(F)(F)F)C1] | 526.1711 | 526 |
| 80 | 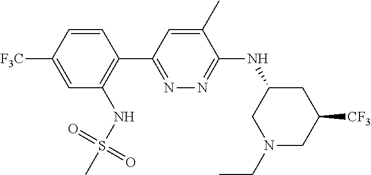 [SMILES: CCN1C[C@H](Nc2nnc(-c3ccc(C(F)(F)F)cc3NS(C)(=O)=O)cc2C)C[C@@H](C(F)(F)F)C1] | 526.1711 | 526 |
| 81 | 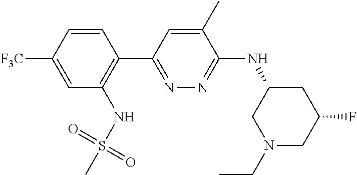 [SMILES: CCN1C[C@@H](F)C[C@@H](Nc2nnc(-c3ccc(C(F)(F)F)cc3NS(C)(=O)=O)cc2C)C1] | 476.1743 | 476 |
| 82 | 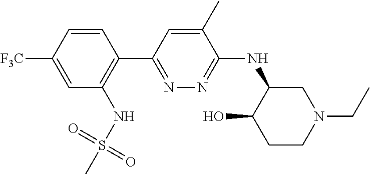 [SMILES: CCN1CC[C@@H](O)[C@@H](Nc2nnc(-c3ccc(C(F)(F)F)cc3NS(C)(=O)=O)cc2C)C1] | 474.1786 | 474 |
| 83 | 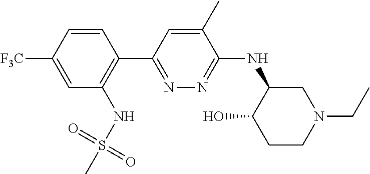 [SMILES: CCN1CC[C@H](O)[C@@H](Nc2nnc(-c3ccc(C(F)(F)F)cc3NS(C)(=O)=O)cc2C)C1] | 474.1786 | 474 |
| 84 | 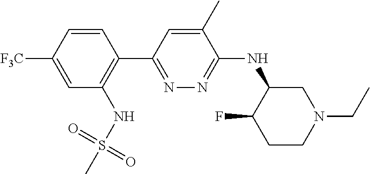 [SMILES: CCN1CC[C@@H](F)[C@@H](Nc2nnc(-c3ccc(C(F)(F)F)cc3NS(C)(=O)=O)cc2C)C1] | 476.1743 | 476 |
| 85 | 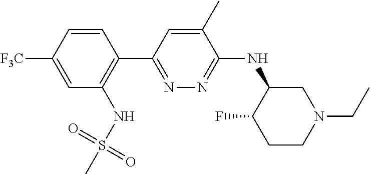 [SMILES: CCN1CC[C@H](F)[C@@H](Nc2nnc(-c3ccc(C(F)(F)F)cc3NS(C)(=O)=O)cc2C)C1] | 476.1743 | 476 |
| 86 | 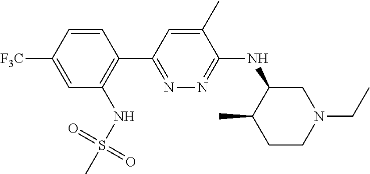 [SMILES: CCN1CC[C@@H](C)[C@@H](Nc2nnc(-c3ccc(C(F)(F)F)cc3NS(C)(=O)=O)cc2C)C1] | 472.1994 | 472 |
| 87 | 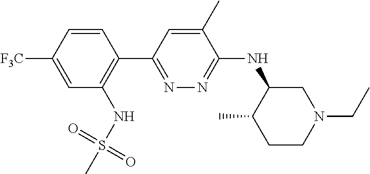 [SMILES: CCN1CC[C@H](C)[C@@H](Nc2nnc(-c3ccc(C(F)(F)F)cc3NS(C)(=O)=O)cc2C)C1] | 472.1994 | 472 |
| 88 | 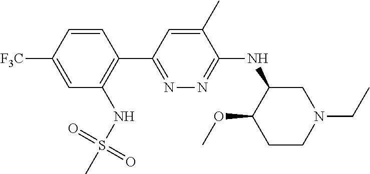 [SMILES: CCN1CC[C@@H](OC)[C@@H](Nc2nnc(-c3ccc(C(F)(F)F)cc3NS(C)(=O)=O)cc2C)C1] | 488.1943 | 488 |
| 89 | 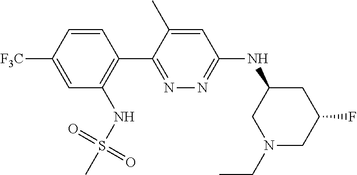 [SMILES: CCN1C[C@@H](F)C[C@H](Nc2cc(C)c(-c3ccc(C(F)(F)F)cc3NS(C)(=O)=O)nn2)C1] | 476.1743 | 476 |
| 90 | 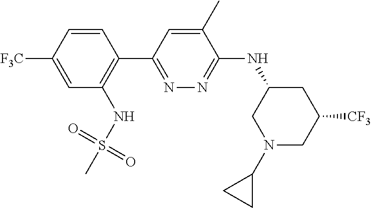 [SMILES: Cc1cc(-c2ccc(C(F)(F)F)cc2NS(C)(=O)=O)nnc1N[C@@H]1C[C@H](C(F)(F)F)CN(C2CC2)C1] | 538.1711 | 538 |
| 91 | 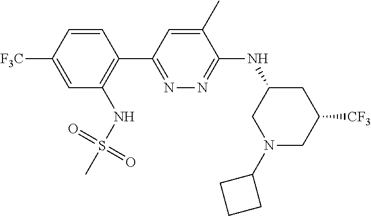 [SMILES: Cc1cc(-c2ccc(C(F)(F)F)cc2NS(C)(=O)=O)nnc1N[C@@H]1C[C@H](C(F)(F)F)CN(C2CCC2)C1] | 552.1868 | 552 |
| 92 | 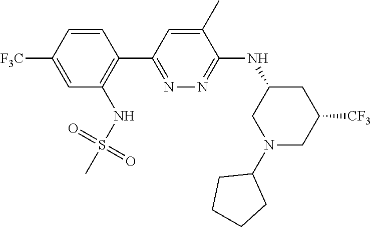 [SMILES: Cc1cc(-c2ccc(C(F)(F)F)cc2NS(C)(=O)=O)nnc1N[C@@H]1C[C@H](C(F)(F)F)CN(C2CCCC2)C1] | 566.2024 | 566 |
| 93 | 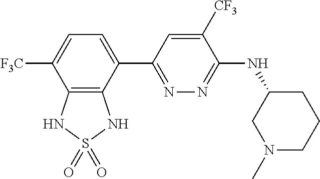 [SMILES: CN1CCC[C@@H](Nc2nnc(-c3ccc(C(F)(F)F)c4c3NS(=O)(=O)N4)cc2C(F)(F)F)C1] | 497.1194 | 497 |
| 94 | 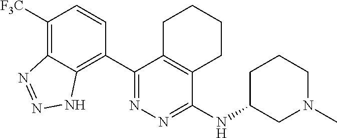 [SMILES: CN1CCC[C@@H](Nc2nnc(-c3ccc(C(F)(F)F)c4nn[nH]c34)c3c2CCCC3)C1] | 432.2123 | 432 |
| 95 | 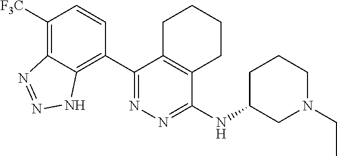 [SMILES: CCN1CCC[C@@H](Nc2nnc(-c3ccc(C(F)(F)F)c4nn[nH]c34)c3c2CCCC3)C1] | 446.228 | 446 |
| 96 | 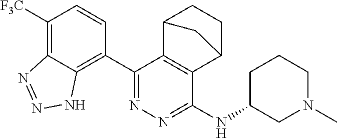 [SMILES: CN1CCC[C@@H](Nc2nnc(-c3ccc(C(F)(F)F)c4nn[nH]c34)c3c2C2CCC3C2)C1] | 444.2123 | 444 |
| 97 | 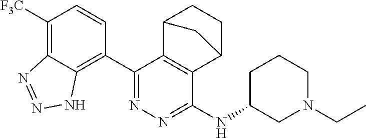 [SMILES: CCN1CCC[C@@H](Nc2nnc(-c3ccc(C(F)(F)F)c4nn[nH]c34)c3c2C2CCC3C2)C1] | 458.228 | 458 |
| 98 | 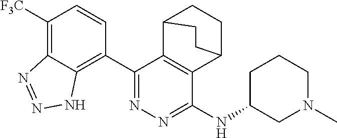 [SMILES: CN1CCC[C@@H](Nc2nnc(-c3ccc(C(F)(F)F)c4nn[nH]c34)c3c2C2CCC3CC2)C1] | 458.228 | 458 |
| 99 | 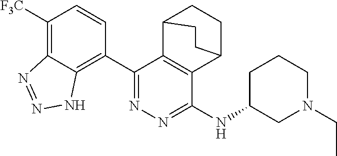 [SMILES: CCN1CCC[C@@H](Nc2nnc(-c3ccc(C(F)(F)F)c4nn[nH]c34)c3c2C2CCC3CC2)C1] | 472.2436 | 472 |
| 100 | 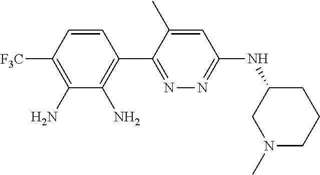 [SMILES: Cc1cc(N[C@@H]2CCCN(C)C2)nnc1-c1ccc(C(F)(F)F)c(N)c1N] | 381.2014 | 381 |
| 101 | 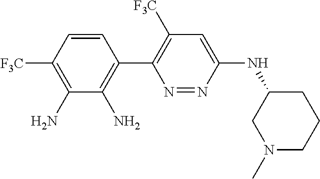 [SMILES: CN1CCC[C@@H](Nc2cc(C(F)(F)F)c(-c3ccc(C(F)(F)F)c(N)c3N)nn2)C1] | 435.1732 | 435 |
| 102 | 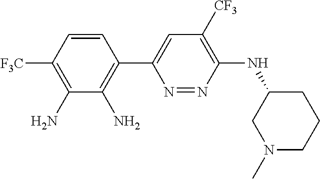 [SMILES: CN1CCC[C@@H](Nc2nnc(-c3ccc(C(F)(F)F)c(N)c3N)cc2C(F)(F)F)C1] | 435.1732 | 435 |
| 103 | 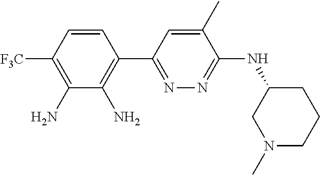 [SMILES: Cc1cc(-c2ccc(C(F)(F)F)c(N)c2N)nnc1N[C@@H]1CCCN(C)C1] | 381.2014 | 381 |
| 104 | 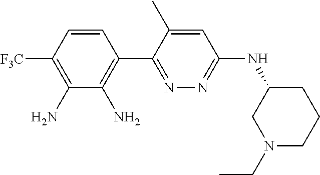 [SMILES: CCN1CCC[C@@H](Nc2cc(C)c(-c3ccc(C(F)(F)F)c(N)c3N)nn2)C1] | 395.2171 | 395 |
| 105 | 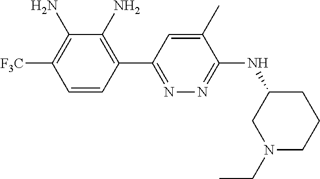 [SMILES: CCN1CCC[C@@H](Nc2nnc(-c3ccc(C(F)(F)F)c(N)c3N)cc2C)C1] | 395.2171 | 395 |
| 106 | 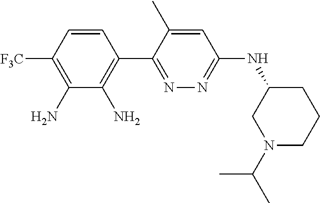 [SMILES: Cc1cc(N[C@@H]2CCCN(C(C)C)C2)nnc1-c1ccc(C(F)(F)F)c(N)c1N] | 409.2327 | 409 |
| 107 | 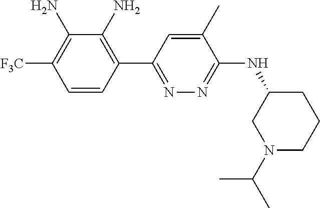 [SMILES: Cc1cc(-c2ccc(C(F)(F)F)c(N)c2N)nnc1N[C@@H]1CCCN(C(C)C)C1] | 409.2327 | 409 |
| 108 | 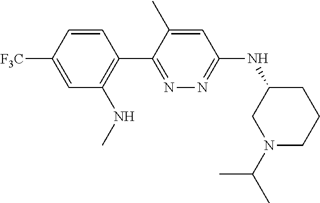 [SMILES: CNc1cc(C(F)(F)F)ccc1-c1nnc(N[C@@H]2CCCN(C(C)C)C2)cc1C] | 408.2375 | 408 |
| 109 | 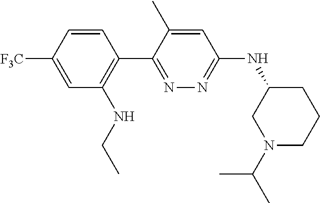 [SMILES: CCNc1cc(C(F)(F)F)ccc1-c1nnc(N[C@@H]2CCCN(C(C)C)C2)cc1C] | 422.2531 | 422 |
| 110 | 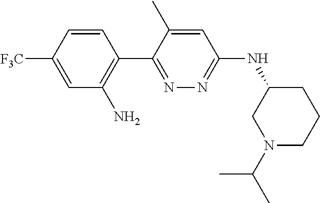 [SMILES: Cc1cc(N[C@@H]2CCCN(C(C)C)C2)nnc1-c1ccc(C(F)(F)F)cc1N] | 394.2218 | 394 |
| 111 | 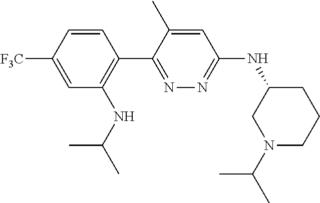 [SMILES: Cc1cc(N[C@@H]2CCCN(C(C)C)C2)nnc1-c1ccc(C(F)(F)F)cc1NC(C)C] | 436.2688 | 436 |
| 112 | 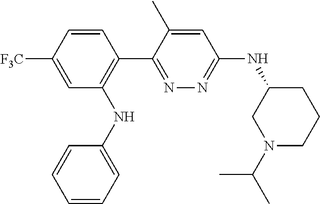 [SMILES: Cc1cc(N[C@@H]2CCCN(C(C)C)C2)nnc1-c1ccc(C(F)(F)F)cc1Nc1ccccc1] | 470.2531 | 470 |
| 113 | 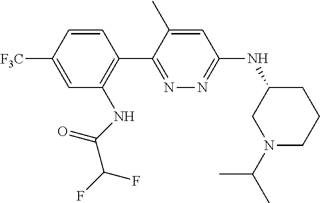 [SMILES: Cc1cc(N[C@@H]2CCCN(C(C)C)C2)nnc1-c1ccc(C(F)(F)F)cc1NC(=O)C(F)F] | 472.2135 | 472 |
| 114 | 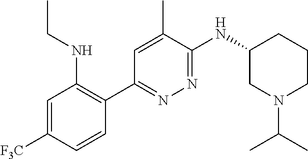 [SMILES: CCNc1cc(C(F)(F)F)ccc1-c1cc(C)c(N[C@@H]2CCCN(C(C)C)C2)nn1] | 422.2531 | 422 |
| 115 | 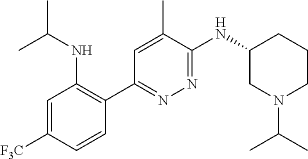 [SMILES: Cc1cc(-c2ccc(C(F)(F)F)cc2NC(C)C)nnc1N[C@@H]1CCCN(C(C)C)C1] | 436.2688 | 436 |
| 116 | 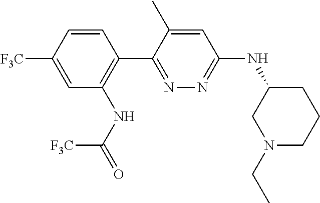 [SMILES: CCN1CCC[C@@H](Nc2cc(C)c(-c3ccc(C(F)(F)F)cc3NC(=O)C(F)(F)F)nn2)C1] | 476.1885 | 476 |
| 117 | 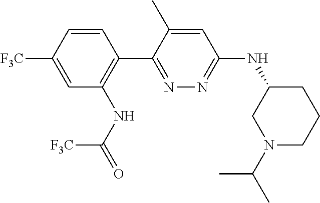 [SMILES: Cc1cc(N[C@@H]2CCCN(C(C)C)C2)nnc1-c1ccc(C(F)(F)F)cc1NC(=O)C(F)(F)F] | 490.2041 | 490 |
| 118 | 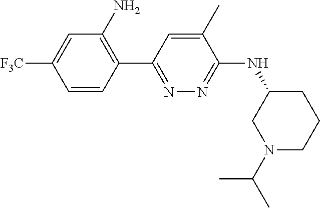 [SMILES: Cc1cc(-c2ccc(C(F)(F)F)cc2N)nnc1N[C@@H]1CCCN(C(C)C)C1] | 394.2218 | 394 |
| 119 | 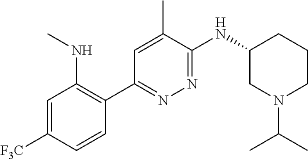 [SMILES: CNc1cc(C(F)(F)F)ccc1-c1cc(C)c(N[C@@H]2CCCN(C(C)C)C2)nn1] | 408.2375 | 408 |
| 120 | 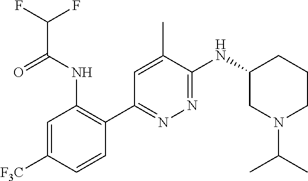 [SMILES: Cc1cc(-c2ccc(C(F)(F)F)cc2NC(=O)C(F)F)nnc1N[C@@H]1CCCN(C(C)C)C1] | 472.2135 | 472 |
| 121 | 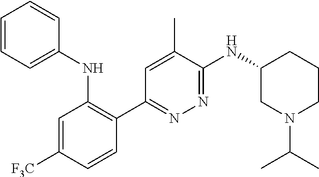 [SMILES: Cc1cc(-c2ccc(C(F)(F)F)cc2Nc2ccccc2)nnc1N[C@@H]1CCCN(C(C)C)C1] | 470.2531 | 470 |
| 122 | 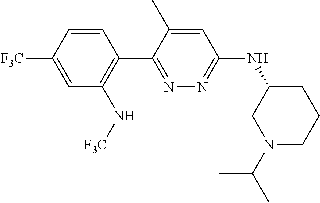 [SMILES: Cc1cc(N[C@@H]2CCCN(C(C)C)C2)nnc1-c1ccc(C(F)(F)F)cc1NC(F)(F)F] | 490.2041 | 490 |
| 123 | 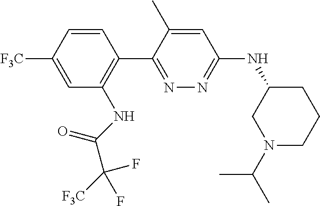 [SMILES: Cc1cc(N[C@@H]2CCCN(C(C)C)C2)nnc1-c1ccc(C(F)(F)F)cc1NC(=O)C(F)(F)C(F)(F)F] | 462.2092 | 462 |
| 124 | 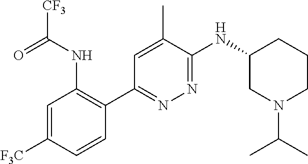 [SMILES: Cc1cc(-c2ccc(C(F)(F)F)cc2NC(=O)C(F)(F)F)nnc1N[C@@H]1CCCN(C(C)C)C1] | 540.2009 | 540 |
| 125 | 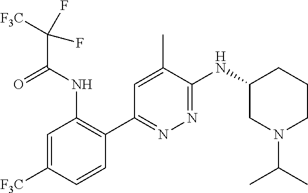 [SMILES: Cc1cc(-c2ccc(C(F)(F)F)cc2NC(=O)C(F)(F)C(F)(F)F)nnc1N[C@@H]1CCCN(C(C)C)C1] | 476.1885 | 476 |
| 126 | 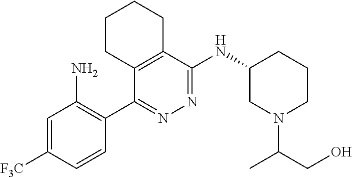 [SMILES: CC(CO)N1CCC[C@@H](Nc2nnc(-c3ccc(C(F)(F)F)cc3N)c3c2CCCC3)C1] | 430.2009 | 540 |
| 127 | 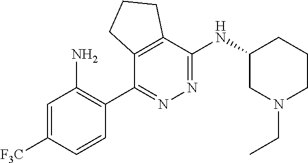 [SMILES: CCN1CCC[C@@H](Nc2nnc(-c3ccc(C(F)(F)F)cc3N)c3c2CCC3)C1] | 450.248 | 450 |
| 128 | 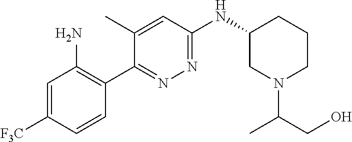 [SMILES: Cc1cc(N[C@@H]2CCCN(C(C)CO)C2)nnc1-c1ccc(C(F)(F)F)cc1N] | 406.2218 | 406 |
| 129 | 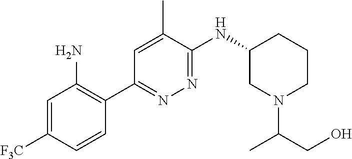 [SMILES: Cc1cc(-c2ccc(C(F)(F)F)cc2N)nnc1N[C@@H]1CCCN(C(C)CO)C1] | 410.2168 | 410 |
| 130 | 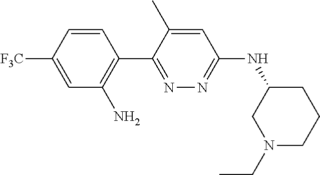 [SMILES: CCN1CCC[C@@H](Nc2cc(C)c(-c3ccc(C(F)(F)F)cc3N)nn2)C1] | 410.2168 | 410 |
| 131 | 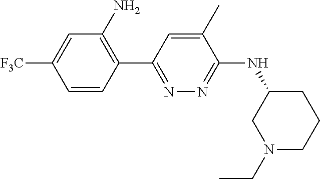 [SMILES: CCN1CCC[C@@H](Nc2nnc(-c3ccc(C(F)(F)F)cc3N)cc2C)C1] | 380.2062 | 380 |
| 132 | 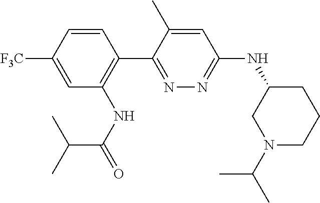 [SMILES: Cc1cc(N[C@@H]2CCCN(C(C)C)C2)nnc1-c1ccc(C(F)(F)F)cc1NC(=O)C(C)C] | 380.2062 | 380 |
| 133 | 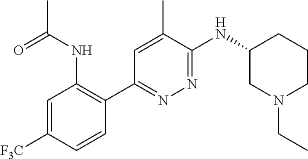 [SMILES: CCN1CCC[C@@H](Nc2nnc(-c3ccc(C(F)(F)F)cc3NC(C)=O)cc2C)C1] | 464.2637 | 464 |
| 134 | 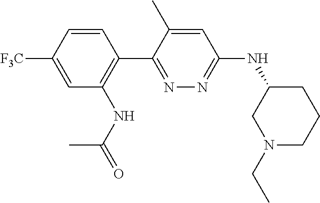 [SMILES: CCN1CCC[C@@H](Nc2cc(C)c(-c3ccc(C(F)(F)F)cc3NC(C)=O)nn2)C1] | 422.2168 | 422 |
| 135 | 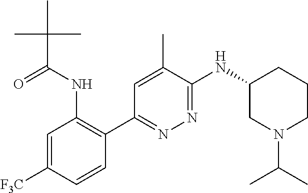 [SMILES: Cc1cc(-c2ccc(C(F)(F)F)cc2NC(=O)C(C)(C)C)nnc1N[C@@H]1CCCN(C(C)C)C1] | 422.2168 | 422 |
| 136 | 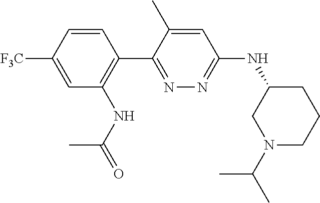 [SMILES: CC(=O)Nc1cc(C(F)(F)F)ccc1-c1nnc(N[C@@H]2CCCN(C(C)C)C2)cc1C] | 478.2793 | 478 |
| 137 | 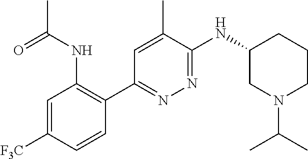 [SMILES: CC(=O)Nc1cc(C(F)(F)F)ccc1-c1cc(C)c(N[C@@H]2CCCN(C(C)C)C2)nn1] | 436.2324 | 436 |
| 138 | 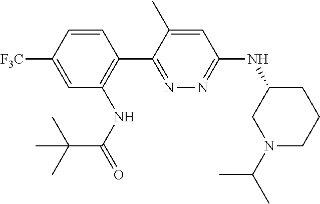 [SMILES: Cc1cc(N[C@@H]2CCCN(C(C)C)C2)nnc1-c1ccc(C(F)(F)F)cc1NC(=O)C(C)(C)C] | 436.2324 | 436 |
| 139 | 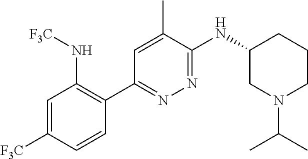 [SMILES: Cc1cc(-c2ccc(C(F)(F)F)cc2NC(F)(F)F)nnc1N[C@@H]1CCCN(C(C)C)C1] | 478.2793 | 478 |
| 140 | 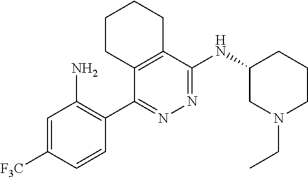 [SMILES: CCN1CCC[C@@H](Nc2nnc(-c3ccc(C(F)(F)F)cc3N)c3c2CCCC3)C1] | 462.2092 | 462 |
| 141 | 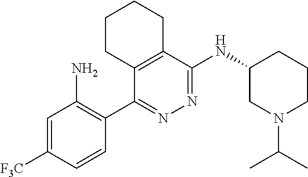 [SMILES: CC(C)N1CCC[C@@H](Nc2nnc(-c3ccc(C(F)(F)F)cc3N)c3c2CCCC3)C1] | 420.2375 | 420 |

### Synthesis of the Representative Examples of the Compound

### Example 1. (R)—N-(2-(4-Methyl-6-((1-methylpiperidin-3-yl)amino)pyridazin-3-yl)-5-(trifluoromethyl)phenyl) methanesulfonamide (Compound 4)

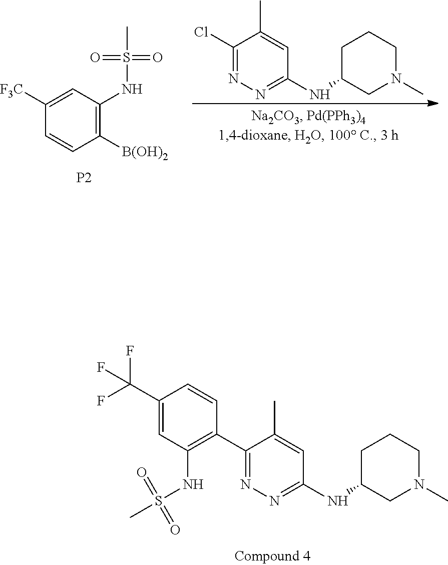

To a stirred solution of (R)-6-chloro-5-methyl-N-(1-methylpiperidin-3-yl)pyridazin-3-amine (200 mg, 0.833 mmol, 1.0 eq) in 1,4-dioxane:H₂O (3:1, 8.0 mL) were added (2-(methylsulfonamido)-4-(trifluoromethyl)phenyl) boronic acid (P2, 0.943 mg, 3.333 mmol, 4.0 eq) followed by Na₂CO₃ (264.9 mg, 2.50 mmol, 3.0 eq) at ambient temperature. The resulting reaction mixture was degassed with argon for 15 min followed by Pd(PPh₃)₄ (48.1 mg, 0.042 mmol, 0.05 eq) was added at ambient temperature and stirred at 100° C. for 2 h. The progress of reaction was monitored by TLC and LCMS. After completion of starting material, reaction mixture was quenched with water (50 mL) and extracted with EtOAc (3× 50 mL). The combined organic layers were dried over anh. Na₂SO₄, filtered and concentrated under reduced pressure. The residue was purified by Prep-HPLC to afford 95 mg (25.7%) of the title compound as an off white solid.

Analytical data: ¹H NMR (400 MHz, DMSO-d₆), δ: 9.38 (br. s, exch. with D₂O, 1H), 7.76 (s, 1H), 7.54-7.48 (m, 2H), 6.80 (d, J=7.6 Hz, 1H), 6.75 (s, 1H), 4.08 (br. s, 1H), 2.94 (s, 4H), 2.67-2.63 (m, 1H), 2.26 (s, 3H), 2.13 (br. s, 1H), 1.99 (s, 4H), 1.86-1.83 (m, 1H), 1.76-1.73 (m, 1H), 1.61-1.55 (m, 1H), 1.34 (d, J=9.6 Hz, 1H). LCMS (ESI): calc. for C₁₉H₂₄F₃N₅O₂S [M+H]⁺ 444.1, found 444.1.

### Example 2. (R)—N-(2-(6-((1-Ethylpiperidin-3-yl)amino)-4-methylpyridazin-3-yl)-5-(trifluoromethyl)phenyl) methanesulfonamide (Compound 5)

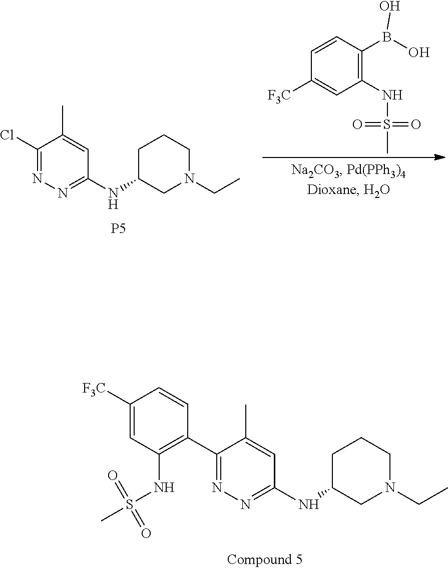

To a stirred solution of (R)-6-chloro-N-(1-ethylpiperidin-3-yl)-5-methylpyridazin-3-amine (P5, 200 mg, 0.787 mmol, 1.0 eq) in 1,4-dioxane/H₂O (3:1, 8.0 mL) were added (2-(methylsulfonamido)-4-(trifluoromethyl)phenyl) boronic acid (P2, 891 mg, 3.150 mmol, 4.0 eq) and Na₂CO₃ (250 mg, 2.36 mmol, 3.0 eq) at ambient temperature. The resulting reaction mixture was degassed under argon for 15 min and then Pd(PPh₃)₄ (45.4 mg, 0.039 mmol, 0.05 eq) was added at ambient temperature and stirred at 100° C. for 2 h. The progress of reaction was monitored by TLC and LCMS. After completion of starting material, reaction mixture was quenched with water (50 mL) and extracted with EtOAc (3×50 mL). The combined organic layers were dried over anh. Na₂SO₄, filtered and concentrated under reduced pressure. The residue was purified by Prep-HPLC to afford 105 mg (29.4%) of the title compound as an off white solid.

Analytical data: ¹H NMR (400 MHz, DMSO-d₆), δ: 10.16 (br. s, exch. with D₂O, 1H), 7.75 (s, 1H), 7.48 (s, 2H), 6.76 (s, 1H), 6.73 (s, 1H), 4.08 (br. s, 1H), 3.04 (d, J=9.6 Hz, 1H), 2.92 (s, 1H), 2.75 (br. s, 1H), 2.43 (d, J=7.2 Hz, 2H), 2.11 (m, 1H), 2.00 (s, 4H), 1.91-1.88 (m, 1H), 1.76-1.73 (m, 1H), 1.57-1.55 (m, 1H), 1.35-1.33 (m, 1H), 1.02 (t, J=7.2 Hz, 3H). LCMS (ESI): calc, for C₁₉H₂₄F₃N₅O₂S [M+H]⁺ 458.1, found 458.2.

### Example 3. (R)—N-(2-(6-((1-(2-Hydroxyethyl) piperidin-3-yl)amino)-4-methylpyridazin-3-yl)-5-(trifluoromethyl)phenyl) methanesulfonamide (Compound 6)

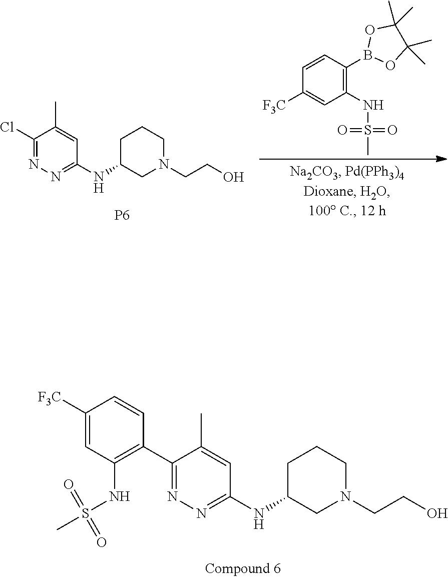

To a stirred solution of (R)-2-(3-((6-chloro-5-methylpyridazin-3-yl)amino) piperidin-1-yl) ethan-1-ol (P6, 200 mg, 0.739 mmol, 1.0 eq) in 1,4-dioxane:H₂O (3:1, 8.0 mL) were added Na₂CO₃ (234 mg, 2.21 mmol, 3.0 eq) and (2-(methylsulfonamido)-4-(trifluoromethyl)phenyl) boronic acid (836 mg, 2.955 mmol, 4.0 eq) at ambient temperature. The reaction mixture degassed with N₂ for 15 min and Pd(PPh₃)₄ (42 mg, 0.037 mmol, 0.1 eq) was added. The resulting reaction mixture was stirred at 100° C. for 12 h. The progress of the reaction was monitored by TLC and LCMS. After completion of starting material, reaction mixture was quenched with water (50 mL) and reaction mass extracted with EtOAc (3×100 mL). The combined organic layers were dried over anh. Na₂SO₄, filtered and concentrated under reduced pressure. The residue was purified by Prep-HPLC, to afford 67 mg (19.0%) of the title compound as an off white solid.

Analytical data: ¹H NMR (400 MHz, DMSO-d₆), δ: 10.05 (br. s, exch. with D₂O, 1H), 7.77 (br. s, 1H), 7.55-7.50 (m, 2H), 6.80 (d, J=7.6 Hz, 1H), 6.74 (br. s, 1H), 4.49 (br. s, 1H), 4.11 (br. s, 1H), 3.52 (t, J=6.0 Hz, 2H), 3.31 (1H, merged with moisture), 3.02 (s, 3H), 2.73 (br. s, 1H), 2.32-2.15 (m, 2H), 2.00 (s, 4H), 2.54 (s, 1H), 1.86-1.84 (m, 1H), 1.75-1.71 (m, 1H), 1.58-1.56 (m, 1H), 1.41-1.36 (m, 1H). LCMS (ESI): calc, for C₂₀H₂₆F₃N₅O₃S [M+H]⁺ 474.1, found 474.2.

### Example 4. (R)—N-(2-(5-Methyl-6-((1-methylpiperidin-3-yl)amino)pyridazin-3-yl)-5-(trifluoromethyl)phenyl) methanesulfonamide (Compound 7)

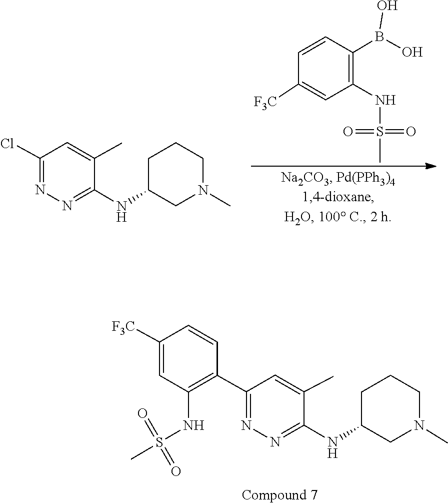 [SMILES: Cc1cc(Cl)nnc1N[C@@H]1CCCN(C)C1 ; CS(=O)(=O)Nc1cc(C(F)(F)F)ccc1B(O)O ; Cc1cc(-c2ccc(C(F)(F)F)cc2NS(C)(=O)=O)nnc1N[C@@H]1CCCN(C)C1]

To a stirred solution of (R)-6-chloro-4-methyl-N-(1-methylpiperidin-3-yl)pyridazin-3-amine (200 mg, 0.833 mmol, 1.0 eq) in 1,4-dioxane:H₂O (3:1, 8.0 mL) were added (2-(methylsulfonamido)-4-(trifluoromethyl)phenyl) boronic acid (P2, 0.943 mg, 3.333 mmol, 4.0 eq) and Na₂CO₃ (264.9 mg, 2.500 mmol, 3.0 eq) at ambient temperature, resulting reaction mixture was degassed with argon for 15 min. Then Pd(PPh₃)₄ (48.1 mg, 0.042 mmol, 0.05 eq) was added at ambient temperature and stirred at 100° C. for 2 h. The progress of reaction was monitored by TLC and LCMS. After completion of starting material, reaction mixture was quenched with water (50 mL) and extracted with EtOAc (3×50 mL). The combined organic layers were dried over anh. Na₂SO₄, filtered and concentrated under reduced pressure. The residue was purified by Prep-HPLC to afford 28 mg (7.5%) of the title compound as an off white solid.

Analytical data: ¹H NMR (400 MHz, DMSO-d₆), δ: 12.76 (br. s, exch. with D₂O, 1H), 8.10 (d, J=8.4 Hz, 1H), 8.00 (br. s, 1H), 7.86 (br. s, 1H), 7.55 (d, J=8.4 Hz, 1H), 6.35 (br. s, 1H), 4.35 (br. s, 1H), 3.12 (s, 3H), 3.00 (br. s, 1H), 2.75 (br. s, 1H), 2.22 (br. s, 6H), 2.14-2.06 (m, 1H), 1.90-1.88 (m, 2H), 1.73 (m, 1H), 1.65-1.56 (m, 1H), 1.51-1.46 (m, 1H). LCMS (ESI): calc, for C₁₉H₂₄F₃N₅O₂S [M+H]⁺ 444.1, found 444.2.

### Example 5. (R)—N-(2-(6-((1-Ethylpiperidin-3-yl)amino)-5-methylpyridazin-3-yl)-5-(trifluoromethyl)phenyl) methanesulfonamide (Compound 8)

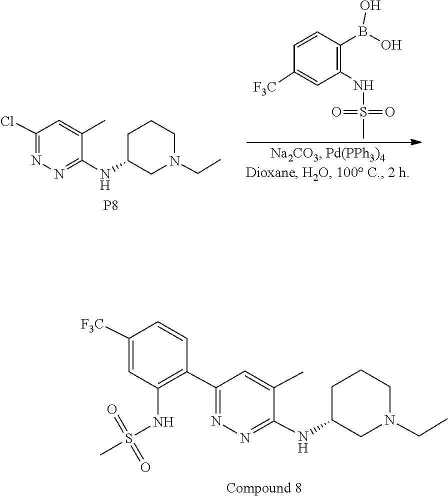

To a stirred solution of (R)-6-chloro-N-(1-ethylpiperidin-3-yl)-5-methylpyridazin-3-amine (P8, 200 mg, 0.787 mmol, 1.0 eq) in 1,4-dioxane/H₂O (3:1, 8.0 mL) were added (2-(methylsulfonamido)-4-(trifluoromethyl)phenyl) boronic acid (P2, 0.891 mg, 3.150 mmol, 4.0 eq) and Na₂CO₃ (250 mg, 2.362 mmol, 3.0 eq) at ambient temperature and degassed with argon for 15 min. Then Pd(PPh₃)₄ (45.4 mg, 0.039 mmol, 0.05 eq) was added at ambient temperature and stirred at 100° C. for 2 h. The progress of reaction was monitored by TLC and LCMS. After completion of starting material, reaction mixture was quenched with water (50 mL) and extracted with EtOAc (3×50 mL). The combined organic layers were dried over anh. Na₂SO₄, filtered and concentrated under reduced pressure. The residue was purified by Prep-HPLC to afford 20 mg (5.2%) of the title compound as an off white solid.

Analytical data: ¹H NMR (400 MHz, DMSO-d₆), δ: 12.85 (br. s, exch. with D₂O, 1H), 8.02 (d, J=8.4 Hz, 1H), 7.97 (br. s, 1H), 7.82 (br. s, 1H), 7.45 (d, J=7.6 Hz, 1H), 6.21 (d, J=7.6 Hz, 1H), 4.34-4.27 (m, 1H), 3.04 (s, 3H), 3.01-2.99 (m, 1H), 2.76-2.73 (m, 1H), 2.36 (q, J=6.8 Hz, 2H), 2.19 (s, 3H), 1.98-1.88 (m, 1H), 1.73-1.70 (m, 1H), 1.60-1.44 (m, 2H), 1.01 (t, J=7.2 Hz, 3H). LCMS (ESI): calc, for C₁₉H₂₄F₃N₅O₂S [M+H]⁺ 458.1, found 458.3.

### Example 6. (R)—N-(2-(6-((1-(2-Hydroxyethyl) piperidin-3-yl)amino)-5-methylpyridazin-3-yl)-5-(trifluoromethyl)phenyl) methanesulfonamide (Compound 9)

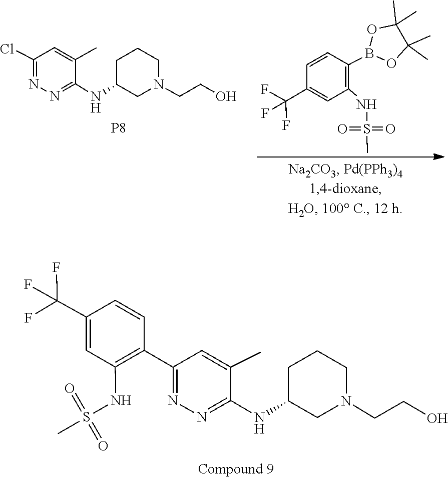

To a stirred solution of (R)-2-(3-((6-Chloro-4-methylpyridazin-3-yl)amino) piperidin-1-yl) ethan-1-ol (P9, 200 mg, 0.739 mmol, 1.0 eq) in 1,4-dioxane:H₂O (3:1, 8.0 mL) were added sodium carbonate (234 mg, 2.21 mmol, 3.0 eq) and (2-(methylsulfonamido)-4-(trifluoromethyl)phenyl) boronic acid (P2, 836 mg, 2.955 mmol, 4.0 eq) at ambient temperature and the reaction mixture degassed with N₂ for 15 min, followed by Pd(PPh₃)₄ (42 mg, 0.037 mmol, 0.1 eq) was added. The resulting reaction mixture was stirred at 100° C. for 12 h. The progress of the reaction was monitored by TLC and LCMS. After completion of starting material, reaction mixture was quenched with water (50 mL) and reaction mass extracted with EtOAc (3× 100 mL). The combined organic layers were dried over anh. Na₂SO₄, filtered and concentrated under reduced pressure. The residue was purified by Prep-HPLC to afford 26 mg (7.43%) of the title compound as an off white solid.

Analytical data: ¹H NMR (400 MHz, DMSO-d₆), δ: 12.59 (br. s, exch. with D₂O, 1H), 8.16 (br. s, 1H), 8.08 (d, J=8.4 Hz, 1H), 7.98 (s, 1H), 7.85 (br. s, 1H), 7.53 (d, J=8.0 Hz, 1H), 6.54 (br. s, 1H), 6.30 (d, J=7.6 Hz, 1H), 4.34-4.30 (m, 1H), 3.51 (t, J=6.4 Hz, 2H), 3.11 (s, 3H), 3.02-2.99 (m, 1H), 2.78-2.75 (m, 1H), 2.47-2.44 (m, 2H), 2.21 (s, 3H), 2.15-2.09 (m, 2H), 1.89-1.86 (m, 1H), 1.70-1.69 (m, 1H), 1.61-1.44 (m, 1H). LCMS (ESI): calc. for C₂₀H₂₆F₃N₅O₃S [M+H]⁺ 474.1, found 474.2.

### Example 7. (R)-5-Methyl-N-(1-methylpiperidin-3-yl)-6-(4-(trifluoromethyl)-1H-indazol-7-yl)pyridazin-3-amine (Compound 14)

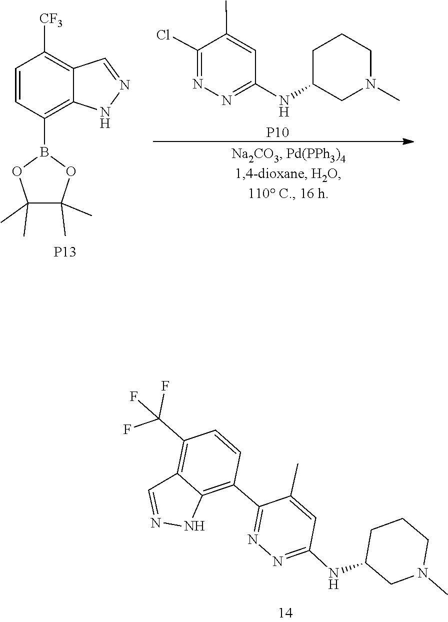

To a stirred solution of (R)-6-chloro-5-Methyl-N-(1-methylpiperidin-3-yl)pyridazin-3-amine (P10, 0.2 g, 0.83 mmol, 1.0 eq) in 1,4-dioxane (4.0 mL) were added 7-(4,4,5,5-tetramethyl-1,3,2-dioxaborolan-2-yl)-4-(trifluoromethyl)-1H-indazole (P13, 0.439 g, 1.6 mmol, 2.0 eq) and Na₂CO₃ (0.26 g, 2.49 mmol, 3.0 eq in 2 mL water) at ambient temperature. The reaction mixture was degassed for 10 min with Ar, followed by tetrakis(triphenylphosphine) palladium (0) (0.096 g, 0.08 mmol, 0.1 eq) was added at ambient temperature and the reaction mixture was stirred for 16 h at 110° C. The progress of the reaction was monitored by TLC. After completion of starting material, reaction mixture was quenched with water (50 mL) and extracted with EtOAc (3×50 mL). The combined organic layers were dried over anh. Na₂SO₄, filtered and concentrated under reduced pressure. The residue was subjected to RP Prep HPLC to afford 36 mg (11.1%) of the title compound (14) as an off white solid.

Analytical data: ¹H NMR (400 MHz, DMSO-d₆), δ: 13.46 (br. s, 1H), 8.23 (br. s, 1H), 7.6 (d, J=7.2 Hz, 1H), 7.52 (d, J=7.2 Hz, 1H), 6.84 (t, J=8.0 Hz, 2H), 4.13-4.11 (m, 1H), 2.88 (d, J=8.4 Hz, 1H), 2.67-2.49 (m, 1H), 2.19 (s, 3H), 2.07-2.0 (m, 4H), 1.93-1.84 (m, 2H), 1.75-1.71 (m, 1H), 1.60-1.50 (m, 1H), 1.38-1.29 (m, 1H). LCMS (ESI): calc. for C₁₉H₂₁F₃N₆ [M+H]⁺ 391.18, found 391.34.

### Example 8 (R)-2-(3-((5-Methyl-6-(4-(trifluoromethyl)-1H-indazol-7-yl) pyridazin-3-yl)amino) piperidin-1-yl) ethan-1-ol (Compound 16)

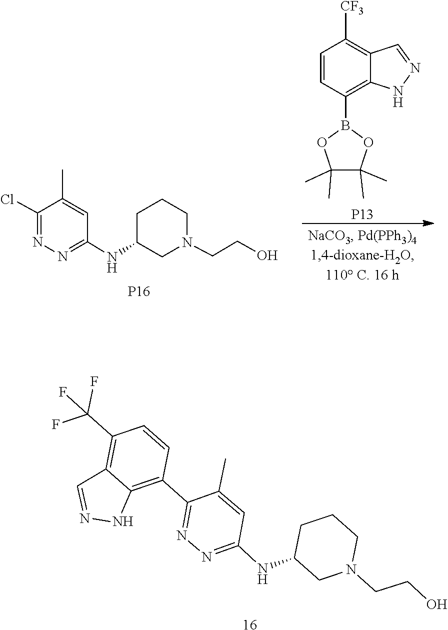

To a stirred solution of (R)-2-(3-((6-chloro-5-methylpyridazin-3-yl)amino) piperidin-1-yl) ethan-1-ol (P16, 0.15 g, 0.55 mmol, 1.0 eq) in 1,4-dioxane (3.0 mL) were added 7-(4,4,5,5-tetramethyl-1,3,2-dioxaborolan-2-yl)-4-(trifluoromethyl)-1H-indazole (P13, 0.346 g, 1.10 mmol, 2.0 eq) and Na₂CO₃ (0.176 g, 1.66 mmol, 3.0 eq in 1.0 mL water) at ambient temperature. The reaction mixture was degassed for 10 min with argon gas, tetrakis (triphenylphosphine) palladium (0) (0.064 g, 0.055 mmol, 0.1 eq) was added at ambient temperature and reaction mixture was stirred for 16 h at 110° C. The progress of the reaction was monitored by TLC. After completion of starting material, reaction mixture was quenched with water (50 mL) and extracted with EtOAc (3×50 mL). The combined organic layers were dried over anh. Na₂SO₄, filtered and concentrated under reduced pressure. The residue was purified by RP Prep HPLC to afford 42 mg (18%) of the title compound 16 as an off white solid.

Analytical data: ¹H NMR (400 MHz, DMSO-d₆), δ: 13.5 (br. s, 1H), 8.23 (br. s, 1H), 7.61 (d, J=8.0 Hz, 1H), 7.52 (d, J=7.2 Hz, 1H), 6.83 (t, J=8.4 Hz, 2H), 4.37 (t, J=5.6 Hz, 1H), 4.11 (br. s, 1H), 3.51 (dd, J=11.6 Hz and 6.0 Hz, 2H), 2.95 (d, J=10.0 Hz, 1H), 2.67-2.63 (m, 1H), 2.32 (t, J=2.0 Hz, 2H), 2.07 (s, 5H), 1.91-1.85 (s, 1H), 1.58-1.50 (m, 1H), 1.42-1.23 (m, 1H), 1.23-1.06. LCMS (ESI): calc, for C₂₀H₂₃F₃N₆O [M+H]⁺ 421.19, found 421.33.

### Example 8. (R)-4-Methyl-N-(1-methylpiperidin-3-yl)-6-(4-(trifluoromethyl)-1H-indazol-7-yl)pyridazin-3-amine (Compound 17)

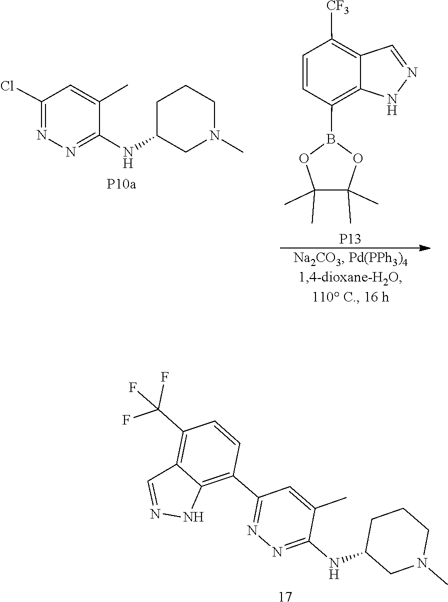

To a stirred solution of (R)-6-chloro-4-methyl-N-(1-methylpiperidin-3-yl)pyridazin-3-amine (P10A, 0.130 g, 0.541 mmol, 1.0 eq) in 1,4-dioxane (2.0 mL) were added 7-(4,4,5,5-tetramethyl-1,3,2-dioxaborolan-2-yl)-4-(trifluoromethyl)-1H-indazole (P13, 0.286 g, 1.08 mmol, 2.0 eq), Na₂CO₃ (0.172 g, 1.62 mmol, 3.0 eq in water 0.65 mL) at ambient temperature and the reaction mixture degassed with Ar for 10 min followed by Pd(PPh₃)₄ (0.063 g, 0.054 mmol, 0.1 eq) was added at ambient temperature. The reaction mixture was stirred for 16 h at 110° C. The progress of the reaction was monitored by TLC and LCMS. After completion of starting material, reaction mixture was quenched with water (50 mL) and extracted with EtOAc (3×50 mL). The combined organic layers were dried over anh. Na₂SO₄, filtered and concentrated under reduced pressure. The residue was purified by RP-Prep HPLC to afford 14 mg (6.6%) as an off white solid.

Analytical data: ¹H NMR (400 MHz, DMSO-d₆), δ: 13.58 (br. s, 1H), 8.23 (d, J=1.2 Hz, 1H), 8.07 (br. s, 1H), 8.03 (d, J=7.6 Hz, 1H), 7.62 (d, J=8.0 Hz, 1H), 6.16 (d, J=8.0 Hz, 1H), 4.43-4.36 (m, 1H), 3.0 (d, J=8.8 Hz, 1H), 2.67-2.54 (m, 1H), 2.23 (s, 3H), 2.2 (s, 3H), 2.20 (t, J=9.6 Hz, 3H), 1.75-1.71 (m, 1H), 1.61-1.55 (m, 1H), 1.51-1.42 (m, 1H). LCMS (ESI): calc, for C₁₉H₂₁F₃N₆ [M+H]⁺ 391.41, found 391.35.

### Example 9. (R)—N-(1-ethylpiperidin-3-yl)-4-methyl-6-(4-(trifluoromethyl)-1H-indazol-7-yl)pyridazin-3-amine (Compound 18)

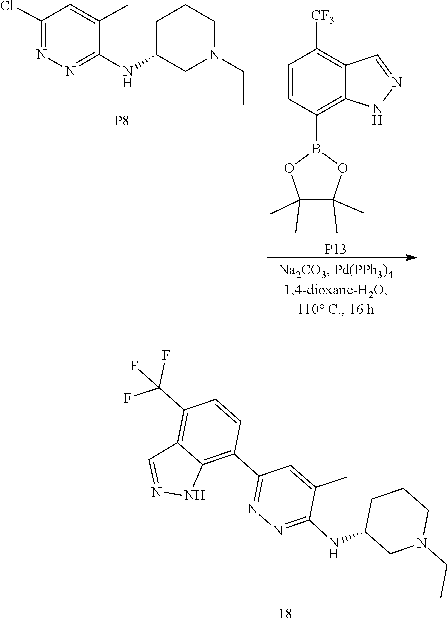

To a stirred solution of (R)-6-chloro-N-(1-ethylpiperidin-3-yl)-4-methylpyridazin-3-amine (P8, 0.15 g, 0.58 mmol, 1.0 eq) in 1,4-dioxane (3.0 mL) were added 7-(4,4,5,5-tetramethyl-1,3,2-dioxaborolan-2-yl)-4-(trifluoromethyl)-1H-indazole (P13, 0.367 g, 1.17 mmol, 2.0 eq) and Na₂CO₃ (0.187 g, 1.76 mmol, 3.0 eq in 1.0 mL water) at ambient temperature. The reaction mixture degassed for 10 min with Ar, tetrakis (triphenylphosphine) palladium (0) (0.068 g, 0.059 mmol, 0.1 eq) was added at ambient temperature and reaction mixture was stirred for 16 h at 110° C. The progress of the reaction was monitored by TLC. After completion of starting material, reaction mixture was quenched with water (50 mL) and extracted with EtOAc (3×50 mL). The combined organic layers were dried over anh. Na₂SO₄, filtered and concentrated under reduced pressure. The residue was purified by RP Prep HPLC to afford 26 mg (10.6%) of the title compound 18 as an off white solid.

Analytical data: ¹H NMR (400 MHz, DMSO-d₆), δ: 13.57 (br. s, 1H), 8.23 (br. s, 1H), 8.05 (t, J=8.4 Hz, 2H), 7.63 (d, J=7.6 Hz, 1H), 7.52-7.50 (m, 1H), 6.18 (br. s, 1H), 4.42 (br. s, 1H), 3.08 (br. s, 1H), 2.77 (br. s, 1H), 2.49-2.30 (m, 2H), 2.23 (s, 3H), 1.96 (br. s, 1H), 1.74 (s, 1H), 1.58-1.49 (m, 2H), 1.23 (s, 1H), 1.03 (s, 3H). LCMS (ESI): calc. for C₂₀H₂₃F₃N₆ [M+H]⁺ 405.19, found 405.33.

### Example 10. (R)—N-(3-methyl-2-(6-((1-methylpiperidin-3-yl)amino)pyridazin-3-yl)-5-(trifluoromethyl)phenyl) methanesulfonamide (Compound 1)

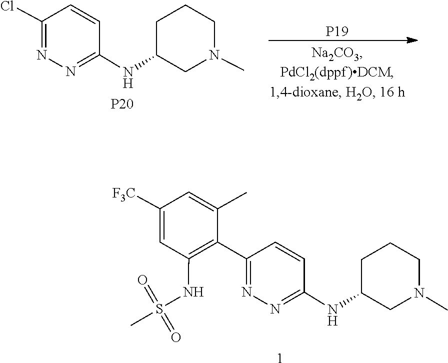

To a stirred solution of P20 (0.2 g, 0.885 mmol, 1.0 eq), P19 (669 mg, 1.77 mmol, 2.0 eq) in 1,4-dioxane (10 mL), water (2 mL) was added Na₂CO₃ (281 mg, 2.655 mmol, 3.0 eq) at ambient temperature. The reaction mixture was degassed with a stream of nitrogen for 15 min. Thereafter, PdCl₂(dppf)·DCM (72 mg, 0.088 mmol, 0.1 eq) was added at this temperature. The mixture was heated to 100° C., stirred for 16 h at 100° C. The progress of the reaction was monitored by TLC and LCMS. The reaction mixture was filtered through celite pad and the pad was washed with 10% MeOH in DCM (30 mL). The filtrate was washed with water (20 mL), brine (15 mL), dried over anh. Na₂SO₄, filtered and concentrated under reduced pressure at 45° C. The residue was purified by reverse phase column chromatography using 12 g C18 column using mixture of 0.1% FA in H₂O (30→50%) and ACN as an eluent to afford 23 mg (6%) of the title compound 1 as a pale brown solid.

Analytical data: ¹H NMR (400 MHz, DMSO-d₆), δ: 7.59 (s, 1H), 7.45 (s, 1H), 7.25 (d, J=9.2 Hz, 1H) 6.92 (d, J=9.2 Hz, 1H), 6.89 (d, exch. with D₂O, J=5.2 Hz, 1H), 4.21-4.12 (m, 1H), 2.97-2.85 (m, 1H), 2.87 (s, 3H), 2.69-2.66 (m, 1H), 2.20 (s, 3H), 2.12 (s, 3H), 2.15-1.95 (m, 2H), 1.89-1.83 (1H), 1.78-1.71 (m, 1H), 1.62-1.51 (m, 1H), 1.37-1.29 (m, 1H); LCMS (ESI): calc, for C₁₉H₂₄F₃N₅O₂S [M+H]⁺ 444.4, found 444.3.

### Example 11. (R)—N-(2-(6-((1-Ethylpiperidin-3-yl)amino)pyridazin-3-yl)-3-methyl-5-(trifluoromethyl)phenyl) methanesulfonamide (Compound 2)

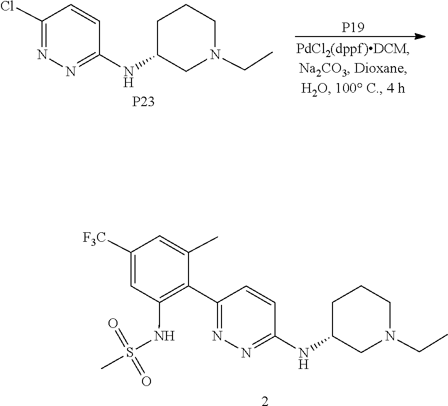

To a stirred solution of P23 (0.3 g, 1.24 mmol, 1.0 eq), P19 (1.41 mg, 3.73 mmol, 3.0 eq) in 1,4-dioxane (10 mL), water (2 mL) was added Na₂CO₃ (396 mg, 3.73 mmol, 3.0 eq) at ambient temperature. The reaction mixture was degassed with a stream of nitrogen for 15 min. Thereafter, PdCl₂ (dppf). DCM (50.8 mg, 0.062 mmol, 0.05 eq) was added at this temperature. The mixture was heated to 100° C., stirred for 4 h at 100° C. The progress of reaction was monitored by TLC and LCMS. The reaction mixture was filtered through celite pad and the pad was washed with 10% MeOH in DCM (30 mL). The filtrate was washed with water (20 mL), brine (15 mL), dried over anh. Na₂SO₄, filtered and concentrated under reduced pressure at 45° C. The residue was purified by reverse phase column chromatography using 12 g C18 column using mixture of 0.1% FA in H₂O (30→50%) and ACN to give 30 mg with HPLC purity: 84%, So further purified by reverse phase prep-HPLC to afford 8.5 mg (1.5%) of the title compound 2 as an off white solid.

Analytical data: ¹H NMR (400 MHz, DMSO-d₆), δ: 9.14 (br. s, exch. with D₂O, 1H), 7.55 (s, 1H), 7.20 (d, J=9.2 Hz, 2H), 6.85 (d, J=9.2 Hz, 1H), 6.73 (br. s, 1H), 4.12-3.97 (m, 1H), 3.13-2.96 (m, 1H), 2.87-2.62 (m, 4H), 2.39-2.32 (m, 2H), 2.08 (s, 3H), 2.03-2.01 (m, 1H), 1.97-1.85 (m, 2H), 1.73-1.70 (m, 1H), 1.58-1.49 (m, 1H), 1.36-1.31 (m, 1H), 1.00 (t, J=6.8 Hz, 3H); LCMS (ESI): calc, for C₂₀H₂₆F₃N₅O₂S [M+H]⁺ 458.18, found 458.10.

### Example 12. (R)-4-(4-Methyl-6-((1-methylpiperidin-3-yl)amino)pyridazin-3-yl)-7-(trifluoromethyl)-1,3-dihydrobenzo[c][1,2,5]thiadiazole 2,2-dioxide (Compound 10)

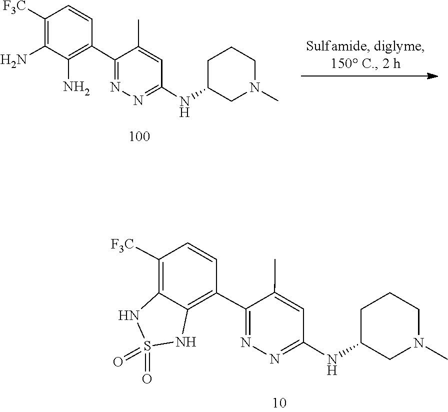 [SMILES: Cc1cc(N[C@@H]2CCCN(C)C2)nnc1-c1ccc(C(F)(F)F)c(N)c1N ; Cc1cc(N[C@@H]2CCCN(C)C2)nnc1-c1ccc(C(F)(F)F)c2c1NS(=O)(=O)N2]

To a stirred solution of (R)-3-(4-methyl-6-((1-methylpiperidin-3-yl)amino)pyridazin-3-yl)-6-(trifluoromethyl)benzene-1,2-diamine (Compound 100, 0.20 g, 0.52 mmol, 1.0 eq) in diglyme (4.0 mL) was added sulfamide (0.076 g, 0.78 mmol, 1.5 eq) at ambient temperature and stirred for 2 h at 150° C. The progress of the reaction was monitored by TLC and LCMS. After completion of starting material, reaction mixture was concentrated under reduced pressure. The residue was purified by PREP-HPLC to afford 40 mg (17%) of the title compound 10 as a pale-brown solid.

Analytical data: ¹H NMR (400 MHz, DMSO-d₆), &: 9.68-9.58 (m, 1H), 9.03 (s, 1H), 6.83-6.19 (m, 4H), 4.35-4.20 (m, 1H), 3.65-3.57 (m, 1H), 3.25-3.10 (m, 1H), 2.85-2.60 (m, 5H), 2.22-1.90 (m, 5H), 1.80-1.65 (m, 1H), 1.52-1.35 (m, 1H). LCMS (ESI): calc. for C₁₈H₂₂F₃N₆O₂S [M+H]⁺ 443.14, found 443.25.

### Example 13. (R)—N-(1-Ethylpiperidin-3-yl)-5-methyl-6-(4-(trifluoromethyl)-1H-indazol-7-yl)pyridazin-3-amine (Compound 15)

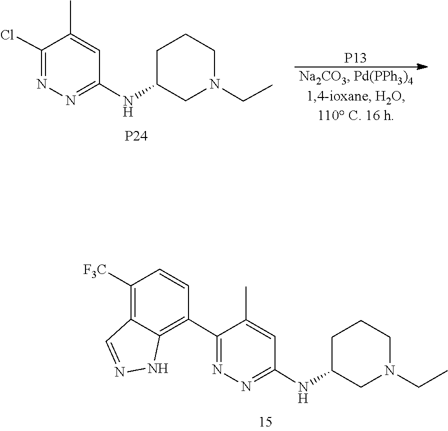

To a stirred solution of P24 (0.15 g, 0.59 mmol, 1.0 eq) in 1,4-dioxane (3.0 mL) were added P13 (0.36 g, 1.18 mmol, 2.0 eq) and Na₂CO₃ (0.188 g, 1.77 mmol, 3.0 eq in 0.75 mL water) at ambient temperature. The reaction mixture was degassed for 10 min with Ar gas, tetrakis(triphenylphosphine) palladium (0) (0.06 g, 0.059 mmol, 0.1 eq) was added at ambient temperature and reaction mixture was stirred for 16 h at 110° C. The progress of the reaction was monitored by TLC and LCMS. After completion of the starting material, reaction mixture was quenched with water (50 mL) and extracted with EtOAc (3× 50 mL). The combined organic layers were dried over anh. Na₂SO₄, filtered and concentrated under reduced pressure. The residue was subjected to RP Prep HPLC to afford 40 mg (16.7%) of the title compound 15 as an off white solid.

Analytical data: ¹H NMR (400 MHz, DMSO-d₆), δ: 13.45 (br. s, 1H), 8.24 (br. s, 1H), 7.62 (dd, J=0.8 Hz and 6.8 Hz, 1H), 7.52 (d, J=7.2 Hz, 1H), 6.82 (br. s, 2H), 4.11-4.07 (m, 1H), 2.99 (d, J=8.8 Hz, 1H), 2.69-2.66 (m, 1H), 2.39-2.32 (m, 2H), 2.07-1.88 (m, 6H), 1.75-1.70 (m, 1H), 1.59-1.51 (m, 1H), 1.39-1.30 (m, 1H), 1.02 (s, 3H). LCMS (ESI): calc, for C₂₀H₂₃F₃N₆ [M+H]⁺ 405.2, found 405.31.

### Example 14. (R)-5-Methyl-N-(1-methylpiperidin-3-yl)-6-(4-(trifluoromethyl)-1H-benzo[d][1,2,3]triazol-7-yl)pyridazin-3-amine (Compound 20)

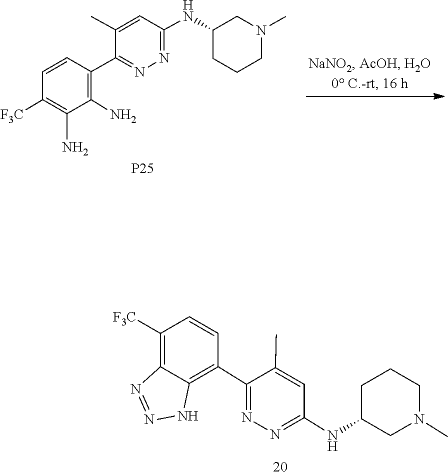

To a stirred solution of P25 (0.10 g, 0.263 mmol, 1.0 eq) in AA (1 mL) was added a solution of NaNO₂ (0.027 g, 0.394 mmol, 1.5 eq) in water (1.5 mL) at 0° C. and stirred at ambient temperature for 16 h. The progress of the reaction was monitored by TLC and LCMS. After completion of starting material, the reaction mixture was concentrated under reduced pressure. The residue was subjected to reverse phase prep-HPLC purification using ammonium carbonate in water and ACN as an eluent to afford 42 mg (42%) of the title compound 20 as a pale-yellow solid.

Analytical data: ¹H NMR (400 MHz, DMSO-d₆), δ: 14.81 (br. s, 1H), 7.85 (d, J=7.6 Hz, 1H), 7.62 (d, J=7.2 Hz, 1H), 6.98 (d, J=7.6 Hz, 1H), 6.84 (s, 1H), 4.25-4.10 (m, 1H), 3.11-3.05 (m, 1H), 2.80-2.72 (m, 1H), 2.36-2.29 (m, 4H), 2.21-2.18 (m, 1H), 2.13 (s, 3H), 1.95-1.85 (m, 1H), 1.85-1.75 (m, 1H), 1.68-1.53 (m, 1H), 1.43-1.38 (m, 1H). LCMS (ESI): calc. for C₁₈H₂₀F₃N₇ [M+H]⁺ 392.17, found 392.15.

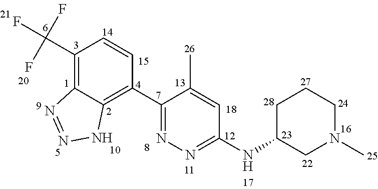 [SMILES: Cc1cc(N[C@@H]2CCCN(C)C2)nnc1-c1ccc(C(F)(F)F)c2nn[nH]c12]

Structure of 20 was confirmed by NOE experimental data: On irradiation of H18 (6.84 ppm) proton giving enhancement of H17 (D20 exchangeable proton) at 6.98 ppm and low enhancement of H23 at 4.17 ppm and no enhancement of aromatic protons.

### Example 15. (R)-4,5-dimethyl-N-(1-methylpiperidin-3-yl)-6-(4-(trifluoromethyl)-1H-benzo[d][1,2,3]triazol-7-yl)pyridazin-3-amine (Compound 22)

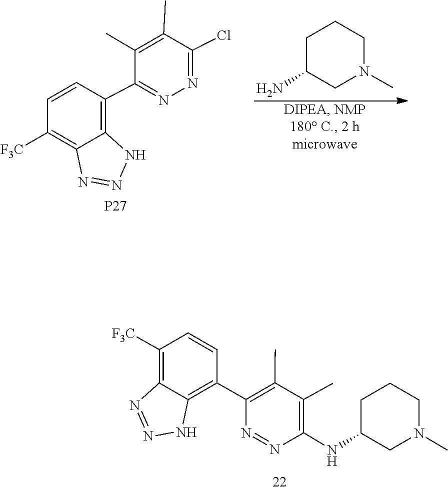

To a stirred solution of P27 (250 mg, 0.76 mmol, 1.0 eq) in NMP (2.0 mL) were added N,N-diisopropylethylamine (0.27 mL, 1.52 mmol, 2.0 eq) and (R)-1-methylpiperidin-3-amine (433 mg, 3.80 mmol, 5.0 eq) at rt and the resulting mixture was stirred for 2 h at 180° C. in microwave. The progress of reaction was monitored by LCMS. After completion of the starting material, the reaction mixture was concentrated under reduced pressure. The residue was purified by reverse phase PREP-HPLC to afford 14 mg (4.5%) of the title compound 22 as a pale-yellow solid.

Analytical data: ¹H NMR (400 MHz, DMSO-d₆), δ: 7.33-7.29 (m, 1H), 6.97 (d, J=6.8 Hz, 1H), 4.51 (t, J=11.2 Hz, 1H), 4.15-4.10 (m, 1H), 3.99-3.92 (m, 1H), 2.95-2.90 (m, 1H), 2.85-2.75 (m, 1H), 2.52 (s, 3H), 2.14 (s, 3H), 1.92 (s, 3H), 1.82-1.72 (m, 3H), 1.65-1.60 (m, 1H). LCMS (ESI): calc, for C₁₉H₂₃F₃N₇ [M+H]⁺ 406.19, found 406.32.

### Example 16. (R)—N(1-Methylpiperidine-3-yl)-5-(trifluoromethyl)-6-(trifluoromethyl)-1H-benzo[d][1,2,3]triazol-7-yl)pyridazin-3-amine (Compound 23)

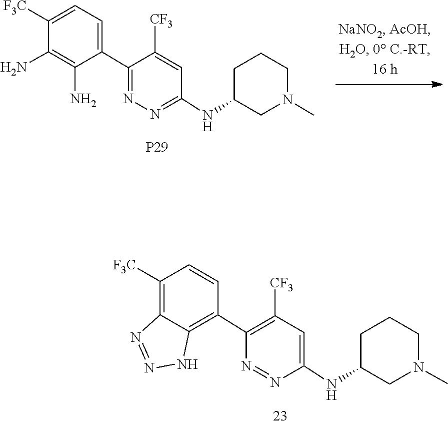

To a stirred solution of P29 (0.15 g, 0.35 mmol, 1.0 eq) in AA (0.08 mL, 1.38 mmol, 4.0 eq) was added a solution of NaNO₂ (47.6 mg, 0.69 mmol, 2.0 eq) in water (0.4 mL) at 0° C. and stirred at ambient temperature for 16 h. The progress of the reaction was monitored by TLC and LCMS. After completion of starting material, the reaction mixture was concentrated under reduced pressure. The residue was subjected to reverse column chromatography using ammonium bicarbonate in water and ACN as an eluant to afford 23 mg (15%) of the title compound 23 as an off-white solid.

Analytical data: ¹H NMR (400 MHz, DMSO-d₆), δ: 7.82 (d, J=7.6 Hz, 1H), 7.58 (d, J=7.6 Hz, 1H), 7.50 (d, J=7.2 Hz, 1H), 7.42 (s, 1H), 4.29-4.21 (m, 1H), 2.89-2.87 (m, 1H), 2.59-2.52 (m, 1H), 2.30-2.20 (m, 5H), 1.90-1.70 (m, 2H), 1.63-1.56 (m, 1H), 1.50-1.40 (m, 1H). LCMS (ESI): calc, for C₁₈H₁₇F₆N₇ [M+H]⁺ 446.14, found 446.27.

### Example 17. (R)—N(1-Methylpiperidine-3-yl)-5-(trifluoromethyl)-6-(trifluoromethyl)-1H-benzo[d][1,2,3]triazol-7-yl)pyridazin-3-amine (Compound 24)

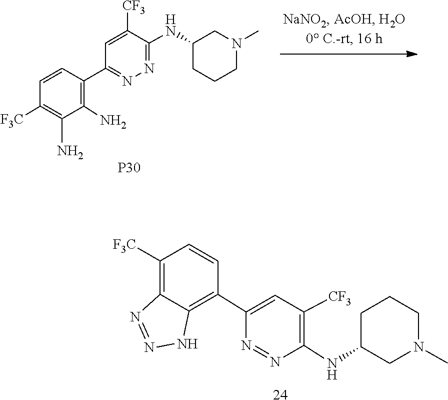

To a stirred solution of P30 (0.10 g, 0.23 mmol, 1.0 eq) in AA (0.2 mL) was added a solution of sodium nitrite (0.032 g, 0.460 mmol, 2.0 eq) in water (0.2 mL) at 0° C. The reaction mixture was stirred for 16 h at ambient temperature. The progress of the reaction was monitored by TLC and LCMS. The reaction mixture concentrated under reduced pressure to get the crude compound. Following the above procedure, another 100 mg of P30 was treated with sodium nitrite in AcOH and both these batches were mixed. The combined crude compound was subjected to reverse phase PREP-HPLC purification using ammonium bicarbonate in water and ACN as an eluant to afford 13 mg (6.3%) of the title compound 24 as an off-white solid.

Analytical data: ¹H NMR (400 MHz, DMSO-d₆), &: 9.19 (br. s, 1H), 8.26 (d, J=8.0 Hz, 1H), 7.75-7.70 (m, 1H) 6.51-6.49 (m, 1H), 4.70-4.60 (m, 1H), 3.32-3.22 (m, 1H), 2.95-2.85 (m, 1H), 2.65-2.55 (m, 1H), 2.40-2.30 (m, 4H), 1.85-1.70 (m, 3H), 1.70-1.58 (m, 1H); LCMS (ESI): calc, for C₁₈H₁₇F₆N₇ [M+H]⁺ 446.14, found 446.23.

### Example 18. (R)—N-(2-(4-((1-Methylpiperidin-3-yl)amino)-5,6,7,8-tetrahydrophthalazin-1-yl)-5-(trifluoromethyl)phenyl) methane sulfonamide (Compound 50)

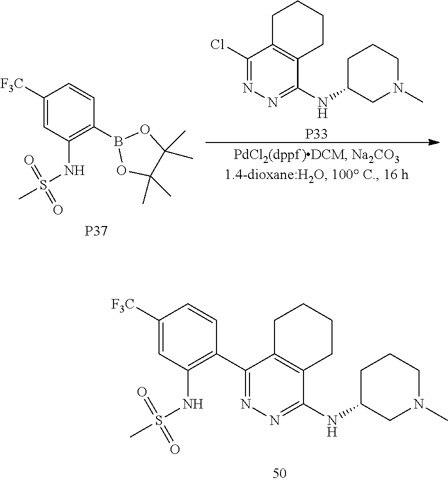 [SMILES: "P37" ; CC1(C)OB(c2ccc(C(F)(F)F)cc2NS(C)(=O)=O)OC1(C)C ; CN1CCC[C@@H](Nc2nnc(-c3ccc(C(F)(F)F)cc3NS(C)(=O)=O)c3c2CCCC3)C1 ; "P33" ; CN1CCC[C@@H](Nc2nnc(Cl)c3c2CCCC3)C1]

To a stirred solution of P37 (0.2 g, 0.71 mmol, 1 eq) and P33 (0.390 g, 1.068 mmol, 1.5 eq) in 1,4-dioxane (4.0 mL) was added a solution of Na₂CO₃ (0.226 g, 2.137 mmol, 3.0 eq) in water (1.0 mL) at ambient temperature. The reaction mixture was degassed for 10 min with argon gas, then PdCl₂(dppf)·DCM (0.058 g, 0.071 mmol, 0.1 eq) was added at ambient temperature. The resulting reaction mixture was stirred for 16 h at 100° C. The progress of the reaction was monitored by TLC and LCMS. After completion of starting material, reaction mixture was quenched with water (50 mL) and extracted with EtOAc (3×50 mL). The combined organic layers were dried over anh. Na₂SO₄, filtered and concentrated under reduced pressure. The residue was purified by RP Prep HPLC to afford 55 mg (16%) of the title compound 50 as a brown solid.

Analytical data: ¹H NMR (400 MHz, DMSO-d₆), δ: 8.18 (br. s, 1H), 7.74 (br. s, 1H), 7.39 (br. s, 2H), 5.65 (d, J=7.6 Hz, 1H), 4.30-4.28 (m, 1H), 3.32-2.87 (m, 4H), 2.67-2.61 (m, 1H), 2.38-2.31 (m, 2H), 2.28-2.22 (m, 2H), 2.21 (s, 3H), 2.04-1.97 (m, 2H), 1.88-1.80 (m, 1H), 1.79-1.70 (m, 3H), 1.61-1.52 (m, 3H), 1.48-1.40 (m, 1H). LCMS (ESI): calc. for C₂₂H₂₈F₃N₅O₂S [M+H]⁺ 484.19, found 484.35.

### Example 19. (R)—N-(2-(4-((1-Ethylpiperidin-3-yl)amino)-5,6,7,8-tetrahydrophthalazin-1-yl)-5-(trifluoromethyl)phenyl) methane sulfonamide (Compound 37)

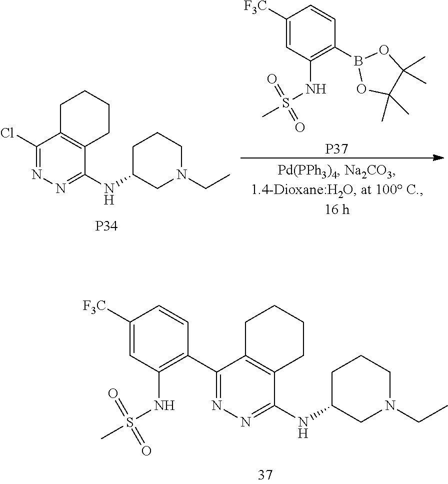 [SMILES: "P34" ; CCN1CCC[C@@H](Nc2nnc(Cl)c3c2CCCC3)C1 ; CCN1CCC[C@@H](Nc2nnc(-c3ccc(C(F)(F)F)cc3NS(C)(=O)=O)c3c2CCCC3)C1 ; "P37" ; CC1(C)OB(c2ccc(C(F)(F)F)cc2NS(C)(=O)=O)OC1(C)C]

To a stirred solution (R)-4-chloro-N-(1-ethylpiperidin-3-yl)-5,6,7,8-tetrahydrophthalazin-1-amine (P34, 0.2 g, 0.678 mmol, 1 eq) in 1,4-dioxane (4.0 mL), N-(2-(4,4,5,5-tetramethyl-1,3,2-dioxaborolan-2-yl)-5-(trifluoromethyl)phenyl) methane sulfonamide (P37, 0.372 g, 1.018 mmol, 1.5 eq) was added a solution of Na₂CO₃ (0.216 g, 2.105 mmol, 3.0 eq) in water (2.0 mL) at ambient temperature. The reaction mixture was degassed for 10 min with argon gas, then followed Pd(PPh₃)₄ (0.039 g, 0.034 mmol, 0.05 eq) at ambient temperature and reaction mixture was stirred for 16 h at 100° C. The progress of the reaction was monitored by TLC and LCMS. After completion of starting material, reaction mixture was quenched with ice water (50 mL) and extracted with EtOAc (3×50 mL). The combined organic layers were dried over anh. Na₂SO₄, filtered and concentrated under reduced pressure. The residue was purified by RP Prep HPLC to get 33 mg (10%) of the title compound 37 as an off-white solid.

Analytical data: ¹H NMR (400 MHz, DMSO-d₆), δ: 9.29 (br. s, exch. with D₂O, 1H), 7.76 (s, 1H), 7.45-7.43 (m, 2H), 5.72 (d, J=7.6 Hz, 1H, exch. with D₂O), 4.32-4.30 (m, 1H), 3.09-3.01 (m, 1H), 2.92 (s, 3H), 2.79-2.75 (m, 1H), 2.51-2.49 (m, 2H), 2.38-2.35 (m, 2H), 2.29-2.20 (m, 2H), 2.07-2.05 (m, 2H), 1.90-1.86 (m, 1H), 1.76-1.72 (m, 3H), 1.60-1.45 (m, 4H), 0.85 (t, J=5.6 Hz, 3H). LCMS (ESI): calc, for C₂₃H₃₁F₃N₅O₂S [M+H]⁺ 498.21, found 498.32.

### Example 20. (R)—N-(2-(4-((1-Ethylpiperidin-3-yl)amino)-5,6,7,8-tetrahydro-5,8-ethanophthalazin-1-yl)-5-(trifluoromethyl)phenyl) methanesulfonamide (Compound 30)

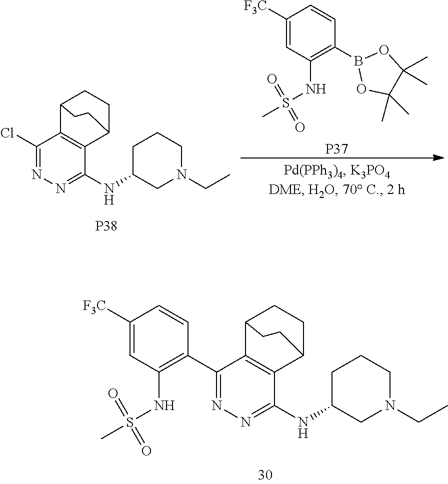 [SMILES: "P38" ; CCN1CCC[C@@H](Nc2nnc(Cl)c3c2C2CCC3CC2)C1 ; CCN1CCC[C@@H](Nc2nnc(-c3ccc(C(F)(F)F)cc3NS(C)(=O)=O)c3c2C2CCC3CC2)C1 ; "P37" ; CC1(C)OB(c2ccc(C(F)(F)F)cc2NS(C)(=O)=O)OC1(C)C]

To a stirred solution of (R)-4-chloro-N-(1-ethylpiperidin-3-yl)-5,6,7,8-tetrahydro-5,8-ethanophthalazin-1-amine (P38, 0.15 g, 0.468 mmol, 1 eq) in DME/water (10 mL, 8:2) was added N-(2-(4,4,5,5-tetramethyl-1,3,2-dioxaborolan-2-yl)-5-(trifluoromethyl)phenyl) methanesulfonamide (P37, 342 mg, 0.937 mmol, 2.0 eq), potassium phosphate (298.3 mg, 1.405 mmol, 3.0 eq) at ambient temperature. The mixture was degassed with a stream of nitrogen for 5 min then added Pd(PPh₃)₄ (0.541 mg, 0.047 mmol, 0.1 eq) at ambient temperature. The resulting reaction mixture was stirred for 2 h at 70° C. The Progress of reaction was monitored by TLC and LCMS. The reaction mixture was filtered, and the filtrate was concentrated under reduced pressure. The residue was purified by prep HPLC to afford 19 mg (7%) of the title compound 30 as a white solid.

Analytical data: ¹H NMR (400 MHz, DMSO-d₆), δ: 9.62 (br. s, exch. with D₂O, 1H), 7.79 (s, 1H), 7.62-7.42 (m, 2H), 6.32-6.18 (m, 1H), 4.32-4.22 (m, 1H), 3.41-3.38 (m, 1H), 3.24-3.16 (m, 1H), 3.04-2.80 (m, 4H), 2.75-2.72 (m, 1H), 2.46-2.38 (m, 2H), 2.12-1.85 (m, 3H), 1.80-1.52 (m, 6H), 1.50-1.16 (m, 5H), 1.03 (t, J=6.8 Hz, 3H). LCMS (ESI): calc. for C₂₃H₃₀F₃N₅O₂S [M+H]⁺ 524.22, found 524.32.

### Example 21. (R)—N-(2-(6-((1-Ethylpiperidin-3-yl)amino)-5-methylpyridazin-3-yl)-5-(trifluoro methyl)phenyl) ethanesulfonamide (Compound 62)

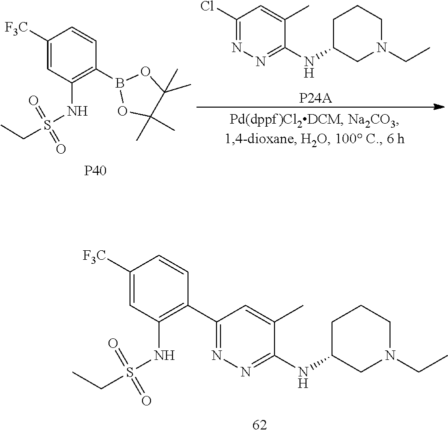

To a stirred solution of (R)-6-chloro-N-(1-ethylpiperidin-3-yl)-4-methylpyridazin-3-amine (P24A, 150 mg, 0.58 mmol, 1.0 eq), N-(2-(4,4,5,5-tetramethyl-1,3,2-dioxaborolan-2-yl)-5-(trifluoromethyl)phenyl) ethanesulfonamide (P40, 558 mg, 1.47 mmol, 2.5 eq) in a mixture of 1,4-dioxane (6.0 mL) and water (1.5 mL) was added Na₂CO₃ (187 mg, 1.76 mmol, 3.0 eq) at ambient temperature. The mixture was degassed with a stream of nitrogen for 5 min. Thereafter, Pd(dppf)Cl₂·DCM (48.0 mg, 0.059 mmol, 0.1 eq) was added. The mixture was heated to 100° C., stirred for 6 h at this temperature. The progress of reaction was monitored by TLC and LCMS. The reaction mixture was filtered through celite pad, the pad was washed with 1,4-dioxane (10 mL). The filtrate was diluted with sat. NaHCO₃ solution (10 mL) and extracted with EtOAc (2×10 mL), dried over anh. Na₂SO₄, and filtered, concentrated under reduced pressure at 45° C. to give residue. The residue was purified by reverse phase Prep-HPLC to afford 33 mg (12%) of the title compound 62 as a pale brown solid.

Analytical data: ¹H NMR (400 MHz, DMSO-d₆), δ: 12.86 (br. s, exch. with D₂O, 1H), 8.10 (d, J=8.4 Hz, 1H), 8.02 (s, 1H), 7.91 (d, J=1.2 Hz, 1H), 7.54 (d, J=8.4 Hz, 1H), 6.32 (d, J=7.2 Hz, 1H), 4.33-4.31 (m, 1H), 3.19 (q, J=7.2 Hz, 2H), 3.02 (br. s, 1H), 2.77 (br. s, 1H), 2.40 (br. s, 2H), 2.22 (s, 3H), 1.99-1.89 (m, 3H), 1.75-1.72 (m, 1H), 1.59-1.46 (m, 2H), 1.07-0.99 (m, 6H); LCMS (ESI): calc. for: C₂₁H₂₉F₃N₅O₂S [M+H]⁺ 472.19, found 472.25.

### Example 22. (R)—N-(2-(6-((1-Ethylpiperidin-3-yl)amino)pyridazin-3-yl)-3-methyl-5-(trifluoromethyl)phenyl) propane-2-sulfonamide (Compound 72)

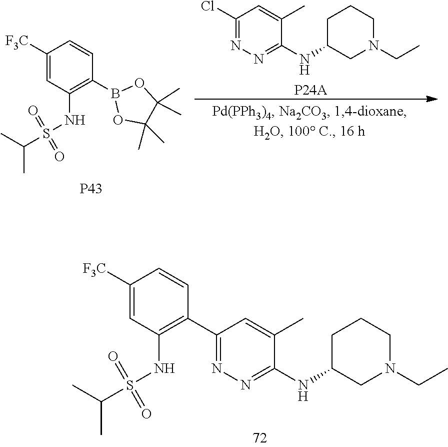

To a stirred solution of (R)-6-chloro-N-(1-ethylpiperidin-3-yl)-4-methylpyridazin-3-amine (P24A, 200 mg, 0.78 mmol, 1.0 eq), N-(2-(4,4,5,5-tetramethyl-1,3,2-dioxaborolan-2-yl)-5-(trifluoromethyl)phenyl) propane-2-sulfonamide (P43, 926 mg, 2.35 mmol, 3.0 eq) in mixture of solvents 1,4-dioxane (8.0 mL) and water (2.0 mL) was added Na₂CO₃ (249 mg, 2.35 mmol, 3 eq) at ambient temperature. The mixture was degassed with a stream of nitrogen for 5 min. Thereafter, Pd(PPh₃)₄ (45.3 mg, 0.039 mmol, 0.05 eq) was added. The mixture was stirred for 16 h at 100° C. The progress of reaction was monitored by TLC and LCMS. The reaction mixture was filtered through celite pad, the pad was washed with 1,4-dioxane (10 mL). The filtrate was diluted with sat. NaHCO₃ solution (10 mL) and extracted with EtOAc (2×10 mL), dried over anh. Na₂SO₄, and filtered, concentrated under reduced pressure at 45° C. to give residue. The residue was purified by reverse phase Prep-HPLC to afford 32 mg (8%) of the title compound 72 as a pale brown solid.

Analytical data: ¹H NMR (400 MHz, DMSO-d₆), δ: 12.92 (br. s, exch. with D₂O, 1H), 8.11 (d, J=7.6 Hz, 1H), 8.03 (s, 1H), 7.97 (s, 1H), 7.52 (br. s, 1H), 6.31 (br. s, 1H), 4.31-4.29 (m, 1H), 3.27 (br. s, 1H) 3.01 (d, J=8.0 Hz, 1H), 2.75 (d, J=10.4 Hz, 1H), 2.40-2.35 (m, 2H), 2.23 (s, 3H), 1.99-1.90 (m, 3H), 1.74-1.71 (m, 1H), 1.58-1.45 (m, 2H), 1.15-1.12 (m, 6H), 1.01 (t, J=7.2 Hz, 3H); LCMS (ESI): calc. for: C₂₂H₃₀F₃N₅O₂S [M+H]⁺ 486.21, found 486.29.

### Example 23. (R)—N-(2-(6-((1-Ethylpiperidin-3-yl)amino)-5-methylpyridazin-3-yl)-5-(trifluoro methyl)phenyl)-1H-imidazole-5-sulfonamide (Compound 68)

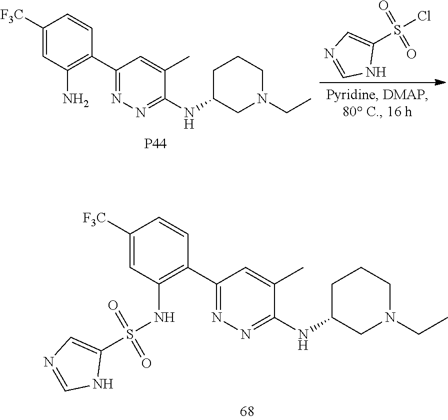

To a stirred solution of (R)-6-(2-amino-4-(trifluoromethyl)phenyl)-N-(1-ethylpiperidin-3-yl)-4-methylpyridazin-3-amine (P44, 280 mg, 0.74 mmol, 1.0 eq) in pyridine (5.6 mL) was added DMAP (9.0 mg, 0.074 mmol, 0.1 eq) followed by 1H-imidazole-5-sulfonyl chloride (147 mg, 0.88 mmol, 1.2 eq) at ambient temperature. The resulting reaction mixture was stirred for 16 h at 80° C. The progress of reaction was monitored by TLC and LCMS. The reaction mixture was concentrated under reduced pressure at 50° C. then sat. NaHCO₃ solution (10 mL) was added. The mixture was stirred for 10 min at ambient temperature, the precipitated solid was collected by filtration, dried to give residue. The residue was purified by reverse phase Prep-HPLC to afford 68 mg (18%) of the title compound 68 as a pale yellow solid.

Analytical data: ¹H NMR (400 MHz, DMSO-d₆), δ: 13.21 (br. s, exch. with D₂O, 1H), 12.81 (br. s, exch. with D₂O, 1H), 7.99-7.89 (m, 4H), 7.73 (s, 1H), 7.41 (d, J=8.0 Hz, 1H), 6.28 (d, J=5.6 Hz, 1H), 4.35-4.33 (m, 1H), 3.02 (d, J=7.2 Hz, 1H), 2.77 (d, J=9.6 Hz, 1H), 2.42-2.40 (m, 2H), 2.20 (s, 3H), 2.04-1.91 (m, 3H), 1.75-1.72 (m, 1H), 1.62-1.47 (m, 2H), 1.05 (t, J=7.2 Hz, 3H); LCMS (ESI): calc. for: C₂₂H₂₆F₃N₇O₂S [M+H]⁺ 510.18, found 510.29.

### Example 24. (R)—N-(2-(5-methyl-6-(piperidin-3-ylamino)pyridazin-3-yl)-5-(trifluoromethyl)phenyl) methanesulfonamide (Compound 67)

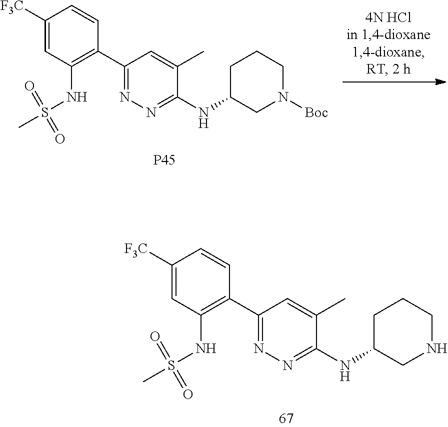

To a stirred solution of tert-butyl (R)-3-((5-methyl-6-(2-(methylsulfonamido)-4-(trifluoromethyl)phenyl) pyridazin-3-yl)amino)piperidine-1-carboxylate (0.44 g, 0.83 mmol, 1.0 eq) in 1,4-dioxane (4 mL) was added 4N HCl in 1,4-dioxane (4 mL) at ambient temperature. Resulting reaction mixture was stirred for 2 h at ambient temperature. Progress of the reaction was monitored by TLC and LCMS. Reaction mixture was concentrated under reduced pressure. Crude was triturated with n-pentane (5 mL) to get 340 mg (crude) of the title compound 67. The crude compound (120 mg) was subjected to reverse phase prep-HPLC purification using ammonium bicarbonate in water and ACN as an eluent to afford 20 mg of the title compound 67 as an off-white solid.

Analytical data: ¹H NMR (400 MHz, DMSO-d₆), δ: 7.97 (d, J=0.8 Hz, 1H), 7.93 (d, J=8.4 Hz, 1H), 7.79 (d, J=0.8 Hz, 1H), 7.32 (d, J=8.0 Hz, 1H), 6.22 (d, J=7.6 Hz, 1H), 4.31-4.29 (m, 1H), 3.32-3.24 (m, 1H), 2.98-2.95 (m, 4H), 2.62-2.55 (m, 2H), 2.19 (s, 3H), 2.01-1.97 (m, 1H), 1.80-1.72 (m, 1H), 1.69-1.52 (m, 2H). LCMS (ESI): calc. for C₁₈H₂₂F₃N₅O₂S [M+H]⁺ 430.14, found 430.35.

### Example 25. (R)—N-(2-(6-((1-Isopropylpiperidin-3-yl)amino)-5-methylpyridazin-3-yl)-5-(trifluoromethyl)phenyl) methanesulfonamide (Compound 25)

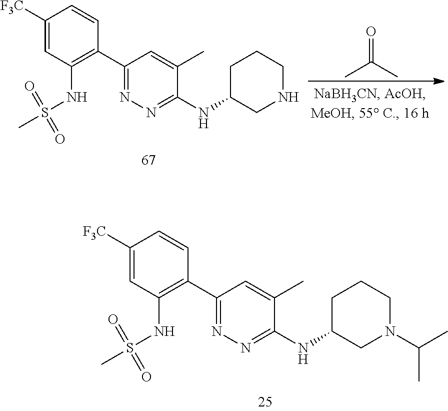

To a stirred solution of (R)—N-(2-(5-methyl-6-(piperidin-3-ylamino)pyridazin-3-yl)-5-(trifluoromethyl)phenyl) methanesulfonamide (0.3 g, 0.699 mmol, 1 eq) in methanol (6 mL) was added acetone (0.686 mL, 6.985 mmol, 10 eq), AA (0.1 mL) and sodium cyanoborohydride (0.088 g, 1.397 mmol, 2 eq) at ambient temperature. Resulting reaction mixture was heated to 55° C., stirred for 16 h. Progress of the reaction was monitored by TLC and LCMS. Reaction mixture was concentrated under reduced pressure. Crude was dissolved in 10% methanol/DCM (15 mL), washed with aq. NaHCO₃ solution (10 mL), water (10 mL) and brine (10 mL) solution. The organic layer was dried over sodium sulfate and concentrated under reduced pressure. The residue was subjected to reverse phase prep-HPLC purification using ammonium bicarbonate in water and ACN as an eluent to afford 65 mg (19%) of the title compound 25 as an off-white solid.

Analytical data: ¹H NMR (400 MHz, DMSO-d₆) δ: 12.76-12.65 (m, 1H), 8.06 (d, J=8.4 Hz, 1H), 7.98 (s, 1H), 7.84 (s, 1H), 7.51 (d, J=6.8 Hz, 1H), 6.25 (d, J=6.8 Hz, 1H), 4.31-4.29 (m, 1H), 3.09 (s, 3H), 2.95 (d, J=8.0 Hz, 1H), 2.80-2.67 (m, 2H), 2.28-2.14 (m, 5H), 1.90-1.88 (m, 1H), 1.74-1.70 (m, 1H), 1.54-1.45 (m, 2H), 0.98 (t, J=6.8 Hz, 6H). LCMS (ESI): calc, for C₂₁H₂₈F₃N₅O₂S [M+H]⁺ 472.19, found 472.30.

### Example 26. (R)—N-(2-(6-((1-Cyclopropylpiperidin-3-yl)amino)-5-methylpyridazin-3-yl)-5-(trifluoromethyl)phenyl) methanesulfonamide (Compound 42)

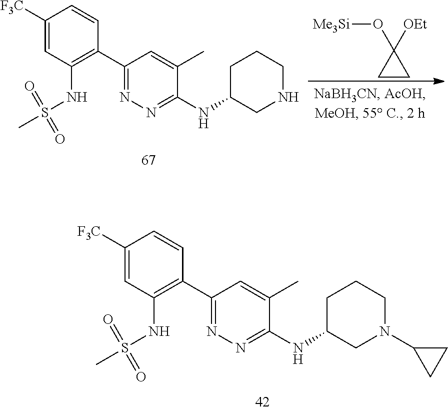 [SMILES: Cc1cc(-c2ccc(C(F)(F)F)cc2NS(C)(=O)=O)nnc1N[C@@H]1CCCNC1 ; CCOC1(O[Si](C)(C)C)CC1 ; "67" ; Cc1cc(-c2ccc(C(F)(F)F)cc2NS(C)(=O)=O)nnc1N[C@@H]1CCCN(C2CC2)C1]

To a stirred solution of (R)—N-(2-(5-methyl-6-(piperidin-3-ylamino)pyridazin-3-yl)-5-(trifluoromethyl)phenyl) methanesulfonamide (compound 67, 0.18 g, 0.419 mmol, 1 eq) in methanol (8 mL) was added (1-ethoxycyclopropoxy) trimethylsilane (0.365 g, 2.096 mmol, 5 eq), AA (0.1 mL) and sodium cyanoborohydride (0.053 g, 0.838 mmol, 2 eq) at ambient temperature. Resulting reaction mixture was stirred at 55° C. for 2 h. Progress of the reaction was monitored by TLC and LCMS. Reaction mixture was concentrated under reduced pressure. Crude was dissolved in 10% methanol/DCM (25 mL), washed with aq. NaHCO₃ solution (10 mL), water (10 mL) and brine (10 mL) solution. The organic layer was dried over sodium sulfate and concentrated under reduced pressure. The residue was subjected to reverse phase prep-HPLC purification using ammonium bicarbonate in water and ACN as an eluent to afford 32 mg (16%) of the title compound 42 as an off-white solid.

Analytical data: ¹H NMR (400 MHz, DMSO-d₆), δ: 12.80 (s, 1H), 8.08 (d, J=8.4 Hz, 1H), 7.98 (s, 1H), 7.85 (s, 1H), 7.56 (d, J=7.6 Hz, 1H), 6.26 (d, J=6.8 Hz, 1H), 4.25-4.23 (m, 1H), 3.11-3.08 (m, 4H), 2.84-2.82 (m, 1H), 2.25-2.21 (m, 5H), 1.91-1.89 (m, 1H), 1.72-1.60 (m, 2H), 1.54-1.48 (m, 2H), 0.42-0.31 (m, 4H). LCMS (ESI): calc, for C₂₁H₂₆F₃N₅O₂S [M+H]⁺ 470.18, found 470.24.

### Example 27. (R)—N-(2-(6-((1-Cyclobutylpiperidin-3-yl)amino)-5-methylpyridazin-3-yl)-5-(trifluoromethyl)phenyl) methanesulfonamide (Compound 31)

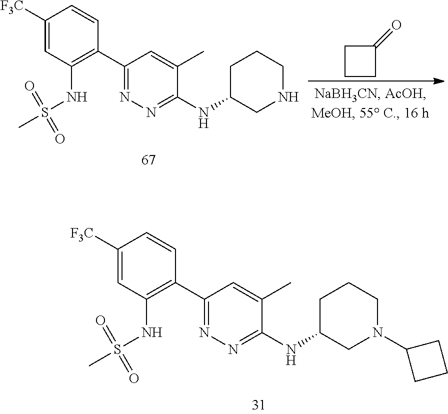 [SMILES: Cc1cc(-c2ccc(C(F)(F)F)cc2NS(C)(=O)=O)nnc1N[C@@H]1CCCNC1 ; O=C1CCC1 ; "67" ; Cc1cc(-c2ccc(C(F)(F)F)cc2NS(C)(=O)=O)nnc1N[C@@H]1CCCN(C2CCC2)C1]

To a stirred solution of (R)—N-(2-(5-methyl-6-(piperidin-3-ylamino)pyridazin-3-yl)-5-(trifluoromethyl)phenyl) methanesulfonamide (compound 67, 0.3 g, 0.699 mmol, 1 eq) in methanol (8 mL) was added cyclobutanone (0.245 g, 3.493 mmol, 5 eq), AA (0.1 mL) and sodium cyanoborohydride (0.088 g, 1.397 mmol, 2 eq) at ambient temperature. Resulting reaction mixture was heated to 55° C., stirred for 16 h. Progress of the reaction was monitored by TLC and LCMS. Reaction mixture was concentrated under reduced pressure. Crude was dissolved in 10% methanol/DCM (15 mL), washed with water (10 mL), aq. NaHCO₃ solution (10 mL) and brine (10 mL) solution. The organic layer was dried over sodium sulfate and concentrated under reduced pressure. The residue was subjected to reverse phase prep-HPLC purification using ammonium bicarbonate in water and ACN as an eluent to afford 100 mg (29%) of the title compound 31 as a pale-yellow solid.

Analytical data: ¹H NMR (400 MHz, DMSO-d₆), δ 12.77 (br. s, 1H), 8.07 (d, J=8.4 Hz, 1H), 7.98 (s, 1H), 7.85 (s, 1H), 7.53 (d, J=8.0 Hz, 1H), 6.29 (d, J=7.6 Hz, 1H), 4.33-4.26 (m, 1H), 3.10 (s, 3H), 2.93-2.90 (m, 1H), 2.75-2.65 (m, 2H), 2.21 (m, 3H), 2.00-1.90 (m, 3H), 1.84-1.71 (m, 5H), 1.62-1.56 (m, 2H), 1.58-1.43 (m, 2H). LCMS (ESI): calc. for C₂₂H₂₈F₃N₅O₂S [M+H]⁺ 484.19, found 484.26.

### Example 28. (R)—N-(2-(6-((1-Cyclopentylpiperidin-3-yl)amino)-5-methylpyridazin-3-yl)-5-(trifluoromethyl)phenyl) methanesulfonamide (Compound 26)

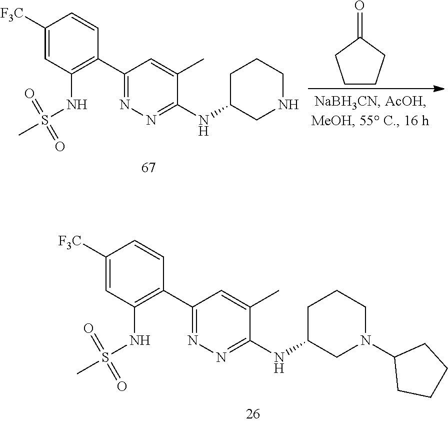 [SMILES: Cc1cc(-c2ccc(C(F)(F)F)cc2NS(C)(=O)=O)nnc1N[C@@H]1CCCNC1 ; O=C1CCCC1 ; "67" ; Cc1cc(-c2ccc(C(F)(F)F)cc2NS(C)(=O)=O)nnc1N[C@@H]1CCCN(C2CCCC2)C1]

To a stirred solution of (R)—N-(2-(5-methyl-6-(piperidin-3-ylamino)pyridazin-3-yl)-5-(trifluoromethyl)phenyl) methanesulfonamide (0.3 g, 0.699 mmol, 1 eq) in methanol (6 mL) was added cyclopentanone (0.294 mL, 3.493 mmol, 5 eq), AA (0.1 mL) and sodium cyanoborohydride (0.088 g, 1.397 mmol, 2 eq) at ambient temperature. Resulting reaction mixture was heated to 55° C., stirred for 16 h. Progress of the reaction was monitored by TLC and LCMS. Reaction mixture was concentrated under reduced pressure. Crude was dissolved in 10% methanol/DCM (15 mL), washed with water (10 mL), aq. NaHCO₃ solution (10 mL) and brine (10 mL) solution. Organic layer was dried over sodium sulfate and concentrated. The residue was subjected to reverse phase prep-HPLC purification using ammonium bicarbonate in water and ACN as an eluent to afford 106 mg (31%) of the title compound 26 as a pale-yellow solid.

Analytical data: ¹H NMR (400 MHz, DMSO-d₆), δ: 12.79 (br. s, 1H), 8.07 (d, J=8.0 Hz, 1H), 7.97 (s, 1H), 7.85 (s, 1H), 7.52 (d, J=6.8 Hz, 1H), 6.28 (d, J=5.2 Hz, 1H), 4.36-4.28 (m, 1H), 3.11-3.04 (m, 4H), 2.85-2.79 (m, 1H), 2.62-2.56 (m, 1H), 2.21 (m, 3H), 2.12-1.98 (m, 2H), 1.94-1.86 (m, 1H), 1.80-1.68 (m, 3H), 1.64-1.33 (m, 8H). LCMS (ESI): calc. for C₂₃H₃₀F₃N₅O₂S [M+H]⁺ 498.21, found 498.29.

### Example 29. (R)—N-(2-(6-((1-Cyclohexylpiperidin-3-yl)amino)-5-methylpyridazin-3-yl)-5-(trifluoromethyl)phenyl) methanesulfonamide (Compound 32)

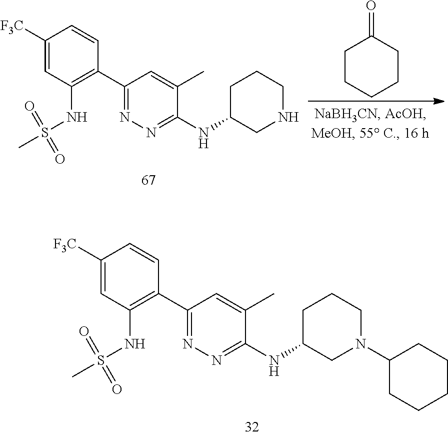 [SMILES: Cc1cc(-c2ccc(C(F)(F)F)cc2NS(C)(=O)=O)nnc1N[C@@H]1CCCNC1 ; O=C1CCCCC1 ; "67" ; Cc1cc(-c2ccc(C(F)(F)F)cc2NS(C)(=O)=O)nnc1N[C@@H]1CCCN(C2CCCCC2)C1]

To a stirred solution of (R)—N-(2-(5-methyl-6-(piperidin-3-ylamino)pyridazin-3-yl)-5-(trifluoromethyl)phenyl) methanesulfonamide (compound 67, 0.3 g, 0.699 mmol, 1.0 eq) in methanol (6 mL) was added cyclohexanone (0.343 g, 3.493 mmol, 5 eq), AA (0.1 mL) and sodium cyanoborohydride (0.088 g, 1.397 mmol, 2 eq) at ambient temperature. Resulting reaction mixture was heated to 55° C., stirred for 16 h. Progress of the reaction was monitored by TLC and LCMS. Reaction mixture was concentrated under reduced pressure. Crude was dissolved in 10% methanol/DCM (15 mL), washed with water (10 mL) and brine (10 mL) solution. The organic layer was dried over sodium sulfate and concentrated under reduced pressure. The residue was subjected to reverse phase prep-HPLC purification using ammonium bicarbonate in water and ACN as an eluant to afford 90 mg (25%) of the title compound 32 as an off-white solid.

Analytical data: ¹H NMR (400 MHz, DMSO-d₆), δ: 12.78 (s, 1H), 8.07 (d, J=8.4 Hz, 1H), 7.98 (s, 1H), 7.85 (s, 1H), 7.52 (d, J=6.8 Hz, 1H), 6.24 (d, J=7.2 Hz, 1H), 4.32-4.26 (m, 1H), 3.09 (s, 3H), 2.99-2.95 (m, 1H), 2.78-2.70 (m, 1H), 2.39-2.29 (m, 3H), 2.20 (s, 3H), 1.90-1.82 (m, 1H), 1.80-1.69 (m, 5H), 1.60-1.48 (m, 3H), 1.30-1.12 (m, 4H), 1.08-1.03 (m, 1H). LCMS (ESI): calc, for C₂₄H₃₂F₃N₅O₂S [M+H]⁺ 512.22, found 512.32.

### Example 30. (R)—N-(2-(5-Methyl-6-((1-(tetrahydro-2H-pyran-4-yl) piperidin-3-yl)amino)pyridazin-3-yl)-5-(trifluoromethyl)phenyl) methanesulfonamide (Compound 38)

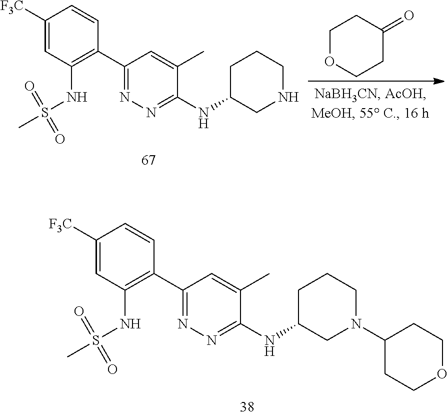 [SMILES: Cc1cc(-c2ccc(C(F)(F)F)cc2NS(C)(=O)=O)nnc1N[C@@H]1CCCNC1 ; O=C1CCOCC1 ; "67" ; Cc1cc(-c2ccc(C(F)(F)F)cc2NS(C)(=O)=O)nnc1N[C@@H]1CCCN(C2CCOCC2)C1]

To a stirred solution of (R)—N-(2-(5-methyl-6-(piperidin-3-ylamino)pyridazin-3-yl)-5-(trifluoromethyl)phenyl) methanesulfonamide (compound 67, 0.3 g, 0.699 mmol, 1.0 eq) in methanol (7.500 mL) was added tetrahydro-4H-pyran-4-one (0.35 g, 3.49 mmol, 5.0 eq), sodium cyanoborohydride (0.087 g, 1.39 mmol, 2 eq) and AA (0.004 mL, 0.070 mmol, 0.1 eq) at rt. The resultant reaction mixture was stirred at 55° C. for 16 h. The progress of reaction was monitored by TLC and LCMS. After completion of starting material, reaction mixture was evaporated under reduced pressure. The residue was diluted with 10% MeOH/DCM and washed with sat. NaHCO₃ (10 mL), water (10 mL), dried over anh. Na₂SO₄ and concentrated under reduced pressure. The residue was purified by prep-HPLC to afford 48 mg (13%) of the title compound 38 as an off-white solid.

Analytical data: ¹H NMR (400 MHz, DMSO-d₆), δ: 12.82 (br. s, exch. with D₂O, 1H), 8.08 (d, J=8.0 Hz, 1H), 7.98 (br. s, 1H), 7.85 (br. s, 1H), 7.54 (d, J=7.6 Hz, 1H), 6.28 (d, J=6.8 Hz, 1H), 4.32-4.23 (m, 1H), 3.88-3.86 (m, 2H), 3.29-3.23 (m, 2H), 3.11 (s, 3H), 3.03 (d, J=8.4 Hz, 1H), 2.78 (d, J=9.2 Hz, 1H), 2.51-2.45 (m, 1H), 2.25-2.20 (m, 5H), 1.90-1.88 (m, 1H), 1.76-1.62 (m, 3H), 1.59-1.39 (m, 4H). LCMS (ESI): calc. for C₂₃H₃₀F₃N₅O₃S [M+H]⁺ 514.20, found 514.30.

### Example 31. N-(2-(5-Methyl-6-(((1R,9aR)-octahydro-2H-quinolizin-1-yl)amino)pyridazin-3-yl)-5-(trifluoromethyl)phenyl) methanesulfonamide (Compound 73)

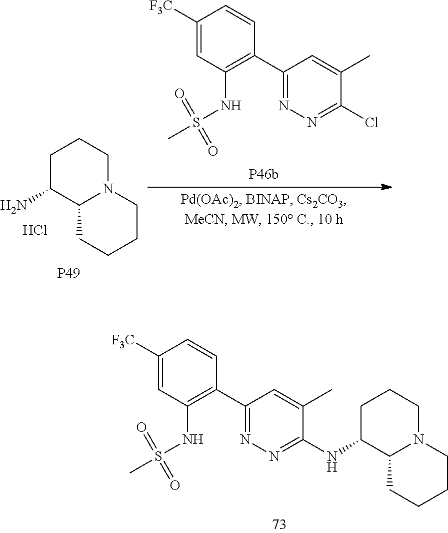 [SMILES: "P49" ; N[C@@H]1CCCN2CCCC[C@H]12 ; Cl ; Cc1cc(-c2ccc(C(F)(F)F)cc2NS(C)(=O)=O)nnc1N[C@@H]1CCCN2CCCC[C@H]12 ; Cc1cc(-c2ccc(C(F)(F)F)cc2NS(C)(=O)=O)nnc1Cl]

To a stirred solution of N-(2-(6-chloro-5-methylpyridazin-3-yl)-5-(trifluoromethyl)phenyl) methanesulfonamide (P46b, 150 mg, 0.41 mmol, 1.0 eq), (1R,9aR)-octahydro-2H-quinolizin-1-amine hydrochloride (P49, 313 mg, 1.64 mmol, 4.0 eq) in ACN (8 mL) was added Cs₂CO₃ (534 mg, 1.64 mmol, 4.0 eq) at ambient temperature. The reaction mixture was degassed with a stream of nitrogen for 15 min. Thereafter, t-BuXPhos Pd G3 (16.3 mg, 0.02 mmol, 0.05 eq) was added at this temperature. The resulting reaction mixture was irradiated under microwave at 150° C. for 10 h. The Progress of reaction was monitored by TLC and LCMS. The reaction mixture was filtered through celite pad and the pad was washed with 10% MeOH in DCM (30 mL). The filtrate concentrated under reduced pressure at 45° C. The residue was purified by flash chromatography over silica gel (24 g, 230-400 mesh) using mixture of MeOH (0→20%) and DCM as an eluent to give 10 mg with LCMS purity 53%. The obtained compound further purified by reverse phase prep-HPLC to afford 1.6 mg (1%) of the title compound 73 as an off-white solid.

Analytical data: ¹H NMR (400 MHz, DMSO-d₆), δ: 12.80 (s, exch. with D₂O, 1H), 8.09 (d, J=7.2 Hz, 1H), 8.04 (s, 1H), 7.86 (s, 1H), 7.55-7.54 (m, 1H), 5.52 (br. s, exch. with D₂O, 1H), 4.34-4.33 (m, 1H), 3.12 (s, 3H), 2.81 (br. s, 2H), 2.28 (s, 3H), 2.12-1.95 (m, 4H), 1.81-1.78 (m, 1H), 1.68-1.65 (m, 1H), 1.59-1.47 (m, 6H), 1.29-1.26 (m, 2H); LCMS (ESI): calc, for C₂₂H₂₈F₃N₅O₂S [M+H]⁺ 484.19, found 484.30.

### Example 32. (R)—N-(2-(6-((1-Ethylpiperidin-3-yl)amino)-4-methylpyridazin-3-yl)-5-(trifluoromethyl)phenyl) cyclopropanesulfonamide (Compound 65)

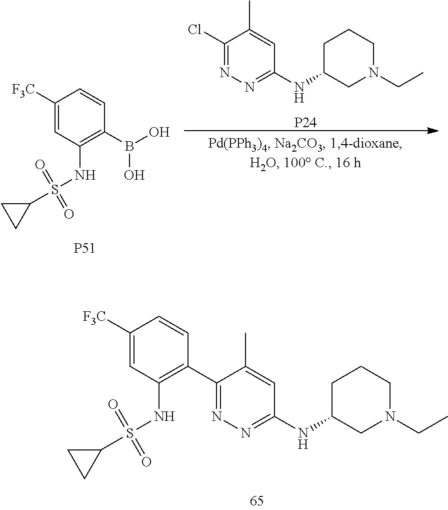

To a stirred solution of (R)-6-chloro-N-(1-ethylpiperidin-3-yl)-5-methylpyridazin-3-amine (P24, 200 mg, 0.78 mmol, 1.0 eq), (2-(cyclopropanesulfonamido)-4-(trifluoromethyl)phenyl) boronic acid (P51, 1.2 g, 3.92 mmol, 5.0 eq) in mixture of 1,4-dioxane (8.0 mL), H₂O (2.0 mL) was added Na₂CO₃ (250 mg, 2.35 mmol, 3.0 eq) at ambient temperature. The reaction mixture was degassed with stream of argon for 10 min. Thereafter, Pd(PPh₃)₄ (90.7 mg, 0.079 mmol, 0.1 equiv.) were added. The resulting reaction mixture was stirred for 16 h at 100° C. The progress of reaction was monitored by TLC and LCMS. After completion of the starting material, the reaction mixture was filtered through a celite pad and the pad was washed with 10% MeOH:DCM (20 mL). The filtrate was diluted with H₂O (20 mL), the layers were separated. The aq. layer was extracted with 10% MeOH:DCM (2×20 mL). The combined organic layer was washed with brine (15 mL), dried over anh. Na₂SO₄, filtered and concentrated under reduced pressure at 45° C. to give residue. The residue was purified by reverse phase Prep-HPLC to afford 27 mg (7%) of the title compound 65 as a pale brown solid.

Analytical data: ¹H NMR (400 MHz, DMSO-d₆), δ: 9.60 (br. s, exch. with D₂O, 1H), 7.79 (s, 1H), 7.52 (br. s, 2H), 6.77-6.74 (m, 1H exch. with D₂O, 2H), 4.10-4.04 (m, 1H), 3.02-3.01 (m, 1H), 2.72-2.66 (m, 1H), 2.59-2.54 (m, 1H), 2.49-2.41 (m, 2H), 2.09-1.99 (m, 5H), 1.90-1.87 (m, 1H), 1.75-1.72 (m, 1H), 1.59-1.51 (m, 1H), 1.37-1.29 (m, 1H), 1.01 (d, J=7.2 Hz, 3H), 0.85-0.80 (m, 4H); LCMS (ESI): calc. for: C₂₂H₂₈F₃N₅O₂S [M+H]⁺ 484.19, found 484.25.

### Example 33 N-(2-(6-(((3R,5R)-1-ethyl-5-fluoropiperidin-3-yl)amino)-5-methylpyridazin-3-yl)-5-(trifluoromethyl)phenyl) methanesulfonamide (Compound 66)

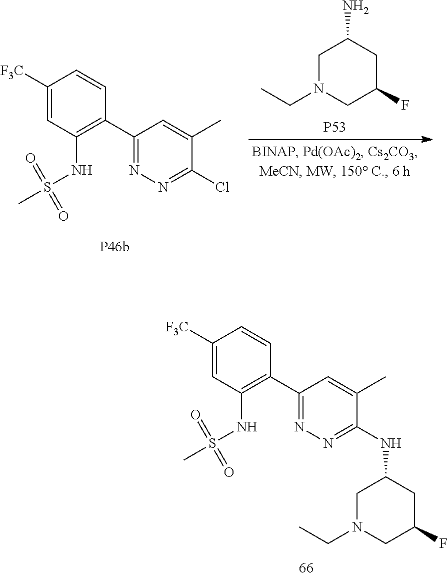

To a stirred solution of P46b (200 mg, 0.54 mmol, 1.0 eq), (3R,5R)-1-ethyl-5-fluoropiperidin-3-amine (P53, 239 mg, 1.62 mmol, 3.0 eq) in ACN (10 mL) was added Cs₂CO₃ (356 mg, 1.09 mmol, 2.0 eq) at ambient temperature. The reaction mixture was degassed with a stream of nitrogen for 15 min. Thereafter, BINAP (34 mg, 0.054 mmol, 0.1 eq) and Pd(OAc)₂ (18.5 mg, 0.027 mmol, 0.05 eq) was added. The resulting reaction mixture was irradiated under microwave for 6 h at 150° C. The progress of reaction was monitored by TLC and LCMS. The reaction mixture was filtered through celite pad and the pad was washed with 10% MeOH in DCM (50 mL). The filtrate concentrated under reduced pressure at 45° C. The residue was purified by reverse phase flash chromatography over C18 (12 g column) using mixture of 0.1% FA in H₂O (0→50%) and ACN to give residue 80 mg with HPLC purity: 37%. Reverse phase prep-HPLC afford 9.5 mg (3%) of the title compound 66 as a pale-yellow solid.

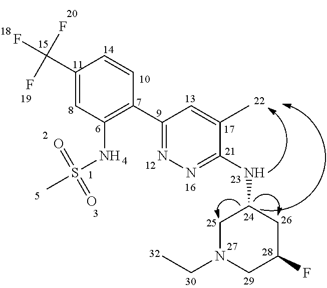 [SMILES: CCN1C[C@H](F)C[C@@H](Nc2nnc(-c3ccc(C(F)(F)F)cc3NS(C)(=O)=O)cc2C)C1]

Structure confirmation of 66: In the one experiment, irradiation of N-H23 proton at 6.23 ppm showed enhancement for H22 proton at 2.18 ppm. On irradiation of H24 proton at 4.26 ppm, enhancement was observed for N-H23 proton at 6.23 ppm, H25 proton at 3.02 ppm, H26 proton at 2.44 ppm, and H22 proton at 2.18 ppm.

Analytical data: ¹H NMR (400 MHz, DMSO-d₆), δ: 8.36 (s, 1H), 7.96 (d, J=0.8 Hz, 1H), 7.95 (s, 1H), 7.80 (d, J=0.8 Hz, 1H), 7.37 (d, J=8.0 Hz, 1H), 6.21 (d, exch. with D₂O, J=8.0 Hz, 1H), 4.96 (d, J=47.6 Hz, 1H), 4.62-4.57 (m, 1H), 3.06-2.99 (m, 5H), 2.42 (q, J=7.2 Hz, 2H), 2.26-2.14 (m, 5H), 2.01 (t, J=10.0 Hz, 1H), 1.89-1.72 (m, 1H), 1.01 (t, J=6.8 Hz, 3H).

### Example 34. (R)—N-(2-(4-Cyclopropyl-6-((1-ethylpiperidin-3-yl)amino)pyridazin-3-yl)-5-(trifluoromethyl)phenyl) methanesulfonamide (43) and (R)—N-(2-(5-Cyclopropyl-6-((1-ethylpiperidin-3-yl)amino)pyridazin-3-yl)-5-(trifluoromethyl)phenyl) methane-sulfonamide (28)

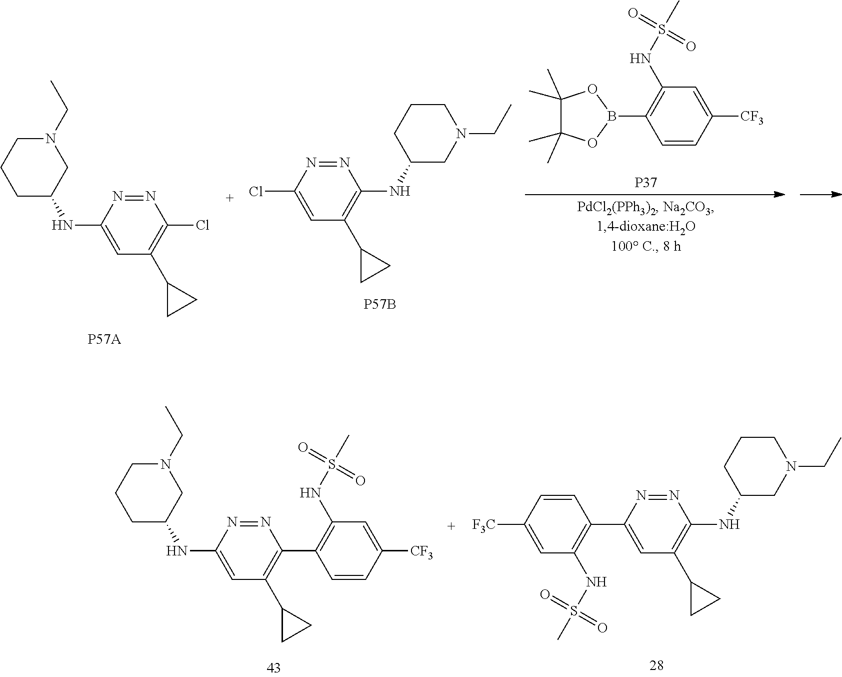

To a stirred solution of mixture of (R)-6-chloro-5-cyclopropyl-N-(1-ethylpiperidin-3-yl)pyridazin-3-amine (P57A) and (R)-6-chloro-4-cyclopropyl-N-(1-ethylpiperidin-3-yl)pyridazin-3-amine (P57B) (0.500 mg, 1.781 mmol, 1.0 eq) and P37 (0.975 g, 2.671 mmol, 1.5 eq) in mixture of 1,4-dioxane (20.000 mL) and water (5.000 mL) was added Na₂CO₃ (0.566 g, 5.342 mmol, 3.0 eq) at ambient temperature. The mixture was degassed with a stream of N₂ for 5 min followed by addition of PdCl₂(PPh₃)₂ (0.124 g, 0.178 mmol, 0.1 eq) at ambient temperature. The reaction mixture was heated to 100° C. and stirred for 8 h. The progress of the reaction was monitored by TLC and LCMS. After completion of reaction, reaction mixture was filtered through a pad of celite, the pad of celite was washed with EtOAc (2×15 mL), the filtrate was concentrated under reduced pressure. The residue was subjected to flash column chromatography using mixture of MeOH (10%→15%) and DCM as an eluent, which was re-purified by Prep HPLC to afford (0.025 g, 5.81%) of the title compound 43 as an off-white solid and (0.029 g, 5.81%) of the title compound 28 an off-white solid.

Analytical data of 43: 1H NMR (400 MHz, DMSO-d₆), δ: 9.38 (br. s, exch. with D₂O, 1H), 7.80 (s, 1H), 7.55-7.49 (m, 2H), 6.62 (d, J=8.0 Hz, 1H), 6.41 (s, 1H), 4.10-4.07 (m, 1H), 2.96 (s, 4H), 2.70-2.66 (m, 1H), 2.42-2.40 (m, 2H), 2.11-1.91 (m, 2H), 1.84-1.83 (m, 1H), 1.74-1.71 (m, 1H), 1.56-1.50 (m, 2H), 1.49-1.43 (m, 1H), 1.01 (t, J=7.2 Hz, 3H), 0.87-0.86 (m, 2H), 0.40-0.20 (m, 2H). LCMS (ESI): calc. for: C₂₂H₂₈F₃N₅O₂S [M+H]⁺ 484.19 found 484.30.

### Structure Confirmation of 43:

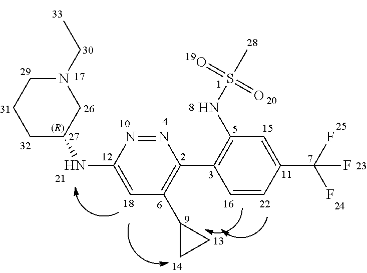

Key points:
In NOE Experiment on irradiation of H16, H22 Protons at 7.55 ppm (Observed those two protons together) giving correlation with H9 Proton at 1.48 ppm.
In NOE Experiment on irradiation of H18 Proton at 6.41 ppm giving correlation with H21 Proton (NH) at 6.63 ppm and H14 Proton at 0.66 ppm.

Analytical data of 28: 1H NMR (400 MHz, DMSO-d₆), δ: 12.55 (br. s, exch. with D₂O, 1H), 8.13 (d, J=8.4 Hz, 1H), 7.83 (s, 1H), 7.60 (s, 1H), 7.49 (d, J=7.6 Hz, 1H), 6.45 (d, J=6.8 Hz, 1H), 4.37-4.35 (m, 1H), 3.09 (s, 3H), 2.95-2.93 (m, 1H), 2.68-2.67 (m, 1H), 2.41-2.40 (m, 2H), 2.15-2.13 (m, 2H), 1.91-1.85 (m, 2H), 1.73-1.71 (m, 1H), 1.59-1.57 (m, 2H), 1.06-1.01 (m, 5H), 0.87-0.86 (m, 1H). LCMS (ESI): calc. for: C₂₂H₂₈F₃N₅O₂S [M+H]⁺ 484.19 found 484.30.

### Structure Confirmation of 28:

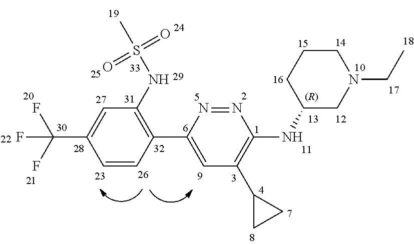

Key point: In NOE Experiment on irradiation of H26 Proton at 8.14 ppm giving correlation with H9 Proton at 7.59 ppm and H23 Proton at 7.47 ppm.

### Example 35. (R)-6-(2-Amino-4-(trifluoromethyl)phenyl)-N-(1-ethylpiperidin-3-yl)-5-methylpyridazin-3-amine (130)

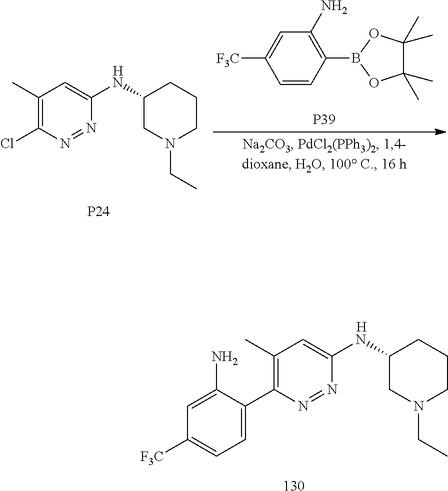

To a stirred solution of (R)-6-chloro-N-(1-ethylpiperidin-3-yl)-5-methylpyridazin-3-amine (P24, 600 mg, 2.35 mmol, 1.0 eq), 2-(4,4,5,5-tetramethyl-1,3,2-dioxaborolan-2-yl)-5-(trifluoromethyl) aniline (P39, 1.69 g, 5.88 mmol, 2.5 eq) in a mixture of solvents 1,4-dioxane (24.0 mL), H₂O (6.0 mL) was added Na₂CO₃ (749 mg, 7.06 mmol, 3.0 eq) at ambient temperature. The reaction mixture was degassed with a stream of nitrogen for 5 min. Thereafter, PdCl₂(PPh₃)₂ (165 mg, 0.23 mmol, 0.05 eq) was added. The resulting reaction mixture was stirred for 16 h at 100° C. The progress of reaction was monitored by TLC and LCMS. The reaction mixture was filtered through celite pad, the pad was washed with 10% MeOH in DCM (30 mL), and the filtrate was washed with water (20 mL), concentrated under reduced pressure to give residue. The residue was subjected to flash chromatography over silica gel (40 g, 230-400 mesh) using a mixture of MeOH (0→10%) and DCM as an eluent to afford 700 mg (78%) of the title compound 130 (slight impure) as a brown solid, 100 mg of impure compound was further purified by RP Prep-HPLC to afford 45 mg of the title compound 130 as a pale yellow solid.

Analytical data: ¹H NMR (400 MHz, DMSO-d₆), δ: 7.14 (d, J=7.6 Hz, 1H), 7.06 (d, J=1.2 Hz, 1H), 6.87 (dd, J=1.2, 8.0 Hz, 1H), 6.72 (d, J=0.8 Hz, 1H), 6.62 (d, exch. with D₂O, J=8.0 Hz, 1H), 5.32 (s, exch. with D₂O, 2H), 4.06-4.00 (m, 1H), 2.97-2.95 (m, 1H), 2.67-2.65 (m, 1H), 2.35 (q, J=7.2 Hz, 2H), 1.99-1.96 (m, 4H), 1.89-1.86 (m, 2H), 1.73-1.68 (m, 1H), 1.56-1.48 (m, 1H), 1.34-1.24 (m, 1H), 1.01 (t, J=7.2 Hz, 3H); LCMS (ESI): calc, for C₁₉H₂₄F₃N₅ [M+H]⁺ 380.20, found 380.33.

### Example 36. (R)—N-(2-(6-((1-ethylpiperidin-3-yl)amino)-4-methylpyridazin-3-yl)-5-(trifluoromethyl)phenyl) acetamide (134)

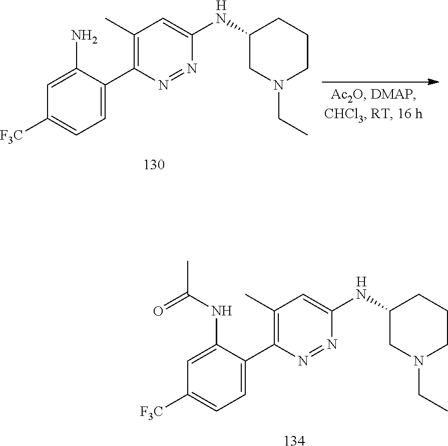

To a stirred solution of (R)-6-(2-amino-4-(trifluoromethyl)phenyl)-N-(1-ethylpiperidin-3-yl)-5-methylpyridazin-3-amine (130, 200 mg, 0.52 mmol, 1.0 eq) in CHCl₃ (6.0 mL) was added DMAP (3.2 mg, 0.026 mmol, 0.05 eq), followed by Ac₂O (0.075 mL, 0.79 mmol, 1.5 eq) dropwise at ambient temperature. The reaction mixture was stirred for 16 h at ambient temperature. The progress of reaction was monitored by TLC and LCMS. After completion of reaction, the reaction mixture was quenched with sat. NaHCO₃ solution (20 mL) and extracted with 10% MeOH in DCM (2×30 mL). The combined organic layer was dried over anh. Na₂SO₄, filtered and evaporated under reduced pressure to give residue. The residue purified by reverse phase Prep-HPLC to afford 66 mg (29%) of the title compound 134 as a white solid.

Analytical data: ¹H NMR (400 MHz, DMSO-d₆), δ: 9.49 (s, exch. with D₂O, 1H), 8.18 (s, 1H), 7.54 (dd, J=1.2 Hz, 8.0 Hz, 1H), 7.49 (d, J=8.0 Hz, 1H), 6.72 (s, 1H), 6.70 (s, exch. with D₂O, 1H), 4.06-4.04 (m, 1H), 2.98-2.94 (m, 1H), 2.67-2.66 (m, 1H), 2.35 (q, J=7.2 Hz, 2H), 2.09-1.87 (m, 9H), 1.75-1.69 (m, 1H), 1.57-1.49 (m, 1H), 1.36-1.27 (m, 1H), 1.01 (t, J=7.2 Hz, 3H); LCMS (ESI): calc, for C₂₁H₂₆F₃N₅O [M+H]⁺ 422.21, found 422.34.

### Example 37. (R)-6-(2-Amino-4-(trifluoromethyl)phenyl)-N-(1-isopropylpiperidin-3-yl)-5-methyl pyridazin-3-amine (110)

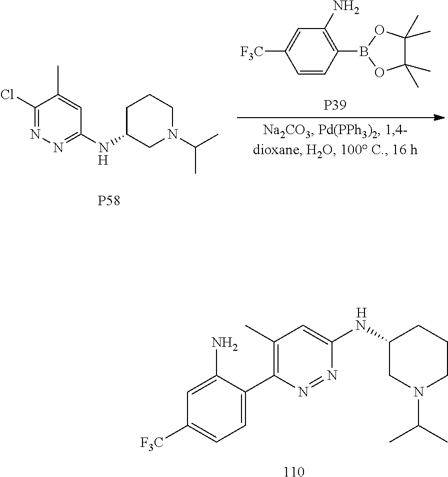

To a stirred solution of (R)-6-chloro-N-(1-isopropylpiperidin-3-yl)-5-methylpyridazin-3-amine (P58, 2 g, 7.44 mmol, 1.0 eq), 2-(4,4,5,5-tetramethyl-1,3,2-dioxaborolan-2-yl)-5-(trifluoromethyl) aniline (P39, 5.340 g, 18.602 mmol, 2.5 eq) in a mixture of solvents 1,4-dioxane (80 mL), H₂O (20 mL) was added Na₂CO₃ (2.36 g, 22.32 mmol, 3.0 eq) at ambient temperature. The reaction mixture was degassed with a stream of nitrogen for 5 min. Thereafter, PdCl₂(PPh₃)₂ (522 mg, 0.74 mmol, 0.1 eq) was added. The reaction mixture was stirred for 16 h at 100° C. The progress of reaction was monitored by TLC and LCMS. The reaction mixture was filtered through celite pad, the pad was washed with 10% MeOH in DCM (50 mL), the filtrate was washed with water (20 mL) and concentrated under reduced pressure to give residue. The residue was subjected to flash chromatography over silica gel (120 g, 230-400 mesh) using a mixture of MeOH (0→10%) and DCM as an eluent to afford 2.1 g (71%) of the title compound 110 as a brown solid. The 100 mg compound was further purified by RP Prep-HPLC to afford 45 mg of the title compound 110 as a pale yellow solid.

Analytical data: ¹H NMR (400 MHz, DMSO-d₆), δ: 7.14 (d, J=7.6 Hz, 1H), 7.06 (d, J=1.6 Hz, 1H), 6.87 (dd, J=1.2 Hz, 8.0 Hz, 1H), 6.71 (d, J=0.8 Hz, 1H), 6.58 (d, exch. with D₂O, J=8.0 Hz, 1H), 5.32 (s, exch. with D₂O, 2H), 4.03-3.96 (m, 1H), 2.96-2.92 (m, 1H), 2.76-2.69 (m, 1H), 2.67-2.62 (m, 1H), 2.20-2.15 (m, 1H), 2.07-1.99 (m, 1H), 1.99 (s, 3H), 1.90-1.84 (m, 1H), 1.72-1.67 (m, 1H), 1.51-1.45 (m, 1H), 1.34-1.25 (m, 1H), 0.97-0.95 (m, 6H). LCMS (ESI): calc, for C₂₀H₂₆F₃N₅ [M+H]⁺ 394.21, found 394.27.

### Example 38. (R)-2,2,2-Trifluoro-N-(2-(6-((1-isopropylpiperidin-3-yl)amino)-4-methylpyridazin-3-yl)-5-(trifluoromethyl)phenyl) acetamide (117)

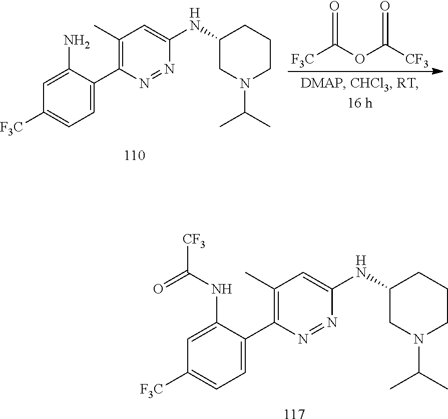

To a stirred solution of (R)-6-(2-amino-4-(trifluoromethyl)phenyl)-N-(1-isopropylpiperidin-3-yl)-5-methylpyridazin-3-amine (compound 110, 200 mg, 0.50 mmol, 1.0 eq) in CHCl₃ (6.0 mL) was added DMAP (3.1 mg, 0.025 mmol, 0.05 eq), followed by 2,2,2-trifluoroacetic anhydride (0.11 mL, 0.76 mmol, 1.5 eq) dropwise at ambient temperature. The reaction mixture was stirred at ambient temperature for 16 h. The progress of reaction was monitored by TLC and LCMS. After completion of reaction, the reaction mixture was quenched with sat. NaHCO₃ solution (20 mL) and extracted with 10% MeOH in DCM (2×30 mL). The combined organic layer was dried over anh. Na₂SO₄, filtered and evaporated under reduced pressure to give residue. The residue was purified by reverse phase Prep-HPLC to afford 34 mg (13%) of the title compound 117 as a white solid.

Analytical data: ¹H NMR (400 MHz, DMSO-d₆), δ: 11.32 (br. s, exch. with D₂O, 1H), 7.98 (s, 1H), 7.68-7.65 (m, 2H), 6.72-6.67 (m, 2H), 4.02 (br. s, 1H), 3.01 (br. s, 1H), 2.79-2.71 (m, 2H), 2.26-2.07 (m, 2H), 2.02 (s, 3H), 1.90-1.86 (m, 1H), 1.72 (br. s, 1H), 1.54-1.51 (m, 1H), 1.33-1.31 (m, 1H), 0.99-0.98 (m, 6H); LCMS (ESI): calc, for C₂₂H₂₅F₆N₅O [M+H]⁺ 490.20, found 490.30.

### Example 39. (R)-2,2,3,3,3-Pentafluoro-N-(2-(6-((1-isopropylpiperidin-3-yl)amino)-4-methylpyridazin-3-yl)-5-(trifluoromethyl)phenyl) propanamide (123)

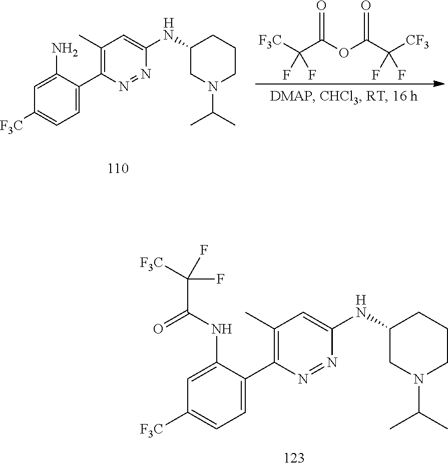

To a stirred solution of (R)-6-(2-amino-4-(trifluoromethyl)phenyl)-N-(1-isopropylpiperidin-3-yl)-5-methylpyridazin-3-amine (compound 110, 200 mg, 0.50 mmol, 1.0 eq) in CHCl₃ (6.0 mL) was added DMAP (3.1 mg, 0.025 mmol, 0.05 eq), followed by 2,2,3,3,3-pentafluoropropanoic anhydride (0.15 mL, 0.76 mmol, 1.5 eq) dropwise at ambient temperature. The reaction mixture was stirred at ambient temperature for 16 h. The progress of reaction was monitored by TLC and LCMS. After completion of reaction, the reaction mixture was quenched with sat. NaHCO₃ solution (20 mL) and extracted with 10% MeOH in DCM (2×30 mL). The combined organic layer was dried over anh. Na₂SO₄, filtered and evaporated under reduced pressure to give residue. The residue was purified by reverse phase Prep-HPLC to afford 84 mg (30%) of the title compound 123 as a white solid.

Analytical data: ¹H NMR (400 MHz, DMSO-d₆), δ: 11.24 (br. s, exch. with D₂O, 1H), 7.98 (br. s, 1H), 7.60 (br. s, 2H), 6.69-6.66 (m, 2H), 4.04 (br. s, 1H), 3.16 (br. s, 1H), 2.85-2.66 (m, 2H), 2.33-2.32 (m, 1H), 2.20-2.07 (m, 1H), 2.02 (s, 3H), 1.90-1.74 (m, 2H), 1.57-1.55 (m, 1H), 1.37-1.34 (m, 1H), 1.02-1.01 (m, 6H); LCMS (ESI): calc, for C₂₃H₂₅F₈N₅O [M+H]⁺ 540.19, found 540.33.

### Example 40. (R)—N-(2-(6-((1-Isopropylpiperidin-3-yl)amino)-4-methylpyridazin-3-yl)-5-(trifluoro methyl)phenyl) isobutyramide (132)

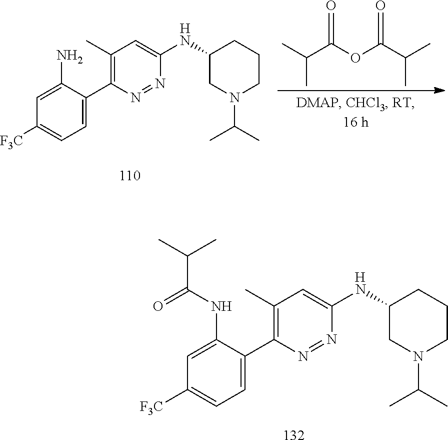

To a stirred solution of (R)-6-(2-amino-4-(trifluoromethyl)phenyl)-N-(1-isopropylpiperidin-3-yl)-5-methylpyridazin-3-amine (compound 110, 150 mg, 0.38 mmol, 1.0 eq) in CHCl₃ (4.5 mL) was added DMAP (2.3 mg, 0.019 mmol, 0.05 eq), followed by isobutyric anhydride (90.4 mg, 0.57 mmol, 1.5 eq) at ambient temperature. The reaction mixture was stirred at ambient temperature for 16 h. The progress of reaction was monitored by TLC and LCMS. After completion of reaction, the reaction mixture was quenched with sat. NaHCO₃ solution (20 mL) and extracted with 10% MeOH in DCM (2×30 mL). The combined organic layer was dried over anh. Na₂SO₄, filtered and evaporated under reduced pressure to give residue. The residue was purified by reverse phase Prep-HPLC to afford 56 mg (31%) of the title compound 132 as an off-white solid.

Analytical data: ¹H NMR (400 MHz, DMSO-d₆), δ: 9.54 (s, exch. with D₂O, 1H), 8.09 (s, 1H), 7.56 (dd, J=1.6 Hz, 8.4 Hz, 1H), 7.52 (d, J=8.4 Hz, 1H), 6.72 (s, 1H), 6.65 (d, exch. with D₂O, J=8.0 Hz, 1H), 4.01-3.99 (m, 1H), 2.97-2.94 (m, 1H), 2.73-2.62 (m, 2H), 2.47-2.45 (m, 1H), 2.17 (t, J=10.0 Hz, 1H), 2.03 (t, J=9.2 Hz, 1H), 1.94 (s, 3H), 1.88-1.84 (m, 1H), 1.71-1.68 (m, 1H), 1.51-1.48 (m, 1H), 1.31-1.29 (m, 1H), 0.97-0.94 (m, 12H); LCMS (ESI): calc, for C₂₄H₃₂F₃N₅O [M+H]⁺ 464.26, found 464.36.

### Example 41. N-(2-(6-(((3R,5S)-1-Cyclopropyl-5-(trifluoromethyl) piperidin-3-yl) amino)-5-methylpyridazin-3-yl)-5-(trifluoromethyl)phenyl) methanesulfonamide (90)

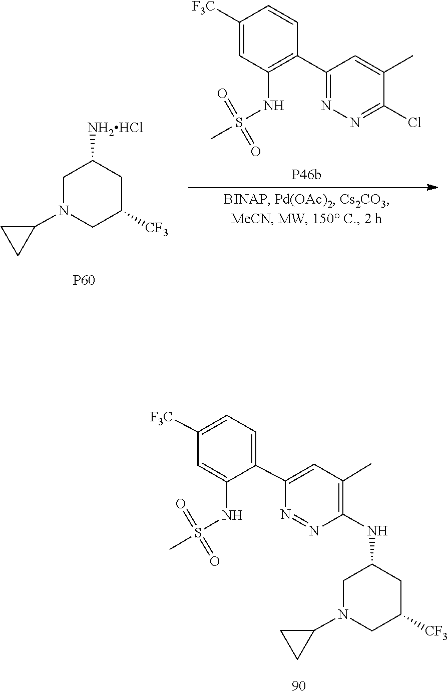

To a stirred solution of N-(2-(6-chloro-5-methylpyridazin-3-yl)-5-(trifluoromethyl)phenyl) methanesulfonamide (P46b, 190 mg, 0.51 mmol, 1.0 eq), (3R,5S)-1-cyclopropyl-5-(trifluoromethyl) piperidin-3-amine hydrochloride (P60, 380 mg, 1.55 mmol, 3.0 eq) in ACN (10 mL) was added Cs₂CO₃ (338 mg, 1.04 mmol, 2.0 eq) at ambient temperature. The reaction mixture was degassed with a stream of nitrogen for 15 min. Thereafter, BINAP (32 mg, 0.051 mmol, 0.1 eq) and Pd(OAc)₂ (17.5 mg, 0.025 mmol, 0.05 eq) was added. The resulting reaction mixture was irradiated under microwave for 2 h at 150° C. The progress of reaction was monitored by TLC and LCMS. The reaction mixture was filtered through celite pad and the pad was washed with 10% MeOH in DCM (50 mL). The filtrate concentrated under reduced pressure at 45° C. The residue was purified by reverse phase flash chromatography over C18 (15 g column) using mixture of 0.1% FA in H₂O (0→40%) and ACN to give residue 70 mg with LCMS purity: 77%. So, it was further purified by reverse phase prep-HPLC to afford 25 mg (8%) of the title compound 90 as a pale yellow solid.

Analytical data: ¹H NMR (400 MHz, DMSO-d₆), δ: 12.68 (s, exch. with D₂O, 1H), 8.09 (d, J=7.6 Hz, 1H), 7.99 (s, 1H), 7.85 (s, 1H), 7.55 (d, J=7.6 Hz, 1H), 6.51 (d, exch. with D₂O, J=7.6 Hz, 1H), 4.40-4.36 (m, 1H), 3.25-3.21 (m, 1H), 3.11 (s, 4H), 2.79-2.66 (m, 1H), 2.22-2.14 (m, 6H), 1.82-1.77 (m, 1H), 1.53 (q, J=12.4 Hz, 1H), 0.47-0.38 (m, 4H); LCMS (ESI): calc, for C₂₂H₂₅F₆N₅O₂S [M+H]⁺ 538.16, found 538.3.

### Example 42. 2-((R)-3-((6-(2-Amino-4-(trifluoromethyl)phenyl)-5-methylpyridazin-3-yl)amino) piperidin-1-yl) propan-1-ol (128)

To a stirred solution of 2-((R)-3-((6-chloro-5-methylpyridazin-3-yl)amino) piperidin-1-yl) propan-1-ol (P61, 200 mg, 0.70 mmol, 1.0 eq), 2-(4,4,5,5-tetramethyl-1,3,2-dioxaborolan-2-yl)-5-(trifluoromethyl) aniline (P39, 403 mg, 1.40 mmol, 2.0 eq) in a mixture of 1,4-dioxane (8 mL) and H₂O (2 mL) was added Na₂CO₃ (223 mg, 2.10 mmol, 3.0 eq) at ambient temperature. The mixture was degassed with a stream of argon for 10 min. Thereafter, Ruphos Pd G2 (27 mg, 0.035 mmol, 0.05 eq) was added at ambient temperature. The resulting reaction mixture was stirred at 100° C. for 2 h. The progress of reaction was monitored by TLC and LCMS. The reaction mixture was filtered through celite pad, the pad was washed with 10% MeOH in DCM (20 mL), the filtrate was washed with water (20 mL) and concentrated under reduced pressure to give residue. The crude residue was purified by reverse phase flash column chromatography over C18 (12 g) using a mixture of 0.1% ammonium bicarbonate in H₂O (0→60%) and ACN as an eluent to give residue. This residue was further purified by RP Prep-HPLC to afford 50 mg (70%) of the title compound 128 as an off white solid.

Analytical data: ¹H NMR (400 MHz, DMSO-d₆), δ: 7.70-7.66 (m, 2H), 7.10-7.06 (m, 3H), 6.86 (dd, J=1.2 Hz, 8.4 Hz, 1H), 5.60-5.96 (m, exch. with D₂O, 1H), 4.29-4.24 (m, 2H), 3.49-3.42 (m, 1H), 3.29-3.27 (m, 1H), 2.96-2.89 (m, 1H), 2.68-2.65 (m, 2H), 2.41-2.30 (m, 2H), 2.17 (s, 3H), 1.87-1.84 (m, 1H), 1.72-1.69 (m, 1H), 1.56-1.45 (m, 2H), 0.94-0.89 (m, 3H). LCMS (ESI): calc, for C₂₀H₂₆F₃N₅O [M+H]⁺ 410.21, found 410.33.

### Example 43. N-(2-(6-(((3R)-1-(sec-Butyl) piperidin-3-yl)amino)-4-methylpyridazin-3-yl)-5-(trifluoromethyl)phenyl) methanesulfonamide (35)

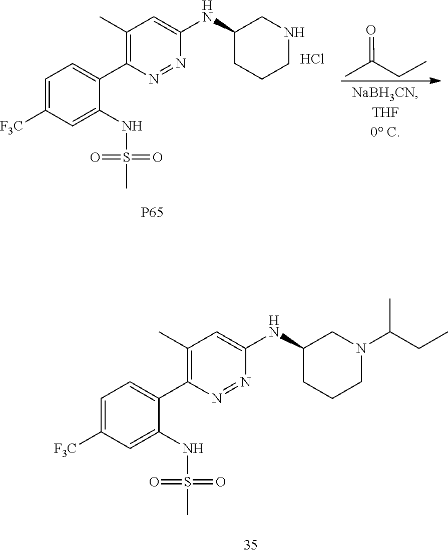

To a solution of (R)—N-(2-(4-methyl-6-(piperidin-3-ylamino)pyridazin-3-yl)-5-(trifluoromethyl)phenyl) methanesulfonamide hydrochloride (P65, 100 mg, 0.197 mmol) and butan-2-one (2 mL) in THF (2 mL) was added sodium cyanoborohydride (49 mg, 0.788 mmol) at 0° C. After stirred at 0° C. for 30 min, the reaction mixture was diluted with water (5 mL) and extracted with EA (5 mL) for three times. The combined organic layers were washed with brine (10 mL), dried over Na₂SO₄ (S), filtered and concentrated. The residue was purified by C18 column (ACN: 0.01% FA aq. solution=05% to 95%) to afford the compound 35 (23.7 mg, 96.01% purity from HPLC, 24% yield) as white solids. LCMS (ESI): R_{T}=0.937 min, mass calc, for C₂₂H₃₀F₃N₅O₂S 485.2, m/z found 486.2 [M+H]⁺. 1H NMR (400 MHz, CD₃OD), δ: 8.54 (s, 0.6H), 7.88-7.87 (s, 1H), 7.61-7.52 (m, 2H), 6.86-6.85 (m, 1H), 4.32-4.26 (m, 1H), 3.49-3.45 (m, 1H), 3.18-3.13 (m, 1H), 3.07-3.02 (m, 1H), 2.95 (s, 3H), 2.89-2.73 (m, 2H), 2.13 (s, 3H), 2.08-2.01 (m, 2H), 1.88-1.78 (m, 2H), 1.67-1.59 (m, 1H), 1.53-1.44 (m, 1H), 1.25-1.23 (m, 3H), 1.01-0.97 (m, 3H). 19F NMR (376 MHz, CD₃OD), δ: −64.35 (s, 3F).

### Example 44. (R)—N-(2-(6-((1-Isobutylpiperidin-3-yl)amino)-4-methylpyridazin-3-yl)-5-(trifluoromethyl)phenyl) methanesulfonamide (46)

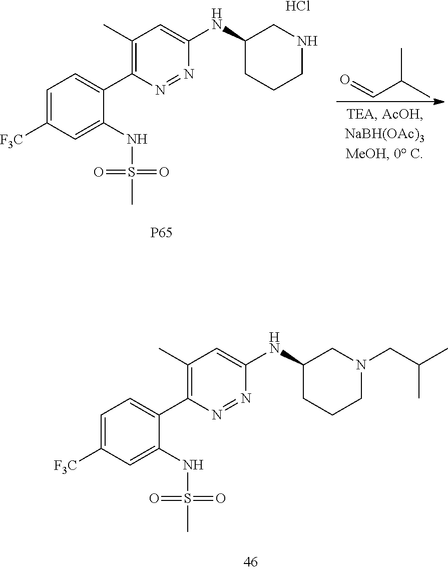

To a solution of (R)—N-(2-(4-methyl-6-(piperidin-3-ylamino)pyridazin-3-yl)-5-(trifluoromethyl)phenyl) methanesulfonamide hydrochloride (P65, 100 mg, 0.195 mmol), TEA (0.1 mL), AA (0.2 mL) and isobutyraldehyde (2 mL) in methanol (2 mL) was added sodium triacetoxyborohydride (165 mg, 0.780 mmol) at 0° C. After stirred at 0° C. for 30 minutes, the reaction mixture diluted with water (5 mL) and extracted with EA (5 mL) for three times. The combined organic layers were washed with brine (10 mL), dried over Na₂SO₄ (S), filtered and concentrated. The residue was purified by silica gel column chromatography (petroleum ether:EA=2:1 to 0:1) to afford the desired product (33.1 mg, 98.36% purity from HPLC, 34% yield) as white solids. LCMS (ESI): R_{T}=0.805 min, mass calc, for C₂₂H₃₀F₃N₅O₂S 485.2, m/z found 486.1 [M+H]⁺. ¹H NMR (400 MHz, CD₃OD), δ: 7.89 (s, 1H), 7.57-7.50 (m, 2H), 6.84 (s, 1H), 4.18-4.12 (m, 1H), 3.03-3.00 (m, 1H), 2.94 (s, 3H), 2.72-2.68 (m, 1H), 2.27-2.15 (m, 4H), 2.11 (s, 3H), 1.99-1.95 (m, 1H), 1.87-1.79 (m, 2H), 1.74-1.67 (m, 1H), 1.50-1.41 (m, 1H), 0.95-0.92 (m, 6H). 1° F. NMR (376 MHz, CD₃OD), δ: −62.82 (s, 3F).

### Example 45. (R)—N-(2-(6-((1-Isobutylpiperidin-3-yl)amino)-4-methylpyridazin-3-yl)-5-(trifluoromethyl)phenyl) methanesulfonamide (47)

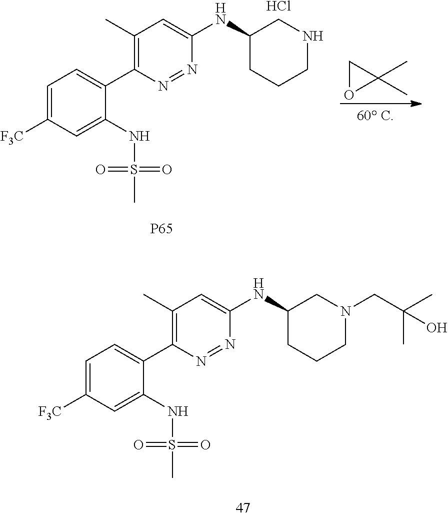

The solution of (R)—N-(2-(4-methyl-6-(piperidin-3-ylamino)pyridazin-3-yl)-5-(trifluoromethyl)phenyl) methanesulfonamide hydrochloride (P65, 70 mg, 0.138 mmol) in 2,2-dimethyloxirane (5 mL) was stirred at 60° C. for 12 h. The reaction mixture was cooled to rt and concentrated. The residue was purified by C18 column (ACN: 0.02% ammonium bicarbonate aq. solution=5% to 95%) to afford the desired compound 47 (31.8 mg, 99.75% purity from HPLC, 46% yield) as white solids. LCMS (ESI): R_{T}=0.775 min, mass calc. for C₂₂H₃₀F₃N₅O₃S 501.2, m/z found 502.2 [M+H]⁺. ¹H NMR (400 MHz, CDCl₃), δ: 8.48 (br. s, 1H), 7.97 (s, 1H), 7.47 (d, J=8.0 Hz, 1H), 7.38 (d, J=8.0 Hz, 1H), 6.62 (s, 1H), 5.32 (s, 1H), 4.10 (s, 1H), 3.01 (s, 3H), 2.93-2.87 (m, 1H), 2.73-2.58 (m, 3H), 2.36 (s, 2H), 2.20 (s, 3H), 1.81 (d, J=7.6 Hz, 2H), 1.73 (s, 2H), 1.20 (d, J=6.8 Hz, 6H). ¹⁹F NMR (376 MHz, CDCl₃), δ: −62.82 (s, 3F).

### Example 45. (R)—N-(2-(6-((1-(3-Hydroxy-3-methylbutyl) piperidin-3-yl)amino)-4-methylpyridazin-3-yl)-5-(trifluoromethyl)phenyl) methanesulfonamide (55)

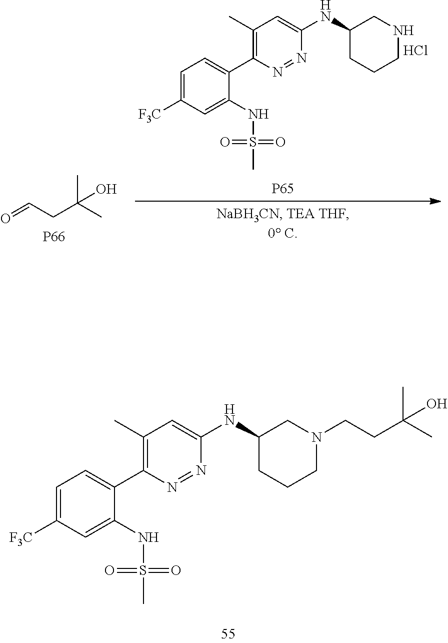

To a solution of (R)—N-(2-(4-methyl-6-(piperidin-3-ylamino)pyridazin-3-yl)-5-(trifluoromethyl)phenyl) methanesulfonamide hydrochloride (P65, 100 mg, 0.198 mmol), TEA (0.1 mL) and 3-hydroxy-3-methylbutanal (P66, 0.1 mL) in THF (2 mL) was added sodium cyanoborohydride (47 mg, 0.747 mmol) at 0° C. After stirred at 0° C. for 30 min, the reaction mixture diluted with water (5 mL) and extracted with EA (5 mL) for three times. The combined organic layers were washed with brine (10 mL), dried over Na₂SO₄ (s) and filtered. The filtrate was concentrated to leave a residue which was purified by C18 column (ACN: 0.02% ammonium bicarbonate aq. solution=5% to 95%) to afford the desired compound 55 (40.3 mg, 97.79% purity from HPLC, 35% yield) as white solids. LCMS (ESI): R_{T}=0.787 min, mass calc, for C₂₃H₃₂F₃N₅O₃S 515.2, m/z found 516.2 [M+H]⁺. 1H NMR (400 MHz, CD₃OD), δ: 7.88 (s, 1H), 7.57-7.50 (m, 2H), 6.83 (s, 1H), 4.17-4.11 (m, 1H), 3.20-3.16 (m, 1H), 2.94 (s, 3H), 2.89-2.86 (m, 1H), 2.68-2.65 (m, 2H), 2.33-2.26 (m, 2H), 2.11 (s, 3H), 2.11-2.03 (m, 1H), 1.88-1.84 (m, 1H), 1.73-1.69 (m, 3H), 1.51-1.42 (m, 1H), 1.22-1.20 (m, 6H). ¹⁹F NMR (376 MHz, CD₃OD), δ: −64.31 (s, 3F).

### Example 46 (R)—N-(2-(6-((1-(2-Methoxyethyl) piperidin-3-yl)amino)-4-methylpyridazin-3-yl)-5-(trifluoromethyl)phenyl) methanesulfonamide (56)

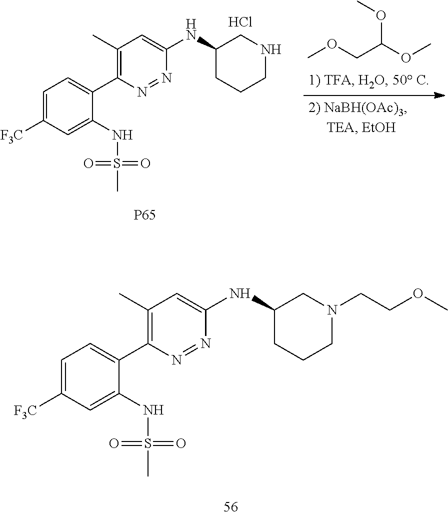

To a solution of 1,1,2-trimethoxyethane (500 mg, 4.16 mmol) in water (5 mL) was added TFA (0.5 mL) at 25° C. After stirred at 50° C. for 1 h, the reaction mixture quenched with sat. sodium carbonate aq. solution (5 mL) and extracted with EA (6 mL) for three times. The combined organic layers were washed with brine (10 mL), dried over Na₂SO₄ (s), filtered, and concentrated to give a residue. To a solution of P65 (100 mg, 0.198 mmol) and above residue in ethanol (5 mL) was added TEA (40 mg, 0.376 mmol). After stirred at 25° C. for 10 min, sodium triacetoxyborohydride (206 mg, 0.970 mmol) was added to the reaction mixture. After stirred at 25° C. for 3 h, the reaction mixture quenched with water (5 mL) and extracted with EA (5 mL) for three times. The combined organic layers were washed with brine (10 mL), dried over Na₂SO₄ (S), filtered and concentrated. The residue was purified by C18 column (ACN: 0.02% ammonium bicarbonate aq. solution=5% to 95%) to afford the desired compound 56 (17.1 mg, 97.83% purity from HPLC, 18% yield) as yellow solids. LCMS (ESI): R_{T}=0.778 min, mass calc, for C₂₁H₂₈F₃N₅O₃S 487.2, m/z found 488.1 [M+H]⁺. ¹H NMR (400 MHz, CDCl₃+D₂O), δ: 7.91 (s, 1H), 7.41-7.30 (m, 2H), 6.62 (s, 1H), 4.70-4.67 (m, 0.5H), 4.60-4.58 (m, 0.5H), 4.14 (br. s, 1H), 3.49-3.48 (m, 2H), 3.30 (s, 3H), 2.92 (s, 3H), 2.73-2.60 (m, 4H), 2.34-2.29 (m, 1H), 2.12 (s, 3H), 1.79-1.60 (m, 4H).

### Example 47. (R)—N-(2-(4-Methyl-6-((1-(3-methylbut-2-en-1-yl) piperidin-3-yl)amino)pyridazin-3-yl)-5-(trifluoromethyl)phenyl) methanesulfonamide (48)

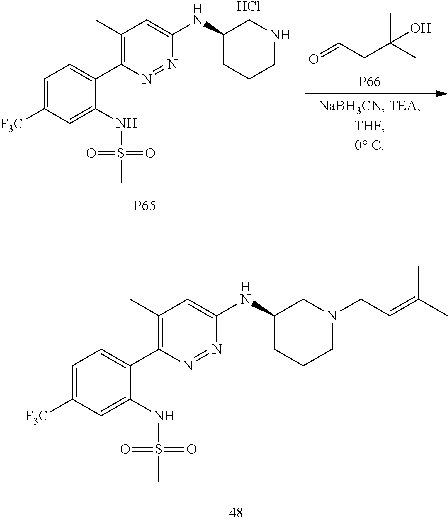

To a solution of (R)—N-(2-(4-methyl-6-(piperidin-3-ylamino)pyridazin-3-yl)-5-(trifluoromethyl)phenyl) methanesulfonamide hydrochloride (P65, 100 mg, 0.198 mmol), TEA (0.1 mL) and 3-hydroxy-3-methylbutanal (P66, 0.1 mL) in THF (2 mL) was added sodium cyanoborohydride (47 mg, 0.747 mmol) at 0° C. After stirred at 0° C. for 30 minutes, the reaction mixture diluted with water (5 mL) and extracted with EA (5 mL) for three times. The combined organic layers were washed with brine (10 mL), dried over Na₂SO₄ (s) and filtered. The filtrate was concentrated to leave a residue which was purified by C18 column (ACN: 0.02% ammonium bicarbonate aq. solution=5% to 95%) to afford the desired product (28.4 mg, 98.11% purity from HPLC, 28% yield) as white solids. LCMS (ESI): R_{T}=0.815 min, mass calc, for C₂₃H₃₀F₃N₅O₂S 497.2, m/z found 498.2 [M+H]⁺. 1H NMR (400 MHz, CD₃OD), δ: 7.88 (s, 1H), 7.55-7.48 (m, 2H), 6.81-6.80 (m, 1H), 5.31-5.27 (m, 1H), 4.18-4.13 (m, 1H), 3.18-3.11 (m, 3H), 2.93 (s, 3H), 2.84-2.81 (m, 1H), 2.32-2.17 (m, 2H), 2.11 (s, 3H), 2.06-2.02 (m, 1H), 1.87-1.82 (m, 1H), 1.76 (s, 3H), 1.73-1.70 (m, 1H), 1.69 (s, 3H), 1.49-1.43 (m, 1H). ¹⁹F NMR (376 MHz, CD₃OD), δ: −64.32 (s, 3F).

### Example 48. Analytical Data of Certain Examples of the Final Compounds

In the Table 4 presented NMR data for certain compounds of the presented invention.

TABLE 4
NMR spectra of certain final compounds.

| # | NMR spectra |
|---|---|
| 3 | ¹H NMR (400 MHz, DMSO-d₆), δ: 9.38 (br. s, exch. with D₂O, 1H), 7.54 (s, 1H), 7.19 (d, J = |
| | 9.2 Hz, 2H), 6.84 (d, J = 9.2 Hz, 1H), 6.70 (br. s, 1H), 4.42-4.31 (m, exch. with D₂O, 1H), 4.17- |
| | 4.02 (m, 1H), 3.50 (t, J = 5.6 Hz, 2H), 2.96 (d, J = 10.0 Hz, 1H), 2.85-2.53 |
| | (m, 4H), 2.41 (t, J = 6.0 Hz, 2H), 2.23-2.13 (m, 2H), 2.07 (s, 3H), 1.88-1.81 (m, |
| | 1H), 1.75-1.63 (m, 1H), 1.59-1.45 (m, 1H), 1.43-1.28 (m, 1H) |
| 12 | ¹H NMR (400 MHz, DMSO-d₆) δ: 9.63 (br. s, 1H), 9.20 (br. s, 1H), 7.54-7.19 |
| | (m, 2H), 6.71-6.69 (m, 1H), 6.12 (br. s, 1H), 4.40-4.30 (m, 1H), 3.73-3.71 (m, |
| | 1H), 3.45-3.40 (m, 1H), 3.10-2.65 (m, 5H), 2.07-1.74 (m, 3H), 1.51-1.49 (m, |
| | 1H) |
| 19 | ¹H NMR (400 MHz, DMSO-d₆), δ: 13.58 (br. s, 1H), 8.23 (br. s, 1H), 8.04 (t, J = |
| | 7.6 Hz, 1H), 7.62 (d, J = 7.6 Hz, 1H), 6.17 (d, J = 8.0 Hz, 1H), 4.40 (t, J = 5.2 |
| | Hz, 2H), 3.52 (dd, J = 11.6 and 6.0 Hz, 2H), 3.06 (d, J = 8.0 Hz, 1H), 2.76 (d, J = |
| | 11.2 Hz, 1H), 2.46-2.42 m, 2H), 2.23 (s, 3H), 2.12-2.03 (m, 2H), 1.93-1.90 (m, |
| | 1H), 1.74-1.71 (m, 1H), 1.57-1.48 (m, 2H) |
| 21 | ¹H NMR (400 MHz, DMSO-d₆), δ: 8.46 (br. s, 1H), 8.11 (d, J = 7.2 Hz, 1H), |
| | 7.71 (d, J = 7.6 Hz, 1H), 6.14 (d, J = 7.6 Hz, 1H), 4.43-4.36 (m, 1H), 3.08-3.01 |
| | (m, 1H), 2.72-2.61 (m, 1H), 2.25 (s, 3H), 2.23 (s, 3H), 2.05-1.92 (m, 3H), 1.77- |
| | 1.74 (m, 1H), 1.61-1.47 (m, 2H) |
| 27 | ¹H NMR (400 MHz, DMSO-d₆), δ: 9.33 (s, exch. with D₂O, 1H), 7.75 (s, 1H), |
| | 7.47 (s, 2H), 6.73 (s, 1H), 6.70 (s, exch. with D₂O, 1H), 4.07 (br. s, 1H), 3.02 |
| | (br. s, 1H), 2.91-2.66 (m, 5H), 2.33-2.29 (m, 1H), 2.16 (br. s, 1H), 2.00 (s, 3H), |
| | 1.90-1.87 (m, 1H), 1.76-1.73 (m, 1H), 1.56-1.53 (m, 1H), 1.38-1.33 (m, 1H), |
| | 1.01-0.99 (m, 6H) |
| 29 | ¹H NMR (400 MHz, DMSO-d₆), δ: 12.35 (s, exch. with D₂O, 1H), 8.76 (d, J = |
| | 1.6 Hz, 1H), 7.96 (d, J = 8.4 Hz, 1H), 7.79 (s, 1H), 7.51 (dd, J = 1.2 Hz, 8.4 Hz, |
| | 1H), 6.16 (d, exch. with D₂O, J = 7.6 Hz, 1H), 4.30-4.26 (m, 1H), 3.02 (d, J = |
| | 8.8 Hz, 1H), 2.75-2.69 (m, 2H), 2.59-2.53 (m, 1H), 2.19 (br. s, 4H), 2.10 (d, J = |
| | 10.0 Hz, 1H), 1.90-1.88 (m, 1H), 1.73-1.70 (m, 1H), 1.52-1.46 (m, 2H), 1.16- |
| | 1.14 (m, 6H), 0.98-0.96 (m, 6H) |
| 33 | ¹H NMR (400 MHz, DMSO-d₆), δ: 9.36 (s, 1H), 7.75 (s, 1H), 7.48 (br. s, 2H), |
| | 6.77-6.68 (m, 2H), 4.12-4.00 (m, 1H), 3.12-3.08 (m, 1H), 2.92 (s, 3H), 2.82-2.75 |
| | (m, 1H), 2.70-2.58 (m, 1H), 2.20-2.10 (m, 1H), 2.08-1.96 (m, 4H), 1.92-1.85 (m, |
| | 1H), 1.82-1.70 (m, 3H), 1.62-1.41 (m, 5H), 1.40-1.28 (m, 3H) |
| 34 | ¹H NMR (400 MHz, DMSO-d₆), δ: 9.37 (s, 1H), 7.77 (s, 1H), 7.60-7.48 (m, 2H), |
| | 6.80-6.68 (m, 2H), 4.10-4.00 (m, 1H), 2.95-2.86 (m, 4H), 2.80-2.72 (m, 1H), |
| | 2.66-2.55 (m, 1H), 2.01-1.85 (m, 7H), 1.82-1.68 (m, 4H), 1.64-1.58 (m, 2H), |
| | 1.58-1.49 (m, 1H), 1.36-1.28 (m, 1H) |
| 36 | ¹H NMR (400 MHz, DMSO-d₆), δ: 9.35 (s, 1H), 7.78 (s, 1H), 7.57-7.53 (m, 2H), |
| | 6.75-6.71 (m, 2H), 4.01-3.92 (m, 1H), 3.09-3.05 (m, 1H), 2.98 (s, 3H), 2.80-2.72 |
| | (m, 1H), 2.30-2.22 (m, 1H), 2.19-2.12 (m, 1H), 1.99 (s, 3H), 1.90-1.82 (m, 1H), |
| | 1.72-1.60 (m, 2H), 1.54-1.42 (m, 1H), 1.40-1.28 (m, 1H), 0.41-0.31 (m, 4H) |
| 39 | ¹H NMR (400 MHz, DMSO-d₆), δ: 9.36 (s, 1H), 7.77 (s, 1H), 7.60-7.48 (m, 2H), |
| | 6.78-6.70 (m, 2H), 4.10-4.00 (m, 1H), 3.88, 3.86 (dd, J = 10.8, 3.6 Hz, 2H), |
| | 3.28-3.23 (m, 2H), 3.12-3.02 (m, 1H), 2.96 (s, 3H), 2.82-2.72 (m, 1H), 2.60-2.54 |
| | (m, 1H), 2.30-2.22 (m, 1H), 2.20-2.08 (m, 1H), 2.00 (s, 3H), 1.92-1.84 (m, 1H), |
| | 1.78-1.60 (m, 3H), 1.58-1.30 (m, 4H) |
| 40 | ¹H NMR (400 MHz, CD₃OD), δ: 7.89 (s, 1H), 7.58-7.50 (m, 2H), 6.84-6.83 |
| | (m, 1H), 4.19-4.14 (m, 1H), 3.09-3.06 (m, 1H), 2.95 (s, 3H), 2.79-2.74 (m, |
| | 1H), 2.31-2.19 (m, 3H), 2.11 (s, 3H), 2.02-1.97 (m, 1H), 1.85-1.75 (m, 3H), |
| | 1.71-1.61 (m, 4H), 1.57-1.46 (m, 2H), 1.26-1.18 (m, 4H), 0.98-0.88 (m, |
| | 2H). ¹⁹F NMR (376 MHz, CD₃OD), δ: −62.81 (s, 3F) |
| 41 | ¹H NMR (400 MHz, DMSO-d₆), δ: 9.35 (br. s, 1H), 7.75 (s, 1H), 7.47 (br. s, |
| | 2H), 6.72-6.65 (m, 2H), 4.12-3.98 (m, 1H), 3.12-3.04 (m, 1H), 2.92 (s, 3H), |
| | 2.82-2.70 (m, 1H), 2.45-2.30 (m, 2H), 2.22-2.16 (m, 1H), 2.00 (s, 3H), 1.92-1.82 |
| | (m, 1H), 1.80-1.69 (m, 5H), 1.60-1.46 (m, 2H), 1.40-1.28 (m, 1H), 1.26-1.12 (m, |
| | 4H), 1.10-0.98 (m, 1H) |
| 44 | ¹H NMR (400 MHz, CD₃OD), δ: 7.89 (s, 1H), 7.58-7.49 (m, 2H), 7.36-7.25 |
| | (m, 5H), 6.81 (s, 1H), 4.18-4.12 (m, 1H), 3.59 (s, 2H), 3.03-2.99 (m, 1H), |
| | 2.93 (s, 3H), 2.71-2.68 (m, 1H), 2.29-2.23 (m, 1H), 2.19-2.15 (m, 1H), 2.09 |
| | (s, 3H), 2.01-1.96 (m, 1H), 1.84-1.78 (m, 1H), 1.72-1.66 (m, 1H), 1.49-1.41 |
| | (m, 1H). ¹⁹F NMR (376 MHz, CD₃OD), δ: −62.82 (s, 3F) |
| 45 | ¹H NMR (400 MHz, DMSO-d₆, VT at 90° C.), δ: 10.73 (br. s, exch. with D₂O, |
| | 1H), 8.22 (br. s, 1H), 7.99 (d, J = 8.0 Hz, 1H), 7.83 (s, 1H), 7.46 (br. s, 1H), 6.15 |
| | (br. s, exch. with D₂O, 1H), 3.05-2.99 (q, J = 7.2 Hz, 3H), 2.66-2.64 (m, 1H), |
| | 2.55-2.40 (m, 5H), 1.78-1.58 (m, 4H), 1.02 (t, J = 7.2 Hz, 3H) |
| 49 | ¹H NMR (400 MHz, DMSO-d₆), δ: 8.17 (s, 1H), 7.80 (s, 1H), 7.38-7.33 (m, 2H), |
| | 7.27 (s, 1H), 4.19 (br. s, 1H), 2.96-2.85 (m, 4H), 2.68-2.61 (m, 1H), 2.37 (q, |
| | J = 6.8 Hz, 2H), 2.15-2.06 (m, 2H), 1.91-1.87 (m, 1H), 1.76-1.73 (m, 1H), 1.60- |
| | 1.51 (m, 1H), 1.44-1.40 (m, 1H), 1.01 (t, J = 6.8 Hz, 3H) |
| 51 | ¹H NMR (400 MHz, DMSO-d₆), δ: 8.16 (br. s, 1H), 7.81 (br. s, 1H), 7.42-7.35 |
| | (m, 3H), 7.28 (s, 1H), 4.25-4.15 (m, 1H), 2.87-2.81 (m, 4H), 2.51-2.48 (m, 1H), |
| | 2.21-2.01 (m, 5H), 1.85-1.73 (m, 2H), 1.60-1.52 (m, 1H), 1.48-1.38 (m, 1H) |
| 52 | ¹H NMR (400 MHz, DMSO-d₆), δ: 10.74 (br. s, exch. with D₂O, 1H), 8.28 (br. |
| | s, 1H), 7.99 (d, J = 8.4 Hz, 1H), 7.81 (s, 1H), 7.49-7.48 (m, 1H), 6.39 (d, J = 6.0 |
| | Hz, exch. with D₂O, 1H), 4.56 (br. s, 1H), 3.01 (s, 3H), 2.76 (br. s, 1H), 2.37- |
| | 2.28 (m, 6H), 1.92-1.72 (m, 3H), 1.59-1.20 (m, 1H) |
| 53 | ¹H NMR (400 MHz, CD₃OD), δ: 7.88 (s, 1H), 7.54-7.48 (m, 2H), 6.82 (s, 1H), |
| | 4.19-4.13 (m, 1H), 3.19 (s, 4H), 2.94 (s, 3H), 2.86-2.82 (m, 1H), 2.58-2.54 |
| | (m, 2H), 2.35-2.22 (m, 2H), 2.12 (s, 3H), 2.06-2.02 (m, 1H), 1.89-1.83 (m, |
| | 1H), 1.78-1.74 (m, 3H), 1.51-1.45 (m, 1H), 1.18 (s, 6H). ¹⁹F NMR (376 MHz, |
| | CD₃OD), δ: −64.30 (s, 3F) |
| 54 | ¹H NMR (400 MHz, DMSO-d₆), δ: 9.63 (br. s, 1H), 7.80 (br. s, 1H), 7.50-7.42 |
| | (m, 2H), 6.28 (d, J = 7.6 Hz, 1H), 4.28-4026 (m, 1H), 3.40 (s, 1H), 3.09-3.06 (m, |
| | 1H), 2.99 (s, 3H), 2.74 (br. s, 2H), 2.27 (br. s, 3H), 2.07-1.90 (m, 3H), 1.75-1.57 |
| | (m, 6H), 1.42-1.20 (m, 5H) |
| 60 | ¹H NMR (400 MHz, DMSO-d₆), δ: 12.84 (s, exch. with D₂O, 1H), 8.09 (d, J = |
| | 8.4 Hz, 1H), 7.99 (s, 1H), 7.86 (s, 1H), 7.54 (d, J = 8.0 Hz, 1H), 6.30 (br. s, |
| | exch. with D₂O, 1H), 4.33 (br. s, 2H), 3.49-3.42 (m, 1H), 3.29 (br. s, 1H), 3.11 |
| | (s, 3H), 2.94-2.90 (m, 1H), 2.67-2.66 (m, 1H), 2.51-2.32 (m, 3H), 2.22 (s, 3H), |
| | 1.91-1.86 (m, 1H), 1.72-1.70 (m, 1H), 1.54-1.48 (m, 2H), 0.95-0.90 (m, 3H) |
| 61 | ¹H NMR (400 MHz, DMSO-d₆), δ: 9.41 (br. s, exch. with D₂O, 1H), 7.77 (s, |
| | 1H), 7.52 (br. s, 2H), 6.80-6.75 (m, 2H), 4.36-4.12 (m, 2H), 3.49-3.43 (m, 2H), |
| | 3.17-2.97 (m, 4H), 2.73-2.70 (m, 1H), 2.51-2.32 (m, 4H), 2.00 (s, 3H), 1.91-1.74 |
| | (m, 2H), 1.56-1.42 (m, 2H), 0.94 (d, J = 5.6 Hz, 3H) |
| 63 | ¹H NMR (400 MHz, DMSO-d₆), δ: 9.41 (br. s, exch. with D₂O, 1H), 7.77 (s, |
| | 1H), 7.55-7.49 (m, 2H), 6.79 (d, J = 7.6 Hz, 1H), 6.75 (s, 1H), 4.10-4.04 (m, |
| | 1H), 3.07-3.01 (m, 3H), 2.76-2.67 (m, 1H), 2.50-2.49 (m, 2H), 2.12-2.00 (m, |
| | 5H), 1.90-1.88 (m, 1H), 1.76-1.73 (m, 1H), 1.58-1.55 (m, 1H), 1.38-1.33 (m, |
| | 1H), 1.07-1.01 (m, 6H) |
| 64 | ¹H NMR (400 MHz, DMSO-d₆), δ: 12.70 (br. s, exch. with D₂O, 1H), 8.07 (d, |
| | J = 8.0 Hz, 1H), 8.01 (s, 1H), 7.88 (s, 1H), 7.55-7.53 (m, 1H), 6.27-6.25 (m, 1H), |
| | 4.31-4.29 (m, 1H), 3.01 (d, J = 8.8 Hz, 1H), 2.77-2.62 (m, 2H), 2.37 (q, J = 7.2 |
| | Hz, 2H), 2.21 (s, 3H), 1.99-1.90 (m, 3H), 1.74-1.70 (m, 1H), 1.58-1.45 (m, 2H), |
| | 1.01 (t, J = 7.2 Hz, 3H), 0.85-0.79 (m, 4H) |
| 69 | ¹H NMR (400 MHz, DMSO-d₆), δ: 12.68 (br. s, exch. with D₂O, 1H), 9.80 (br. |
| | s, exch. with D₂O, 1H), 7.84-7.39 (m, 5H), 6.70 (br. s, 2H), 4.08 (br. s, 1H), 3.03 |
| | (d, J = 6.0 Hz, 1H), 2.73 (br. s, 1H), 2.44-2.42 (m, 2H), 2.12-1.89 (m, 6H), 1.76- |
| | 1.73 (m, 1H), 1.60-1.54 (m, 1H), 1.38-1.33 (m, 1H), 1.03 (t, J = 7.2 Hz, 3H) |
| 70 | ¹H NMR (400 MHz, DMSO-d₆), δ: 7.81 (s, 1H), 7.52-7.49 (m, 2H), 6.79 (d, J = |
| | 8.0 Hz, 1H), 6.76 (s, 1H), 4.09-4.07 (m, 1H), 3.20-3.13 (m, 1H), 3.03-3.00 (m, |
| | 1H), 2.74-2.72 (m, 1H), 2.42 (q, J = 7.2 Hz, 2H), 2.13-2.07 (m, 1H), 2.02-1.97 |
| | (m, 4H), 1.90-1.87 (m, 1H), 1.77-1.72 (m, 1H), 1.60-1.51 (m, 1H), 1.38-1.28 (m, |
| | 1H), 1.11-1.00 (m, 9H) |
| 71 | ¹H NMR (400 MHz, DMSO-d₆), δ: 8.20-7.80 (br. s, 1H), 7.63 (d, J = 1.2 Hz, |
| | 1H), 7.21 (d, J = 7.6 Hz, 1H), 7.04 (d, J = 7.6 Hz, 1H), 6.71 (d, J = 6.8 Hz, 1H), |
| | 6.63 (d, J = 0.8 Hz, 1H), 4.17-4.08 (m, 1H), 3.40-3.37 (m, 1H), 3.10-3.05 (m, |
| | 1H), 2.80-2.74 (m, 1H), 2.69-2.64 (m, 4H), 2.04-1.97 (m, 4H), 1.93-1.83 (m, |
| | 1H), 1.69-1.50 (m, 2H) |
| 93 | ¹H NMR (400 MHz, TFA-d), δ: 8.78 (br. s, 1H), 7.68 (d, J = 8.8 Hz, 1H), 7.55 |
| | (d, J = 8.8 Hz, 1H), 7.55 (d, J = 8.0 Hz, 1H), 4.98-4.79 (m, 1H), 4.19-4.10 (m, |
| | 1H), 3.87-3.84 (m, 1H), 3.42-3.31 (m, 1H), 3.21-3.00 (m, 4H), 2.54-2.51 (m, |
| | 1H), 2.38-2.10 (m, 3H) |
| 100 | ¹H NMR (400 MHz, DMSO-d₆), δ: 9.68 (s, 1H), 7.67-7.0 (m, 2H), 6.80 (d, J = |
| | 8.0 Hz, 1H), 6.44 (d, J = 8.4 Hz, 1H), 5.80-4.50 (m, 3H), 4.30-4.11 (m, 1H), |
| | 3.48-3.43 (m, 1H), 2.98-2.70 (m, 6H), 2.10-1.60 (m, 7H) |
| 101 | ¹H NMR (400 MHz, DMSO-d₆), δ: 7.30-7.28 (m, 2H), 6.72 (d, J = 8.4 Hz, 1H), |
| | 6.37 (d, J = 8.0 Hz, 1H), 4.99 (s, 2H), 4.60 (s, 2H), 4.19-4.17 (m, 1H), 2.85-2.78 |
| | (m, 1H), 2.48-2.45 (m, 1H), 2.19-2.02 (m, 5H), 1.90-1.72 (m, 3H), 1.59-1.51 (m, |
| | 1H), 1.39-1.37 (m, 1H) |
| 102 | ¹H NMR (400 MHz, DMSO-d₆), δ: 7.95 (s, 1H), 6.96 (d, J = 8.4 Hz, 1H), 6.78 |
| | (d, J = 8.4 Hz, 1H), 6.28 (br. s, 2H), 6.17 (d, J = 8.0 Hz, 1H), 5.12 (s, 2H), 4.53- |
| | 4.46 (m, 1H), 2.68-2.66 (m, 1H), 2.40-2.21 (m, 6H), 1.69-1.51 (m, 4H) |
| 103 | ¹H NMR (400 MHz, DMSO-d₆), δ: 7.61 (s, 1H), 6.94 (d, J = 8.4 Hz, 1H), 6.76 |
| | (d, J = 8.8 Hz, 1H), 6.59 (s, 2H), 5.92 (d, J = 8.0 Hz, 1H), 5.03 (s, 2H), 4.31- |
| | 4.22 (m, 1H), 2.93 (d, J = 8.0 Hz, 1H), 2.64 (d, J = 10.4 Hz, 1H), 2.19 (s, 3H), |
| | 2.16 (s, 3H), 1.96-1.86 (m, 3H), 1.72-1.68 (m, 1H), 1.59-1.49 (m, 1H), 1.48-1.35 |
| | (m, 1H). |
| | Structure of 103 was confirmed by NOE & HSQC studies: |
| | 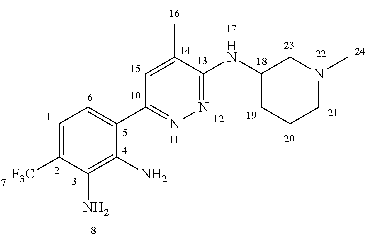 [SMILES: Cc1cc(-c2ccc(C(F)(F)F)c(N)c2N)nnc1NC1CCCN(C)C1] |
| | NOE Experimental data (performed at 90° C.): On irradiation of H17 (5.61 ppm) |
| | showing enhancement of H18 (4.31 ppm) and H16 (2.17 ppm) protons |
| 108 | ¹H NMR (400 MHz, CD₃OD), δ: 7.15 (d, J = 7.6 Hz, 1H), 6.98 (d, J = 7.6 Hz, |
| | 1H), 6.88 (s, 1H), 6.81 (s, 1H), 4.19-4.06 (m, 1H), 3.23-3.16 (m, 1H), 2.93- |
| | 2.83 (m, 2H), 2.78 (s, 3H), 2.49-2.18 (m, 2H), 2.08-2.04 (m, 4H), 1.96-1.91 |
| | (m, 1H), 1.79-1.77 (m, 1H), 1.51-1.38 (m, 1H), 1.15-1.10 (m, 6H) |
| 109 | ¹H NMR (400 MHz, DMSO-d₆), δ: 7.27 (d, J = 8.0 Hz, 1H), 7.22 (s, 1H), 7.04 |
| | (d, J = 7.6 Hz, 1H), 6.96 (s, 1H), 4.20 (bs, 1H), 3.59-3.49 (m, 2H), 3.39-3.37 |
| | (m, 1H), 3.15 (dd, J = 14.4, 7.2 Hz, 2H), 2.94 (t, J = 12.0 Hz, 1H), 2.79 (t, J = |
| | 12.0 Hz, 1H), 2.14-2.04 (m, 5H), 1.87-1.78 (m, 1H), 1.62-1.53 (m, 1H), 1.29 |
| | (d, J = 6.8 Hz, 6H), 1.11 (t, J = 7.2 Hz, 3H) |
| 111 | ¹H NMR (400 MHz, CD₃OD), δ: 7.18-7.16 (m, 1H), 6.97-6.95 (m, 2H), 6.82 |
| | (s, 1H), 4.14-4.08 (m, 1H), 3.70-3.63 (m, 1H), 3.14-3.10 (m, 1H), 2.84-2.76 |
| | (m, 2H), 2.37-2.20 (m, 2H), 2.06 (s, 3H), 2.04-2.00 (m, 1H), 1.83-1.66 (m, |
| | 2H), 1.46-1.37 (m, 1H), 1.16-1.14 (m, 6H), 1.10-1.08 (m, 6H). ¹⁹F NMR |
| | (376 MHz, CD₃OD), δ: −64.42 (s, 3F) |
| 112 | ¹H NMR (400 MHz, CD₃OD), δ: 7.48 (s, 1H), 7.41-7.39 (m, 1H), 7.25-7.19 |
| | (m, 3H), 7.01-6.99 (m, 2H), 6.93-6.90 (m, 1H), 6.71 (s, 1H), 4.14-4.07 (m, |
| | 1H), 3.17-3.15 (m, 1H), 2.88-2.78 (m, 2H), 2.41-2.20 (m, 2H), 2.11 (s, 3H), |
| | 2.03-1.99 (m, 1H), 1.85-1.67 (m, 2H), 1.46-1.39 (m, 1H), 1.12-1.10 (m, |
| | 6H). ¹⁹F NMR (376 MHz, CD₃OD), δ: −64.47 (s, 3F) |
| 113 | ¹H NMR (400 MHz, DMSO-d₆), δ: 10.61 (s, exch. with D₂O, 1H), 8.16 (s, 1H), |
| | 7.69 (d, J = 8.0 Hz, 1H), 7.63 (d, J = 8.0 Hz, 1H), 6.75 (s, 1H), 6.73 (s, exch. |
| | with D₂O, 1H), 6.29 (t, J = 53.6 Hz, 1H), 4.01-3.99 (m, 1H), 2.95 (d, J = 8.0 Hz, |
| | 1H), 2.75-2.62 (m, 2H), 2.18 (t, J = 10.4 Hz, 1H), 2.04 (t, J = 9.2 Hz, 1H), 1.98 |
| | (s, 3H), 1.90-1.82 (m, 1H), 1.72-1.68 (m, 1H), 1.54-1.45 (m, 1H), 1.34-1.23 (m, |
| | 1H), 0.97-0.95 (m, 6H) |
| 114 | ¹H NMR (400 MHz, CD₃OD), δ: 7.64-7.62 (m, 2H), 6.92-6.91 (m, 2H), 4.44- |
| | 4.38 (m, 1H), 3.29-3.23 (m, 2H), 3.10-3.07 (m, 1H), 2.91-2.75 (m, 2H), 2.53- |
| | 2.45 (m, 2H), 2.26 (s, 3H), 1.99-1.81 (m, 2H), 1.76-1.57 (m, 2H), 1.34-1.30 |
| | (m, 3H), 1.13-1.12 (m, 6H). ¹⁹F NMR (376 MHz, CD₃OD), δ: −64.53 (s, 3F) |
| 115 | ¹H NMR (400 MHz, CD₃OD, δ: 7.63-7.61 (m, 2H), 6.94-6.88 (m, 2H), 4.41- |
| | 4.38 (m, 1H), 3.76-3.73 (m, 1H), 3.05-3.02 (m, 1H), 2.82-2.72 (m, 2H), 2.49- |
| | 2.41 (m, 2H), 2.25 (s, 3H), 1.98-1.94 (m, 1H), 1.82-1.58 (m, 3H), 1.27 (d, J = |
| | 6.4 Hz, 6H), 1.11 (d, J = 6.4 Hz, 6H) |
| 116 | ¹H NMR (400 MHz, DMSO-d₆), δ: 11.23 (br. s, exch. with D₂O, 1H), 7.96 (s, |
| | 1H), 7.70-7.64 (m, 2H), 6.72 (br. s, 1H is exch. with D₂O, 2H), 4.04-4.03 (m, |
| | 1H), 3.03 (br. s, 1H), 2.74 (br. s, 1H), 2.44 (br. s, 2H), 2.09-1.86 (m, 6H), 1.75- |
| | 1.72 (m, 1H), 1.57-1.54 (m, 1H), 1.34-1.31 (m, 1H), 1.01 (t, J = 7.2 Hz, 3H) |
| 118 | ¹H NMR (400 MHz, DMSO-d₆), δ: 7.70 (d, J = 0.8 Hz, 1H), 7.66 (d, J = 8.4 Hz, |
| | 1H), 7.09 (d, J = 1.2 Hz, 1H), 7.05 (s, exch. with D₂O, 2H), 6.85 (dd, J = 1.2 Hz, |
| | 8.4 Hz, 1H), 5.94 (d, J = 8.0 Hz, exch. with D₂O, 1H), 4.26-4.24 (m, 1H), 2.95- |
| | 2.92 (m, 1H), 2.77-2.66 (m, 2H), 2.17 (br. s, 5H), 1.90-1.87 (m, 1H), 1.72-1.69 |
| | (m, 1H), 1.55-1.41 (m, 2H), 0.99-0.96 (m, 6H) |
| 119 | ¹H NMR (400 MHz, CD₃OD), δ: 7.90 (s, 1H), 7.68 (d, J = 8.0 Hz, 1H), 7.03 (d, |
| | J = 8.0 Hz, 1H), 6.94 (s, 1H), 4.53-4.45 (m, 1H), 3.58-3.50 (m, 2H), 3.39 (d, J = |
| | 14.0 Hz, 1H), 2.97-2.85 (m, 5H), 2.28 (s, 3H), 2.15-2.04 (m, 2H), 1.90- |
| | 1.77 (m, 1H), 1.71-1.62 (m, 1H), 1.30 (d, J = 6.4 Hz, 6H). ¹⁹F NMR (376 MHz, |
| | CD₃OD), δ: −61.60 (s, 3F), −74.01 (s, 6F) |
| 120 | ¹H NMR (400 MHz, DMSO-d₆), δ: 13.55 (s, exch. with D₂O, 1H), 8.77 (s, 1H), |
| | 8.07 (d, J = 8.4 Hz, 1H), 7.89 (s, 1H), 7.65 (dd, J = 1.2 Hz, 8.4 Hz, 1H), 6.49 (t, |
| | J = 53.6 Hz, 1H), 6.23 (d, exch. with D₂O, J = 8.0 Hz, 1H), 4.30-4.24 (m, 1H), |
| | 3.01-2.99 (m, 1H), 2.77-2.66 (m, 2H), 2.20-2.10 (m, 5H), 1.90-1.87 (m, 1H), |
| | 1.73-1.69 (m, 1H), 1.57-1.43 (m, 2H), 0.98-0.96 (m, 6H) |
| 121 | ¹H NMR (400 MHz, CD₃OD, δ: 7.77 (d, J = 8.0 Hz, 1H), 7.69 (s, 1H), 7.53 (s, |
| | 1H), 7.30 (t, J = 8.4 Hz, 2H), 7.17-7.13 (m, 3H), 7.00 (t, J = 8.4 Hz, 1H), 4.57 |
| | (s, 2H), 4.40 (s, 1H), 3.13-3.04 (m, 1H), 2.82 (s, 1H), 2.74 (s, 1H), 2.45 (d, J = |
| | 10.8 Hz, 2H), 2.25 (s, 3H), 1.97 (s, 1H), 1.79 (s, 1H), 1.71-1.60 (m, 2H), 1.11 |
| | (d, J = 6.8 Hz, 6H). ¹⁹F NMR (376 MHz, CD₃OD), δ: −64.59 (s, 3F) |
| 122 | ¹H NMR (400 MHz, DMSO-d₆), δ: 14.41 (br. s, exch. with D₂O, 1H), 8.65 (s, |
| | 1H), 8.13 (d, J = 8.4 Hz, 1H), 7.94 (s, 1H), 7.70 (d, J = 7.6 Hz, 1H), 6.27 (br. s, |
| | exch. with D₂O, 1H), 4.26 (br. s, 1H), 3.03 (br. s, 1H), 2.70-2.66 (m, 2H), 2.21- |
| | 2.07 (m, 5H), 1.90 (br. s, 1H), 1.72 (br. s, 1H), 1.51 (br. s, 2H), 1.07-0.91 (m, 6H) |
| 124 | ¹H NMR (400 MHz, DMSO-d₆), δ: 14.34 (br. s, exch. with D₂O, 1H), 8.64 (s, |
| | 1H), 8.13 (d, J = 8.4 Hz, 1H), 7.94 (s, 1H), 7.71 (d, J = 7.6 Hz, 1H), 6.30 (br. s, |
| | exch. with D₂O, 1H), 4.31 (br. s, 1H), 3.09 (br. s, 1H), 2.77 (br. s, 1H), 2.43-2.40 |
| | (m, 2H), 2.20 (s, 3H), 2.04-1.90 (m, 3H), 1.72 (br. s, 1H), 1.59-1.48 (m, 2H), |
| | 1.02 (t, J = 6.8 Hz, 3H) |
| 125 | ¹H NMR (400 MHz, DMSO-d₆), δ: 14.75 (br. s, exch. with D₂O, 1H), 8.63 (br. |
| | s, 1H), 8.12 (br. s, 1H), 7.95 (s, 1H), 7.72-7.69 (m, 1H), 6.26 (br. s, exch. with |
| | D₂O, 1H), 4.24-4.22 (m, 1H), 3.08 (br. s, 1H), 2.71-2.66 (m, 2H), 2.19-2.05 (m, |
| | 5H), 1.90 (br. s, 1H), 1.72 (br. s, 1H), 1.52 (br. s, 2H), 0.98 (br. s, 6H) |
| 129 | ¹H NMR (400 MHz, DMSO-d₆), δ: 7.16-7.14 (m, 1H), 7.06 (d, J = 1.2 Hz, 1H), |
| | 6.88 (dd, J = 1.2 Hz, 8.0 Hz, 1H), 6.73 (s, 1H), 6.67-6.62 (m, 1H), 5.32 (s, 2H), |
| | 4.24 (s, 1H), 4.04 (br. s, 1H), 3.45-3.43 (m, 1H), 3.27-3.26 (m, 1H), 2.94-2.88 |
| | (m, 1H), 2.67-2.66 (m, 2H), 2.42-2.40 (m, 1H), 2.33-2.26 (m, 1H), 1.99 (s, 3H), |
| | 1.81-1.69 (m, 2H), 1.55-1.37 (m, 2H), 0.94 (d, J = 6.4 Hz, 3H) |
| 131 | ¹H NMR (400 MHz, DMSO-d₆), δ: 7.71 (s, 1H), 7.67 (d, J = 8.0 Hz, 1H), 7.09 |
| | (d, J = 1.2 Hz, 1H), 7.06 (s, exch. with D₂O, 2H), 6.85 (dd, J = 1.2 Hz, 8.0 Hz, |
| | 1H), 5.99 (d, exch. with D₂O, J = 6.4 Hz, 1H), 4.28 (br. s, 1H), 3.02 (br. s, 1H), |
| | 2.75 (br. s, 1H), 2.39 (br. s, 2H), 2.17 (s, 3H), 1.98-1.89 (m, 3H), 1.74-1.71 (m, |
| | 1H), 1.59-1.44 (m, 2H), 1.01 (t, J = 6.8 Hz, 3H) |
| 133 | ¹H NMR (400 MHz, DMSO-d₆), δ: 11.91 (br. s, exch. with D₂O, 1H), 8.69 (s, |
| | 1H), 7.91 (d, J = 8.4 Hz, 1H), 7.75 (s, 1H), 7.52 (dd, J = 1.2 Hz, 8.0 Hz, 1H), |
| | 6.17 (d, exch. with D₂O, J = 8.0 Hz, 1H), 4.33-4.31 (m, 1H), 3.02 (d, J = 7.6 Hz, |
| | 1H), 2.74 (d, J = 10.4 Hz, 1H), 2.36 (q, J = 7.2 Hz, 2H), 2.19 (s, 3H), 2.10 (s, |
| | 3H), 1.97-1.89 (m, 3H), 1.74-1.70 (m, 1H), 1.60-1.45 (m, 2H), 1.00 (t, J = 7.2 |
| | Hz, 3H) |
| 135 | ¹H NMR (400 MHz, DMSO-d₆), δ: 12.91 (s, exch. with D₂O, 1H), 8.90 (s, 1H), |
| | 8.00 (d, J = 8.0 Hz, 1H), 7.86 (s, 1H), 7.50 (dd, J = 1.2 Hz, 8.4 Hz, 1H), 6.18 (d, |
| | exch. with D₂O, J = 7.6 Hz, 1H), 4.23-4.21 (m, 1H), 3.09 (d, J = 6.8 Hz, 1H), |
| | 2.76-2.66 (m, 2H), 2.21-2.17 (m, 4H), 2.05 (t, J = 9.6 Hz, 1H), 1.90-1.88 (m, |
| | 1H), 1.72-1.69 (m, 1H), 1.53-1.47 (m, 2H), 1.24 (s, 9H), 0.99-0.94 (m, 6H) |
| 136 | ¹H NMR (400 MHz, DMSO-d₆), δ: 9.50 (s, exch. with D₂O, 1H), 8.18 (s, 1H), |
| | 7.54 (dd, J = 0.8 Hz, 8.0 Hz, 1H), 7.49 (d, J = 8.0 Hz, 1H), 6.71 (d, J = 0.8 Hz, |
| | 1H), 6.67 (d, exch. with D₂O, J = 8.0 Hz, 1H), 4.03-4.00 (m, 1H), 2.96-2.94 (m, |
| | 1H), 2.76-2.69 (m, 1H), 2.67-2.62 (m, 1H), 2.18 (t, J = 10.4 Hz, 1H), 2.18 (t, J = |
| | 9.6 Hz, 1H), 1.93 (s, 3H), 1.91 (s, 3H), 1.91-1.85 (m, 1H), 1.73-1.69 (m, 1H), |
| | 1.54-1.45 (m, 1H), 1.35-1.26 (m, 1H), 0.97-0.95 (m, 6H) |
| 137 | ¹H NMR (400 MHz, DMSO-d₆), δ: 11.91 (s, exch. with D₂O, 1H), 8.69 (s, 1H), |
| | 7.92 (d, J = 8.0 Hz, 1H), 7.76 (s, 1H), 7.52 (dd, J = 1.2 Hz, 8.0 Hz, 1H), 6.17 |
| | (br. s, exch. with D₂O, 1H), 4.32 (br. s, 1H), 2.98-2.96 (m, 1H), 2.74-2.66 (m, |
| | 2H), 2.19-2.10 (m, 8H), 1.93-1.90 (m, 1H), 1.73 (br. s, 1H), 1.58-1.52 (m, 2H), |
| | 0.99 (br. s, 6H) |
| 138 | ¹H NMR (400 MHz, DMSO-d₆), δ: 9.85 (s, exch. with D₂O, 1H), 8.23 (d, J = 1.2 |
| | Hz, 1H), 7.63 (d, J = 8.0 Hz, 1H), 7.56 (dd, J = 1.2 Hz, 8.0 Hz, 1H), 6.79 (s, |
| | 1H), 6.76 (d, exch. with D₂O, J = 8.0 Hz, 1H), 3.99-3.97 (m, 1H), 2.96 (d, J = |
| | 7.6 Hz, 1H), 2.74-2.62 (m, 2H), 2.17 (t, J = 10.0 Hz, 1H), 2.07 (s, 3H), 2.01 (t, J = |
| | 10.0 Hz, 1H), 1.88-1.84 (m, 1H), 1.71-1.68 (m, 1H), 1.54-1.45 (m, 1H), 1.35- |
| | 1.29 (m, 1H), 1.21 (s, 9H), 0.99-0.94 (m, 6H) |

### Biological Assays

### Example A. Primary Assays Used to Determine IL1b Release THP-1 (ELISA)

### Materials and Equipment:

Microbiological safety cabinet, classII (NuAire, USA)
CO₂ incubator (VWR Science, USA)
Countess Automated Cell Counter (Invitrogen, USA, cat. number C10227)
Centrifuge CM 6M (Elmi, Latvia)
Serological pipettes 2, 5, 10, 25, 50, 100 ml sterile (Santa Cruz Biotechnology, Inc., USA, sc-200277, sc-200279, sc-200281, sc-200283, Sc-213234, sc-516777)
Single and multichannel pipettes 0.2-10 μL, 50-1200 μL (Eppendorf AG-Hamburg, Germany)
50 mL PP sterile tubes (Greiner, cat. Number 210261).

### Reagents and Solutions:

RPMI-1640 (PanEco, C330)
Sodium pyruvate (Paneco, cat. number Φ023)
Non-Essential amino acids 50× (Paneco, Φ116)
Essential amino acids 50× (Paneco, Φ115/50)
FBS (Hyclone Laboratories Inc, USA, SV30160.03)
Antibiotic-antimycotic (Gibco, USA, 15240-062)
2-mercaptoethanol 1000×55 mM (Gibco, USA, 21985-023)
LPS-EB ultrapure (invivogen, cat. number tlrl-3pelps)
Nigericin (Sigma, cat. number N7143)
ELISA kit, IL-1beta (Cat #A-8766, Interleukin-1 beta-EIA-BEST, Vector Best).
   LPS Preparation (5 mg/ml):
1. Add 1 ml of endotoxin-free water according manufacturer's protocol
2. Vortex until complete solubilization
3. Aliquote and store at 20° C. for long term storage.

### Nigericin Preparation (10 mM):

1. Add methanol to 10 mg of powder to produce the 10 mM Nigericin solution
2. Vortex until complete solubilization
3. Store at 4° C.

### Cells Propagation:

THP-1 cells were propagated in RPMI-1640 (10% FBS, Antibiotic-antimycotic 1×, non-Essential amino acids 1×, Essential amino acids 1×, Sodium pyruvate 2×, 2-mercaptoethanol 0.05 mM) at 37° C. and at 5% CO₂ in an incubator.

### Assay Steps:

1. THP-1 cells were spined at 100 g for 5 min in 50 ml tube.
2. THP-1 cells were resuspended in warm culture medium (w/o FBS, w/o Antibiotic-antimycotic, non-Essential amino acids 1×, Essential amino acids 1×, Sodium pyruvate 2×) to the concentration 1,3×10⁶ cells.
3. THP-1 cells were primed in tube with 1 μg/ml of LPS for 4 h at 37° C. and at 5% CO₂ in an incubator.
4. THP-1 cells were stimulated with 10 μM of Nigericin (Cat #N7143-10 MG, Sigma Aldrich) for 2 h at 37° C. and at 5% CO₂ in an incubator.
5. After the full activation, the supernatant was collected and the levels of IL-1β were assessed via ELISA according manufacturer instructions (Cat #A-8766, Vector Best).

The average EC₅₀ values are shown in Table A, wherein “A” corresponds to EC₅₀: A≤0.5 μM, “B” corresponds to 0.5 μM<EC₅₀≤1.0 μM, “C” 1.0 μM<EC₅₀≤5.0 μM, “D” corresponds to 5.0 μM<EC₅₀≤30 μM and “E” corresponds to EC₅₀>30 μM.

TABLE A
THP 1 IL 1b

| | THP 1 IL 1b, |
|---|---|
| Compound | EC₅₀, μM |
| 1 | C |
| 2 | B |
| 4 | B |
| 5 | A |
| 6 | B |
| 7 | A |
| 8 | A |
| 9 | B |
| 10 | D |
| 14 | C |
| 15 | C |
| 16 | C |
| 17 | D |
| 18 | E |
| 19 | E |
| 20 | E |
| 21 | D |
| 22 | D |
| 23 | E |
| 24 | E |
| 25 | A |
| 26 | A |
| 27 | A |
| 28 | A |
| 29 | C |
| 30 | A |
| 31 | A |
| 32 | A |
| 33 | A |
| 34 | A |
| 35 | A |
| 36 | A |
| 37 | A |
| 38 | A |
| 39 | A |
| 40 | A |
| 41 | A |
| 42 | A |
| 43 | A |
| 44 | B |
| 45 | B |
| 46 | B |
| 47 | B |
| 48 | B |
| 49 | C |
| 50 | C |
| 51 | C |
| 52 | C |
| 53 | C |
| 54 | D |
| 55 | C |
| 56 | C |
| 62 | A |
| 63 | A |
| 64 | A |
| 65 | A |
| 66 | B |
| 67 | C |
| 68 | D |
| 69 | C |
| 70 | C |
| 71 | C |
| 72 | D |
| 93 | D |
| 100 | A |
| 101 | C |
| 102 | D |
| 103 | D |
| 108 | A |
| 109 | A |
| 110 | A |
| 111 | A |
| 112 | A |
| 113 | A |
| 114 | A |
| 115 | A |
| 116 | A |
| 117 | A |
| 118 | A |
| 119 | A |
| 120 | A |
| 121 | B |
| 122 | B |
| 123 | C |
| 124 | C |
| 125 | C |
| 130 | A |
| 131 | A |
| 132 | A |
| 133 | B |
| 134 | B |
| 135 | B |
| 136 | B |
| 137 | C |
| 138 | C |

*EC₅₀ is the concentration of a drug, antibody or toxicant which induces a response halfway between the baseline and maximum after a specified exposure time. EC₅₀: A ≤ 0.5 μM; 0.5 μM < B ≤ 1.0 μM; 1.0 μM < C ≤ 5.0 μM; 5.0 μM < D ≤ 30 μM; E > 30 μM

### Example B. Primary Assays Used to Determine IL1b-Release PBMC (ELISA)

### Materials and Equipment:

Microbiological safety cabinet, classII (NuAire, USA)
CO₂ incubator (VWR Science, USA)
Countess Automated Cell Counter (Invitrogen, USA, cat. number C10227)
Centrifuge CM 6M (Elmi, Latvia)
Serological pipettes 2, 5, 10, 25, 50, 100 ml sterile (Santa Cruz Biotechnology, Inc., USA, sc-200277, sc-200279, sc-200281, sc-200283, sc-213234, sc-516777)
Single and multichannel pipettes 0.2-10 μL, 50-1200 μL (Eppendorf AG-Hamburg, Germany)
50 mL PP sterile tubes (Greiner, cat. Number 210261).

### Reagents and Solutions:

RPMI-1640 (PanEco, C330)
Sodium pyruvate (Paneco, cat. number Φ023)
Non-Essential amino acids 50× (Paneco, Φ116)
Essential amino acids 50× (Paneco, Φ115/50)
FBS (Hyclone Laboratories Inc, USA, SV30160.03)
Antibiotic-antimycotic (Gibco, USA, 15240-062)
LPS-EB ultrapure (invivogen, cat. number tlrl-3pelps)
Nigericin (Sigma, cat. number N7143)
ELISA kit, IL-1beta (Cat #A-8766, Interleukin-1 beta-EIA-BEST, Vector Best).
   LPS Preparation (5 mg/ml):
1. Add 1 ml of endotoxin-free water according manufacturer's protocol
2. Vortex until complete solubilization
3. Aliquote and store at 20° C. for long term storage.

### Nigericin Preparation (10 mM):

1. Add methanol to 10 mg of powder to produce the 10 mM Nigericin solution
2. Vortex until complete solubilization
3. Store at 4° C.

### Cells Preparation:

PBMC were thawed and inoculated immediately in RPMI-1640 (Antibiotic-antimycotic 1×, non-Essential amino acids 1×, Essential amino acids 1×, Sodium pyruvate 2×).

Isolation of Mononuclear Cells from Human Peripheral Blood by Density Gradient Centrifugation:
Freezing solution I: RPMI 1640+GlutaMAX+Pen/Strep+Pyr-Na (PBMC assay medium).
Freezing solution II: 80% heat inactivated FBS+20% DMSO (Sigma, cat #D2650).

### Protocol:

Collect 45 mL blood to 50 mL Falcon tube with 5 mL Heparin Na (500 ME/mL).
Dilute cell with 2V of PBS.
Prepare 50 mL Falcon tubes with Ficoll: add 15 mL Ficoll-Paque per tube.
Carefully layer 35 mL of diluted blood over 15 mL of Ficoll-Paque in 50 mL sterile tube.
Centrifugate at Allegra 6R at 400×g (1350 RPM for GH-3.8 bucket rotor) 30 min 20° C. without brake.
Aspirate the upper layer leaving the mononuclear cell layer undisturbed at the interphase.
Carefully transfer the mononuclear cell layer to a new 50 mL sterile tube.
Fill the conical tube with buffer, mix, and centrifugate 300×g (1150 RPM) 10 min 20° C. Carefully remove the supernatant completely.
Resuspend cell pellet in each 50 mL Falcon tube in 2-3 mL PBS. Combine cell pellets from 5-7 50 mL Falcon tubes to one 15 mL Falcon tube.
Centrifugate tubes at 200×g (950 RPM) for 15 minutes 20° C. Carefully remove the supernatant completely.
Resuspend cell pellet in 5-10 mL Freezing medium I. Count cells. Dilute cells to 2× freezing concentration with Freezing solution I.
Dropwise add the same volume of Freezing solution II.
Aliquot 1 mL or 2 mL cell suspension to each cryotube, firmly close the lid and put the tubes to freezing boxes.
Put freezing boxes to −80.
After overnight incubation transfer the cryotubes to Dewar's vessel with liquid nitrogen.

### Assay Steps

PBMC were spined at 100 g for 5 min in 50 ml tube.
PBMC were resuspended in warm culture medium (w/o FBS, w/o Antibiotic-antimycotic, non-Essential amino acids 1×, Essential amino acids 1×, Sodium pyruvate 2×) to the concentration 1,3×10⁶ cells.
PBMC were primed in tube with 1 μg/ml of LPS for 2.5 h at 37° C. and at 5% CO₂ in an incubator. After the incubation cells were transferred into 96-well plate with V-bottom.
1000× stocks of compounds in DMSO were added to cells using the dilution scheme:
1 ul of 1000× compounds was added to 49 ul of RPMI to obtain 20× compound's working solution. Then 20× compounds working solution was added to cells to 1× concentration. Final DMSO content was 0.1%.
PBMC were incubated with compounds for 30 min at 37° C. and at 5% CO₂ in an incubator.
Then PBMC were stimulated with 10 μM of Nigericin (Cat #N7143-10 MG, Sigma Aldrich) for 1 h at 37° C. and at 5% CO₂ in an incubator.
Plates with cells were frozen at −80° C. overnight.
After thawing plates were spined at 300 g for 3 min, the supernatant was collected and the levels of IL-1B were assessed via ELISA with sample dilution 1:50 (Cat #A-8766, Vector Best). The more beneficial approach is freezing supernatant without cells.

### Controls:

K−-non-stimulated cells without compounds addition
K+-stimulated cells without compounds addition.

### Example C. Inflammasome Antagonists Test, CRCs

### The Assay Principle:

The Human Interleukin1-β (IL1β) assay (Cisbio) is based on HTRF® technology (Homogeneous Time-Resolved Fluorescence). IL1β is detected by an anti-IL1β MAb labeled with XL665, the second MAb is labeled with Cryptate. These mouse MAbs recognize distinct epitopes of human IL1β. They do not crossreact with IL1β from other species. Specific signal (i.e. energy transfer) is proportional to the concentration of IL1β in the sample or calibrator. The Human Interleukin1-β assay (Cisbio) is used as a functional assay to determine NALP3 inflammasome antagonists by measuring IL1β secretion in peripheral blood mononuclear cells (PBMC) upon stimulation by LPS and Nigericin in the presence of compounds tested.

### Materials and Equipment:

Biomek FX Laboratory Automated Workstation (Beckman Coulter Inc., Fullerton, CA)
Biomek 2000 (Beckman Coulter Inc., Fullerton, CA)
Microscope Axiovert 40 (Zeiss, USA)
Microbiological safety cabinet, classII (NuAire, USA)
CO₂ incubator (VWR Science, USA)
Countess Automated Cell Counter (Invitrogen, USA, cat. number C10227)
Centrifuge Allegra 6R (Beckman Coulter Inc., Fullerton, CA) with GH-3.8 bucket rotor
Centrifuge Eppendorf 5810 (Eppendorf, Germany) with A-4-62 rotor
Centrifuge CM 6M (Elmi, Latvia)
UltraSonic Bath (Elmasonic, cat. number S30H) Single and multichannel pipettes 0.2-10 μL, 50-1200 μL (Eppendorf AG-Hamburg, Germany)
Multilabel multitask plate reader Wallac 1420 Victor³ (PerkinElmer, USA)
50 mL PP sterile tubes (Greiner, cat. Number 210261)
Cryo.s, PP sterile cryovials (Greiner, cat. Number 2015-10)
Nunc reservoir TRAY RECT W/LID CLR STRL CS90 (Greiner, 73520-160)
V-shaped Biomek reservoirs (Beckman, cat. number 372790)
384 well plates 120 μL/well, clear, diamond bottom (Greiner, cat. number 47743-468)
Biomek AP384 P30 pipette tips (Beckman Coulter, cat. number 719222)
Biomek 2000 P250 pipette tips (Beckman Coulter, cat. number 372654)
Biomek 2000 P20 pipette tips (Beckman Coulter, cat. number 609044)
OptiPlate-384, White Opaque 384-well MicroPlate, 200 plates/case (Perkin Elmer, cat. number 6007299).

### Reagents and Solutions:

DMSO (Panreac, cat. number 141954.1608) (for cpds dilution)
DMSO (Sigma, cat. number D2650) (for cells freezing)
Human IL1β detection Kit (Cisbio, cat. number 62ILIPEC)
Phosphate buffer (Amresco, cat. number E404)
Ficoll-Paque Premium (density 1.077±0.001 g/ml) (GE Healthcare, cat. number 17-5442-03)
Heparin Na 5000 ME/mL (OAO [symbol] )
RPMI 1640 (Gibco, cat. number 21870)
GlutaMAX (Gibco, cat. number 35050)
NEAA, non-essential amino acids (Paneco, cat. number Φ115)
Pen-strep (Paneco, cat. number A065)
FBS (HyClone, cat. number SH30070.03)
Sodium pyruvate (Paneco, cat. number Φ023)
LPS-EB ultrapure (invivogen, cat. number tlrl-pelps)
Nigericin (Sigma, cat. number N7143).
   Human IL1β Detection Kit (Cisbio, Cat. Number 62ILIPEC) Reagents Preparation:

Reconstitute 1 vial of anti-IL1b-Cryptate and 1 vial of anti-IL1b-XL665 with 5 mL of distilled water each. Mix gently. Store reconstituted reagents at +4° C. for short time (1 week), for long storage prepare aliquots of reagents and store at −20° C. For preparation 10000 μg/mL IL1b calibrator solution dilute 1 vial of IL1b calibrator with appropriate volume of PBMC assay medium (see label indications on vial). For long storage prepare aliquots (54 μL) and store at −20° C.

Isolation of Mononuclear Cells from Human Peripheral Blood by Density Gradient Centrifugation:
Freezing solution I: RPMI 1640+GlutaMAX+Pen/Strep+Pyr-Na (PBMC assay medium)
Freezing solution II: 80% heat inactivated FBS+20% DMSO (Sigma, cat #D2650).

### Protocol:

1) Collect 45 mL blood to 50 mL Falcon tube with 5 mL Heparin Na (500 ME/mL).
2) Dilute cell with 2V of PBS.
3) Prepare 50 mL Falcon tubes with Ficoll: add 15 mL Ficoll-Paque per tube.
4) Carefully layer 35 mL of diluted blood over 15 mL of Ficoll-Paque in 50 mL sterile tube.
5) Centrifugate at Allegra 6R at 400×g (1350 RPM for GH-3.8 bucket rotor) 30 min 20° C. without brake.
6) Aspirate the upper layer leaving the mononuclear cell layer undisturbed at the interphase.
7) Carefully transfer the mononuclear cell layer to a new 50 mL sterile tube.
8) Fill the conical tube with buffer, mix, and centrifugate at 300 g (1150 RPM) for 10 min at 20° C. Carefully remove the supernatant completely.
9) Resuspend cell pellet in each 50 mL Falcon tube in 2-3 mL PBS. Combine cell pellets from 5-7 50 mL Falcon tubes to one 15 mL Falcon tube.
10) Centrifugate tubes at 200×g (950 RPM) for 15 minutes 20° C. Carefully remove the supernatant completely.
11) Resuspend cell pellet in 5-10 mL Freezing medium I. Count cells. Dilute cells to 2× freezing concentration with Freezing solution I.
12) Dropwise add the same volume of Freezing solution II.
13) Aliquot 1 mL or 2 mL cell suspension to each cryotube, firmly close the lid and put the tubes to freezing boxes.
14) Put freezing boxes to −80.
15) After overnight incubation transfer the cryotubes to Dewar's vessel with liquid nitrogen.

### Compounds Plate Preparation:

Dissolve compounds in DMSO (Panreac) up to 10 mM, incubate 5 min in UltraSonic Bath, RT. Add 85 μL cpds stocks in DMSO in column 3 of 384 well PP plate (Greiner, cat. number 47743-468). Prepare cpds serial dilutions in DMSO by Biomek 2000. Max cpds concentration in DMSO 10 μM, max cpds final concentration 40 μM. Dilution step=2. Store cpds serial dilution plates at −20° C.

### Assay Steps:

1. Thaw primary human PBMCs in a water bath at 37° C. and put vial content in 50 ml Falcon tube.
2. Add prewarmed RPMI 1640+GlutaMAX+Pen/Strep+Pyr-Na (PBMC assay medium). Add 10 ml per vial thawed, i.e. for 3 vials in 1 flacon tube, add 30 ml of medium.
3. Centrifuge at centrifuge CM 6M at 1500 RPM during 10 minutes.
4. Discard supernatant and resuspend the pellet in PBMC assay medium (1 ml medium per 1.5-2 ml cell).
5. Count cells, then add PBMC assay medium to final cell concentration of 1.3*10⁶ cells/ml.
6. For non-stimulated cells control transfer aliquot of cell suspension to new tube (~200 μL suspension for each plate).
7. Add to the cell suspension 1/50V of 50 ng/ml ultra-pure LPS stock (50×) to final concentration of 1000 pg/ml, mix carefully. (50× (50 ng/ml) LPS solution preparation: dilute LPS stock (5 mg/mL) two times in 316 fold: solution1 (15.82 μg/mL): 945 μL medium+3 μL LPS stock (5 mg/mL); solution2 (50 ng/ml): 945 μL medium+3 μL solution1).
8. By Biomek2000 add cells suspension with LPS to columns 1-22 of 384-well OptiPlate (Assay plate), 6 μl of cell suspension/well.
9. Add 6 μL/well cell suspension without LPS to column 23 of Assay plate.
10. Centrifuge plates at Eppendorf 5810 1 minute at 500 RPM.
11. Incubate 2 hours at 37° C. 5% CO₂.
12. Prepare intermediate PBMC medium plates: by Biomek FX add 55.35 μL PBMC assay medium to columns 1-23 of 384 well PP plate (Greiner, cat. number 47743-468). One intermediate plate for each cpds plate.
13. By Biomek FX dilute cpds in PBMC assay medium in 62.5 times (0.9 μL cpds+55.35 μL medium in intermediate plate) and add 2 μL of these solutions to assay plate.
14. Centrifuge plates at Eppendorf 5810 1 minute at 500 RPM.
15. Incubate 30 minutes at 37° C. 5% CO₂.
16. Prepare intermediate 5× Nigericin solution (20 μM): dilute in 500 times Nigericin stock (10 mM in Methanol). 1 mL 5× Nigericin solution per plate+8 mL dead volume.
17. By Biomek 2000 add 2 μL of Nigericin 5× stock to columns 1-22 of 384 well PP plate (Greiner, cat. number 47743-468). Add 2 μL PBMC assay medium to column 23 Assay plate.
18. Centrifuge plates at Eppendorf 5810 1 minute at 500 RPM.
19. Incubate 3 h at 37° C. 5% CO₂.
20. In 96 well PP plate prepare serial dilution of IL1b calibrator in PBMC assay medium: max IL1b concentration 10000 μg/mL, dilution step=2 (add 54 μL IL1b calibrator to well A. Add 27 μL PBMC assay medium to wells B-H. 6 Serial transfers 27 μL from well A to G, well H PBMC assay medium only). Add 10 μL of each solution to column #24 of assay plate, each concentration ×2 replicates.
21. Prepare anti-IL1b-Cryptate and anti-IL1b-XL665 conjugates mix. Mix volume (Vmix)=4 mL×assay plates number+dead volume (dead volume for 1-2 plates: 2 mL, for 3-4 plates: 3 mL, for 10 plates: 5-6 mL). To 50 mL tube add 38/40Vmix Reconstitution Buffer (from kit)+1/40Vmix anti-IL1b-Cryptate+1/40Vmix anti-IL1b-XL665, mix gently.
22. By Biomek 2000 add 10 μL of anti-IL 1b-Cryptate and anti-IL1b-XL665 conjugates mix (from step 21) to all wells of Assay plate.
23. Centrifuge plates at Eppendorf 5810 1 minute at 500 RPM.
24. Incubate 1 hr at RT.
25. Measure HTRF signal on Victor3V at 665 nm and 615 nm, protocol name LANCE High Count_665/615_OptiPlate.
26. Calculate 665 nm/615 nm signals ratio for each plate.
27. Calculate cpds IC50 by Grand_IC50_Script_0.0.6. Column 22-0% inhibition control, column 23 (non-stimulated cells)-100% inhibition control.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A compound of Formula (A): 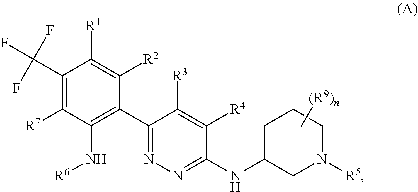
or a pharmaceutically acceptable salt, prodrug, stereoisomer, solvate, or tautomer thereof, wherein:
R¹ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R² is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R¹ and R² together with the atoms to which they are attached and any intervening atoms, form a 5-8 membered cycloalkane, a 5-14 membered heterocycle, arene, or heteroarene wherein the cycloalkane, heterocycle, arene, or heteroarene is optionally substituted with one or more substituents independently selected from halogen, OH, CN, C₁-C₆ alkyl, C₁-C₆ alkoxy;
R³ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R⁴ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R³ and R⁴ together with the atoms to which they are attached and any intervening atoms, form a 5-8 membered cycloalkane, a 5-14 membered heterocycle, wherein the cycloalkane, heterocycle, is optionally substituted with one or more substituents independently selected from halogen, OH, CN, C₁-C₆ alkyl, C₁-C₆ alkoxy;
R⁵ is selected from H, C₁-C₆ alkyl, C₃-C₁₀ cycloalkyl, C₂-C₆ alkenyl, C₂-C₆ alkynyl, C₁-C₆ acyl, SO₂—C₁-C₆ alkyl, 4-10 membered heterocyclyl, wherein the alkyl, cycloalkyl, alkenyl, alkynyl, acyl, or heterocyclyl is optionally substituted with one or more substituents independently selected from halogen, oxo, OH, CN, C₁-C₆ alkoxy, C₃-C₁₀ cycloalkyl, aryl;
R⁶ is selected from H, SO₂R⁸, NH₂, C₁-C₆ alkyl, C₁-C₆ alkyl-C(O)—, aryl, wherein alkyl or aryl is optionally substituted with one or more substituents independently selected from halogen, CN, OH;
R⁷ is H, C₁-C₆ alkyl, or NH₂; or
R⁶ and R⁷ together with the atoms to which they are attached and any intervening atoms, form a 5-6 membered heterocycle or heteroarene;
R⁸ is selected from C₁-C₆ alkyl, C₃-C₈ cycloalkyl, 5-6 membered heteroaryl, wherein alkyl, cycloalkyl or heteroaryl is optionally substituted with one or more substituents independently selected from halogen, CN, OH;
each R⁹ is independently selected from halogen, OH, C₁-C₆ alkyl, C₁-C₆ alkoxy, wherein alkyl or alkoxy optionally substituted with one or more halogen, OH; or
two R⁹ together with the atoms to which they are attached and any intervening atoms, form a 5-6 membered heterocycle; or
R⁵ and R⁹ together with the atoms to which they are attached and any intervening atoms, form a 5-6 membered heterocycle;
n is an integer selected from 0, 1, and 2;
provided that when R⁵ is methyl then R⁶ is not H or CH₃CO—.

2. The compound of claim 1, wherein the compound is of Formula (I): 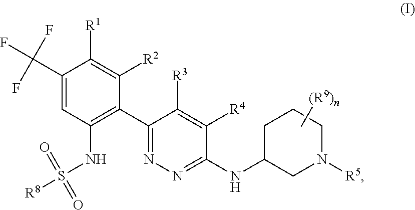
or a pharmaceutically acceptable salt, prodrug, stereoisomer, solvate, or tautomer thereof, wherein:
R¹ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R² is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R¹ and R² together with the atoms to which they are attached and any intervening atoms, form a 5-8 membered cycloalkane, a 5-14 membered heterocycle, arene, or heteroarene wherein the cycloalkane, heterocycle, arene, or heteroarene is optionally substituted with one or more substituents independently selected from halogen, OH, CN, C₁-C₆ alkyl, C₁-C₆ alkoxy;
R³ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R⁴ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R³ and R⁴ together with the atoms to which they are attached and any intervening atoms, form a 5-8 membered cycloalkane, a 5-14 membered heterocycle, wherein the cycloalkane, heterocycle, is optionally substituted with one or more substituents independently selected from halogen, OH, CN, C₁-C₆ alkyl, C₁-C₆ alkoxy;
R⁵ is selected from H, C₁-C₆ alkyl, C₃-C₁₀ cycloalkyl, C₂-C₆ alkenyl, C₂-C₆ alkynyl, C₁-C₆ acyl, SO₂—C₁-C₆ alkyl, 4-10 membered heterocyclyl, wherein the alkyl, cycloalkyl, alkenyl, alkynyl, acyl, or heterocyclyl is optionally substituted with one or more substituents independently selected from halogen, oxo, OH, CN, C₁-C₆ alkoxy, C₃-C₁₀ cycloalkyl, aryl;
R⁸ is selected from C₁-C₆ alkyl, C₃-C₈ cycloalkyl, 5-6 membered heteroaryl, wherein alkyl, cycloalkyl or heteroaryl is optionally substituted with one or more substituents independently selected from halogen, CN, OH;
each R⁹ is independently selected from halogen, OH, C₁-C₆ alkyl, C₁-C₆ alkoxy, wherein alkyl or alkoxy optionally substituted with one or more halogen, OH; or
two R⁹ together with the atoms to which they are attached and any intervening atoms, form a 5-6 membered heterocycle; or
R⁵ and R⁹ together with the atoms to which they are attached and any intervening atoms, form a 5-6 membered heterocycle;
n is an integer selected from 0, 1, and 2.

3. The compound of claim 1, wherein the compound is of Formula (II): 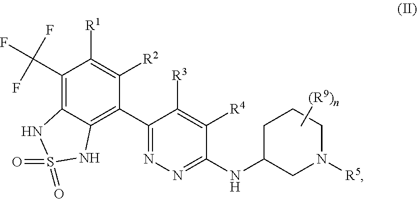
or a pharmaceutically acceptable salt, prodrug, stereoisomer, solvate, or tautomer thereof, wherein:
R¹ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R² is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R¹ and R² together with the atoms to which they are attached and any intervening atoms, form a 5-8 membered cycloalkane, a 5-14 membered heterocycle, arene, or heteroarene wherein the cycloalkane, heterocycle, arene, or heteroarene is optionally substituted with one or more substituents independently selected from halogen, OH, CN, C₁-C₆ alkyl, C₁-C₆ alkoxy;
R³ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R⁴ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R³ and R⁴ together with the atoms to which they are attached and any intervening atoms, form a 5-8 membered cycloalkane, a 5-14 membered heterocycle, wherein the cycloalkane, heterocycle, is optionally substituted with one or more substituents independently selected from halogen, OH, CN, C₁-C₆ alkyl, C₁-C₆ alkoxy;
R⁵ is selected from H, C₁-C₆ alkyl, C₃-C₁₀ cycloalkyl, C₂-C₆ alkenyl, C₂-C₆ alkynyl, C₁-C₆ acyl, SO₂—C₁-C₆ alkyl, 4-10 membered heterocyclyl, wherein the alkyl, cycloalkyl, alkenyl, alkynyl, acyl, or heterocyclyl is optionally substituted with one or more substituents independently selected from halogen, oxo, OH, CN, C₁-C₆ alkoxy, C₃-C₁₀ cycloalkyl, aryl;
each R⁹ is independently selected from halogen, OH, C₁-C₆ alkyl, C₁-C₆ alkoxy, wherein alkyl or alkoxy optionally substituted with one or more halogen, OH; or
two R⁹ together with the atoms to which they are attached and any intervening atoms, form a 5-6 membered heterocycle; or
R⁵ and R⁹ together with the atoms to which they are attached and any intervening atoms, form a 5-6 membered heterocycle;
n is an integer selected from 0, 1, and 2.

4. The compound of claim 1, wherein the compound is of Formula (III): 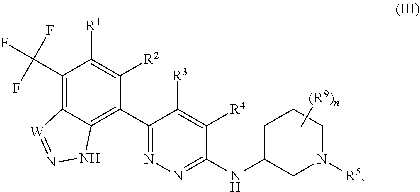
or a pharmaceutically acceptable salt, prodrug, stereoisomer, solvate, or tautomer thereof, wherein:
W is selected from CH, N;
R¹ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R² is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R¹ and R² together with the atoms to which they are attached and any intervening atoms, form a 5-8 membered cycloalkane, a 5-14 membered heterocycle, arene, or heteroarene wherein the cycloalkane, heterocycle, arene, or heteroarene is optionally substituted with one or more substituents independently selected from halogen, OH, CN, C₁-C₆ alkyl, C₁-C₆ alkoxy;
R³ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R⁴ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R³ and R⁴ together with the atoms to which they are attached and any intervening atoms, form a 5-8 membered cycloalkane, a 5-14 membered heterocycle, wherein the cycloalkane, heterocycle, is optionally substituted with one or more substituents independently selected from halogen, OH, CN, C₁-C₆ alkyl, C₁-C₆ alkoxy;
R⁵ is selected from H, C₁-C₆ alkyl, C₃-C₁₀ cycloalkyl, C₂-C₆ alkenyl, C₂-C₆ alkynyl, C₁-C₆ acyl, SO₂—C₁-C₆ alkyl, 4-10 membered heterocyclyl, wherein the alkyl, cycloalkyl, alkenyl, alkynyl, acyl, or heterocyclyl is optionally substituted with one or more substituents independently selected from halogen, oxo, OH, CN, C₁-C₆ alkoxy, C₃-C₁₀ cycloalkyl, aryl;
each R⁹ is independently selected from halogen, OH, C₁-C₆ alkyl, C₁-C₆ alkoxy, wherein alkyl or alkoxy optionally substituted with one or more halogen, OH; or
two R⁹ together with the atoms to which they are attached and any intervening atoms, form a 5-6 membered heterocycle; or
R⁵ and R⁹ together with the atoms to which they are attached and any intervening atoms, form a 5-6 membered heterocycle;
n is an integer selected from 0, 1, and 2.

5. The compound of claim 4, wherein the compound is of Formula (III-A): 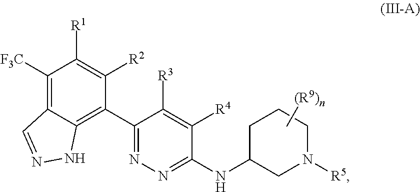
or a pharmaceutically acceptable salt, stereoisomer, solvate, or tautomer thereof.

6. The compound of claim 4, wherein the compound is of Formula (III-B): 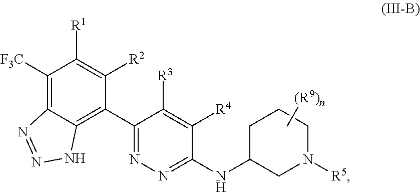
or a pharmaceutically acceptable salt, stereoisomer, solvate, or tautomer thereof.

7. The compound of claim 1, wherein the compound is of Formula (IV): 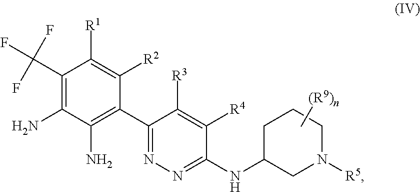
or a pharmaceutically acceptable salt, prodrug, stereoisomer, solvate, or tautomer thereof, wherein:
R¹ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R² is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R¹ and R² together with the atoms to which they are attached and any intervening atoms, form a 5-8 membered cycloalkane, a 5-14 membered heterocycle, arene, or heteroarene wherein the cycloalkane, heterocycle, arene, or heteroarene is optionally substituted with one or more substituents independently selected from halogen, OH, CN, C₁-C₆ alkyl, C₁-C₆ alkoxy;
R³ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R⁴ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R³ and R⁴ together with the atoms to which they are attached and any intervening atoms, form a 5-8 membered cycloalkane, a 5-14 membered heterocycle, wherein the cycloalkane, heterocycle, is optionally substituted with one or more substituents independently selected from halogen, OH, CN, C₁-C₆ alkyl, C₁-C₆ alkoxy;
R⁵ is selected from H, C₁-C₆ alkyl, C₃-C₁₀ cycloalkyl, C₂-C₆ alkenyl, C₂-C₆ alkynyl, C₁-C₆ acyl, SO₂—C₁-C₆ alkyl, 4-10 membered heterocyclyl, wherein the alkyl, cycloalkyl, alkenyl, alkynyl, acyl, or heterocyclyl is optionally substituted with one or more substituents independently selected from halogen, oxo, OH, CN, C₁-C₆ alkoxy, C₃-C₁₀ cycloalkyl, aryl;
each R⁹ is independently selected from halogen, OH, C₁-C₆ alkyl, C₁-C₆ alkoxy, wherein alkyl or alkoxy optionally substituted with one or more halogen, OH; or
two R⁹ together with the atoms to which they are attached and any intervening atoms, form a 5-6 membered heterocycle; or
R⁵ and R⁹ together with the atoms to which they are attached and any intervening atoms, form a 5-6 membered heterocycle;
n is an integer selected from 0, 1, and 2.

8. The compound of claim 1, wherein the compound is of Formula (V): 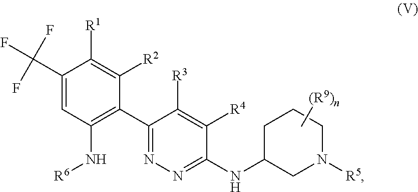
or a pharmaceutically acceptable salt, prodrug, stereoisomer, solvate, or tautomer thereof, wherein:
R¹ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R² is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R¹ and R² together with the atoms to which they are attached and any intervening atoms, form a 5-8 membered cycloalkane, a 5-14 membered heterocycle, arene, or heteroarene wherein the cycloalkane, heterocycle, arene, or heteroarene is optionally substituted with one or more substituents independently selected from halogen, OH, CN, C₁-C₆ alkyl, C₁-C₆ alkoxy;
R³ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R⁴ is selected from H, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R³ and R⁴ together with the atoms to which they are attached and any intervening atoms, form a 5-8 membered cycloalkane, a 5-14 membered heterocycle, wherein the cycloalkane, heterocycle, is optionally substituted with one or more substituents independently selected from halogen, OH, CN, C₁-C₆ alkyl, C₁-C₆ alkoxy;
R⁵ is selected from H, C₁-C₆ alkyl, C₃-C₁₀ cycloalkyl, C₂-C₆ alkenyl, C₂-C₆ alkynyl, C₁-C₆ acyl, SO₂—C₁-C₆ alkyl, 4-10 membered heterocyclyl, wherein the alkyl, cycloalkyl, alkenyl, alkynyl, acyl, or heterocyclyl is optionally substituted with one or more substituents independently selected from halogen, oxo, OH, CN, C₁-C₆ alkoxy, C₃-C₁₀ cycloalkyl, aryl;
R⁶ is selected from H, C₁-C₆ alkyl, C₁-C₆ alkyl-C(O)—, aryl, wherein alkyl or aryl is optionally substituted with one or more substituents independently selected from halogen, CN, OH;
each R⁹ is independently selected from halogen, OH, C₁-C₆ alkyl, C₁-C₆ alkoxy, wherein alkyl or alkoxy optionally substituted with one or more halogen, OH; or
two R⁹ together with the atoms to which they are attached and any intervening atoms, form a 5-6 membered heterocycle; or
R⁵ and R⁹ together with the atoms to which they are attached and any intervening atoms, form a 5-6 membered heterocycle;
n is an integer selected from 0, 1, and 2;
provided that when R⁵ is methyl then R⁶ is not H or CH₃CO—.

9. A compound is selected from:

| # | Structure | IUPAC Name |
|---|---|---|
| 1 | 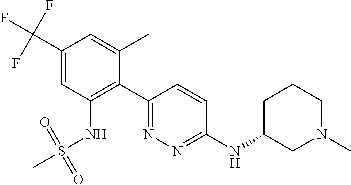 [SMILES: Cc1cc(C(F)(F)F)cc(NS(C)(=O)=O)c1-c1ccc(N[C@@H]2CCCN(C)C2)nn1] | N-[3-methyl-2-[6-[(1-methyl-3- piperidyl)amino]pyridazin-3-yl]-5- (trifluoromethyl)phenyl]methane- sulfonamide |
| 2 | 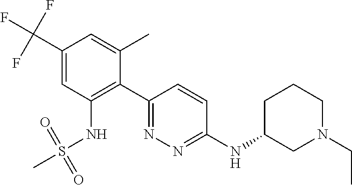 [SMILES: CCN1CCC[C@@H](Nc2ccc(-c3c(C)cc(C(F)(F)F)cc3NS(C)(=O)=O)nn2)C1] | N-[2-[6-[(1-ethyl-3- piperidyl)amino]pyridazin-3-yl]-3- methyl-5- (trifluoromethyl)phenyl]methane- sulfonamide |
| 3 | 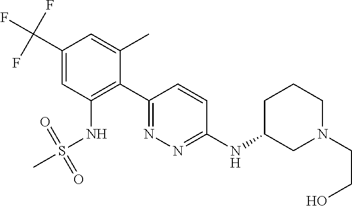 [SMILES: Cc1cc(C(F)(F)F)cc(NS(C)(=O)=O)c1-c1ccc(N[C@@H]2CCCN(CCO)C2)nn1] | N-[2-[6-[[1-(2-hydroxyethyl)-3- piperidyl]amino]pyridazin-3-yl]-3- methyl-5- (trifluoromethyl)phenyl]methane- sulfonamide |
| 4 | 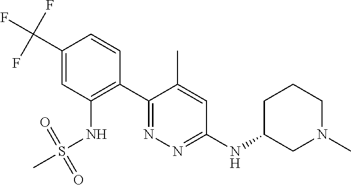 [SMILES: Cc1cc(N[C@@H]2CCCN(C)C2)nnc1-c1ccc(C(F)(F)F)cc1NS(C)(=O)=O] | N-[2-[4-methyl-6-[(1-methyl-3- piperidyl)amino]pyridazin-3-yl]-5- (trifluoromethyl)phenyl]methane- sulfonamide |
| 5 | 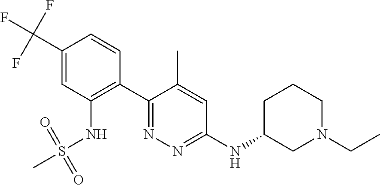 [SMILES: CCN1CCC[C@@H](Nc2cc(C)c(-c3ccc(C(F)(F)F)cc3NS(C)(=O)=O)nn2)C1] | N-[2-[6-[(1-ethyl-3-piperidyl)amino]- 4-methyl-pyridazin-3-yl]-5- (trifluoromethyl)phenyl]methane- sulfonamide |
| 6 | 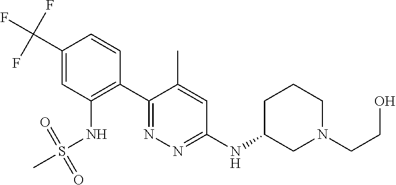 [SMILES: Cc1cc(N[C@@H]2CCCN(CCO)C2)nnc1-c1ccc(C(F)(F)F)cc1NS(C)(=O)=O] | N-[2-[6-[[1-(2-hydroxyethyl)-3- piperidyl]amino]-4-methyl-pyridazin- 3-yl]-5- (trifluoromethyl)phenyl]methane- sulfonamide |
| 7 | 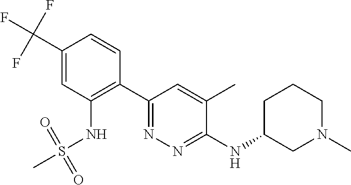 [SMILES: Cc1cc(-c2ccc(C(F)(F)F)cc2NS(C)(=O)=O)nnc1N[C@@H]1CCCN(C)C1] | N-[2-[5-methyl-6-[[(3R)-1-methyl-3- piperidyl]amino]pyridazin-3-yl]-5- (trifluoromethyl)phenyl]methane- sulfonamide |
| 8 | 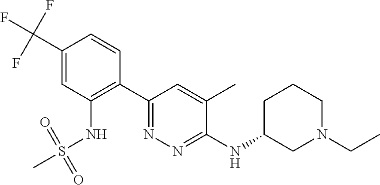 [SMILES: CCN1CCC[C@@H](Nc2nnc(-c3ccc(C(F)(F)F)cc3NS(C)(=O)=O)cc2C)C1] | N-[2-[6-[[(3R)-1-ethyl-3- piperidyl]amino]-5-methyl-pyridazin- 3-yl]-5- (trifluoromethyl)phenyl]methane- sulfonamide |
| 9 | 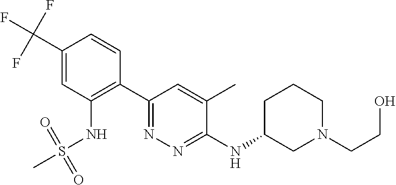 [SMILES: Cc1cc(-c2ccc(C(F)(F)F)cc2NS(C)(=O)=O)nnc1N[C@@H]1CCCN(CCO)C1] | N-[2-[6-[[(3R)-1-(2-hydroxyethyl)-3- piperidyl]amino]-5-methyl-pyridazin- 3-yl]-5- (trifluoromethyl)phenyl]methane- sulfonamide |
| 10 | 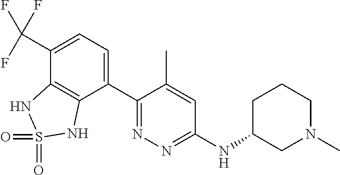 [SMILES: Cc1cc(N[C@@H]2CCCN(C)C2)nnc1-c1ccc(C(F)(F)F)c2c1NS(=O)(=O)N2] | 6-[2,2-dioxo-4-(trifluoromethyl)-1,3- dihydro-2lambda6,1,3- benzothiadiazol-7-yl]-5-methyl-N-(1- methyl-3-piperidyl)pyridazin-3- amine |
| 11 | 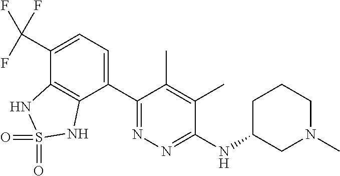 [SMILES: Cc1c(N[C@@H]2CCCN(C)C2)nnc(-c2ccc(C(F)(F)F)c3c2NS(=O)(=O)N3)c1C] | 6-[2,2-dioxo-4-(trifluoromethyl)-1,3- dihydro-2lambda6,1,3- benzothiadiazol-7-yl]-4,5-dimethyl- N-(1-methyl-3-piperidyl)pyridazin-3- amine |
| 12 | 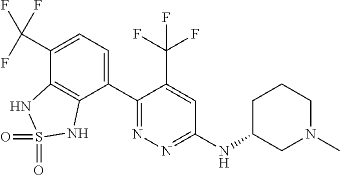 [SMILES: CN1CCC[C@@H](Nc2cc(C(F)(F)F)c(-c3ccc(C(F)(F)F)c4c3NS(=O)(=O)N4)nn2)C1] | 6-[2,2-dioxo-4-(trifluoromethyl)-1,3- dihydro-2lambda6,1,3- benzothiadiazol-7-yl]-N-[(3R)-1- methyl-3-piperidyl]-5- (trifluoromethyl)pyridazin-3-amine |
| 13 | 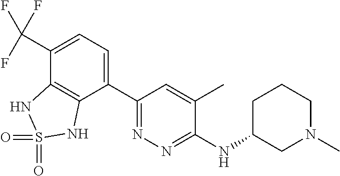 [SMILES: Cc1cc(-c2ccc(C(F)(F)F)c3c2NS(=O)(=O)N3)nnc1N[C@@H]1CCCN(C)C1] | 6-[2,2-dioxo-4-(trifluoromethyl)-1,3- dihydro-2lambda6,1,3- benzothiadiazol-7-yl]-4-methyl-N- [(3R)-1-methyl-3- piperidyl]pyridazin-3-amine |
| 14 | 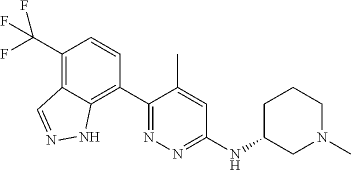 [SMILES: Cc1cc(N[C@@H]2CCCN(C)C2)nnc1-c1ccc(C(F)(F)F)c2cn[nH]c12] | 5-methyl-N-[(3R)-1-methyl-3- piperidyl]-6-[4-(trifluoromethyl)-1H- indazol-7-yl]pyridazin-3-amine |
| 15 | 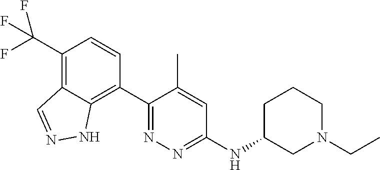 [SMILES: CCN1CCC[C@@H](Nc2cc(C)c(-c3ccc(C(F)(F)F)c4cn[nH]c34)nn2)C1] | N-[(3R)-1-ethyl-3-piperidyl]-5- methyl-6-[4-(trifluoromethyl)-1H- indazol-7-yl]pyridazin-3-amine |
| 16 | 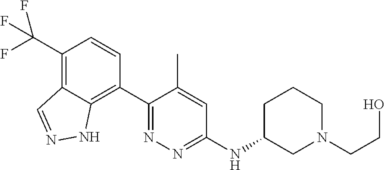 [SMILES: Cc1cc(N[C@@H]2CCCN(CCO)C2)nnc1-c1ccc(C(F)(F)F)c2cn[nH]c12] | 2-[(3R)-3-[5-methyl-6-[4- (trifluoromethyl)-1H-indazol-7- yl]pyridazin-3-yl]amino]-1- piperidyl]ethanol |
| 17 | 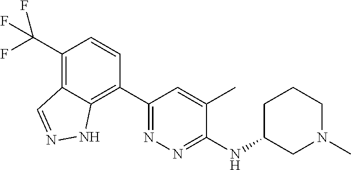 [SMILES: Cc1cc(-c2ccc(C(F)(F)F)c3cn[nH]c23)nnc1N[C@@H]1CCCN(C)C1] | 4-methyl-N-[(3R)-1-methyl-3- piperidyl]-6-[4-(trifluoromethyl)-1H- indazol-7-yl]pyridazin-3-amine |
| 18 | 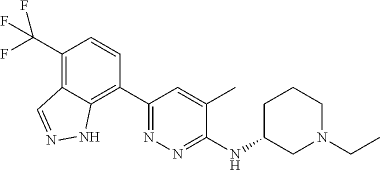 [SMILES: CCN1CCC[C@@H](Nc2nnc(-c3ccc(C(F)(F)F)c4cn[nH]c34)cc2C)C1] | N-[(3R)-1-ethyl-3-piperidyl]-4- methyl-6-[4-(trifluoromethyl)-1H- indazol-7-yl]pyridazin-3-amine |
| 19 | 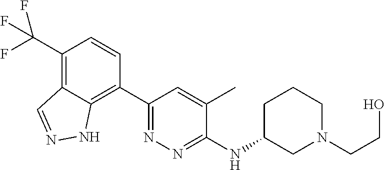 [SMILES: Cc1cc(-c2ccc(C(F)(F)F)c3cn[nH]c23)nnc1N[C@@H]1CCCN(CCO)C1] | 2-[(3R)-3-[[4-methyl-6-[4- (trifluoromethyl)-1H-indazol-7- yl]pyridazin-3-yl]amino]-1- piperidyl]ethanol |
| 20 | 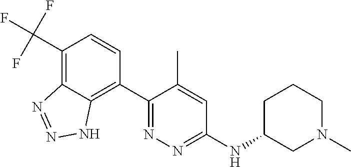 [SMILES: Cc1cc(N[C@@H]2CCCN(C)C2)nnc1-c1ccc(C(F)(F)F)c2nn[nH]c12] | 5-methyl-N-[(3R)-1-methyl-3- piperidyl]-6-[7-(trifluoromethyl)-3H- benzotriazol-4-yl]pyridazin-3-amine |
| 21 | 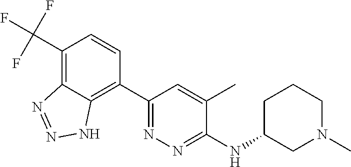 [SMILES: Cc1cc(-c2ccc(C(F)(F)F)c3nn[nH]c23)nnc1N[C@@H]1CCCN(C)C1] | 4-methyl-N-[(3R)-1-methyl-3- piperidyl]-6-[7-(trifluoromethyl)-3H- benzotriazol-4-yl]pyridazin-3-amine |
| 22 | 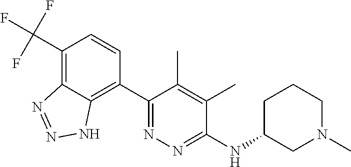 [SMILES: Cc1c(N[C@@H]2CCCN(C)C2)nnc(-c2ccc(C(F)(F)F)c3nn[nH]c23)c1C] | 4,5-dimethyl-N-[(3R)-1-methyl-3- piperidyl]-6-[7-(trifluoromethyl)-3H- benzotriazol-4-yl]pyridazin-3-amine |
| 23 | 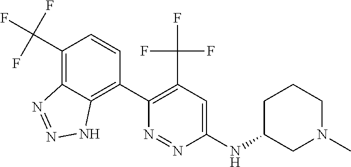 [SMILES: CN1CCC[C@@H](Nc2cc(C(F)(F)F)c(-c3ccc(C(F)(F)F)c4nn[nH]c34)nn2)C1] | N-[(3R)-1-methyl-3-piperidyl]-5- (trifluoromethyl)-6-[7- (trifluoromethyl)-3H-benzotriazol-4- yl]pyridazin-3-amine |
| 24 | 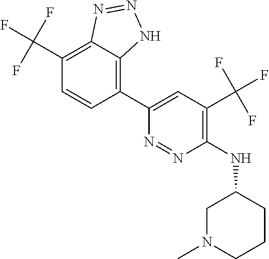 [SMILES: CN1CCC[C@@H](Nc2nnc(-c3ccc(C(F)(F)F)c4nn[nH]c34)cc2C(F)(F)F)C1] | N-[(3R)-1-methyl-3-piperidyl]-4- (trifluoromethyl)-6-[7- (trifluoromethyl)-3H-benzotriazol-4- yl]pyridazin-3-amine |
| 25 | 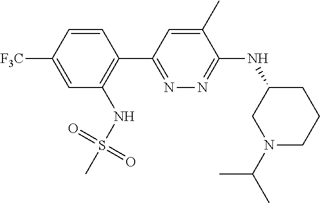 [SMILES: Cc1cc(-c2ccc(C(F)(F)F)cc2NS(C)(=O)=O)nnc1N[C@@H]1CCCN(C(C)C)C1] | N-[2-[6-[(3R)-1-isopropyl-3- piperidyl]amino]-5-methyl-pyridazin- 3-yl]-5- (trifluoromethyl)phenyl]- methanesulfonamide |
| 26 | 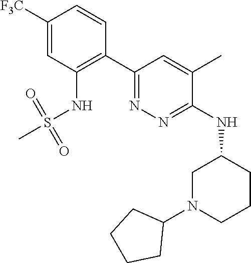 [SMILES: Cc1cc(-c2ccc(C(F)(F)F)cc2NS(C)(=O)=O)nnc1N[C@@H]1CCCN(C2CCCC2)C1] | N-[2-[6-[[(3R)-1-cyclopentyl-3- piperidyl]amino]-5-methyl-pyridazin- 3-yl]-5- (trifluoromethyl)phenyl]- methanesulfonamide |
| 27 | 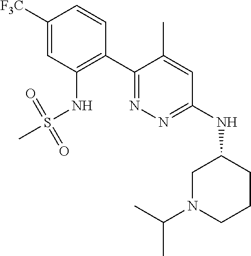 [SMILES: Cc1cc(N[C@@H]2CCCN(C(C)C)C2)nnc1-c1ccc(C(F)(F)F)cc1NS(C)(=O)=O] | N-[2-[6-[[(3R)-1-isopropyl-3- piperidyl]amino]-4-methyl-pyridazin- 3-yl]-5- (trifluoromethyl)phenyl]- methanesulfonamide |
| 28 | 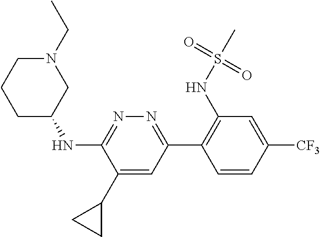 [SMILES: CCN1CCC[C@@H](Nc2nnc(-c3ccc(C(F)(F)F)cc3NS(C)(=O)=O)cc2C2CC2)C1] | N-[2-[5-cyclopropyl-6-[[(3R)-1- ethyl-3-piperidyl]amino]pyridazin-3- yl]-5- (trifluoromethyl)phenyl]- methanesulfonamide |
| 29 | 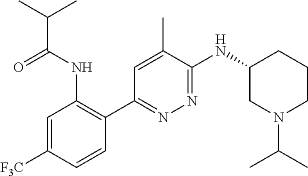 [SMILES: Cc1cc(-c2ccc(C(F)(F)F)cc2NC(=O)C(C)C)nnc1N[C@@H]1CCCN(C(C)C)C1] | N-[2-[6-[[(3R)-1-isopropyl-3- piperidyl]amino]-5-methyl-pyridazin- 3-yl]-5-(trifluoromethyl)phenyl]-2- methyl-propanamide |
| 30 | 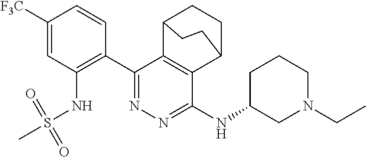 [SMILES: CCN1CCC[C@@H](Nc2nnc(-c3ccc(C(F)(F)F)cc3NS(C)(=O)=O)c3c2C2CCC3CC2)C1] | N-[2-[6-[[(3R)-1-ethyl-3- piperidyl]amino]-4,5- diazatricyclo[6.2.2.02,7]dodeca- 2(7),3,5-trien-3-yl]-5- (trifluoromethyl)phenyl]- methanesulfonamide |
| 31 | 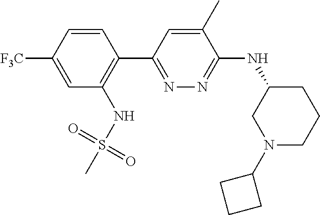 [SMILES: Cc1cc(-c2ccc(C(F)(F)F)cc2NS(C)(=O)=O)nnc1N[C@@H]1CCCN(C2CCC2)C1] | N-[2-[6-[(3R)-1-cyclobutyl-3- piperidyl]amino]-5-methyl-pyridazin- 3-yl]-5- (trifluoromethyl)phenyl]- methanesulfonamide |
| 32 | 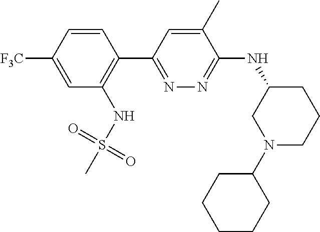 [SMILES: Cc1cc(-c2ccc(C(F)(F)F)cc2NS(C)(=O)=O)nnc1N[C@@H]1CCCN(C2CCCCC2)C1] | N-[2-[6-[(3R)-1-cyclohexyl-3- piperidyl]amino]-5-methyl-pyridazin- 3-yl]-5- (trifluoromethyl)phenyl]- methanesulfonamide |
| 33 | 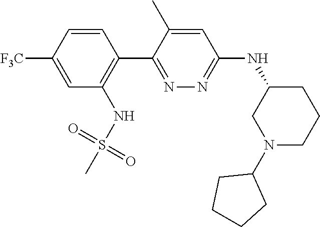 [SMILES: Cc1cc(N[C@@H]2CCCN(C3CCCC3)C2)nnc1-c1ccc(C(F)(F)F)cc1NS(C)(=O)=O] | N-[2-[6-[(3R)-1-cyclopentyl-3- piperidyl]amino]-4-methyl-pyridazin- 3-yl]-5- (trifluoromethyl)phenyl]- methanesulfonamide |
| 34 | 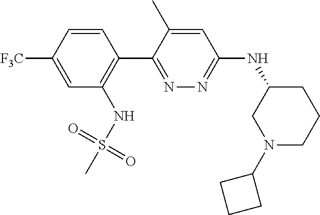 [SMILES: Cc1cc(N[C@@H]2CCCN(C3CCC3)C2)nnc1-c1ccc(C(F)(F)F)cc1NS(C)(=O)=O] | N-[2-[6-[(3R)-1-cyclobutyl-3- piperidyl]amino]-4-methyl-pyridazin- 3-yl]-5- (trifluoromethyl)phenyl]- methanesulfonamide |
| 35 | 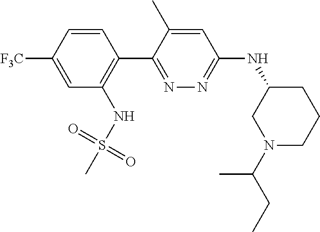 [SMILES: CCC(C)N1CCC[C@@H](Nc2cc(C)c(-c3ccc(C(F)(F)F)cc3NS(C)(=O)=O)nn2)C1] | N-[2-[4-methyl-6-[[(3R)-1-sec-butyl- 3-piperidyl]amino]pyridazin-3-yl]-5- (trifluoromethyl)phenyl]- methanesulfonamide |
| 36 | 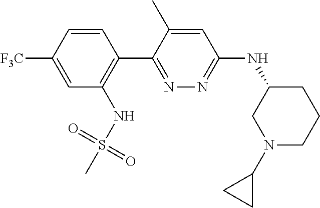 [SMILES: Cc1cc(N[C@@H]2CCCN(C3CC3)C2)nnc1-c1ccc(C(F)(F)F)cc1NS(C)(=O)=O] | N-[2-[6-[[(3R)-1-cyclopropyl-3- piperidyl]amino]-4-methyl-pyridazin- 3-yl]-5- (trifluoromethyl)phenyl]- methanesulfonamide |
| 37 | 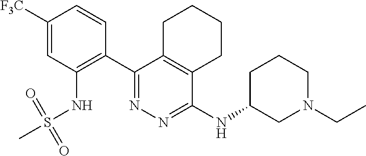 [SMILES: CCN1CCC[C@@H](Nc2nnc(-c3ccc(C(F)(F)F)cc3NS(C)(=O)=O)c3c2CCCC3)C1] | N-[2-[4-[[(3R)-1-ethyl-3- piperidyl]amino]-5,6,7,8- tetrahydrophthalazin-1-yl]-5- (trifluoromethyl)phenyl]- methanesulfonamide |
| 38 | 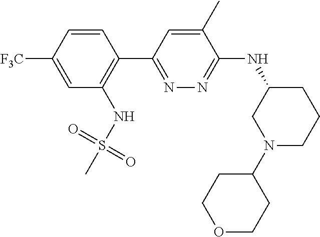 [SMILES: Cc1cc(-c2ccc(C(F)(F)F)cc2NS(C)(=O)=O)nnc1N[C@@H]1CCCN(C2CCOCC2)C1] | N-[2-[5-methyl-6-[[(3R)-1- tetrahydropyran-4-yl-3- piperidyl]amino]pyridazin-3-yl]-5- (trifluoromethyl)phenyl]- methanesulfonamide |
| 39 | 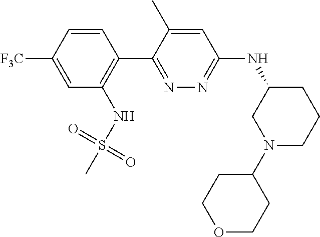 [SMILES: Cc1cc(N[C@@H]2CCCN(C3CCOCC3)C2)nnc1-c1ccc(C(F)(F)F)cc1NS(C)(=O)=O] | N-[2-[4-methyl-6-[(3R)-1- tetrahydropyran-4-yl-3- piperidyl]amino]pyridazin-3-yl]-5- (trifluoromethyl)phenyl]- methanesulfonamide |
| 40 | 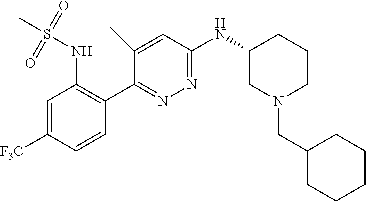 [SMILES: Cc1cc(N[C@@H]2CCCN(CC3CCCCC3)C2)nnc1-c1ccc(C(F)(F)F)cc1NS(C)(=O)=O] | N-[2-[6-[[(3R)-1-(cyclohexylmethyl)- 3-piperidyl]amino]-4-methyl- pyridazin-3-yl]-5- (trifluoromethyl)phenyl]- methanesulfonamide |
| 41 | 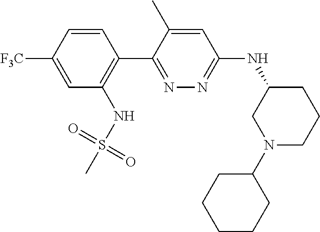 [SMILES: Cc1cc(N[C@@H]2CCCN(C3CCCCC3)C2)nnc1-c1ccc(C(F)(F)F)cc1NS(C)(=O)=O] | N-[2-[6-[[(3R)-1-cyclohexyl-3- piperidyl]amino]-4-methyl-pyridazin- 3-yl]-5- (trifluoromethyl)phenyl]- methanesulfonamide |
| 42 | 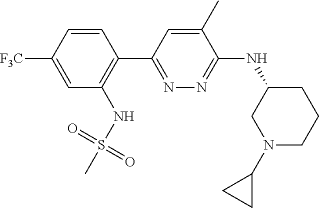 [SMILES: Cc1cc(-c2ccc(C(F)(F)F)cc2NS(C)(=O)=O)nnc1N[C@@H]1CCCN(C2CC2)C1] | N-[2-[6-[(3R)-1-cyclopropyl-3- piperidyl]amino]-5-methyl-pyridazin- 3-yl]-5- (trifluoromethyl)phenyl]- methanesulfonamide |
| 43 | 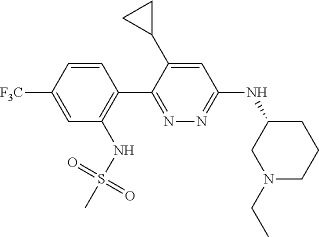 [SMILES: CCN1CCC[C@@H](Nc2cc(C3CC3)c(-c3ccc(C(F)(F)F)cc3NS(C)(=O)=O)nn2)C1] | N-[2-[4-cyclopropyl-6-[[(3R)-1- ethyl-3-piperidyl]amino]pyridazin-3- yl]-5- (trifluoromethyl)phenyl]- methanesulfonamide |
| 44 | 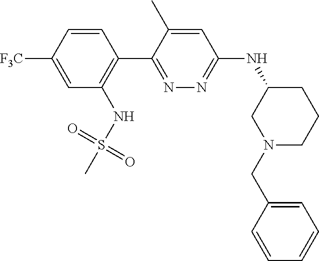 [SMILES: Cc1cc(N[C@@H]2CCCN(Cc3ccccc3)C2)nnc1-c1ccc(C(F)(F)F)cc1NS(C)(=O)=O] | N-[2-[6-[[(3R)-1-benzyl-3- piperidyl]amino]-4-methyl-pyridazin- 3-yl]-5- (trifluoromethyl)phenyl]- methanesulfonamide |
| 45 | 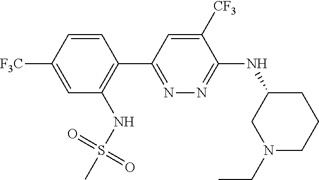 [SMILES: CCN1CCC[C@@H](Nc2nnc(-c3ccc(C(F)(F)F)cc3NS(C)(=O)=O)cc2C(F)(F)F)C1] | N-[2-[6-[[(3R)-1-ethyl-3- piperidyl]amino]-5- (trifluoromethyl)pyridazin-3-yl]-5- (trifluoromethyl)phenyl]- methanesulfonamide |
| 46 | 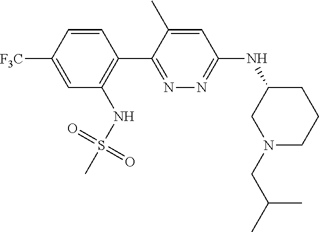 [SMILES: Cc1cc(N[C@@H]2CCCN(CC(C)C)C2)nnc1-c1ccc(C(F)(F)F)cc1NS(C)(=O)=O] | N-[2-[6-[[(3R)-1-isobutyl-3- piperidyl]amino]-4-methyl-pyridazin- 3-yl]-5- (trifluoromethyl)phenyl]- methanesulfonamide |
| 47 | 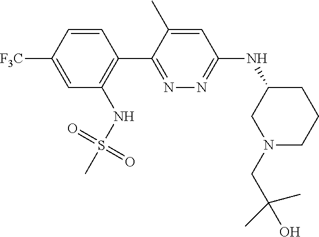 [SMILES: Cc1cc(N[C@@H]2CCCN(CC(C)(C)O)C2)nnc1-c1ccc(C(F)(F)F)cc1NS(C)(=O)=O] | N-[2-[6-[(3R)-1-(2-hydroxy-2- methyl-propyl)-3-piperidyl]amino]-4- methyl-pyridazin-3-yl]-5- (trifluoromethyl)phenyl]- methanesulfonamide |
| 48 | 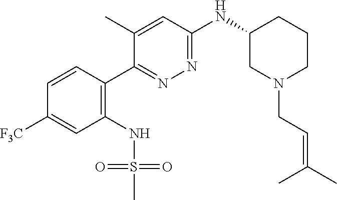 [SMILES: CC(C)=CCN1CCC[C@@H](Nc2cc(C)c(-c3ccc(C(F)(F)F)cc3NS(C)(=O)=O)nn2)C1] | N-[2-[4-methyl-6-[(3R)-1-(3- methylbut-2-enyl)-3- piperidyl]amino]pyridazin-3-yl]-5- (trifluoromethyl)phenyl]- methanesulfonamide |
| 49 | 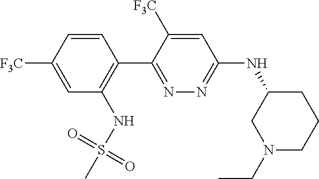 [SMILES: CCN1CCC[C@@H](Nc2cc(C(F)(F)F)c(-c3ccc(C(F)(F)F)cc3NS(C)(=O)=O)nn2)C1] | N-[2-[6-[[(3R)-1-ethyl-3- piperidyl]amino]-4- (trifluoromethyl)pyridazin-3-yl]-5- (trifluoromethyl)phenyl]- methanesulfonamide |
| 50 | 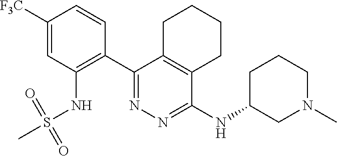 [SMILES: CN1CCC[C@@H](Nc2nnc(-c3ccc(C(F)(F)F)cc3NS(C)(=O)=O)c3c2CCCC3)C1] | N-[2-[4-[[(3R)-1-methyl-3- piperidyl]amino]-5,6,7,8- tetrahydrophthalazin-1-yl]-5- (trifluoromethyl)phenyl]- methanesulfonamide |
| 51 | 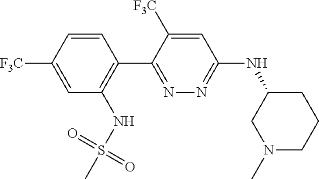 [SMILES: CN1CCC[C@@H](Nc2cc(C(F)(F)F)c(-c3ccc(C(F)(F)F)cc3NS(C)(=O)=O)nn2)C1] | N-[2-[6-[(3R)-1-methyl-3- piperidyl]amino]-4- (trifluoromethyl)pyridazin-3-yl]-5- (trifluoromethyl)phenyl]- methanesulfonamide |
| 52 | 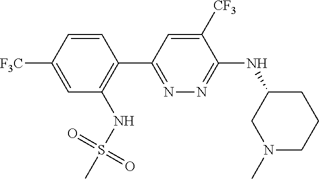 [SMILES: CN1CCC[C@@H](Nc2nnc(-c3ccc(C(F)(F)F)cc3NS(C)(=O)=O)cc2C(F)(F)F)C1] | N-[2-[6-[[(3R)-1-methyl-3- piperidyl]amino]-5- (trifluoromethyl)pyridazin-3-yl]-5- (trifluoromethyl)phenyl]- methanesulfonamide |
| 53 | 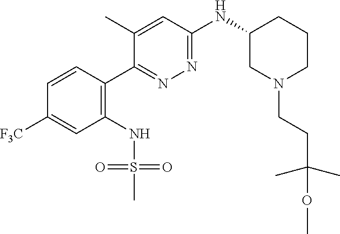 [SMILES: COC(C)(C)CCN1CCC[C@@H](Nc2cc(C)c(-c3ccc(C(F)(F)F)cc3NS(C)(=O)=O)nn2)C1] | N-[2-[6-[[(3R)-1-(3-methoxy-3- methyl-butyl)-3-piperidyl]amino]-4- methyl-pyridazin-3-yl]-5- (trifluoromethyl)phenyl]- methanesulfonamide |
| 54 | 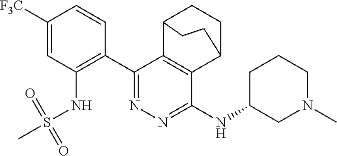 [SMILES: CN1CCC[C@@H](Nc2nnc(-c3ccc(C(F)(F)F)cc3NS(C)(=O)=O)c3c2C2CCC3CC2)C1] | N-[2-[6-[[(3R)-1-methyl-3- piperidyl]amino]-4,5- diazatricyclo[6.2.2.02,7]dodeca- 2(7),3,5-trien-3-yl]-5- (trifluoromethyl)phenyl]- methanesulfonamide |
| 55 | 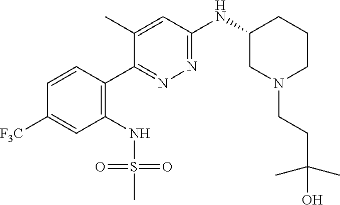 [SMILES: Cc1cc(N[C@@H]2CCCN(CCC(C)(C)O)C2)nnc1-c1ccc(C(F)(F)F)cc1NS(C)(=O)=O] | N-[2-[6-[[(3R)-1-(3-hydroxy-3- methyl-butyl)-3-piperidyl]amino]-4- methyl-pyridazin-3-yl]-5- (trifluoromethyl)phenyl]- methanesulfonamide |
| 56 | 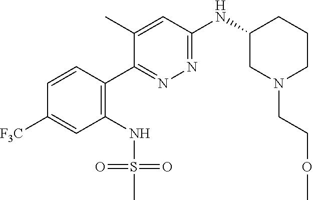 [SMILES: COCCN1CCC[C@@H](Nc2cc(C)c(-c3ccc(C(F)(F)F)cc3NS(C)(=O)=O)nn2)C1] | N-[2-[6-[[(3R)-1-(2-methoxyethyl)-3- piperidyl]amino]-4-methyl-pyridazin- 3-yl]-5- (trifluoromethyl)phenyl]- methanesulfonamide |
| 57 | 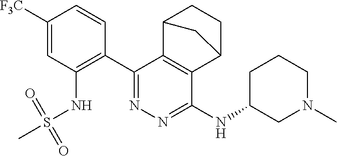 [SMILES: CN1CCC[C@@H](Nc2nnc(-c3ccc(C(F)(F)F)cc3NS(C)(=O)=O)c3c2C2CCC3C2)C1] | N-[2-[6-[[(3R)-1-methyl-3- piperidyl]amino]-4,5- diazatricyclo[6.2.1.02,7]undeca- 2(7),3,5-trien-3-yl]-5- (trifluoromethyl)phenyl]- methanesulfonamide |
| 58 | 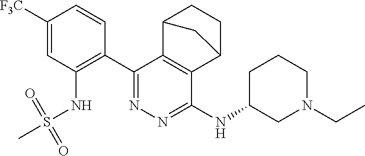 [SMILES: CCN1CCC[C@@H](Nc2nnc(-c3ccc(C(F)(F)F)cc3NS(C)(=O)=O)c3c2C2CCC3C2)C1] | N-[2-[6-[[(3R)-1-ethyl-3- piperidyl]amino]-4,5- diazatricyclo[6.2.1.02,7]undeca- 2(7),3,5-trien-3-yl]-5- (trifluoromethyl)phenyl]- methanesulfonamide |
| 59 | 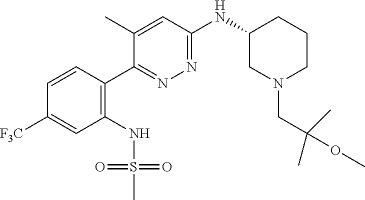 [SMILES: COC(C)(C)CN1CCC[C@@H](Nc2cc(C)c(-c3ccc(C(F)(F)F)cc3NS(C)(=O)=O)nn2)C1] | N-[2-[6-[[(3R)-1-(2-methoxy-2- methyl-propyl)-3-piperidyl]amino]-4- methyl-pyridazin-3-yl]-5- (trifluoromethyl)phenyl]- methanesulfonamide |
| 60 | 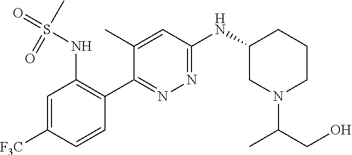 [SMILES: Cc1cc(N[C@@H]2CCCN(C(C)CO)C2)nnc1-c1ccc(C(F)(F)F)cc1NS(C)(=O)=O] | N-[2-[6-[[(3R)-1-(2-hydroxy-1- methyl-ethyl)-3-piperidyl]amino]-4- methyl-pyridazin-3-yl]-5- (trifluoromethyl)phenyl]- methanesulfonamide |
| 61 | 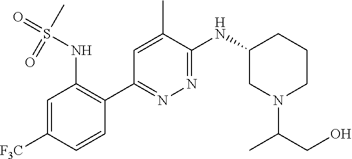 [SMILES: Cc1cc(-c2ccc(C(F)(F)F)cc2NS(C)(=O)=O)nnc1N[C@@H]1CCCN(C(C)CO)C1] | N-[2-[6-[[(3R)-1-(2-hydroxy-1- methyl-ethyl)-3-piperidyl]amino]-5- methyl-pyridazin-3-yl]-5- (trifluoromethyl)phenyl]- methanesulfonamide |
| 62 | 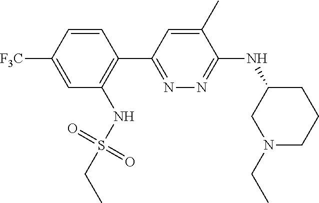 [SMILES: CCN1CCC[C@@H](Nc2nnc(-c3ccc(C(F)(F)F)cc3NS(=O)(=O)CC)cc2C)C1] | N-[2-[6-[[(3R)-1-ethyl-3- piperidyl]amino]-5-methyl-pyridazin- 3-yl]-5- (trifluoromethyl)phenyl]- ethanesulfonamide |
| 63 | 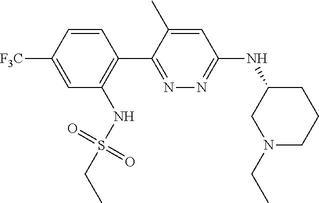 [SMILES: CCN1CCC[C@@H](Nc2cc(C)c(-c3ccc(C(F)(F)F)cc3NS(=O)(=O)CC)nn2)C1] | N-[2-[6-[[(3R)-1-ethyl-3- piperidyl]amino]-4-methyl-pyridazin- 3-yl]-5- (trifluoromethyl)phenyl]- ethanesulfonamide |
| 64 | 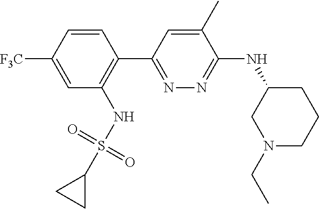 [SMILES: CCN1CCC[C@@H](Nc2nnc(-c3ccc(C(F)(F)F)cc3NS(=O)(=O)C3CC3)cc2C)C1] | N-[2-[6-[[(3R)-1-ethyl-3- piperidyl]amino]-5-methyl-pyridazin- 3-yl]-5- (trifluoromethyl)phenyl]cyclopropane sulfonamide |
| 65 | 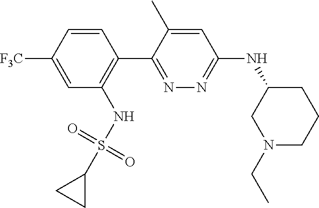 [SMILES: CCN1CCC[C@@H](Nc2cc(C)c(-c3ccc(C(F)(F)F)cc3NS(=O)(=O)C3CC3)nn2)C1] | N-[2-[6-[[(3R)-1-ethyl-3- piperidyl]amino]-4-methyl-pyridazin- 3-yl]-5- (trifluoromethyl)phenyl]cyclopropane sulfonamide |
| 66 | 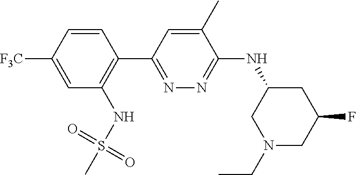 [SMILES: CCN1C[C@H](F)C[C@@H](Nc2nnc(-c3ccc(C(F)(F)F)cc3NS(C)(=O)=O)cc2C)C1] | N-[2-[6-[[(3R,5R)-1-ethyl-5-fluoro- 3-piperidyl]amino]-5-methyl- pyridazin-3-yl]-5- (trifluoromethyl)phenyl]- methanesulfonamide |
| 67 | 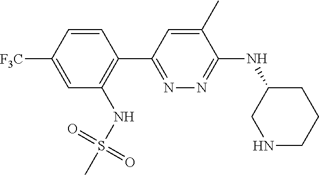 [SMILES: Cc1cc(-c2ccc(C(F)(F)F)cc2NS(C)(=O)=O)nnc1N[C@@H]1CCCNC1] | N-[2-[5-methyl-6-[(3R)-3- piperidyl]amino]pyridazin-3-yl]-5- (trifluoromethyl)phenyl]- methanesulfonamide |
| 68 | 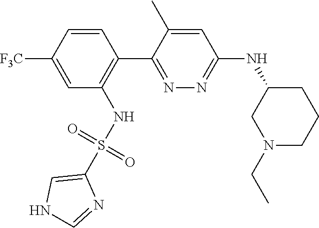 [SMILES: CCN1CCC[C@@H](Nc2cc(C)c(-c3ccc(C(F)(F)F)cc3NS(=O)(=O)c3c[nH]cn3)nn2)C1] | N-[2-[6-[(3R)-1-ethyl-3- piperidyl]amino]-5-methyl-pyridazin- 3-yl]-5-(trifluoromethyl)phenyl]-1H- imidazole-4-sulfonamide |
| 69 | 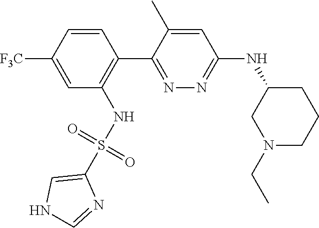 [SMILES: CCN1CCC[C@@H](Nc2cc(C)c(-c3ccc(C(F)(F)F)cc3NS(=O)(=O)c3c[nH]cn3)nn2)C1] | N-[2-[6-[[(3R)-1-ethyl-3- piperidyl]amino]-4-methyl-pyridazin- 3-yl]-5-(trifluoromethyl)phenyl]-1H- imidazole-4-sulfonamide |
| 70 | 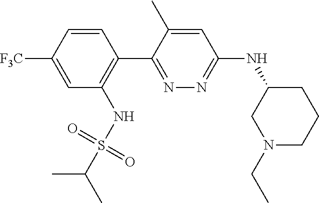 [SMILES: CCN1CCC[C@@H](Nc2cc(C)c(-c3ccc(C(F)(F)F)cc3NS(=O)(=O)C(C)C)nn2)C1] | N-[2-[6-[(3R)-1-ethyl-3- piperidyl]amino]-4-methyl-pyridazin- 3-yl]-5- (trifluoromethyl)phenyl]propane-2- sulfonamide |
| 71 | 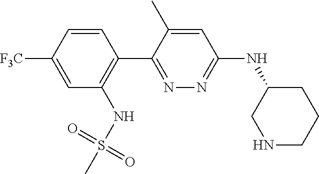 [SMILES: Cc1cc(N[C@@H]2CCCNC2)nnc1-c1ccc(C(F)(F)F)cc1NS(C)(=O)=O] | N-[2-[4-methyl-6-[[(3R)-3- piperidyl]amino]pyridazin-3-yl]-5- (trifluoromethyl)phenyl]- methanesulfonamide |
| 72 | 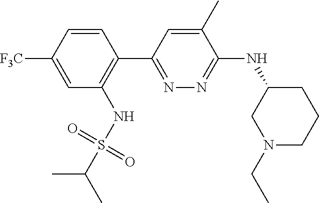 [SMILES: CCN1CCC[C@@H](Nc2nnc(-c3ccc(C(F)(F)F)cc3NS(=O)(=O)C(C)C)cc2C)C1] | N-[2-[6-[[(3R)-1-ethyl-3- piperidyl]amino]-5-methyl-pyridazin- 3-yl]-5- (trifluoromethyl)phenyl]propane-2- sulfonamide |
| 73 | 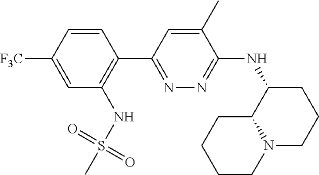 [SMILES: Cc1cc(-c2ccc(C(F)(F)F)cc2NS(C)(=O)=O)nnc1N[C@@H]1CCCN2CCCC[C@H]12] | N-[2-[6-[[(1R,9aR)-2,3,4,6,7,8,9,9a- octahydro-1H-quinolizin-1- yl]amino]-5-methyl-pyridazin-3-yl]- 5- (trifluoromethyl)phenyl]- methanesulfonamide |
| 74 | 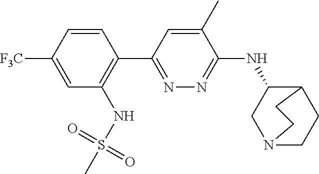 [SMILES: Cc1cc(-c2ccc(C(F)(F)F)cc2NS(C)(=O)=O)nnc1N[C@H]1CN2CCC1CC2] | N-[2-[5-methyl-6-[[(3R)-quinuclidin- 3-yl]amino]pyridazin-3-yl]-5- (trifluoromethyl)phenyl]- methanesulfonamide |
| 75 | 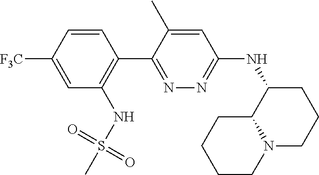 [SMILES: Cc1cc(N[C@@H]2CCCN3CCCC[C@H]23)nnc1-c1ccc(C(F)(F)F)cc1NS(C)(=O)=O] | N-[2-[6-[[(1R,9aR)-2,3,4,6,7,8,9,9a- octahydro-1H-quinolizin-1- yl]amino]-4-methyl-pyridazin-3-yl]- 5- (trifluoromethyl)phenyl]- methanesulfonamide |
| 76 | 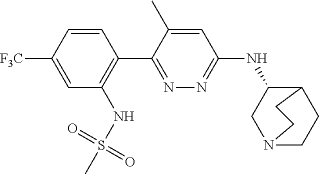 [SMILES: Cc1cc(N[C@H]2CN3CCC2CC3)nnc1-c1ccc(C(F)(F)F)cc1NS(C)(=O)=O] | N-[2-[4-methyl-6-[[(3R)-quinuclidin- 3-yl]amino]pyridazin-3-yl]-5- (trifluoromethyl)phenyl]- methanesulfonamide |
| 77 | 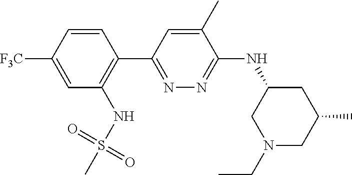 [SMILES: CCN1C[C@@H](C)C[C@@H](Nc2nnc(-c3ccc(C(F)(F)F)cc3NS(C)(=O)=O)cc2C)C1] | N-[2-[6-[[(3R,5S)-1-ethyl-5-methyl- 3-piperidyl]amino]-5-methyl- pyridazin-3-yl]-5- (trifluoromethyl)phenyl]- methanesulfonamide |
| 78 | 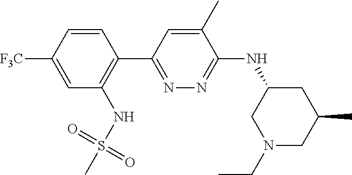 [SMILES: CCN1C[C@H](C)C[C@@H](Nc2nnc(-c3ccc(C(F)(F)F)cc3NS(C)(=O)=O)cc2C)C1] | N-[2-[6-[[(3R,5R)-1-ethyl-5-methyl- 3-piperidyl]amino]-5-methyl- pyridazin-3-yl]-5- (trifluoromethyl)phenyl]- methanesulfonamide |
| 79 | 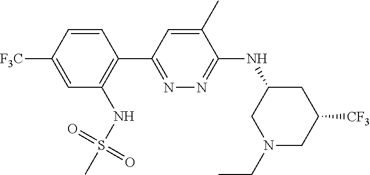 [SMILES: CCN1C[C@H](Nc2nnc(-c3ccc(C(F)(F)F)cc3NS(C)(=O)=O)cc2C)C[C@H](C(F)(F)F)C1] | N-[2-[6-[(3R,5S)-1-ethyl-5- (trifluoromethyl)-3-piperidyl]amino]- 5-methyl-pyridazin-3-yl]-5- (trifluoromethyl)phenyl]- methanesulfonamide |
| 80 | 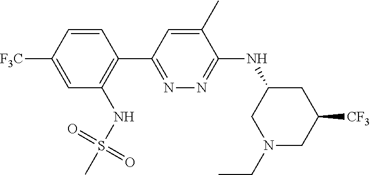 [SMILES: CCN1C[C@H](Nc2nnc(-c3ccc(C(F)(F)F)cc3NS(C)(=O)=O)cc2C)C[C@@H](C(F)(F)F)C1] | N-[2-[6-[[(3R,5R)-1-ethyl-5- (trifluoromethyl)-3-piperidyl]amino]- 5-methyl-pyridazin-3-yl]-5- (trifluoromethyl)phenyl]- methanesulfonamide |
| 81 | 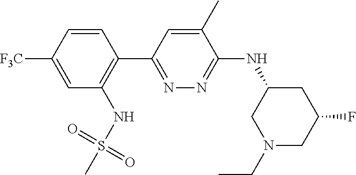 [SMILES: CCN1C[C@@H](F)C[C@@H](Nc2nnc(-c3ccc(C(F)(F)F)cc3NS(C)(=O)=O)cc2C)C1] | N-[2-[6-[[(3R,5S)-1-ethyl-5-fluoro-3- piperidyl]amino]-5-methyl-pyridazin- 3-yl]-5- (trifluoromethyl)phenyl]- methanesulfonamide |
| 82 | 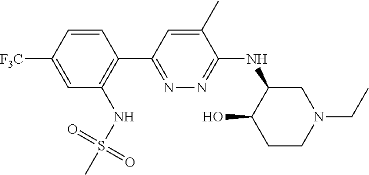 [SMILES: CCN1CC[C@@H](O)[C@@H](Nc2nnc(-c3ccc(C(F)(F)F)cc3NS(C)(=O)=O)cc2C)C1] | N-[2-[6-[[(3S,4R)-1-ethyl-4-hydroxy- 3-piperidyl]amino]-5-methyl- pyridazin-3-yl]-5- (trifluoromethyl)phenyl]- methanesulfonamide |
| 83 | 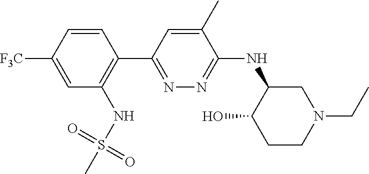 [SMILES: CCN1CC[C@H](O)[C@@H](Nc2nnc(-c3ccc(C(F)(F)F)cc3NS(C)(=O)=O)cc2C)C1] | N-[2-[6-[[(3S,4S)-1-ethyl-4-hydroxy- 3-piperidyl]amino]-5-methyl- pyridazin-3-yl]-5- (trifluoromethyl)phenyl]- methanesulfonamide |
| 84 | 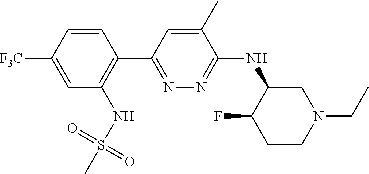 [SMILES: CCN1CC[C@@H](F)[C@@H](Nc2nnc(-c3ccc(C(F)(F)F)cc3NS(C)(=O)=O)cc2C)C1] | N-[2-[6-[(3S,4R)-1-ethyl-4-fluoro-3- piperidyl]amino]-5-methyl-pyridazin- 3-yl]-5- (trifluoromethyl)phenyl]- methanesulfonamide |
| 85 | 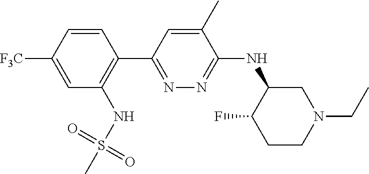 [SMILES: CCN1CC[C@H](F)[C@@H](Nc2nnc(-c3ccc(C(F)(F)F)cc3NS(C)(=O)=O)cc2C)C1] | N-[2-[6-[[(3S,4S)-1-ethyl-4-fluoro-3- piperidyl]amino]-5-methyl-pyridazin- 3-yl]-5- (trifluoromethyl)phenyl]- methanesulfonamide |
| 86 | 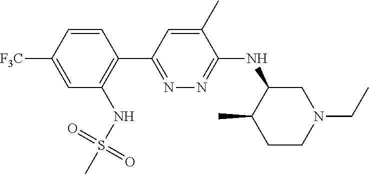 [SMILES: CCN1CC[C@@H](C)[C@@H](Nc2nnc(-c3ccc(C(F)(F)F)cc3NS(C)(=O)=O)cc2C)C1] | N-[2-[6-[[(3R,4R)-1-ethyl-4-methyl- 3-piperidyl]amino]-5-methyl- pyridazin-3-yl]-5- (trifluoromethyl)phenyl]- methanesulfonamide |
| 87 | 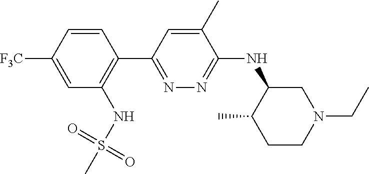 [SMILES: CCN1CC[C@H](C)[C@@H](Nc2nnc(-c3ccc(C(F)(F)F)cc3NS(C)(=O)=O)cc2C)C1] | N-[2-[6-[[(3R,4S)-1-ethyl-4-methyl- 3-piperidyl]amino]-5-methyl- pyridazin-3-yl]-5- (trifluoromethyl)phenyl]- methanesulfonamide |
| 88 | 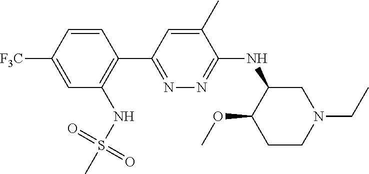 [SMILES: CCN1CC[C@@H](OC)[C@@H](Nc2nnc(-c3ccc(C(F)(F)F)cc3NS(C)(=O)=O)cc2C)C1] | N-[2-[6-[[(3S,4R)-1-ethyl-4- methoxy-3-piperidyl]amino]-5- methyl-pyridazin-3-yl]-5- (trifluoromethyl)phenyl]- methanesulfonamide |
| 89 | 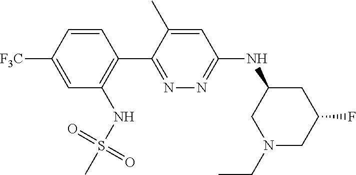 [SMILES: CCN1C[C@@H](F)C[C@H](Nc2cc(C)c(-c3ccc(C(F)(F)F)cc3NS(C)(=O)=O)nn2)C1] | N-[2-[6-[[(3S,5S)-1-ethyl-5-fluoro-3- piperidyl]amino]-4-methyl-pyridazin- 3-yl]-5- (trifluoromethyl)phenyl]- methanesulfonamide |
| 90 | 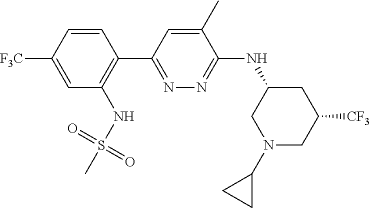 [SMILES: Cc1cc(-c2ccc(C(F)(F)F)cc2NS(C)(=O)=O)nnc1N[C@@H]1C[C@H](C(F)(F)F)CN(C2CC2)C1] | N-[2-[6-[[(3R,5S)-1-cyclopropyl-5- (trifluoromethyl)-3-piperidyl]amino]- 5-methyl-pyridazin-3-yl]-5- (trifluoromethyl)phenyl]- methanesulfonamide |
| 91 | 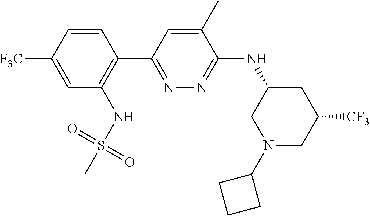 [SMILES: Cc1cc(-c2ccc(C(F)(F)F)cc2NS(C)(=O)=O)nnc1N[C@@H]1C[C@H](C(F)(F)F)CN(C2CCC2)C1] | N-[2-[6-[(3R,5S)-1-cyclobutyl-5- (trifluoromethyl)-3-piperidyl]amino]- 5-methyl-pyridazin-3-yl]-5- (trifluoromethyl)phenyl]- methanesulfonamide |
| 92 | 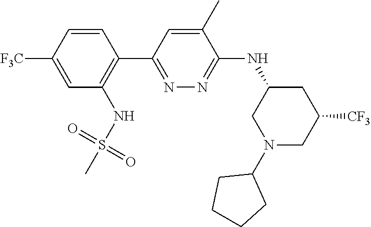 [SMILES: Cc1cc(-c2ccc(C(F)(F)F)cc2NS(C)(=O)=O)nnc1N[C@@H]1C[C@H](C(F)(F)F)CN(C2CCCC2)C1] | N-[2-[6-[(3R,5S)-1-cyclopentyl-5- (trifluoromethyl)-3-piperidyl]amino]- 5-methyl-pyridazin-3-yl]-5- (trifluoromethyl)phenyl]- methanesulfonamide |
| 93 | 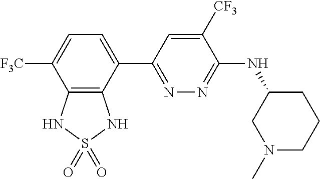 [SMILES: CN1CCC[C@@H](Nc2nnc(-c3ccc(C(F)(F)F)c4c3NS(=O)(=O)N4)cc2C(F)(F)F)C1] | 6-[2,2-dioxo-4-(trifluoromethyl)-1,3- dihydro-2lambda6,1,3- benzothiadiazol-7-yl]-N-[(3R)-1- methyl-3-piperidyl]-4- (trifluoromethyl)pyridazin-3-amine |
| 94 | 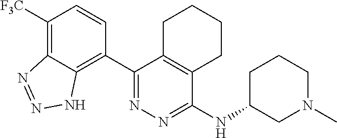 [SMILES: CN1CCC[C@@H](Nc2nnc(-c3ccc(C(F)(F)F)c4nn[nH]c34)c3c2CCCC3)C1] | N-[(3R)-1-methyl-3-piperidyl]-4-[7- (trifluoromethyl)-3H-benzotriazol-4- yl]-5,6,7,8-tetrahydrophthalazin-1- amine |
| 95 | 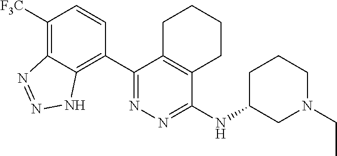 [SMILES: CCN1CCC[C@@H](Nc2nnc(-c3ccc(C(F)(F)F)c4nn[nH]c34)c3c2CCCC3)C1] | N-[(3R)-1-ethyl-3-piperidyl]-4-[7- (trifluoromethyl)-3H-benzotriazol-4- yl]-5,6,7,8-tetrahydrophthalazin-1- amine |
| 96 | 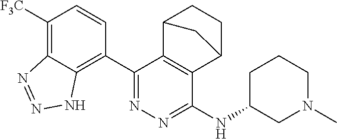 [SMILES: CN1CCC[C@@H](Nc2nnc(-c3ccc(C(F)(F)F)c4nn[nH]c34)c3c2C2CCC3C2)C1] | N-[(3R)-1-methyl-3-piperidyl]-6-[7- (trifluoromethyl)-3H-benzotriazol-4- yl]-4,5- diazatricyclo[6.2.1.02,7]undeca- 2(7),3,5-trien-3-amine |
| 97 | 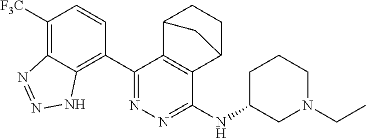 [SMILES: CCN1CCC[C@@H](Nc2nnc(-c3ccc(C(F)(F)F)c4nn[nH]c34)c3c2C2CCC3C2)C1] | N-[(3R)-1-ethyl-3-piperidyl]-6-[7- (trifluoromethyl)-3H-benzotriazol-4- yl]-4,5- diazatricyclo[6.2.1.02,7]undeca- 2(7),3,5-trien-3-amine |
| 98 | 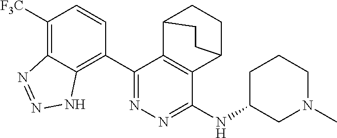 [SMILES: CN1CCC[C@@H](Nc2nnc(-c3ccc(C(F)(F)F)c4nn[nH]c34)c3c2C2CCC3CC2)C1] | N-[(3R)-1-methyl-3-piperidyl]-6-[7- (trifluoromethyl)-3H-benzotriazol-4- yl]-4,5- diazatricyclo[6.2.2.02,7]dodeca- 2(7),3,5-trien-3-amine |
| 99 | 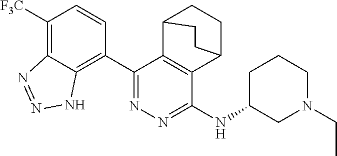 [SMILES: CCN1CCC[C@@H](Nc2nnc(-c3ccc(C(F)(F)F)c4nn[nH]c34)c3c2C2CCC3CC2)C1] | N-[(3R)-1-ethyl-3-piperidyl]-6-[7- (trifluoromethyl)-3H-benzotriazol-4- yl]-4,5- diazatricyclo[6.2.2.02,7]dodeca- 2(7),3,5-trien-3-amine |
| 100 | 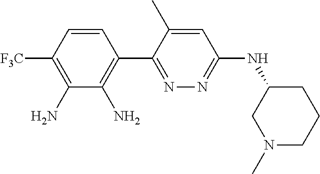 [SMILES: Cc1cc(N[C@@H]2CCCN(C)C2)nnc1-c1ccc(C(F)(F)F)c(N)c1N] | 3-[4-methyl-6-[[(3R)-1-methyl-3- piperidyl]amino]pyridazin-3-yl]-6- (trifluoromethyl)benzene-1,2-diamine |
| 101 | 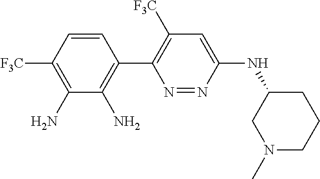 [SMILES: CN1CCC[C@@H](Nc2cc(C(F)(F)F)c(-c3ccc(C(F)(F)F)c(N)c3N)nn2)C1] | 3-[6-[[(3R)-1-methyl-3- piperidyl]amino]-4- (trifluoromethyl)pyridazin-3-yl]-6- (trifluoromethyl)benzene-1,2-diamine |
| 102 | 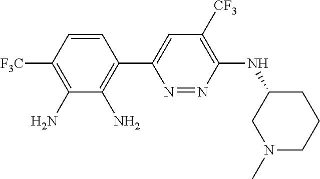 [SMILES: CN1CCC[C@@H](Nc2nnc(-c3ccc(C(F)(F)F)c(N)c3N)cc2C(F)(F)F)C1] | 3-[6-[[(3R)-1-methyl-3- piperidyl]amino]-5- (trifluoromethyl)pyridazin-3-yl]-6- (trifluoromethyl)benzene-1,2-diamine |
| 103 | 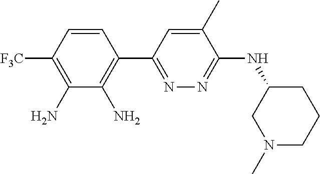 [SMILES: Cc1cc(-c2ccc(C(F)(F)F)c(N)c2N)nnc1N[C@@H]1CCCN(C)C1] | 3-[5-methyl-6-[[(3R)-1-methyl-3- piperidyl]amino]pyridazin-3-yl]-6- (trifluoromethyl)benzene-1,2-diamine |
| 104 | 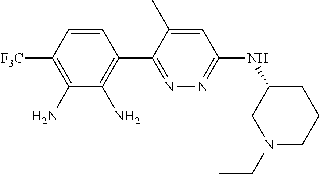 [SMILES: CCN1CCC[C@@H](Nc2cc(C)c(-c3ccc(C(F)(F)F)c(N)c3N)nn2)C1] | 3-[6-[[(3R)-1-ethyl-3- piperidyl]amino]-4-methyl-pyridazin- 3-yl]-6-(trifluoromethyl)benzene-1,2- diamine |
| 105 | 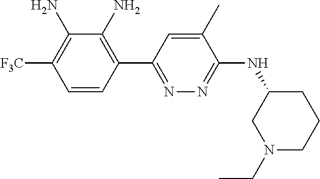 [SMILES: CCN1CCC[C@@H](Nc2nnc(-c3ccc(C(F)(F)F)c(N)c3N)cc2C)C1] | 3-[6-[[(3R)-1-ethyl-3- piperidyl]amino]-5-methyl-pyridazin- 3-yl]-6-(trifluoromethyl)benzene-1,2- diamine |
| 106 | 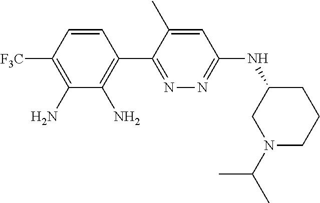 [SMILES: Cc1cc(N[C@@H]2CCCN(C(C)C)C2)nnc1-c1ccc(C(F)(F)F)c(N)c1N] | 3-[6-[[(3R)-1-isopropyl-3- piperidyl]amino]-4-methyl-pyridazin- 3-yl]-6-(trifluoromethyl)benzene-1,2- diamine |
| 107 | 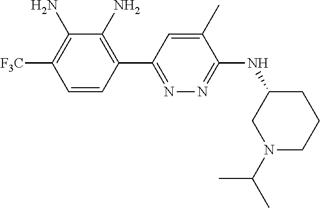 [SMILES: Cc1cc(-c2ccc(C(F)(F)F)c(N)c2N)nnc1N[C@@H]1CCCN(C(C)C)C1] | 3-[6-[[(3R)-1-isopropyl-3- piperidyl]amino]-5-methyl-pyridazin- 3-yl]-6-(trifluoromethyl)benzene-1,2- diamine |
| 108 | 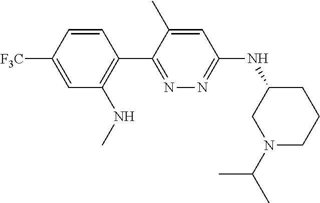 [SMILES: CNc1cc(C(F)(F)F)ccc1-c1nnc(N[C@@H]2CCCN(C(C)C)C2)cc1C] | N-[(3R)-1-isopropyl-3-piperidyl]-5- methyl-6-[2-(methylamino)-4- (trifluoromethyl)phenyl]pyridazin-3- amine |
| 109 | 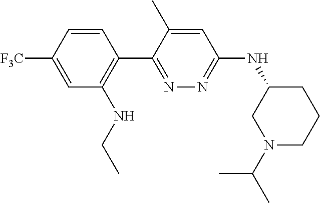 [SMILES: CCNc1cc(C(F)(F)F)ccc1-c1nnc(N[C@@H]2CCCN(C(C)C)C2)cc1C] | 6-[2-(ethylamino)-4- (trifluoromethyl)phenyl]-N-[(3R)-1- isopropyl-3-piperidyl]-5-methyl- pyridazin-3-amine |
| 110 | 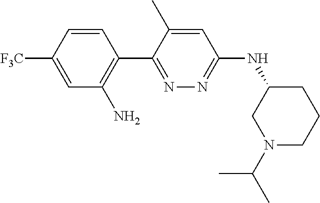 [SMILES: Cc1cc(N[C@@H]2CCCN(C(C)C)C2)nnc1-c1ccc(C(F)(F)F)cc1N] | 6-[2-amino-4- (trifluoromethyl)phenyl]-N-[(3R)-1- isopropyl-3-piperidyl]-5-methyl- pyridazin-3-amine |
| 111 | 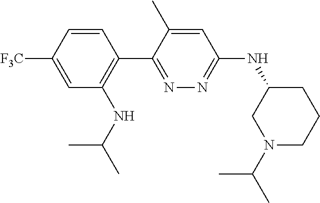 [SMILES: Cc1cc(N[C@@H]2CCCN(C(C)C)C2)nnc1-c1ccc(C(F)(F)F)cc1NC(C)C] | 6-[2-(isopropylamino)-4- (trifluoromethyl)phenyl]-N-[(3R)-1- isopropyl-3-piperidyl]-5-methyl- pyridazin-3-amine |
| 112 | 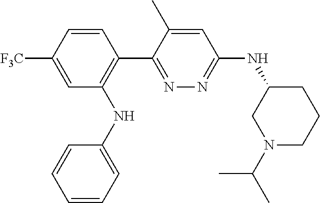 [SMILES: Cc1cc(N[C@@H]2CCCN(C(C)C)C2)nnc1-c1ccc(C(F)(F)F)cc1Nc1ccccc1] | 6-[2-anilino-4- (trifluoromethyl)phenyl]-N-[(3R)-1- isopropyl-3-piperidyl]-5-methyl- pyridazin-3-amine |
| 113 | 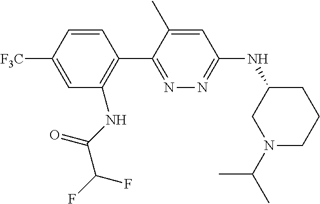 [SMILES: Cc1cc(N[C@@H]2CCCN(C(C)C)C2)nnc1-c1ccc(C(F)(F)F)cc1NC(=O)C(F)F] | 2,2-difluoro-N-[2-[6-[(3R)-1- isopropyl-3-piperidyl]amino]-4- methyl-pyridazin-3-yl]-5- (trifluoromethyl)phenyl]acetamide |
| 114 | 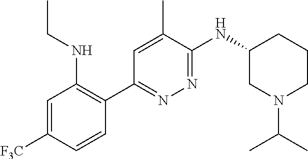 [SMILES: CCNc1cc(C(F)(F)F)ccc1-c1cc(C)c(N[C@@H]2CCCN(C(C)C)C2)nn1] | 6-[2-(ethylamino)-4- (trifluoromethyl)phenyl]-N-[(3R)-1- isopropyl-3-piperidyl]-4-methyl- pyridazin-3-amine |
| 115 | 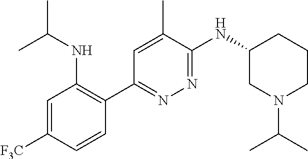 [SMILES: Cc1cc(-c2ccc(C(F)(F)F)cc2NC(C)C)nnc1N[C@@H]1CCCN(C(C)C)C1] | 6-[2-(isopropylamino)-4- (trifluoromethyl)phenyl]-N-[(3R)-1- isopropyl-3-piperidyl]-4-methyl- pyridazin-3-amine |
| 116 | 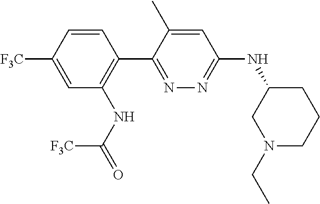 [SMILES: CCN1CCC[C@@H](Nc2cc(C)c(-c3ccc(C(F)(F)F)cc3NC(=O)C(F)(F)F)nn2)C1] | N-[2-[6-[(3R)-1-ethyl-3- piperidyl]amino]-4-methyl-pyridazin- 3-yl]-5-(trifluoromethyl)phenyl]- 2,2,2-trifluoro-acetamide |
| 117 | 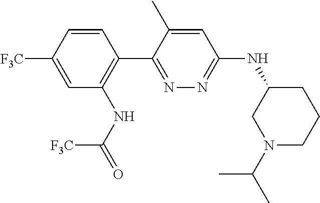 [SMILES: Cc1cc(N[C@@H]2CCCN(C(C)C)C2)nnc1-c1ccc(C(F)(F)F)cc1NC(=O)C(F)(F)F] | 2,2,2-trifluoro-N-[2-[6-[(3R)-1- isopropyl-3-piperidyl]amino]-4- methyl-pyridazin-3-yl]-5- (trifluoromethyl)phenyl]acetamide |
| 118 | 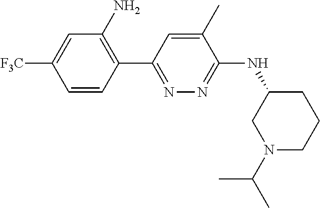 [SMILES: Cc1cc(-c2ccc(C(F)(F)F)cc2N)nnc1N[C@@H]1CCCN(C(C)C)C1] | 6-[2-amino-4- (trifluoromethyl)phenyl]-N-[(3R)-1- isopropyl-3-piperidyl]-4-methyl- pyridazin-3-amine |
| 119 | 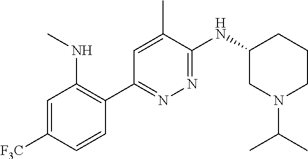 [SMILES: CNc1cc(C(F)(F)F)ccc1-c1cc(C)c(N[C@@H]2CCCN(C(C)C)C2)nn1] | N-[(3R)-1-isopropyl-3-piperidyl]-4- methyl-6-[2-(methylamino)-4- (trifluoromethyl)phenyl]pyridazin-3- amine |
| 120 | 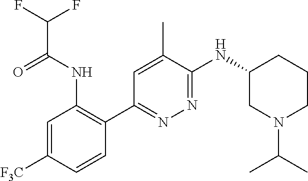 [SMILES: Cc1cc(-c2ccc(C(F)(F)F)cc2NC(=O)C(F)F)nnc1N[C@@H]1CCCN(C(C)C)C1] | 2,2-difluoro-N-[2-[6-[(3R)-1- isopropyl-3-piperidyl]amino]-5- methyl-pyridazin-3-yl]-5- (trifluoromethyl)phenyl]acetamide |
| 121 | 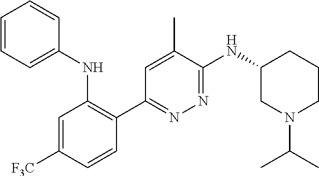 [SMILES: Cc1cc(-c2ccc(C(F)(F)F)cc2Nc2ccccc2)nnc1N[C@@H]1CCCN(C(C)C)C1] | 6-[2-anilino-4- (trifluoromethyl)phenyl]-N-[(3R)-1- isopropyl-3-piperidyl]-4-methyl- pyridazin-3-amine |
| 122 | 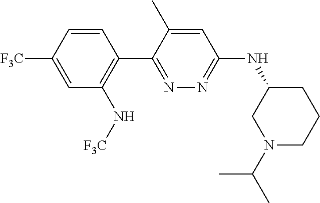 [SMILES: Cc1cc(N[C@@H]2CCCN(C(C)C)C2)nnc1-c1ccc(C(F)(F)F)cc1NC(F)(F)F] | N-[(3R)-1-isopropyl-3-piperidyl]-5- methyl-6-[4-(trifluoromethyl)-2- (trifluoromethylamino)phenyl]- pyridazin-3-amine |
| 123 | 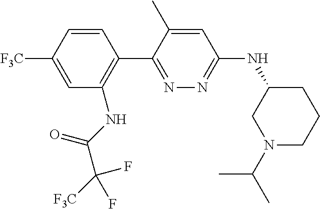 [SMILES: Cc1cc(N[C@@H]2CCCN(C(C)C)C2)nnc1-c1ccc(C(F)(F)F)cc1NC(=O)C(F)(F)C(F)(F)F] | 2,2,3,3,3-pentafluoro-N-[2-[6-[[(3R)- 1-isopropyl-3-piperidyl]amino]-4- methyl-pyridazin-3-yl]-5- (trifluoromethyl)phenyl]propanamide |
| 124 | 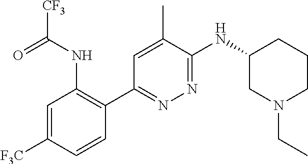 [SMILES: CCN1CCC[C@@H](Nc2nnc(-c3ccc(C(F)(F)F)cc3NC(=O)C(F)(F)F)cc2C)C1] | N-[2-[6-[[(3R)-1-ethyl-3- piperidyl]amino]-5-methyl-pyridazin- 3-yl]-5-(trifluoromethyl)phenyl]- 2,2,2-trifluoro-acetamide |
| 125 | 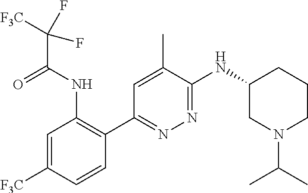 [SMILES: Cc1cc(-c2ccc(C(F)(F)F)cc2NC(=O)C(F)(F)C(F)(F)F)nnc1N[C@@H]1CCCN(C(C)C)C1] | 2,2,3,3,3-pentafluoro-N-[2-[6-[(3R)- 1-isopropyl-3-piperidyl]amino]-5- methyl-pyridazin-3-yl]-5- (trifluoromethyl)phenyl]propanamide |
| 126 | 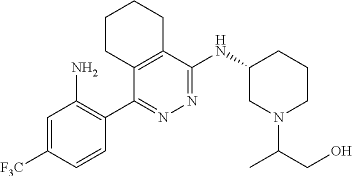 [SMILES: CC(CO)N1CCC[C@@H](Nc2nnc(-c3ccc(C(F)(F)F)cc3N)c3c2CCCC3)C1] | 2-[(3R)-3-[[4-[2-amino-4- (trifluoromethyl)phenyl]-5,6,7,8- tetrahydrophthalazin-1-yl]amino]-1- piperidyl]propan-1-ol |
| 127 | 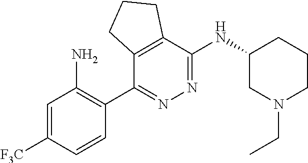 [SMILES: CCN1CCC[C@@H](Nc2nnc(-c3ccc(C(F)(F)F)cc3N)c3c2CCC3)C1] | 1-[2-amino-4- (trifluoromethyl)phenyl]-N-[(3R)-1- ethyl-3-piperidyl]-6,7-dihydro-5H- cyclopenta[d]pyridazin-4-amine |
| 128 | 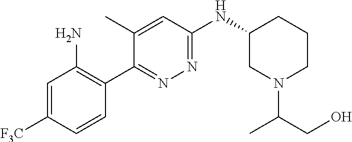 [SMILES: Cc1cc(N[C@@H]2CCCN(C(C)CO)C2)nnc1-c1ccc(C(F)(F)F)cc1N] | 2-[(3R)-3-[[6-[2-amino-4- (trifluoromethyl)phenyl]-5-methyl- pyridazin-3-yl]amino]-1- piperidyl]propan-1-ol |
| 129 | 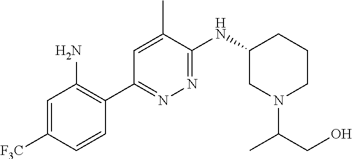 [SMILES: Cc1cc(-c2ccc(C(F)(F)F)cc2N)nnc1N[C@@H]1CCCN(C(C)CO)C1] | 2-[(3R)-3-[[6-[2-amino-4- (trifluoromethyl)phenyl]-4-methyl- pyridazin-3-yl]amino]-1- piperidyl]propan-1-ol |
| 130 | 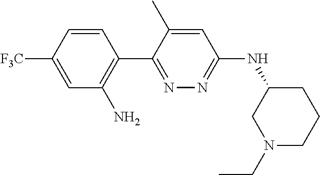 [SMILES: CCN1CCC[C@@H](Nc2cc(C)c(-c3ccc(C(F)(F)F)cc3N)nn2)C1] | 6-[2-amino-4- (trifluoromethyl)phenyl]-N-[(3R)-1- ethyl-3-piperidyl]-5-methyl- pyridazin-3-amine |
| 131 | 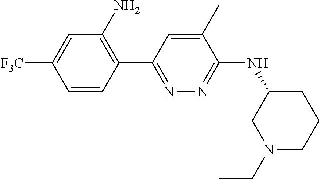 [SMILES: CCN1CCC[C@@H](Nc2nnc(-c3ccc(C(F)(F)F)cc3N)cc2C)C1] | 6-[2-amino-4- (trifluoromethyl)phenyl]-N-[(3R)-1- ethyl-3-piperidyl]-4-methyl- pyridazin-3-amine |
| 132 | 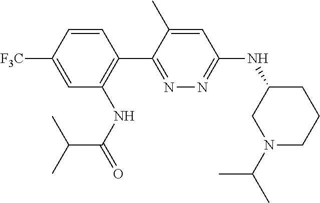 [SMILES: Cc1cc(N[C@@H]2CCCN(C(C)C)C2)nnc1-c1ccc(C(F)(F)F)cc1NC(=O)C(C)C] | N-[2-[6-[[(3R)-1-isopropyl-3- piperidyl]amino]-4-methyl-pyridazin- 3-yl]-5-(trifluoromethyl)phenyl]-2- methyl-propanamide |
| 133 | 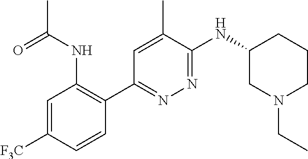 [SMILES: CCN1CCC[C@@H](Nc2nnc(-c3ccc(C(F)(F)F)cc3NC(C)=O)cc2C)C1] | N-[2-[6-[[(3R)-1-ethyl-3- piperidyl]amino]-5-methyl-pyridazin- 3-yl]-5- (trifluoromethyl)phenyl]acetamide |
| 134 | 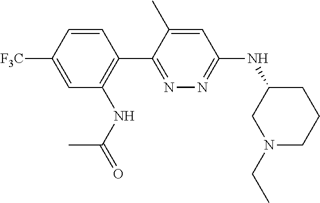 [SMILES: CCN1CCC[C@@H](Nc2cc(C)c(-c3ccc(C(F)(F)F)cc3NC(C)=O)nn2)C1] | N-[2-[6-[[(3R)-1-ethyl-3- piperidyl]amino]-4-methyl-pyridazin- 3-yl]-5- (trifluoromethyl)phenyl]acetamide |
| 135 | 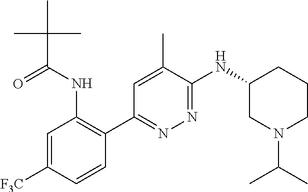 [SMILES: Cc1cc(-c2ccc(C(F)(F)F)cc2NC(=O)C(C)(C)C)nnc1N[C@@H]1CCCN(C(C)C)C1] | N-[2-[6-[[(3R)-1-isopropyl-3- piperidyl]amino]-5-methyl-pyridazin- 3-yl]-5-(trifluoromethyl)phenyl]-2,2- dimethyl-propanamide |
| 136 | 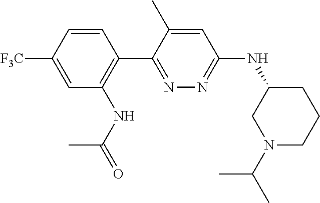 [SMILES: CC(=O)Nc1cc(C(F)(F)F)ccc1-c1nnc(N[C@@H]2CCCN(C(C)C)C2)cc1C] | N-[2-[6-[[(3R)-1-isopropyl-3- piperidyl]amino]-4-methyl-pyridazin- 3-yl]-5- (trifluoromethyl)phenyl]acetamide |
| 137 | 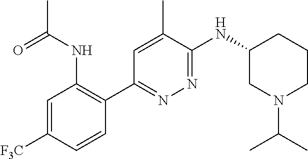 [SMILES: CC(=O)Nc1cc(C(F)(F)F)ccc1-c1cc(C)c(N[C@@H]2CCCN(C(C)C)C2)nn1] | N-[2-[6-[[(3R)-1-isopropyl-3- piperidyl]amino]-5-methyl-pyridazin- 3-yl]-5- (trifluoromethyl)phenyl]acetamide |
| 138 | 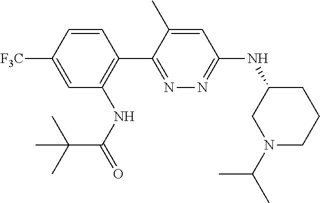 [SMILES: Cc1cc(N[C@@H]2CCCN(C(C)C)C2)nnc1-c1ccc(C(F)(F)F)cc1NC(=O)C(C)(C)C] | N-[2-[6-[[(3R)-1-isopropyl-3-piperidyl]amino]- 4-methyl-pyridazin- 3-yl]-5- (trifluoromethyl)phenyl]-2,2- dimethyl-propanamide |
| 139 | 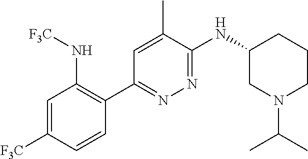 [SMILES: Cc1cc(-c2ccc(C(F)(F)F)cc2NC(F)(F)F)nnc1N[C@@H]1CCCN(C(C)C)C1] | N-[(3R)-1-isopropyl-3-piperidyl]-4 methyl-6- [4-(trifluoromethyl)-2- (trifluoromethylamino)phenyl]pyrida zin-3-amine |
| 140 | 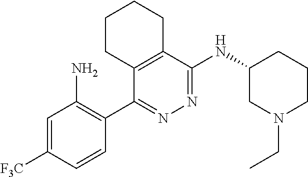 [SMILES: CCN1CCC[C@@H](Nc2nnc(-c3ccc(C(F)(F)F)cc3N)c3c2CCCC3)C1] | 4-[2-amino-4-(trifluoromethyl)phenyl]-N-[(3R)-1- ethyl-3-piperidyl]-5,6,7,8- tetrahydrophthalazin-1-amine |
| 141 | 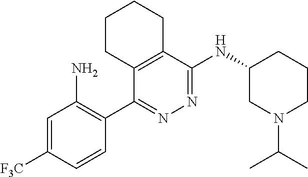 [SMILES: CC(C)N1CCC[C@@H](Nc2nnc(-c3ccc(C(F)(F)F)cc3N)c3c2CCCC3)C1] | 4-[2-amino-4-(trifluoromethyl)phenyl]-N-[(3R)-1- isopropyl-3-piperidyl]-5,6,7,8- tetrahydrophthalazin-1-amine |

or a pharmaceutically acceptable salt, stereoisomer, solvate, or tautomer thereof.

10. A pharmaceutical composition comprising the compound of any one of claims 1-9 or a pharmaceutically acceptable salt, stereoisomer, solvate, or tautomer thereof, and a pharmaceutically acceptable carrier.

11. The pharmaceutical composition of claim 10, further comprising one or more additional pharmaceutically active agents.

12. A method of inhibiting of NLRP3 in a cell, comprising contacting the cell with a compound of any one of claims 1-9 or a pharmaceutical composition of claim 10 or 11.

13. The method of claim 12, wherein the contacting is in vitro or in vivo.

14. A method for the treatment or prevention of a disease or disorder associated with NLRP3 comprising administering to a subject in need thereof a compound of any one of claims 1-9 or a pharmaceutical composition of claim 10 or 11.

15. The method of claim 14, wherein the disease or disorder is selected from the group consisting of Acne (ACNE); Acrokeratoderma, Hereditary Papulotranslucent; Adult-Onset Still's Disease (AOSD); Aicardi-Goutieres Syndrome (AGS); Alzheimer Disease, Familial, 1 (AD1); Amyloidosis; Amyotrophic Lateral Sclerosis 1 (ALS1); Anthracosis; Aphthous Stomatitis; Arthritis; Arthropathy; Aseptic Meningitis; Asthma (ASTHMA); Autoimmune Disease of Cardiovascular System; Autoimmune Disease of Gastrointestinal Tract; Autoinflammatory Syndrome; Behcet Syndrome (BD); Blau Syndrome (BLAUS); Body Mass Index Quantitative Trait Locus 11 (BMIQ11); Bone Inflammation Disease; Brain Edema; Brucellosis; Candidiasis; Cervical Adenitis; Chondrocalcinosis; Chronic Meningitis; Chronic Recurrent Multifocal Osteomyelitis (CRMO); Chronic Urticaria; Cinca Syndrome (CINCA); Conjunctival Disease; Conjunctivitis; Connective Tissue Disease; Corneal Disease; Coronavirus Infectious Disease; Covid-19; Crohn's Disease; Cryopyrin-Associated Periodic Syndrome (CAPS); Cystic Fibrosis (CF); Deafness, Autosomal Dominant 34, with or Without Inflammation (DFNA34); Dermatitis; Diabetes Mellitus; Diabetic Encephalopathy; Exanthem (RASH); Eye Disease; Familial Cold Autoinflammatory Syndrome (FCU); Familial Cold Autoinflammatory Syndrome 1 (FCAS1); Familial Cold Autoinflammatory Syndrome 2 (FCAS2); Familial Mediterranean Fever (FMF); Gout (GOUT); Hereditary Periodic Fever Syndrome; Hidradenitis; Hidradenitis Suppurativa; Hypertension, Essential (EHT); Hyperuricemia; Intellectual Developmental Disorder, Autosomal Dominant 30, with Speech Delay and Behavioral Abnormalities (MRD30); Interstitial Lung Disease 2 (ILD2); Keratitis, Hereditary (KERH); Keratoendotheliitis Fugax Hereditaria (KEFH); Lipoprotein Quantitative Trait Locus (LPAQTL); Lymph Node Disease; Macular Degeneration, Age-Related, 1 (ARMD1); Malaria (CM); Meningitis; Mevalonic Aciduria (MEVA); Muckle-Wells Syndrome (MWS); Mycobacterium Kansasii; Myocardial Infarction (MCI1); Nephrolithiasis; Nervous System Disease; Non-Alcoholic Steatohepatitis (NASH); Optic Papillitis; Osteomyelitis; Osteoporosis (OSTEOP); Otitis Media (OMS); Papilledema; Pericardial Effusion; Pericardium Disease; Pharyngitis; Polyradiculoneuropathy; Primary Bacterial Infectious Disease; Proteasome-Associated Autoinflammatory Syndrome 1 (PRAAS1); Pyoderma; Pyoderma Gangrenosum; Pyogenic Sterile Arthritis, Pyoderma Gangrenosum, and Acne (PAPAS); Respiratory Failure; Salmonellosis; Schnitzler Syndrome; Sebaceous Gland Disease; Sensorineural Hearing Loss; Serum Amyloid a Amyloidosis; Silicosis; Skin Disease; Stomatitis; Type 2 Diabetes Mellitus (T2D); Ureteral Disease; Urticaria (HIVES); Vulvovaginal Candidiasis; Wells Syndrome.

16. The method of claim 14, wherein the disease or disorder is Autoimmune Disease.

17. The method of claim 14, wherein the disease or disorder is Inflammation Disease.

18. The method of claim 14, wherein the disease or disorder is Coronavirus Infectious Disease.

19. The method of any one of claims 12-18, wherein the subject is a mammal.

20. The method of claim 19, wherein the subject is a human.
